(19) United States
(12) Patent Application Publication
(10) Pub. No.: US 2026/0201071 A1
(43) Pub. Date: Jul. 16, 2026

(54) ANTI-SCLEROSTIN CONSTRUCTS AND USES THEREOF

(71) Applicant: Angitia Incorporated Limited, Westlake Village, CA (US)

(72) Inventors: Xiaofeng LIU, Guangzhou, Guangdong (CN); Kunfeng LIU, Guangzhou, Guangdong (CN); Baozhi YUAN, Guangzhou, Guangdong (CN); Muyu LI, Guangzhou, Guangdong (CN); Hua Zhu KE, Guangzhou, Guangdong (CN)

(21) Appl. No.: 19/573,405

(22) Filed: Mar. 20, 2026

Related U.S. Application Data
(63) Continuation of application No. 17/982,481, filed on Nov. 7, 2022, now Pat. No. 12,630,651.
Application No. 17/982,481 is a continuation of application No. PCT/CN2021/120612, filed on Sep. 26, 2021.

(30) Foreign Application Priority Data
Sep. 28, 2020 (WO) PCT/CN2020/118387

(51) Int. Cl. C07K 16/46; A61P 19/00

(52) U.S. Cl. CPC C07K 16/468; A61P 19/00; C07K 2317/31; C07K 2317/52; C07K 2317/565; C07K 2317/622

(57) ABSTRACT

The present application provides anti-Sclerostin constructs that bind to Sclerostin, nucleic acid molecules encoding an amino acid sequence of the anti-Sclerostin construct, vectors comprising the nucleic acid molecules, host cells containing the vectors, methods of preparing the anti-Sclerostin construct, pharmaceutical compositions containing the anti-Sclerostin construct, and methods of using the anti-Sclerostin construct or compositions.

### CROSS-REFERENCE TO RELATED APPLICATIONS

This application is a continuation of U.S. Application No. 17,982,481, filed on Nov. 7, 2022, which is a continuation of International Application No. PCT/CN2021/120612, filed on Sep. 26, 2021, which claims priority to International Application No. PCT/CN2020/118387, filed on Sep. 28, 2020, the contents of each of which are incorporated herein by reference in their entirety for all purposes.

### TECHNICAL FIELD

The present disclosure relates to anti-Sclerostin constructs (such as anti-Sclerostin antibodies) and the uses thereof.

### REFERENCE TO AN ELECTRONIC SEQUENCE LISTING

The content of the electronic sequence listing (210912000202SeqList.xml; Size: 406,570 bytes; and Date of Creation: Jan. 16, 2026) is herein incorporated by reference in its entirety.

### BACKGROUND OF THE APPLICATION

The function of the SOST gene product, Sclerostin, as an inhibitor of bone formation in humans was discovered by genetic mapping studies that pinpointed loss-of-function mutations in the SOST gene as causative in the high bone mass (HBM) disorder sclerosteosis (Balemans et al., 2001 Hum. Mol. Genet. 10, 537-543; Brunkow et al., 2001 Am. J. Hum. Genet. 68, 577-589). In mice, deletion of the SOST gene causes an increase in bone mass and strength due to increased bone formation, while overexpression of a human Sclerostin transgene result in low bone mass and decreased bone strength (Ke et al., 2012 Endocr. Rev. 33, 747-783.).

The disclosures of all publications, patents, patent applications and published patent applications referred to herein are hereby incorporated herein by reference in their entirety.

### BRIEF SUMMARY OF THE APPLICATION

The following summary is illustrative only and is not intended to be limiting in any way. That is, the following summary is provided to introduce highlights, benefits and advantages of the novel molecules and the uses thereof. Thus, the following summary is not intended to identify essential features of the claimed subject matter, nor is it intended for use in determining the scope of the claimed subject matter.

The present application in one aspect provides anti-Sclerostin constructs comprising an antibody moiety that specifically recognizes Sclerostin (such as human Sclerostin) comprising a heavy chain variable region (V_{H}) and a light chain variable region (V_{L}), such as any of the anti-Sclerostin constructs described herein. In some embodiments, the antibody moiety is an antibody or antigen-binding fragment thereof selected from the group consisting of a full-length antibody, a bispecific antibody, a single-chain Fv (scFv) fragment, a Fab fragment, a Fab′ fragment, a F(ab′)₂ an Fv fragment, a disulfide stabilized Fv fragment (dsFv), a dsscFv, a (dsFv)₂, a Fv-Fc fusion, a scFv-Fc fusion, a scFv-Fv fusion, a diabody, a tribody, and a tetrabody. In some embodiments, the construct is a full-length antibody comprising an Fc fragment. In some embodiments, the antibody moiety is a scFv fragment.

The present application in another aspect provides anti-Sclerostin constructs comprising an antibody moiety that specifically recognizes Sclerostin (such as human Sclerostin), and a second moiety. In some embodiments, the second moiety comprises a half-life extending moiety (such as an Fc fragment). In some embodiments, the second moiety comprises an agent selected from the group consisting of a parathyroid hormone (PTH), a selective estrogen receptor modulator (SERM), a bisphosphonate, prostaglandin E (PGE) receptor agonists, Vascular endothelial growth factor (VEGF), transforming growth factor-β (TGFβ), growth factor (myostatin) and calcitonin.

In some embodiments, the second moiety comprises a second antibody moiety that specifically recognizes an antigen. In some embodiments, there is provided anti-Sclerostin constructs comprising a first antibody moiety that specifically recognizes Sclerostin (including but not limited to anti-Sclerostin antibody moieties described herein), and a second antibody moiety that specifically recognizes Dickkopf-1 (DKK1) or receptor activator of nuclear factor kappa beta ligand (RANKL). Antibody moieties that recognize DKK1 or RANKL can be any anti-DKK1 antibody moiety or anti-RANKL antibody moiety (including but not limited to those described herein). In some embodiments, the second antibody moiety is a full-length antibody, a Fab, a Fab′, a (Fab′)₂, an Fv, a single chain Fv (scFv) fragment, a scFv-scFv, a minibody, a diabody, or an sdAb. In some embodiments, the second antibody moiety is a full-length antibody comprising an Fc fragment, and wherein the anti-Sclerostin antibody moiety is a single chain Fv (scFv) fragment. In some embodiments, the second antibody moiety is a scFv fragment, and wherein the anti-Sclerostin antibody moiety is a full-length antibody comprising an Fc fragment. In some embodiments, the scFv fragment is fused to (e.g., N-terminus and/or C-terminus of) both of the heavy chains and/or light chains of the full-length antibody (via a linker or without a linker). In some embodiments, the construct comprises: a) a first polypeptide comprising a first light chain comprising, from N-terminus to C-terminus, i) the V_{L}, ii) a first light chain constant domain (“first CL domain”); b) a second polypeptide comprising a first heavy chain comprising, from N-terminus to C-terminus, i) the V_{H}, ii) a first heavy chain constant domain (“first CH1 domain”), and iii) a first Fc domain; c) a third polypeptide comprising a second heavy chain comprising, from N-terminus to C-terminus, i) the V_{H-2}, ii) a second heavy chain constant domain (“second CH1 domain”), and iii) a second Fc domain; and d) a fourth polypeptide comprising a second light chain comprising, from N-terminus to C-terminus, i) the V_{L-2}, ii) ii) a second light chain constant domain (“second CL domain”), wherein the first and the second Fc domains form an Fc fragment. The first or second CH1 and/or the first or second Fc domain may have various modifications as described herein.

The present application in another aspect provides anti-Sclerostin constructs that specifically bind to Sclerostin competitively with any of the anti-Sclerostin constructs described herein.

The present application in another aspect provides pharmaceutical compositions comprising any of the anti-Sclerostin constructs described herein and a pharmaceutically acceptable carrier. In some embodiments, the composition further comprises an agent selected from the group consisting of a parathyroid hormone (PTH), a selective estrogen receptor modulator (SERM), VEGF, TGFβ, growth factor (myostatin) and calcitonin.

The present application in another aspect provides isolated nucleic acids encoding any of the anti-Sclerostin constructs described herein or a portion thereof (e.g., one or more polypeptides thereof).

The present application in another aspect provides vectors comprising any of the isolated nucleic acids described herein.

The present application in another aspect provides isolated host cells comprising any of the isolated nucleic acids and/or any of the vectors described herein.

The present application in another aspect provides methods of producing an anti-Sclerostin construct comprising: a) culturing any of the isolated host cells described herein under conditions effective to express the anti-Sclerostin construct or a portion thereof (e.g., one or more polypeptides thereof); and b) obtaining the expressed anti-Sclerostin construct or a portion thereof from the host cells.

The present application in another aspect provides methods of treating a disease or condition in an individual, comprising administering to the individual an effective mount of an anti-Sclerostin construct such as any of the anti-Sclerostin constructs described herein, or any of the pharmaceutical compositions described herein. In some embodiments, the disease or condition is a bone-related disorder. In some embodiments, the bone-related disorder is osteogenesis imperfecta, osteopetrosis, osteoporosis (in men and/or women), senile osteoporosis, delay bone healing, delayed or non-union bone fractures, Paget's disease, immobilization-induced bone loss, glucocorticoid-induced bone loss, inflammation-induced bone loss including arthritis-induced bone loss or other disease or condition associated with a) bone loss of either quantity or quality or both and/or b) abnormality of bone structure and quality. In some embodiments, the anti-Sclerostin construct or the pharmaceutical composition is administered parenterally into the individual. In some embodiments, the method further comprises administering a second agent or therapy (e.g., an anti-DKK1 antibody or an anti-RANKL antibody). In some embodiments, the second agent or therapy comprises an agent selected from the group consisting of a parathyroid hormone (PTH), a selective estrogen receptor modulator (SERM), a bisphosphonate, a prostaglandin E (PGE) receptor agonist, VEGF, and TGFβ, growth factor (myostatin) and calcitonin. In some embodiments, the individual is a human.

### BRIEF DESCRIPTION OF THE DRAWINGS

FIG. 1 shows the sequences (SOST_HUMAN_Q9BQB4:_SEQ ID NO: 183; SOST_MACFA_A0A2K5VUE5: SEQ ID NO: 184; SOST_MACMU_F6WYL4: SEQ ID NO: 189; SOST_MOUSE_Q99P68: SEQ ID NO: 190; and SOST_RAT_Q99P67: SEQ ID NO: 191) and conformation ‘IGRGKWWR’ motif (SEQ ID NO: 186) on the second loop of Sclerostin to illustrate the interaction between Sclerostin and LDL Receptor Related Protein 6 (LRP6).

FIG. 2 shows Sclerostin epitope mapping of monoclonal Ab (mAb) 93B1B7 using synthetic peptide fragments (Pep1: SEQ ID NO: 185; Pep2: SEQ ID NO: 192; Pep3: SEQ ID NO: 193; Pep4: SEQ ID NO: 194; Pep5: SEQ ID NO: 187; Pep6: SEQ ID NO: 188; Pep7 SEQ ID NO: 195; Pep8: SEQ ID NO: 196; Pep9: SEQ ID NO: 197; Pep10: SEQ ID NO: 198) on the second loop of Sclerostin.

FIG. 3 shows that Romosozumab does not recognize peptides from the second loop of human Sclerostin bound by mAb 93B1B7.

FIG. 4 shows that 93B1B7 and Romosozumab occupy different Sclerostin epitopes and do not cross-block each other's binding to sclerostin.

FIG. 5 shows schematic diagrams of exemplary bispecific antibody structures.

FIG. 6 shows results from BioLayer Interferometry (BLI) sensorgram that demonstrate the incremental binding of human Sclerostin and DKK1 to bispecific antibodies;

FIG. 7 shows serum concentrations of bispecific antibodies following 30 mg/kg subcutaneous administration in cynomolgus monkeys.

### DETAILED DESCRIPTION OF THE APPLICATION

The present application provides novel anti-Sclerostin constructs that specifically bind to Sclerostin (such as anti-Sclerostin monoclonal antibodies or multispecific antibodies), methods of preparing the anti-Sclerostin constructs, methods of using the constructs (e.g., methods of treating a disease or condition). The exemplary anti-Sclerostin constructs described herein achieved advantageous effects. For examples, exemplary anti-Sclerostin constructs exhibited higher binding affinity to Sclerostin as compared to Romosozumab. See Examples 3, 5, and 6 (Tables 5, 7, and 8).

### I. Definitions

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antibody moiety” refers to a full-length antibody or an antigen-binding fragment thereof.

A full-length antibody comprises two heavy chains and two light chains. The variable regions of the light and heavy chains are responsible for antigen binding. The variable domains of the heavy chain and light chain may be referred to as “V_{H}” and “V_{L}”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), lgA1 (α1 heavy chain), or lgA2 (α2 heavy chain).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain Fv (scFv), a dsscFv, an scFv dimer (bivalent diabody), a multispecific antibody formed from a portion of an antibody comprising one or more CDRs, a camelized single domain antibody, a Nanobody®, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above-cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein. In some embodiments, the CDR sequences provided herein are based on IMGT definition. For example, the CDR sequences may be determined by the VBASE2 tool (see also Retter I, Althaus H H, Munch R, Müller W: VBASE2, an integrative V gene database. Nucleic Acids Res. 2005 Jan. 1; 33 (Database issue): D671-4, which is incorporated herein by reference in its entirety).

The term “osteoporosis” as used herein refers to premenopausal idiopathic osteoporosis, postmenopausal osteoporosis, menopausal osteoporosis, postoophorectomy osteoporosis, osteoporosis of disuse, drug-induced osteoporosis, osteoporosis due to malabsorption, post-surgical malabsorption osteoporosis and/or senile osteoporosis.

The term “osteopenia” as used herein refers to premenopausal idiopathic osteopenia, postmenopausal osteopenia, senile osteopenia, drug-induced osteopenia, osteopenia of disuse, neonatal osteopenia and/or spaceflight osteopenia caused by reduced gravity.

The term “metabolic bone diseases” as used herein includes but not limit to renal osteodystrophy, primary and secondary hyperparathyroidism, familial hyperparathyroidism syndromes, parathyroid disorders, osteodystrophy, osteochondrosis, hyperphosphatasia.

The term “osteonecrosis” as used herein refers to avascular necrosis of bone, avascular necrosis secondary to diving, osteonecrosis of jaw.

The term “bone loss” as used herein refers to postmenopausal bone loss, Immobilization-induced bone loss, Weightlessness induced bone loss, Disease associated facial bone loss, Disease associated cranial bone loss, Disease associated bone loss of the jaw, Disease associated bone loss of the skull, bone loss associated with space travel, glucocorticoid-induced bone loss, Drug-induced bone loss, Organ transplant related bone loss, Kidney transplant related bone loss, HIV associated bone loss, bone loss associated with loss of growth hormone, bone loss associated with cystic fibrosis, Chemotherapy associated bone loss, Tumor induced bone loss, Cancer-related bone loss, Hormone ablative bone loss, Oral bone loss, Heparin-induced bone loss, Inflammation-induced bone loss including arthritis-induced bone loss or other disease or condition associated with a) bone loss of either quantity or quality or both and/or b) abnormality of bone structure and quality. bone loss caused by reduced gravity.

The term “nonunion” or “delay bone healing” as used herein refers to delayed or non-union bone fractures, hip fracture, pseudoarthritis after fusion or arthrodesis, osteolysis, postsurgical osteolysis, nonunion after spinal arthrodesis, enhancement/acceleration of spinal fusion, chronic pain after arthroplasty.

The term “osteomalacia” as used herein refers to Vitamin-D-resistant osteomalacia, calcium deficiency, sarcopenia, cancer sarcopenia, tumor-induced osteomalacia.

The term “fracture” as used herein includes but not limited to compression fracture, fragility fracture, pathologic fracture, stress fracture, hip fracture, fracture of femoral neck, atypical hip fracture, femoral intertrochanter fracture, fracture of bone in neoplastic disease.

The term “hypercalcemia” as used herein includes hypercalcemia of malignancy, myopathy due to hypercalcemia, hypercalcemia in chronic kidney disease.

The term “multiple myeloma related bone disorders” as used herein refers to multiple myeloma bone disease and ore osteoporosis in multiple myelomatosis.

The term “primary bone tumor” as used herein includes osteosarcoma, osteochondroma, osteoblastoma, osteochondromyxoma, osteoclastoma, osteoma, osteoid osteoma, chondrosarcoma, chondroblastoma, chondromyxoid fibroma, myxoid chondrosarcoma, sarcoma, ewing sarcoma, kaposi sarcoma, periosteal sarcoma, glomangiosarcoma, giant cell tumor, giant cell sarcoma, giant cell angiofibroma, haemangioendothelial sarcoma, undifferentiated sarcoma, fibrosarcoma, bone cyst, aneurysmal bone cyst, multiple endocrine neoplasia.

The term “malignancies” for “bone metastasis of malignancies” includes breast cancer, lung cancer, hepatic cancer, ovarian cancer, pancreatic cancer, colorectal cancer, gastric cancer, prostate cancer, thyroid cancer, thymus cancer.

The term “inflammatory or infectious bone disease” as used herein refers to osteomyelitis, pyogenic osteomyelitis, ankylosing spondylitis.

The term “bone marrow or haemotological disordersdiseases” as used herein refers to leukemia, malignant lymphoma, haematological malignancy, haematologic disease, bone marrow disease.

The term “musculoskeletal rare disease” as used herein includes Osteogenesis imperfecta, Albers-Schonberg disease, congenital pseudarthrosis of the tibia, enchondromatosis, fibrous dysplasia, Gaucher's Disease, Marfan's syndrome, multiple hereditary exotoses, neurofibromatosis, osteogenesis imperfecta, osteopetrosis, osteopoikilosis, sclerotic lesions, pseudoarthrosis, melorheostosis, Juvenile arthritides, thalassemia, mucopolysaccharidoses, turner syndrome, Pown Syndrome, Klinefelter Syndrome, leprosy, Perthes' Disease, adolescent idiopathic scoliosis, Winchester Syndrome, Menkes Disease, ischemic bone disease (such as Legg-Calve-Perthes disease, regional migratory osteoporosis), Idiopathic infantile hypercalcemia, Acromegaly, Hypogonadism, Albright-McCune-Sternberg syndrome, Aluminium bone disease, Camurati-Engelmann disease, Osteopetrosis and infantile neuroaxonal dystrophy, Dysosteosclerosis, Pycnodysostosis, Gorham-Stout syndrome, Cystic angiomatosis, Paget's disease, Juvenile Paget's disease, Osteoporosis-oculocutaneous-hypopigmentation syndrome, Osteoporosis in classical or atypical cystic fibrosis, Bowed tibiae-radial anomalies-osteopenia-fractures, X-linked hypophosphatemic osteomalacia, Familial expansile osteolysis, Osteopoikilosis, Melorheostosis, Craniometaphyseal dysplasia, Osteoporosis-pseudoglioma syndrome, Cleidocranial dysplasia, Hajdu-Cheney syndrome, Winchester-Torg syndrome, Cole-Carpenter syndrome, Hypophosphatasia, Hereditary hyperphosphatasia, Fibrodysplasia ossificans progressive, Familial hypocalciuric hypercalcemia, Pseudohypoparathyroidism, Acrodysostosis, Eiken syndrome, Multiple enchondromatosis, Vitamin D hydroxylation-deficient rickets, Hypophosphatemic rickets.

The term “cartilage-related disorder” as used herein includes but not limited to Chondromatosis, Chondrodysplasia, Chondrodystrophic myotonia, Juxtacortical chondroma, Tear of cartilage of knee, Osteoarthritis, Osteochondrodystrophy.

The term “mscle-related disorder” as used herein includes sarcopenia and cancer sarcopenia.

The term “surgeries” as used in “facilitation of heal after bone or joint surgeries” refers to orthopedic procedures, dental procedures, implant surgery, joint replacement, joint-preserving surgery, distraction osteogenesis, bone lengthening, bone grafting, bone cosmetic surgery and bone repair such as fracture healing, nonunion healing, delayed union healing and facial reconstruction.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or hypervariable region (HVR) of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

“Framework” or “FR” residues are those variable-domain residues other than the CDR residues as herein defined.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, See Jones et al., Nature 321:522-525 (1986); Riechmann et al., Nature 332:323-329 (1988); and Presta, Curr. Op. Struct. Biol. 2:593-596 (1992).

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, MegAlign® (DNASTAR®), or MUSCLE™ software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE™ (Edgar, R. C., Nucleic Acids Research 32(5):1792-1797, 2004; Edgar, R. C., BMC Bioinformatics 5(1):113, 2004).

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the CHL (or C_{L}) domain of the light chain.

The “light chains” of antibodies (immunoglobulins) from any mammalian species can be assigned to one of two clearly distinct types, called kappa (“κ”) and lambda (“λ”), based on the amino acid sequences of their constant domains.

The “C_{H}1 domain” (also referred to as “C1” of “H1” domain) usually extends from about amino acid 118 to about amino acid 215 (EU numbering system).

“Hinge region” is generally defined as a region in IgG corresponding to Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “C_{H}2 domain” of a human IgG Fc region (also referred to as “C2” domain) usually extends from about amino acid 231 to about amino acid 340. The C_{H}2 domain is unique in that it is not closely paired with another domain. Rather, two N-linked branched carbohydrate chains are interposed between the two C_{H}2 domains of an intact native IgG molecule. It has been speculated that the carbohydrate may provide a substitute for the domain-domain pairing and help stabilize the C_{H}2 domain. Burton, Molec Immunol. 22:161-206 (1985).

The “C_{H}3 domain” (also referred to as “C3” domain) comprises the stretch of residues C-terminal to a C_{H}2 domain in an Fc region (i.e. from about amino acid residue 341 to the C-terminal end of an antibody sequence, typically at amino acid residue 446 or 447 of an IgG).

The term “Fc region” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Fc receptor” or “FcR” describes a receptor that binds the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Daeron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody or fragment thereof “competes” for binding to a target antigen with a second antibody or fragment thereof when the first antibody or fragment thereof inhibits the target antigen binding of the second antibody of fragment thereof by at least about 50% (such as at least about any one of 55%, 60%, 65%, 70%, 75%, 80%, 85%, 90%, 95%, 98% or 99%) in the presence of an equimolar concentration of the first antibody or fragment thereof, or vice versa. A high throughput process for “binning” antibodies based upon their cross-competition is described in PCT Publication No. WO 03/48731.

As use herein, the terms “specifically binds,” “specifically recognizing,” and “is specific for” refer to measurable and reproducible interactions, such as binding between a target and an antibody or antibody moiety, which is determinative of the presence of the target in the presence of a heterogeneous population of molecules, including biological molecules. For example, an antibody or antibody moiety that specifically recognizes a target (which can be an epitope) is an antibody or antibody moiety that binds this target with greater affinity, avidity, more readily, and/or with greater duration than its bindings to other targets. In some embodiments, the extent of binding of an antibody to an unrelated target is less than about 10% of the binding of the antibody to the target as measured, e.g., by a radioimmunoassay (RIA). In some embodiments, an antibody that specifically binds a target has a dissociation constant (K_{D}) of ≤10⁻⁵ M, ≤10⁻⁶ M, ≤10⁻⁷ M, ≤10⁻⁸ M, ≤10⁻⁹ M, ≤10⁻¹⁰ M, 10⁻¹¹ M, or ≤10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA-, RIA-, ECL-, IRMA-, EIA-, BLI-, BIACORE™-tests and peptide scans.

An “isolated” antibody (or construct) is one that has been identified, separated and/or recovered from a component of its production environment (e.g., natural or recombinant). Preferably, the isolated polypeptide is free of association with all other components from its production environment.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, and may contain mutations. Mutant progeny that has the same function or biological activity as screened or selected for in the originally transformed cell are included herein.

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease, preventing or delaying the spread of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. The methods of the application contemplate any one or more of these aspects of treatment.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to that of a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or patient.

As used herein, “delaying development of a disease” means to defer, hinder, slow, retard, stabilize, suppress and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease.

“Preventing” as used herein, includes providing prophylaxis with respect to the occurrence or recurrence of a disease in an individual that may be predisposed to the disease but has not yet been diagnosed with the disease.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

The terms “subject,” “individual,” and “patient” are used interchangeably herein to refer to a mammal, including, but not limited to, human, bovine, horse, feline, canine, rodent, or primate. In some embodiments, the individual is a human.

An “effective amount” of an agent refers to an amount effective, at dosages and for periods of time necessary, to achieve the desired therapeutic or prophylactic result. The specific dose may vary depending on one or more of: the particular agent chosen, the dosing regimen to be followed, whether it is administered in combination with other compounds, timing of administration, the tissue to be imaged, and the physical delivery system in which it is carried.

The terms “pharmaceutical formulation” and “pharmaceutical composition” refer to a preparation which is in such form as to permit the biological activity of the active ingredient(s) to be effective, and which contains no additional components which are unacceptably toxic to an individual to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to an individual. A pharmaceutically acceptable carrier is non-toxic to recipients at the dosages and concentrations employed and is compatible with other ingredients of the formulation. The pharmaceutically acceptable carrier is appropriate for the formulation employed. Pharmaceutically acceptable carriers or excipients have preferably met the required standards of toxicological and manufacturing testing and/or are included on the Inactive Ingredient Guide prepared by the U.S. Food and Drug administration.

A “sterile” formulation is aseptic or essentially free from living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about 60 minutes, such as no more than about any of 30, 15, 10, 5, or 1 minutes.

The term “sequentially” is used herein to refer to administration of two or more therapeutic agents where the administration of one or more agent(s) continues after discontinuing the administration of one or more other agent(s). For example, administration of the two or more therapeutic agents are administered with a time separation of more than about 15 minutes, such as about any of 20, 30, 40, 50, or 60 minutes, 1 day, 2 days, 3 days, 1 week, 2 weeks, or 1 month, or longer.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder, or a probe for specifically detecting a biomarker described herein. In certain embodiments, the manufacture or kit is promoted, distributed, or sold as a unit for performing the methods described herein.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

The term “about X-Y” used herein has the same meaning as “about X to about Y.”

As used herein and in the appended claims, the singular forms “a,” “or,” and “the” include plural referents unless the context clearly dictates otherwise.

### II. Anti-Sclerostin Constructs

The present application provides anti-Sclerostin constructs comprising an anti-Sclerostin antibody moiety that specifically binds to Sclerostin (product of SOST) as described herein.

Sclerostin is a secreted glycoprotein with a C-terminal cysteine knot-like (CTCK) domain and sequence similarity to the DAN (differential screening-selected gene aberrative in neuroblastoma) family of bone morphogenetic protein (BMP) antagonists. Loss-of-function mutations in this gene are associated with an autosomal-recessive disorder, sclerosteosis, which causes progressive bone overgrowth. A deletion downstream of this gene, which causes reduced Sclerostin expression, is associated with a milder form of the disorder called van Buchem disease.

In some embodiments, there is provided an anti-Sclerostin construct comprising an antibody moiety that specifically recognizes Sclerostin, wherein the antibody moiety binds to an epitope on Sclerostin, wherein the epitope comprises the amino acid sequence set forth in SEQ ID NO: 186.

In some embodiments, the anti-Sclerostin construct comprises an antibody moiety comprising a heavy chain variable region (V_{H}) and a light chain variable region (V_{L}), wherein the antibody moiety competes for a binding epitope of Sclerostin with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9, and the V_{L-2} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of SEQ ID NO: 20.

In some embodiments, the anti-Sclerostin construct comprises an antibody moiety comprising a heavy chain variable region (V_{H}) and a light chain variable region (V_{L}), wherein the antibody moiety competes for a binding epitope of Sclerostin with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 1, the HC-CDR2 comprising the amino acid sequence of SEQ ID NO: 6, and the HC-CDR3 comprising the amino acid sequence of SEQ ID NO: 10, and the V_{L-2} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of SEQ ID NO: 20.

In some embodiments, the anti-Sclerostin construct comprises an antibody moiety comprising a heavy chain variable region (V_{H}) and a light chain variable region (V_{L}), wherein the antibody moiety competes for a binding epitope of Sclerostin with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 2, the HC-CDR2 comprising the amino acid sequence of SEQ ID NO: 7, and the HC-CDR3 comprising the amino acid sequence of SEQ ID NO: 10, and the V_{L-2} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of SEQ ID NO: 20.

In some embodiments, the anti-Sclerostin construct comprises an antibody moiety comprising a heavy chain variable region (V_{H}) and a light chain variable region (V_{L}), wherein the antibody moiety competes for a binding epitope of Sclerostin with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 2, the HC-CDR2 comprising the amino acid sequence of SEQ ID NO: 6, and the HC-CDR3 comprising the amino acid sequence of SEQ ID NO: 10, and the V_{L-2} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of SEQ ID NO: 20.

In some embodiments, the anti-Sclerostin construct comprises an antibody moiety comprising a heavy chain variable region (V_{H}) and a light chain variable region (V_{L}), wherein the antibody moiety competes for a binding epitope of Sclerostin with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 10, and the V_{L-2} comprises the LC-CDR1 comprising the amino acid sequence of SEQ ID NO: 16, the LC-CDR2 comprising the amino acid sequence of SEQ ID NO: 18, and the LC-CDR3 comprising the amino acid sequence of SEQ ID NO: 20.

In some embodiments, the anti-Sclerostin construct comprises an antibody moiety comprising a heavy chain variable region (V_{H}) and a light chain variable region (V_{L}), wherein the antibody moiety competes for a binding epitope of Sclerostin with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 3, the HC-CDR2 comprising the amino acid sequence of SEQ ID NO: 7, and the HC-CDR3 comprising the amino acid sequence of SEQ ID NO: 10, and the V_{L-2} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of SEQ ID NO: 20.

In some embodiments, the anti-Sclerostin construct comprises an antibody moiety comprising a heavy chain variable region (V_{H}) and a light chain variable region (V_{L}), wherein the antibody moiety competes for a binding epitope of Sclerostin with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 10, and the V_{L-2} comprises the LC-CDR1 comprising the amino acid sequence of SEQ ID NO: 16, the LC-CDR2 comprising the amino acid sequence of SEQ ID NO: 18, and the LC-CDR3 comprising the amino acid sequence of SEQ ID NO: 20.

In some embodiments, the anti-Sclerostin construct comprises an antibody moiety comprising a heavy chain variable region (V_{H}) and a light chain variable region (V_{L}), wherein the antibody moiety competes for a binding epitope of Sclerostin with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 4, the HC-CDR2 comprising the amino acid sequence of SEQ ID NO: 8, and the HC-CDR3 comprising the amino acid sequence of SEQ ID NO: 11, and the V_{L-2} comprises the LC-CDR1 comprising the amino acid sequence of SEQ ID NO: 17, the LC-CDR2 comprising the amino acid sequence of SEQ ID NO: 19, and the LC-CDR3 comprising the amino acid sequence of SEQ ID NO: 21.

In some embodiments, the anti-Sclerostin construct comprises an antibody moiety comprising a heavy chain variable region (V_{H}) and a light chain variable region (V_{L}), wherein the antibody moiety competes for a binding epitope of Sclerostin with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 2, the HC-CDR2 comprising the amino acid sequence of SEQ ID NO: 6, and the HC-CDR3 comprising the amino acid sequence of SEQ ID NO: 10, and the V_{L-2} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of SEQ ID NO: 20.

In some embodiments, the anti-Sclerostin construct comprises an antibody moiety comprising a heavy chain variable region (V_{H}) and a light chain variable region (V_{L}), wherein the antibody moiety competes for a binding epitope of Sclerostin with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9, and the V_{L-2} comprises the LC-CDR1 comprising the amino acid sequence of SEQ ID NO: 85, the LC-CDR2 comprising the amino acid sequence of SEQ ID NO: 18, and the LC-CDR3 comprising the amino acid sequence of SEQ ID NO: 20.

In some embodiments, the anti-Sclerostin construct comprises an antibody moiety comprising a heavy chain variable region (V_{H}) and a light chain variable region (V_{L}), wherein the antibody moiety competes for a binding epitope of Sclerostin with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9, and the V_{L-2} comprises the LC-CDR1 comprising the amino acid sequence of SEQ ID NO: 86, the LC-CDR2 comprising the amino acid sequence of SEQ ID NO: 18, and the LC-CDR3 comprising the amino acid sequence of SEQ ID NO: 20.

In some embodiments, the V_{H} comprises i) the HC-CDR1 comprising the amino acid sequence of any one of SEQ ID NOs: 1-4 and 12, ii) the HC-CDR2 comprising the amino acid sequence of any one of SEQ ID NOs: 5-8 and 13, and iii) the HC-CDR3 comprising the amino acid sequence any one of SEQ ID NOs: 9-11 and 14, or a variant thereof comprising up to 5, 4, 3, 2, or 1 amino acid substitutions in the HC-CDRs, and the V_{L} comprises i) the LC-CDR1 comprising the amino acid sequence of any one of SEQ ID NOs: 15-17, ii) the LC-CDR2 comprising the amino acid sequence of any one of SEQ ID NOs: 18 and 19, and iii) the LC-CDR3 comprising the amino acid sequence of any one of SEQ ID NOs: 20 and 21, or a variant thereof comprising up to 5, 4, 3, 2, or 1 amino acid substitutions in the LC-CDRs.

In some embodiments, the anti-Sclerostin construct comprises an antibody moiety comprising a heavy chain variable region (V_{H}) and a light chain variable region (V_{L}), wherein the V_{H} comprises the HC-CDR1 comprising the amino acid sequence of SEQ ID NO: 12, the HC-CDR2 comprising the amino acid sequence of SEQ ID NO: 13, and the HC-CDR3 comprising the amino acid sequence of SEQ ID NO: 14; and the V_{L} comprises the LC-CDR1 comprising the amino acid sequence of SEQ ID NO: 15 or 16, the LC-CDR2 comprising the amino acid sequence of SEQ ID NO: 18, and the LC-CDR3 comprising the amino acid sequence of any one of SEQ ID NOs: 20.

In some embodiments, the anti-Sclerostin construct comprises an antibody moiety comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 9, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 15, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application. In some embodiments, the V_{H} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 9, and the V_{L} comprises i) the LC-CDR1 comprising the amino acid sequence of SEQ ID NO: 15, ii) the LC-CDR2 comprising the amino acid sequence of SEQ ID NO: 18, and iii) the LC-CDR3 comprising the amino acid sequence of SEQ ID NO: 20. In some embodiments, the V_{H} comprises an amino acid sequence of SEQ ID NO: 87, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 88, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 22, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 23, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 26, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 27, or a variant comprising an amino acid sequence having at least about 80% (such as at least about any one of 80%, 85%, 90%, 95%, 96%, 97%, 98%, or 99%) sequence identity.

In some embodiments, the anti-Sclerostin construct comprises an antibody moiety comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 6, and iii) the HC-CDR3 comprising the amino acid sequence of SEQ ID NO: 10, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 15, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application. In some embodiments, the V_{H} comprises an amino acid sequence of SEQ ID NO: 24, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 25, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 36, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 37, or a variant comprising an amino acid sequence having at least about 80% (such as at least about any one of 80%, 85%, 90%, 95%, 96%, 97%, 98%, or 99%) sequence identity.

In some embodiments, the anti-Sclerostin construct comprises an antibody moiety comprising a heavy chain variable region (V_{H}) and a light chain variable region (V_{L}), wherein the V_{H} comprises i) the HC-CDR1 comprising the amino acid sequence of SEQ ID NO: 2, ii) the HC-CDR2 comprising the amino acid sequence of SEQ ID NO: 7, and iii) the HC-CDR3 comprising the amino acid sequence of SEQ ID NO: 10, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 15, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application. In some embodiments, the V_{H} comprises an amino acid sequence of SEQ ID NO: 28, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 29, or a variant comprising an amino acid sequence having at least about 80% (such as at least about any one of 80%, 85%, 90%, 95%, 96%, 97%, 98%, or 99%) sequence identity.

In some embodiments, the anti-Sclerostin construct comprises an antibody moiety comprising a heavy chain variable region (V_{H}) and a light chain variable region (V_{L}), wherein the V_{H} comprises i) the HC-CDR1 comprising the amino acid sequence of SEQ ID NO: 2, ii) the HC-CDR2 comprising the amino acid sequence of SEQ ID NO: 6, and iii) the HC-CDR3 comprising the amino acid sequence of SEQ ID NO: 10, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 15, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application. In some embodiments, the V_{H} comprises an amino acid sequence of SEQ ID NO: 30, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 31, or a variant comprising an amino acid sequence having at least about 80% (such as at least about any one of 80%, 85%, 90%, 95%, 96%, 97%, 98%, or 99%) sequence identity.

In some embodiments, the anti-Sclerostin construct comprises an antibody moiety comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 10, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 16, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application. In some embodiments, the V_{H} comprises an amino acid sequence of SEQ ID NO: 32, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 33, or a variant comprising an amino acid sequence having at least about 80% (such as at least about any one of 80%, 85%, 90%, 95%, 96%, 97%, 98%, or 99%) sequence identity.

In some embodiments, the anti-Sclerostin construct comprises an antibody moiety comprising a heavy chain variable region (V_{H}) and a light chain variable region (V_{L}), wherein the V_{H} comprises i) the HC-CDR1 comprising the amino acid sequence of SEQ ID NO: 3, ii) the HC-CDR2 comprising the amino acid sequence of SEQ ID NO: 7, and iii) the HC-CDR3 comprising the amino acid sequence of SEQ ID NO: 10, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 15, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application. In some embodiments, the V_{H} comprises an amino acid sequence of SEQ ID NO: 34, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 35, or a variant comprising an amino acid sequence having at least about 80% (such as at least about any one of 80%, 85%, 90%, 95%, 96%, 97%, 98%, or 99%) sequence identity.

In some embodiments, the anti-Sclerostin construct comprises an antibody moiety comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 10, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 16, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application. In some embodiments, the V_{H} comprises an amino acid sequence of SEQ ID NO: 38, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 39, or a variant comprising an amino acid sequence having at least about 80% (such as at least about any one of 80%, 85%, 90%, 95%, 96%, 97%, 98%, or 99%) sequence identity.

In some embodiments, the anti-Sclerostin construct comprises an antibody moiety comprising a heavy chain variable region (V_{H}) and a light chain variable region (V_{L}), wherein the V_{H} comprises i) the HC-CDR1 comprising the amino acid sequence of SEQ ID NO: 4, ii) the HC-CDR2 comprising the amino acid sequence of SEQ ID NO: 8, and iii) the HC-CDR3 comprising the amino acid sequence of SEQ ID NO: 11, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 17, ii) the LC-CDR2 comprising the amino acid sequence of SEQ ID NO: 19, and iii) the LC-CDR3 comprising the amino acid sequence of SEQ ID NO: 21, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application. In some embodiments, the V_{H} comprises an amino acid sequence of SEQ ID NO: 40, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 41, or a variant comprising an amino acid sequence having at least about 80% (such as at least about any one of 80%, 85%, 90%, 95%, 96%, 97%, 98%, or 99%) sequence identity.

In some embodiments, the anti-Sclerostin construct comprises an antibody moiety comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 9, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 85, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application. In some embodiments, the V_{H} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 9, and the V_{L} comprises i) the LC-CDR1 comprising the amino acid sequence of SEQ ID NO: 85, ii) the LC-CDR2 comprising the amino acid sequence of SEQ ID NO: 18, and iii) the LC-CDR3 comprising the amino acid sequence of SEQ ID NO: 20. In some embodiments, the V_{H} comprises an amino acid sequence of SEQ ID NO: 87, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 89, or a variant comprising an amino acid sequence having at least about 80% (such as at least about any one of 80%, 85%, 90%, 95%, 96%, 97%, 98%, or 99%) sequence identity.

In some embodiments, the anti-Sclerostin construct comprises an antibody moiety comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 9, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 86, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application. In some embodiments, the V_{H} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 9, and the V_{L} comprises i) the LC-CDR1 comprising the amino acid sequence of SEQ ID NO: 86, ii) the LC-CDR2 comprising the amino acid sequence of SEQ ID NO: 18, and iii) the LC-CDR3 comprising the amino acid sequence of SEQ ID NO: 20. In some embodiments, the V_{H} comprises an amino acid sequence of SEQ ID NO: 87, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 90, or a variant comprising an amino acid sequence having at least about 80% (such as at least about any one of 80%, 85%, 90%, 95%, 96%, 97%, 98%, or 99%) sequence identity.

In some embodiments, there is provided an anti-Sclerostin construct comprising an antibody moiety that specifically recognizes Sclerostin, wherein the antibody moiety comprises a heavy chain variable region (V_{H}) and a light chain variable region (V_{L}), wherein:
a) a HC-CDR1, a HC-CDR2, and a HC-CDR3, respectively comprising the amino acid sequences of a CDR1, a CDR2, and a CDR3 within the V_{H} having the sequence set forth in SEQ ID NO: 22, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 23;
b) a HC-CDR1, a HC-CDR2, and a HC-CDR3, respectively comprising the amino acid sequences of a CDR1, a CDR2, and a CDR3 within the V_{H} having the sequence set forth in SEQ ID NO: 24, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 25;
c) a HC-CDR1, a HC-CDR2, and a HC-CDR3, respectively comprising the amino acid sequences of a CDR1, a CDR2, and a CDR3 within the V_{H} having the sequence set forth in SEQ ID NO: 26, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 27;
d) a HC-CDR1, a HC-CDR2, and a HC-CDR3, respectively comprising the amino acid sequences of a CDR1, a CDR2, and a CDR3 within the V_{H} having the sequence set forth in SEQ ID NO: 28, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 29;
e) a HC-CDR1, a HC-CDR2, and a HC-CDR3, respectively comprising the amino acid sequences of a CDR1, a CDR2, and a CDR3 within the V_{H} having the sequence set forth in SEQ ID NO: 30, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 31;
f) a HC-CDR1, a HC-CDR2, and a HC-CDR3, respectively comprising the amino acid sequences of a CDR1, a CDR2, and a CDR3 within the V_{H} having the sequence set forth in SEQ ID NO: 32, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 33;
g) a HC-CDR1, a HC-CDR2, and a HC-CDR3, respectively comprising the amino acid sequences of a CDR1, a CDR2, and a CDR3 within the V_{H} having the sequence set forth in SEQ ID NO: 34, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 35;
h) a HC-CDR1, a HC-CDR2, and a HC-CDR3, respectively comprising the amino acid sequences of a CDR1, a CDR2, and a CDR3 within the V_{H} having the sequence set forth in SEQ ID NO: 36, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 37;
i) a HC-CDR1, a HC-CDR2, and a HC-CDR3, respectively comprising the amino acid sequences of a CDR1, a CDR2, and a CDR3 within the V_{H} having the sequence set forth in SEQ ID NO: 38, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 39;
j) a HC-CDR1, a HC-CDR2, and a HC-CDR3, respectively comprising the amino acid sequences of a CDR1, a CDR2, and a CDR3 within the V_{H} having the sequence set forth in SEQ ID NO: 40, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 41;
k) a HC-CDR1, a HC-CDR2, and a HC-CDR3, respectively comprising the amino acid sequences of a CDR1, a CDR2, and a CDR3 within the V_{H} having the sequence set forth in SEQ ID NO: 87, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 88;
l) a HC-CDR1, a HC-CDR2, and a HC-CDR3, respectively comprising the amino acid sequences of a CDR1, a CDR2, and a CDR3 within the V_{H} having the sequence set forth in SEQ ID NO: 87, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 89; or
m) a HC-CDR1, a HC-CDR2, and a HC-CDR3, respectively comprising the amino acid sequences of a CDR1, a CDR2, and a CDR3 within the V_{H} having the sequence set forth in SEQ ID NO: 87, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 90.

In some embodiments, the construct comprises or is an antibody or antigen-binding fragment thereof selected from the group consisting of a full-length antibody, a bispecific antibody, a single-chain Fv (scFv) fragment, a Fab fragment, a Fab′ fragment, a F(ab′)2, an Fv fragment, a disulfide stabilized Fv fragment (dsFv), a dsscFv, a (dsFv)₂, a V_{H}H, a Fv-Fc fusion, a scFv-Fc fusion, a scFv-Fv fusion, a diabody, a tribody, and a tetrabody.

In some embodiments, the anti-Sclerostin antibody moiety is a full-length antibody.

In some embodiments, the anti-Sclerostin antibody moiety is a scFv or dsscFv.

In some embodiments, the anti-Sclerostin antibody moiety described above comprises an Fc fragment of an immunoglobulin selected from the group consisting of IgG, IgA, IgD, IgE, IgM, and combinations and hybrids thereof. In some embodiments, the anti-Sclerostin antibody moiety or the full-length antibody described above comprises an Fc fragment of an immunoglobulin selected from the group consisting of IgG1, IgG2, IgG3, IgG4, and combinations and hybrids thereof. In some embodiments, the Fc fragment has a reduced effector function as compared to the corresponding wildtype Fc fragment. In some embodiments, the Fc fragment has an enhanced effector function as compared to the corresponding wildtype Fc fragment.

In some embodiments, the antibody moiety comprises a humanized antibody of any of the antibody moiety described herein.

In some embodiments, the anti-Sclerostin construct comprises or is an anti-Sclerostin fusion protein.

In some embodiments, the anti-Sclerostin construct comprises or is a multispecific anti-Sclerostin construct (such as a bispecific antibody).

In some embodiments, the Sclerostin is a human Sclerostin.

### a) Antibody Affinity

Binding specificity of the antibody moieties can be determined experimentally by methods known in the art. Such methods comprise, but are not limited to Western blots, ELISA-, RIA-, ECL-, IRMA-, EIA-, BLI-, BIACORE™-tests and peptide scans.

In some embodiments, the K_{D} of the binding between the antibody moiety and Sclerostin is about 10⁻⁷M to about 10⁻¹² M, about 10⁻⁷ M to about 10⁻⁸M, about 10⁻⁸M to about 10⁻⁹ M, about 10⁻⁹ M to about 10⁻¹⁰ M, about 10⁻¹⁰ M to about 10⁻¹¹ M, about 10⁻¹¹ M to about 10⁻¹² M, about 10⁻⁷ M to about 10⁻¹² M, about 10⁻⁸ M to about 10⁻¹² M, about 10⁻⁹ M to about 10⁻¹² M, about 10⁻¹⁰ M to about 10⁻¹² M, about 10⁻⁷ M to about 10⁻¹¹ M, about 10⁻⁸ M to about 10⁻¹¹ M, about 10⁻⁹ M to about 10⁻¹¹ M, about 10⁻⁷ M to about 10⁻¹⁰ M, about 10⁻⁸ M to about 10⁻¹⁰ M, or about 10⁻⁷ M to about 10⁻⁹ M. In some embodiments, the K_{D} of the binding between the antibody moiety and Sclerostin is stronger than about any one of 10⁻⁷ M, 108 M, 10⁻⁹ M, 10⁻¹⁰ M, 10⁻¹¹ M, or 10⁻¹² M. In some embodiments, the Sclerostin is a human Sclerostin. In some embodiments, Sclerostin is cynomolgus Sclerostin.

In some embodiments, the Kₒₙ of the binding between the antibody moiety and Sclerostin is about 10³ M⁻¹s⁻¹ to about 10⁸ M⁻¹s⁻¹, about 10³ M⁻¹s⁻¹ to about 10⁴ M⁻¹s⁻¹, about 10⁴ M⁻¹s⁻¹ to about 10⁵ M⁻¹s⁻¹, about 10⁵ M⁻¹s⁻¹ to about 10⁶ M⁻¹s⁻¹, about 10⁶ M⁻¹s⁻¹ to about 10⁷ M⁻¹s⁻¹, or about 10⁷ M⁻¹s⁻¹ to about 10⁸ M⁻¹s⁻¹. In some embodiments, the Kₒₙ of the binding between the antibody moiety and Sclerostin is about 10³ M⁻¹s⁻¹ to about 10⁵ M⁻¹s⁻¹, about 10⁴ M⁻¹s⁻¹ to about 10⁶ M⁻¹s⁻¹, about 10⁵ M⁻¹s⁻¹ to about 10⁷ M⁻¹s⁻¹, about 10⁶ M⁻¹s⁻¹ to about 10⁸ M⁻¹s⁻¹, about 10⁴ M⁻¹s⁻¹ to about 10⁷ M⁻¹s⁻¹, or about 10⁵ M⁻¹s⁻¹ to about 10⁸ M⁻¹s⁻¹. In some embodiments, the Kₒₙ of the binding between the antibody moiety and Sclerostin is no more than about any one of 10³ M⁻¹s⁻¹, 10⁴ M⁻¹s⁻¹, 10⁵ M⁻¹s⁻¹, 10⁶ M⁻¹s⁻¹, 10⁷ M⁻¹s⁻¹ or 10⁸ M⁻¹s⁻¹. In some embodiments, Sclerostin is human Sclerostin. In some embodiments, Sclerostin is cynomolgus Sclerostin.

In some embodiments, the K_{off} of the binding between the antibody moiety and Sclerostin is about 1 s⁻¹ to about 10⁻⁶ s⁻¹, about 1 s⁻¹ to about 10⁻² s⁻¹, about 10⁻² s⁻¹ to about 10⁻³ s⁻¹, about 10⁻³ s⁻¹ to about 10⁻⁴ s⁻¹, about 10⁻⁴ s⁻¹ to about 10⁻⁵ s⁻¹, about 10⁻⁵ s⁻¹ to about 10⁻⁶ s⁻¹, about 1 s⁻¹ to about 10⁻⁵ s⁻¹, about 10⁻² s⁻¹ to about 10⁻⁶ s⁻¹, about 10⁻³ s⁻¹ to about 10⁻⁶ s⁻¹, about 10⁻⁴ s⁻¹ to about 10⁻⁶ s⁻¹, about 10⁻² s⁻¹ to about 10⁻⁵ s⁻¹, or about 10⁻³ s⁻¹ to about 10⁻⁵ s⁻¹. In some embodiments, the K_{off} of the binding between the antibody moiety and Sclerostin is at least about any one of 1 s⁻¹, 10⁻² s⁻¹, 10⁻³ s⁻¹, 10⁻⁴ s⁻¹, 10⁻⁵ s⁻¹ or 10⁻⁶ s⁻¹. In some embodiments, Sclerostin is human Sclerostin. In some embodiments, Sclerostin is cynomolgus Sclerostin.

In some embodiments, the binding affinity of the anti-Sclerostin antibody moiety or anti-Sclerostin construct are higher (for example, has a smaller K_{D} value) than an existing anti-Sclerostin antibody (e.g., Romosozumab).

### b) Chimeric or Humanized Antibodies

In some embodiments, the anti-Sclerostin construct (e.g., the anti-Sclerostin antibody moiety) is a chimeric antibody. Certain chimeric antibodies are described, e.g., in U.S. Pat. No. 4,816,567; and Morrison et al., Proc. Natl. Acad. Sci. USA, 81:6851-6855 (1984)). In some embodiments, a chimeric antibody comprises a non-human variable region (e.g., a variable region derived from mouse) and a human constant region. In some embodiments, a chimeric antibody is a “class switched” antibody in which the class or subclass has been changed from that of the parent antibody. Chimeric antibodies include antigen-binding fragments thereof.

In some embodiments, the anti-Sclerostin construct (e.g., the anti-Sclerostin antibody moiety) is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J Immunol. 151:2296 (1993)); Framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

It is understood that the humanization of mouse derived antibodies is a common and routinely used art. It is therefore understood that a humanized format of any and all of the anti-Sclerostin antibodies disclosed in Sequence Table can be used in a preclinical or clinical setting. In cases where a humanized format of any of the referenced anti-Sclerostin antibodies or their antigen-binding regions thereof is used in such a preclinical or clinical setting, the then humanized format is expected to bear the same or similar biological activities and profiles as the original non-humanized format.

### c) Human Antibodies

In some embodiments, the anti-Sclerostin construct (e.g., the anti-Sclerostin antibody moiety) is a human antibody (known as human domain antibody, or human dAb). Human antibodies can be produced using various techniques known in the art. Human antibodies are described generally in van Dijk and van de Winkel, Curr. Opin. Pharmacol. 5: 368-74 (2001), Lonberg, Curr. Opin. Immunol. 20:450-459 (2008), and Chen, Mol. Immunol. 47(4):912-21 (2010). Transgenic mice or rats capable of producing fully human single-domain antibodies (or sdAb) are known in the art. See, e.g., US20090307787A1, U.S. Pat. No. 8,754,287, US20150289489A1, US20100122358A1, and WO2004049794.

Human antibodies (e.g., human dAbs) may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies (e.g., human dAbs) can also be made by hybridoma-based methods. Human myeloma and mouse-human heteromyeloma cell lines for the production of human monoclonal antibodies have been described (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies (e.g., human dAbs) may also be generated by isolating Fv clone variable domain sequences selected from human-derived phage display libraries. Such variable domain sequences may then be combined with a desired human constant domain. Techniques for selecting human antibodies from antibody libraries are described below.

### d) Library-Derived Antibodies

The anti-Sclerostin antibody moieties described herein may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004). Methods for constructing single-domain antibody libraries have been described, for example, See U.S. Pat. No. 7,371,849.

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically displays antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### e) Substitution, Insertion, Deletion and Variants

In some embodiments, antibody variants having one or more amino acid substitutions are provided. Sites of interest for substitutional mutagenesis include the HVRs (or CDRs) and FRs. Conservative substitutions are shown in Table 2 under the heading of “Preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2
Amino acid substitutions

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) maybe made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or CDRs.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### f) Glycosylation Variants

In some embodiments, the anti-Sclerostin construct (e.g., the anti-Sclerostin antibody moiety) is altered to increase or decrease the extent to which the construct is glycosylated. Addition or deletion of glycosylation sites to an antibody may be conveniently accomplished by altering the amino acid sequence such that one or more glycosylation sites is created or removed.

Where the antibody moiety comprises an Fc region, the carbohydrate attached thereto may be altered. Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the antibody moiety may be made in order to create antibody variants with certain improved properties.

In some embodiments, the anti-Sclerostin construct (e.g., the anti-Sclerostin antibody moiety) has a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In some embodiments, the anti-Sclerostin construct (e.g., the anti-Sclerostin antibody moiety) has bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### g) Fc Region Variants and Light Chain Constant Region Variants

In some embodiments, the anti-Sclerostin construct (e.g., the anti-Sclerostin antibody moiety) comprises an Fc fragment.

The term “Fc region,” “Fc domain,” “Fc fragment” or “Fc” refers to a C-terminal non-antigen binding region of an immunoglobulin heavy chain that contains at least a portion of the constant region. The term includes native Fc regions and variant Fc regions. In some embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. Unless otherwise specified herein, numbering of amino acid residues in the IgG or Fc region is according to the EU numbering system for antibodies, also called the EU index, as described in Kabat et al., Sequences of Proteins of Immunological Interest, 5th Ed. Public Health Service, National Institutes of Health, Bethesda, MD, 1991.

In some embodiments, the Fc fragment is from an immunoglobulin selected from the group consisting of IgG, IgA, IgD, IgE, IgM, and combinations and hybrids thereof. In some embodiments, the Fc fragment is from an immunoglobulin selected from the group consisting of IgG1, IgG2, IgG3, IgG4, and combinations and hybrids thereof.

In some embodiments, the Fc fragment has a reduced effector function as compared to corresponding wildtype Fc fragment (such as at least about 30%, 40%, 50%, 60%, 70%, 80%, 85%, 90%, or 95% reduced effector function as measured by the level of antibody-dependent cellular cytotoxicity (ADCC)).

In some embodiments, the Fc fragment is an IgG1 Fc fragment. In some embodiments, the IgG1 Fc fragment comprises a L234A mutation and/or a L235A mutation. In some embodiments, the Fc fragment is an IgG2 or IgG4 Fc fragment. In some embodiments, the Fc fragment is an IgG4 Fc fragment comprising a S228P, F234A, and/or a L235A mutation. In some embodiments, the Fc fragment comprises a N297A mutation. In some embodiments, the Fc fragment comprises a N297G mutation.

In some embodiments, one or more amino acid modifications may be introduced into the Fc region of the antibody moiety, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions. In some embodiments, the Fc fragment is derived from a rat Fc region sequence (e.g., a rat IgG2 Fc) or a mouse Fc region sequence (e.g., a mouse IgG1 Fc).

In some embodiments, the Fc fragment comprises a human IgG2 Fc region.

In some embodiments, the Fc fragment comprises a human IgG4 Fc region. In some embodiments, the Fc fragment comprises a modified human IgG4 heavy chain Fc region comprising one or more (such as two, three, or four) substitutions selected from the group consisting of S228P, T366W, and optional H435R, and optional Y436F. The numberings of the modifications described herein are according to the EU index unless otherwise noted. In some embodiments, the Fc fragment comprises a modified human IgG4 heavy chain Fc region comprising S228P, T366W, and optional H435R, and optional Y436F. In some embodiments, the Fc fragment comprises a modified human IgG4 heavy chain Fc region comprising one or more (such as two, three, four, five or six) substitutions selected from the group consisting of F126C, L128C, C131S, F170C, P161C, V173C, S228P, T366S, L368A, and Y407V, and optional H435R, and optional Y436F. In some embodiments, the Fc fragment comprises a modified human IgG4 heavy chain Fc region comprising a) C131S, S228P, T366S, L368A, and Y407V, and b) one of the substitutions selected from the group consisting of F126C, L128C, F170C, P161C, and V173C, and c) optional H435R, and optional Y436F. In some embodiments, the Fc fragment comprises a modified human IgG4 heavy chain Fc region comprising one or more (such as two, three, four, five or six) substitutions selected from the group consisting of F126C, C131S, S228P, T366S, L368A, and Y407V, and optional H435R, and optional Y436F. In some embodiments, the Fc fragment comprises a modified human IgG4 heavy chain Fc region comprising F126C, C131S, S228P, T366S, L368A, and Y407V, and optional H435R, and optional Y436F. In some embodiments, the Fc fragment comprises a modified human IgG4 heavy chain Fc region comprising L128C, C131S, S228P, T366S, L368A, and Y407V, and optional H435R, and optional Y436F. In some embodiments, the Fc fragment comprises a modified human IgG4 heavy chain Fc region comprising C131S, F170C, S228P, T366S, L368A, and Y407V, and optional H435R, and optional Y436F. In some embodiments, the Fc fragment comprises a modified human IgG4 heavy chain Fc region comprising C131S, V173C, S228P, T366S, L368A, and Y407V, and optional H435R, and optional Y436F. In some embodiments, the Fc fragment comprises a modified human IgG4 heavy chain Fc region comprising C131S, P171C, S228P, T366S, L368A, and Y407V, and optional H435R, and optional Y436F.

In some embodiments, the anti-Sclerostin construct comprises a Fc fragment comprising a) a first modified human IgG4 heavy chain Fc region comprising S228P, T366W, and b) a second modified human IgG4 heavy chain Fc region comprising a) C131S, S228P, T366S, L368A, and Y407V, and optional H435R, and optional Y436F, and b) one of the substitutions selected from the group consisting of F126C, L128C, F170C, P161C, and V173C.

In some embodiments, the anti-Sclerostin construct comprises a Fc fragment comprising a) a first modified human IgG4 heavy chain Fc region comprising S228P, T366W, and b) a second modified human IgG4 heavy chain Fc region comprising F126C, C131S, S228P, T366S, L368A, Y407V, H435R, and Y436F.

In some embodiments, the anti-Sclerostin construct comprises a Fc fragment comprising a) a first modified human IgG4 heavy chain Fc region comprising S228P, T366W, and b) a second modified human IgG4 heavy chain Fc region comprising L128C, C131S, S228P, T366S, L368A, Y407V, H435R, and Y436F.

In some embodiments, the anti-Sclerostin construct comprises a Fc fragment comprising a) a first modified human IgG4 heavy chain Fc region comprising S228P, T366W, and b) a second modified human IgG4 heavy chain Fc region comprising C131S, F170C, S228P, T366S, L368A, Y407V, H435R, and Y436F.

In some embodiments, the anti-Sclerostin construct comprises a Fc fragment comprising a) a first modified human IgG4 heavy chain Fc region comprising S228P, T366W, and b) a second modified human IgG4 heavy chain Fc region comprising C131S, V173C, S228P, T366S, L368A, Y407V, H435R, and Y436F.

In some embodiments, the anti-Sclerostin construct comprises a Fc fragment comprising a) a first modified human IgG4 heavy chain Fc region comprising S228P, T366W, and b) a second modified human IgG4 heavy chain Fc region comprising C131S, P171C, S228P, T366S, L368A, Y407V, H435R, and Y436F.

In some embodiments, the anti-Sclerostin construct comprises a human Ig kappa light chain constant region. In some embodiments, the anti-Sclerostin construct comprises a modified human Ig kappa light chain constant region comprising F118C, S121C, Q160C, S162C, S176C, and/or C214S. In some embodiments, the anti-Sclerostin construct comprises a modified human Ig kappa light chain constant region comprising S121C and C214S. In some embodiments, the anti-Sclerostin construct comprises a modified human Ig kappa light chain constant region comprising F118C and C214S. In some embodiments, the anti-Sclerostin construct comprises a modified human Ig kappa light chain constant region comprising S176C and C214S. In some embodiments, the anti-Sclerostin construct comprises a modified human Ig kappa light chain constant region comprising Q160C and C214S. In some embodiments, the anti-Sclerostin construct comprises a modified human Ig kappa light chain constant region comprising S162C and C214S.

In some embodiments, the Fc fragment possesses some but not all effector functions, which make it a desirable candidate for applications in which the half-life of the antibody moiety in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (See Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). In some embodiments, the Fc fragment comprises a N297A mutation. In some embodiments, the Fc fragment comprises a N297G mutation.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, the Fc fragment is an IgG1 Fc fragment. In some embodiments, the IgG1 Fc fragment comprises a L234A mutation and/or a L235A mutation. In some embodiments, the Fc fragment is an IgG2 or IgG4 Fc fragment. In some embodiments, the Fc fragment is an IgG4 Fc fragment comprising a S228P, F234A, and/or a L235A mutation.

In some embodiments, the antibody moiety comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In some embodiments, the antibody moiety variant comprising a variant Fc region comprising one or more amino acid substitutions which alters half-life and/or changes binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J Immunol. 24:249 (1994)), are described in US2005/0014934A1 (Hinton et al.). Those antibodies comprise an Fc region with one or more substitutions therein which alters binding of the Fc region to FcRn. Such Fc variants include those with substitutions at one or more of Fc region residues at positions 250, 252, 254, 256, 307, 308, 428, 434 (U.S. Pat. No. 7,371,826), including the so-called “LS” Fc mutant comprising M428L and N434S (WO 2009/086320), and so-called “YTE” Fc mutant comprising M252Y, S254T and T256E (WO 2002/060919).

See also Duncan & Winter, Nature 322:738-40 (1988); U.S. Pat. Nos. 5,648,260; 5,624,821; and WO 94/29351 concerning other examples of Fc region variants.

### h) Cysteine Engineered Antibody Variants

In some embodiments, it may be desirable to create cysteine engineered antibody moieties, e.g., “thioMAbs™,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an antibody-drug conjugate, as described further herein. In some embodiments, any one or more of the following residues may be substituted with cysteine: Al 18 (EU numbering) of the heavy chain; and S400 (EU numbering) of the heavy chain Fc region. Cysteine engineered antibody moieties may be generated as described, e.g., in U.S. Pat. No. 7,521,541.

### i) Antibody Derivatives

In some embodiments, the antibody moiety described herein may be further modified to comprise additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in diagnosis under defined conditions, etc.

In some embodiments, the antibody moiety may be further modified to comprise one or more biologically active protein, polypeptides or fragments thereof. “Bioactive” or “biologically active”, as used herein interchangeably, means showing biological activity in the body to carry out a specific function. For example, it may mean the combination with a particular biomolecule such as protein, DNA, etc., and then promotion or inhibition of the activity of such biomolecule. In some embodiments, the bioactive protein or fragments thereof include proteins and polypeptides that are administered to patients as the active drug substance for prevention of or treatment of a disease or condition, as well as proteins and polypeptides that are used for diagnostic purposes, such as enzymes used in diagnostic tests or in vitro assays, as well as proteins and polypeptides that are administered to a patient to prevent a disease such as a vaccine.

### Multispecific Anti-Sclerostin Constructs

The anti-Sclerostin constructs in some embodiments comprise a multispecific (e.g., bispecific) anti-Sclerostin construct comprising an anti-Sclerostin antibody moiety according to any one of the anti-Sclerostin antibody moieties described herein, and a second binding moiety (such as a second antibody moiety) specifically recognizing a second antigen. In some embodiments, the multispecific anti-Sclerostin molecule comprises an anti-Sclerostin antibody moiety and a second antibody moiety specifically recognizing a second antigen. In some embodiments, the second antigen is an immune checkpoint molecule. In some embodiments, the second antigen is DKK1 (Dickkopf WNT Signaling Pathway Inhibitor 1) or RANKL (Receptor Activator of NF-κB Ligand).

### Multispecific Constructs Targeting Both Sclerostin and RANKL

The present application provides multispecific constructs targeting both Sclerostin and RANKL. In some embodiments, there is provided a multispecific construct comprising a first antibody moiety that specifically recognizes Sclerostin and a second antibody moiety that specifically recognizes RANKL. In some embodiments, the Sclerostin is a human Sclerostin. In some embodiments, the RANKL is a human RANKL.

RANKL (Receptor activator of nuclear factor kappa-B ligand), also known as tumor necrosis factor ligand superfamily member 11 (TNFSF11), is a ligand for osteoprotegerin and functions as a key factor for osteoclast differentiation and activation. This protein was shown to be a dendritic cell survival factor and is involved in the regulation of T cell-dependent immune response. T cell activation was reported to induce expression of this gene and lead to an increase of osteoclastogenesis and bone loss. This protein was shown to activate antiapoptotic kinase AKT/PKB through a signaling complex involving SRC kinase and tumor necrosis factor receptor-associated factor (TRAF6), which indicated this protein may have a role in the regulation of cell apoptosis. Targeted disruption of the related gene in mice led to severe osteopetrosis and a lack of osteoclasts. The deficient mice exhibited defects in early differentiation of T and B lymphocytes, and failed to form lobulo-alveolar mammary structures during pregnancy.

### Exemplary Anti-RANKL Antibody Moieties

In some embodiments, the anti-RANKL antibody moiety (such as an scFv) used in multispecific anti-Sclerostin constructs described herein comprises an antibody moiety comprising a heavy chain variable region (V_{H}) and a light chain variable region (V_{L}), wherein the antibody moiety competes for a binding epitope of RANKL with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 66, the HC-CDR2 comprising the amino acid sequence of SEQ ID NO: 67, and the HC-CDR3 comprising the amino acid sequence of SEQ ID NO: 68, and the V_{L-2} comprises the LC-CDR1 comprising the amino acid sequence of SEQ ID NO: 69, the LC-CDR2 comprising the amino acid sequence of SEQ ID NO: 70, and the LC-CDR3 comprising the amino acid sequence of SEQ ID NO: 71.

In some embodiments, the anti-RANKL antibody moiety (such as an scFv) used in multispecific anti-Sclerostin constructs comprises an antibody moiety comprising a heavy chain variable region (V_{H}) and a light chain variable region (V_{L}), wherein the V_{H} comprises the HC-CDR1 comprising the amino acid sequence of SEQ ID NO: 66, the HC-CDR2 comprising the amino acid sequence of SEQ ID NO: 67, and the HC-CDR3 comprising the amino acid sequence of SEQ ID NO: 68, and the V_{L} comprises the LC-CDR1 comprising the amino acid sequence of SEQ ID NO: 69, the LC-CDR2 comprising the amino acid sequence of SEQ ID NO: 70, and the LC-CDR3 comprising the amino acid sequence of SEQ ID NO: 71. In some embodiments, the V_{H} comprises an amino acid sequence of SEQ ID NO: 72, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 73, or a variant comprising an amino acid sequence having at least about 80% (such as at least about any one of 80%, 85%, 90%, 95%, 96%, 97%, 98%, or 99%) sequence identity.

In some embodiments, the anti-RANKL moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 72; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 73.

In some embodiments, there is provided a multispecific construct comprising a first antibody moiety that specifically recognizes RANKL and a second antibody moiety that specifically recognizes Sclerostin, wherein the first antibody moiety comprises an anti-RANKL single domain antibody (sdAb) moiety, and wherein the second antibody moiety comprises a full-length antibody comprising a heavy chain variable region (V_{H}) and a second light chain variable region (V_{L}) and an Fc fragment. In some embodiments, the anti-RANKL sdAb is fused to both of the heavy chains of the full-length antibody comprising an Fc fragment. In some embodiments, the anti-RANKL sdAb is fused to both of the light chains of the full-length antibody. In some embodiments, the anti-RANKL sdAb is fused to N-terminus of both heavy or light chains of the full-length antibody. In some embodiments, the anti-RANKL sdAb is fused to C-terminus of both heavy or light chains of the full-length antibody. In some embodiments, the anti-RANKL sdAb is fused to the full-length antibody via a linker (such as any of the linkers described herein). In some embodiments, the anti-RANKL sdAb is fused to the full-length antibody without a linker.

In some embodiments, there is provided a multispecific construct comprising a first antibody moiety that specifically recognizes RANKL and a second antibody moiety that specifically recognizes Sclerostin, wherein the first antibody moiety comprises an anti-RANKL single domain antibody (sdAb) moiety, wherein the second antibody moiety comprises a heavy chain variable region (V_{H}) and a second light chain variable region (V_{L}), wherein the construct comprises: a) two chimeric heavy chains each comprising, from N-terminus to C-terminus, the i) the V_{H}, ii) a first heavy chain constant domain (“C_{H}1 domain”), iii) the anti-RANKL sdAb, and iv) an Fc domain, wherein the two Fc domains form an Fc fragment; b) two light chains comprising the V_{L} and a light chain constant domain (“C_{L} domain”). In some embodiments, the anti-RANKL sdAb is fused to the Fc domain via a first linker. In some embodiments, the anti-RANKL sdAb is fused to the V_{H} via a second linker.

In some embodiments, there is provided a multispecific construct comprising a first antibody moiety that specifically recognizes RANKL and a second antibody moiety that specifically recognizes Sclerostin, wherein the first antibody moiety comprises an anti-RANKL single domain antibody (sdAb) moiety, wherein the second antibody moiety comprises a heavy chain variable region (V_{H}) and a second light chain variable region (V_{L}), and wherein the construct comprises: a) a first heavy chain comprising, from N-terminus to C-terminus, i) the anti-RANKL sdAb, and ii) a first Fc domain; b) a second heavy chain comprising, from N-terminus to C-terminus, i) the V_{H}, ii) a first heavy chain constant domain (“C_{H}1 domain”), and iii) a second Fc domain; and c) a light chain comprising the V_{L} and a light chain constant domain (“C_{L} domain”), wherein the first and the second Fc domains form an Fc fragment.

In some embodiments, one of the first and the second Fc domains comprises a T366W mutation, and optionally a S354C mutation, and wherein the other Fc domain comprises a T366S mutation, a L368A mutation, a Y407V mutation, and optionally a Y349C mutation.

In some embodiments, wherein the second antibody moiety competes for a binding epitope of RANKL with a third antibody moiety comprising a second heavy chain variable region (V_{H-2}) and a second light chain variable region (V_{L-2}), wherein the V_{H-2} comprises the HC-CDR1 comprising the amino acid sequence of SEQ ID NO: 66, the HC-CDR2 comprising the amino acid sequence of SEQ ID NO: 67, and the HC-CDR3 comprising the amino acid sequence of SEQ ID NO: 68, and the V_{L-2} comprises the LC-CDR1 comprising the amino acid sequence of SEQ ID NO: 69, the LC-CDR2 comprising the amino acid sequence of SEQ ID NO: 70, and the LC-CDR3 comprising the amino acid sequence of SEQ ID NO: 71. In some embodiments, the V_{H} comprises a HC-CDR1 comprising the amino acid sequence of SEQ ID NO: 66, a HC-CDR2 comprising the amino acid sequence of SEQ ID NO: 67, and a HC-CDR3 comprising the amino acid sequence of SEQ ID NO: 68, and the V_{L} comprises a LC-CDR1 comprising the amino acid sequence of SEQ ID NO: 69, a LC-CDR2 comprising the amino acid sequence of SEQ ID NO: 70, and a LC-CDR3 comprising the amino acid sequence of SEQ ID NO: 71.

In some embodiments, the anti-Sclerostin construct is a multispecific (e.g., bispecific) anti-Sclerostin construct comprising a) an anti-Sclerostin antibody moiety according to any one of the anti-Sclerostin antibody moieties described herein; b) a second antibody moiety specifically recognizing RANKL (an anti-RANKL antibody moiety such as any of the anti-RANKL antibody moieties described herein). In some embodiments, the anti-Sclerostin V_{H} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 9, or a variant thereof comprising up to 5, 4, 3, 2, or 1 amino acid substitutions in the HC-CDRs, and the anti-Sclerostin V_{L} comprises i) the LC-CDR1 comprising the amino acid sequence of SEQ ID NO: 15, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs.

In some embodiments, the anti-Sclerostin V_{H} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 9, or a variant thereof comprising up to 5, 4, 3, 2, or 1 amino acid substitutions in the HC-CDRs, and the anti-Sclerostin V_{L} comprises i) the LC-CDR1 comprising the amino acid sequence of SEQ ID NO: 85, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs.

In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-Sclerostin construct is a multispecific (e.g., bispecific) anti-Sclerostin construct comprising a) an anti-Sclerostin full-length antibody comprising two heavy chains and two light chains, wherein the two heavy chains each comprises a heavy chain variable region (V_{H}) and the two light chains each comprises a light chain variable region (V_{L}), b) an anti-RANKL antibody moiety (such as any of the antibody moiety described herein) fused to at least both of the heavy chains of the anti-Sclerostin full-length antibody. In some embodiments, the anti-RANKL antibody moiety is fused to N-terminus of both heavy chains. In some embodiments, the anti-RANKL antibody moiety is fused to C-terminus of both heavy chains.

In some embodiments, the anti-Sclerostin construct is a multispecific (e.g., bispecific) anti-Sclerostin construct comprising a) an anti-RANKL antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-Sclerostin antibody moiety (such as any of the anti-Sclerostin antibody moiety described herein) fused to at least one or both of the heavy chains of the anti-RANKL full-length antibody. In some embodiments, the anti-Sclerostin antibody moiety is fused to N-terminus of both heavy chains. In some embodiments, the anti-Sclerostin antibody moiety is fused to C-terminus of both heavy chains.

In some embodiments, the anti-Sclerostin construct is a multispecific (e.g., bispecific) anti-Sclerostin construct comprising a) an anti-Sclerostin full-length antibody comprising two heavy chains and two light chains, wherein the two heavy chains each comprises a heavy chain variable region (V_{H}) and the two light chains each comprises a light chain variable region (V_{L}), b) an anti-RANKL antibody moiety (such as any of the antibody moiety described herein) fused to at least both of the light chains of the anti-Sclerostin full-length antibody. In some embodiments, the anti-RANKL antibody moiety is fused to N-terminus of both light chains. In some embodiments, the anti-RANKL antibody moiety is fused to C-terminus of both light chains.

In some embodiments, the anti-Sclerostin construct is a multispecific (e.g., bispecific) anti-Sclerostin construct comprising a) an anti-RANKL antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-Sclerostin antibody moiety (such as any of the antibody moiety described herein) fused to at least both of the light chains of the anti-RANKL full-length antibody. In some embodiments, the anti-Sclerostin antibody moiety is fused to N-terminus of both light chains. In some embodiments, the anti-Sclerostin antibody moiety is fused to C-terminus of both light chains.

In some embodiments, there is provided a multispecific construct comprising a first antibody moiety that specifically recognizes Sclerostin and a second antibody moiety that specifically recognizes RANKL, wherein the first antibody moiety comprises single chain Fv fragment (scFv) comprising a first heavy chain variable region (V_{H-1}) and a first light chain variable region (V_{L-1}), and wherein the second antibody moiety is a full-length antibody comprising a second heavy chain variable region (V_{H-2}), a second light chain variable region (V_{L-2}) and an Fc fragment. In some embodiments, the first antibody moiety is fused to one or both of the heavy chains of the full-length antibody. In some embodiments, the first antibody moiety is fused to one or both of the light chains of the full-length antibody. In some embodiments, the first antibody moiety is fused to N-terminus of the one or both of the heavy chains or light chains of the full-length antibody. In some embodiments, the first antibody moiety is fused to C-terminus of the one or both of the heavy chains or light chains of the full-length antibody. In some embodiments, the first antibody moiety is fused to the full-length antibody via a first linker (such as any of the linkers described herein). In some embodiments, the first antibody moiety is fused to the full-length antibody without a linker. In some embodiments, the V_{H-1} is fused with the V_{L-1} via a second linker (such as any of the linkers described herein).

In some embodiments, there is provided a multispecific construct comprising a first antibody moiety that specifically recognizes Sclerostin and a second antibody moiety that specifically recognizes RANKL, wherein the first antibody moiety is a full-length antibody comprising a first heavy chain variable region (V_{H-1}), a first light chain variable region (V_{L-1}) and an Fc fragment, and wherein the second antibody moiety comprises single chain Fv fragment (scFv) comprising a second heavy chain variable region (V_{H-2}) and a second light chain variable region (V_{L-2}). In some embodiments, the second antibody moiety is fused to both of the heavy chains of the full-length antibody. In some embodiments, the second antibody moiety is fused to both of the light chains of the full-length antibody. In some embodiments, the second antibody moiety is fused to N-terminus of both of the heavy chains or light chains of the full-length antibody. In some embodiments, the second antibody moiety is fused to C-terminus of the both of the heavy chains or light chains of the full-length antibody. In some embodiments, the second antibody moiety is fused to the full-length antibody via a first linker (such as any of the linkers described herein). In some embodiments, the second antibody moiety is fused to the full-length antibody without a linker. In some embodiments, the V_{H-2} is fused with the V_{L-2} via a second linker (such as any of the linkers described herein), to enable correct scFv assembling. In some embodiments, the V_{H-2} is fused with the V_{L-2} without a linker.

In some embodiments, there is provided a multispecific construct specifically recognizing Sclerostin and RANKL, comprising a first antibody moiety and a second antibody, wherein the first antibody moiety comprises a first heavy chain variable region (V_{H-1}) and a first light chain variable region (V_{L-1}), and wherein the second antibody moiety comprises a second heavy chain variable region (V_{H-2}) and a second light chain variable region (V_{L-2}), wherein the construct comprises: a) a first polypeptide comprising a first light chain comprising, from N-terminus to C-terminus, i) the V_{L-1}, ii) a first light chain constant domain (“first CL domain”); b) a second polypeptide comprising a first heavy chain, from N-terminus to C-terminus, i) the V_{H-1}, ii) a first heavy chain constant domain (“first CH1 domain”), and iii) a first Fc domain; c) a third polypeptide comprising a second heavy chain comprising, from N-terminus to C-terminus, i) the V_{H-2}, ii) a second heavy chain constant domain (“second CH1 domain”), and iii) a second Fc domain; and d) a fourth polypeptide comprising a second light chain, from N-terminus to C-terminus, i) the V_{L-2}, ii) ii) a first light chain constant domain (“the second CL domain”), wherein the first and the second Fc domains form a Fc fragment. In some embodiments, the first antibody moiety specifically recognizes Sclerostin, and the second antibody moiety specifically recognizes RANKL. In some embodiments, the first antibody moiety specifically recognizes RANKL, and the second antibody moiety specifically recognizes Sclerostin.

In some embodiments, one of the first and the second Fc domains comprises a T366W mutation, and optionally a S354C mutation, and wherein the other Fc domain comprises a T366S mutation, a L368A mutation, a Y407V mutation, and optionally a Y349C mutation, wherein numbering is according to the EU index.

In some embodiments, either i) the first CH1 domain and the first CL domain or ii) the second CH1 domain and the second CL domain are selected from the group consisting of: a) a CH1 domain wherein the amino acid at position 141 is substituted for cysteine and the cysteine at position 131 (e.g., in IgG2 or IgG4) or 220 (e.g., in IgG1) is substituted for a non-cysteine amino acid; and a CL domain wherein the amino acid at position 116 is substituted for cysteine and the cysteine at position 214 is substituted for a non-cysteine amino acid; b) a CH1 domain wherein the amino acid at position 168 is substituted for cysteine and the cysteine position 131 (e.g., in IgG2 or IgG4) or 220 (e.g., in IgG1) is substituted for a non-cysteine amino acid; and a CL domain wherein the amino acid at position 164 is substituted for cysteine and the cysteine at position 214 is substituted for a non-cysteine amino acid; c) a CH1 domain wherein the amino acid at position 126 is substituted for cysteine and the cysteine at position 131 (e.g., in IgG2 or IgG4) or 220 (e.g., in IgG1) is substituted for a non-cysteine amino acid; and a CL domain wherein the amino acid at position 121 is substituted for cysteine and the cysteine at position 214 is substituted for a non-cysteine amino acid; d) a CH1 domain wherein the amino acid at position 128 is substituted for cysteine and the cysteine at position 131 (e.g., in IgG2 or IgG4) or 220 (e.g., in IgG1) is substituted for a non-cysteine amino acid; and a CL domain wherein the amino acid at position 118 is substituted for cysteine and the cysteine at position 214 is substituted for a non-cysteine amino acid; e) a CH1 domain wherein the amino acid at position 170 is substituted for cysteine and the cysteine at position 131 (e.g., in IgG2 or IgG4) or 220 (e.g., in IgG1) is substituted for a non-cysteine amino acid; and a CL domain wherein the amino acid at position 176 is substituted for cysteine and the cysteine at position 214 is substituted for a non-cysteine amino acid; f) a CH1 domain wherein the amino acid at position 171 is substituted for cysteine and the cysteine at position 131 (e.g., in IgG2 or IgG4) or 220 (e.g., in IgG1) is substituted for a non-cysteine amino acid; and a CL domain wherein the amino acid at position 162 is substituted for cysteine and the cysteine at position 214 is substituted for a non-cysteine amino acid; g) a CH1 domain wherein the amino acid at position 173 is substituted for cysteine and the cysteine at position 131 (e.g., in IgG2 or IgG4) or 220 (e.g., in IgG1) is substituted for a non-cysteine amino acid; and a CL domain wherein the amino acid at position 160 is substituted for cysteine and the cysteine at position 214 is substituted for a non-cysteine amino acid; wherein numbering is according to the EU index.

In some embodiments, there is provided a multispecific construct comprising a first antibody moiety that specifically recognizes Sclerostin and a second antibody moiety that specifically recognizes RANKL, wherein the first antibody moiety is an anti-Sclerostin full-length antibody comprising two heavy chains and two light chains, wherein the two heavy chains each comprises a first heavy chain variable region (V_{H-1}), wherein the two light chains each comprises a first light chain variable region (V_{L-1}), and wherein the second antibody moiety comprises an anti-RANKL single chain Fv fragment (scFv) comprising a second heavy chain variable region (V_{H-2}) and a second light chain variable region (V_{L-2}), wherein the second antibody moiety is fused to C-terminus of both heavy chains of the anti-Sclerostin full-length antibody. In some embodiments, the V_{H-1} comprises an amino acid sequence of SEQ ID NO: 22, or a variant comprising an amino acid sequence having at least about 80% (such as at least about any one of 80%, 85%, 90%, 95%, 96%, 97%, 98%, or 99%) sequence identity; and the V_{L-1} comprises an amino acid sequence of SEQ ID NO: 23, or a variant comprising an amino acid sequence having at least about 80% (such as at least about any one of 80%, 85%, 90%, 95%, 96%, 97%, 98%, or 99%) sequence identity. In some embodiments, the V_{H-2} comprises the HC-CDR1 comprising the amino acid sequence of SEQ ID NO: 66, the HC-CDR2 comprising the amino acid sequence of SEQ ID NO: 67, and the HC-CDR3 comprising the amino acid sequence of SEQ ID NO: 68, and the V_{L-2} comprises the LC-CDR1 comprising the amino acid sequence of SEQ ID NO: 69, the LC-CDR2 comprising the amino acid sequence of SEQ ID NO: 70, and the LC-CDR3 comprising the amino acid sequence of SEQ ID NO: 71. In some embodiments, the V_{H-2} comprises an amino acid sequence of SEQ ID NO: 72, or a variant comprising an amino acid sequence having at least about 80% (such as at least about any one of 80%, 85%, 90%, 95%, 96%, 97%, 98%, or 99%) sequence identity; and the V_{L-1} comprises an amino acid sequence of SEQ ID NO: 73, or a variant comprising an amino acid sequence having at least about 80% (such as at least about any one of 80%, 85%, 90%, 95%, 96%, 97%, 98%, or 99%) sequence identity.

In some embodiments, there is provided a multispecific construct specifically recognizing Sclerostin and RANKL, comprising a first antibody moiety and a second antibody, wherein the first antibody moiety comprises a first heavy chain variable region (V_{H-1}) and a first light chain variable region (V_{L-1}), and wherein the second antibody moiety comprises a second heavy chain variable region (V_{H-2}) and a second light chain variable region (V_{L-2}), wherein the construct comprises: a) a first polypeptide comprising a first light chain comprising, from N-terminus to C-terminus, i) the V_{L-1}, ii) a first light chain constant domain (“first CL domain”); b) a second polypeptide comprising a first heavy chain, from N-terminus to C-terminus, i) the V_{H-1}, ii) a first heavy chain constant domain (“first CH1 domain”), and iii) a first Fc domain; c) a third polypeptide comprising a second heavy chain comprising, from N-terminus to C-terminus, i) the V_{H-2}, ii) a second heavy chain constant domain (“second CH1 domain”), and iii) a second Fc domain; and d) a fourth polypeptide comprising a second light chain, from N-terminus to C-terminus, i) the V_{L-2}, ii) ii) a first light chain constant domain (“the second CL domain”), wherein the first and the second Fc domains form a Fc fragment, wherein the first antibody moiety specifically recognizes Sclerostin, and wherein the second antibody moiety specifically recognizes RANKL. In some embodiments, the V_{H-1} comprises an amino acid sequence of SEQ ID NO: 22, or a variant comprising an amino acid sequence having at least about 80% (such as at least about any one of 80%, 85%, 90%, 95%, 96%, 97%, 98%, or 99%) sequence identity; and the V_{L-1} comprises an amino acid sequence of SEQ ID NO: 23, or a variant comprising an amino acid sequence having at least about 80% (such as at least about any one of 80%, 85%, 90%, 95%, 96%, 97%, 98%, or 99%) sequence identity. In some embodiments, the V_{H-2} comprises the HC-CDR1 comprising the amino acid sequence of SEQ ID NO: 66, the HC-CDR2 comprising the amino acid sequence of SEQ ID NO: 67, and the HC-CDR3 comprising the amino acid sequence of SEQ ID NO: 68, and the V_{L-2} comprises the LC-CDR1 comprising the amino acid sequence of SEQ ID NO: 69, the LC-CDR2 comprising the amino acid sequence of SEQ ID NO: 70, and the LC-CDR3 comprising the amino acid sequence of SEQ ID NO: 71. In some embodiments, the V_{H-2} comprises an amino acid sequence of SEQ ID NO: 72, or a variant comprising an amino acid sequence having at least about 80% (such as at least about any one of 80%, 85%, 90%, 95%, 96%, 97%, 98%, or 99%) sequence identity; and the V_{L-1} comprises an amino acid sequence of SEQ ID NO: 73, or a variant comprising an amino acid sequence having at least about 80% (such as at least about any one of 80%, 85%, 90%, 95%, 96%, 97%, 98%, or 99%) sequence identity.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 134; b) a first light chain comprising a first light chain variable region (V_{L-1}) and a first light chain constant region, wherein the first light chain comprises an amino acid sequence of SEQ ID NO: 135; c) a second heavy chain comprising a second variable heavy chain variable region (V_{H-2}) and a second heavy chain constant region, wherein the second heavy chain comprises the amino acid sequence of SEQ ID NO: 156; and d) a second light chain comprising a second light chain variable region (V_{L-2}) and a second light chain constant region, wherein the second light chain comprises the amino acid sequence of SEQ ID NO: 157, wherein the V_{H-1} and the V_{L-1} comprises a first antibody moiety specifically recognizing Sclerostin, wherein the V_{H-2} and the V_{L-2} comprises a second antibody moiety specifically recognizing RANKL.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 134; b) a first light chain comprising a first light chain variable region (V_{L-1}) and a first light chain constant region, wherein the first light chain comprises an amino acid sequence of SEQ ID NO: 135; c) a second heavy chain comprising a second variable heavy chain variable region (V_{H-2}) and a second heavy chain constant region, wherein the second heavy chain comprises the amino acid sequence of SEQ ID NO: 158; and d) a second light chain comprising a second light chain variable region (V_{L-2}) and a second light chain constant region, wherein the second light chain comprises the amino acid sequence of SEQ ID NO: 159, wherein the V_{H-1} and the V_{L-1} comprises a first antibody moiety specifically recognizing Sclerostin, wherein the V_{H-2} and the V_{L-2} comprises a second antibody moiety specifically recognizing RANKL. In some embodiments, the V_{H-1} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9; and the V_{L-1} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of any one of SEQ ID NOs: 20. In some embodiments,

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 134; b) a first light chain comprising a first light chain variable region (V_{L-1}) and a first light chain constant region, wherein the first light chain comprises an amino acid sequence of SEQ ID NO: 135; c) a second heavy chain comprising a second variable heavy chain variable region (V_{H-2}) and a second heavy chain constant region, wherein the second heavy chain comprises the amino acid sequence of SEQ ID NO: 160; and d) a second light chain comprising a second light chain variable region (V_{L-2}) and a second light chain constant region, wherein the second light chain comprises the amino acid sequence of SEQ ID NO: 161, wherein the V_{H-1} and the V_{L-1} comprises a first antibody moiety specifically recognizing Sclerostin, wherein the V_{H-2} and the V_{L-2} comprises a second antibody moiety specifically recognizing RANKL.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 134; b) a first light chain comprising a first light chain variable region (V_{L-1}) and a first light chain constant region, wherein the first light chain comprises an amino acid sequence of SEQ ID NO: 135; c) a second heavy chain comprising a second variable heavy chain variable region (V_{H-2}) and a second heavy chain constant region, wherein the second heavy chain comprises the amino acid sequence of SEQ ID NO: 162; and d) a second light chain comprising a second light chain variable region (V_{L-2}) and a second light chain constant region, wherein the second light chain comprises the amino acid sequence of SEQ ID NO: 163, wherein the V_{H-1} and the V_{L-1} comprises a first antibody moiety specifically recognizing Sclerostin, wherein the V_{H-2} and the V_{L-2} comprises a second antibody moiety specifically recognizing RANKL.

In some embodiments, there is provided a multispecific (e.g., bispecific) anti-Sclerostin construct comprising a) a first antibody moiety that specifically recognizes Sclerostin comprising a first heavy chain variable region (V_{H-1}) and a first light chain variable region (V_{L-1}), wherein the V_{H-1} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9, and the V_{L-1} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of SEQ ID NO: 20, and b) a second antibody moiety that specifically recognizes RANKL comprising a second heavy chain variable region (V_{H-2}) and a second light chain variable region (V_{L-2}), wherein the V_{H-2} comprises the HC-CDR1 comprising the amino acid sequence of SEQ ID NO: 66, the HC-CDR2 comprising the amino acid sequence of SEQ ID NO: 67, and the HC-CDR3 comprising the amino acid sequence of SEQ ID NO: 68, and the V_{L-2} comprises the LC-CDR1 comprising the amino acid sequence of SEQ ID NO: 69, the LC-CDR2 comprising the amino acid sequence of SEQ ID NO: 70, and the LC-CDR3 comprising the amino acid sequence of SEQ ID NO: 71. In some embodiments, the V_{H-1} comprises an amino acid sequence of SEQ ID NO: 22, and the V_{L-1} comprises an amino acid sequence of SEQ ID NO: 23. In some embodiments, the V_{H-1} comprises an amino acid sequence of SEQ ID NO: 87 or 168, and the V_{L-1} comprises an amino acid sequence of SEQ ID NO: 88 or 169. In some embodiments, the V_{H-1} comprises an amino acid sequence of SEQ ID NO: 87, and the V_{L-1} comprises an amino acid sequence of SEQ ID NO: 88. In some embodiments, the V_{H-1} comprises an amino acid sequence of SEQ ID NO: 168, and the V_{L-1} comprises an amino acid sequence of SEQ ID NO: 169. In some embodiments, the V_{H-2} comprises an amino acid sequence of SEQ ID NO: 72, and the V_{L-2} comprises an amino acid sequence of SEQ ID NO: 73. In some embodiments, the first antibody moiety is a full-length antibody comprising two heavy chains and two light chains, and the second antibody moiety is a scFv comprising the V_{H-2} and V_{L-2}. In some embodiments, the V_{H-2} is fused to the N-terminus of the V_{L}-2. In some embodiments, the V_{H-2} is fused to the C-terminus of the V_{L-2} optionally with a single alanine amino acid appended to the C-terminus of V_{H-2}. In some embodiments, the V_{H-2} and the V_{L-2} are fused via a linker (e.g., a GS linker, e.g., (GGGGS)₄). In some embodiments, the V_{H-1} and the V_{L-1} are fused without a linker. In some embodiments, the scFv is a dsscFv optionally comprising a) a G44C mutation in the V_{H} or V_{H-2} according to the numbering of SEQ ID NO: 60, and b) a G100C mutation in the V_{L} or V_{L-2} according to the numbering of SEQ ID NO: 61. In some embodiments, the second antibody moiety is fused to N-terminus of two heavy chains of the full-length antibody. In some embodiments, the second antibody moiety is fused to C-terminus of two heavy chains of the full-length antibody. In some embodiments, the second antibody moiety is fused to N-terminus of two light chains of the full-length antibody. In some embodiments, the second antibody moiety is fused to C-terminus of two light chains of the full-length antibody. In some embodiments, the second antibody moiety is a full-length antibody comprising two heavy chains and two light chains, and the first antibody moiety is a scFv comprising the V_{H-1} and the V_{L-1}. In some embodiments, the V_{H-1} is fused to the N-terminus of the V_{L-1}. In some embodiments, the V_{H-1} is fused to the C-terminus of the V_{L-1} optionally with a single alanine amino acid appended to the C-terminus of V_{H-1}. In some embodiments, the V_{H-1} and the V_{L-1} are fused via a linker (e.g., a GS linker, e.g., (GGGGS)₄). In some embodiments, the scFv is a dsscFv optionally comprising a) a G44C mutation in the V_{H} or V_{H-2} according to the numbering of SEQ ID NO: 60, and b) a G100C mutation in the V_{L} or V_{L-2} according to the numbering of SEQ ID NO: 61. In some embodiments, the first antibody moiety is fused to N-terminus of one or two heavy chains of the full-length antibody. In some embodiments, the first antibody moiety is fused to C-terminus of one or two heavy chains of the full-length antibody. In some embodiments, the first antibody moiety is fused to N-terminus of one or two light chains of the full-length antibody. In some embodiments, the first antibody moiety is fused to C-terminus of one or two light chains of the full-length antibody. In some embodiments, the first antibody moiety and the second antibody moiety are fused via a linker (e.g., a GS linker, e.g., (GGGGS)₃). In some embodiments, the first antibody moiety and the second antibody moiety are fused without a linker.

In some embodiments, there is provided a multispecific (e.g., bispecific) construct comprising a first antibody moiety that specifically recognizes Sclerostin and a second antibody moiety that specifically recognizes RANKL, wherein the multispecific construct comprises two heavy chains and two light chains, wherein the two heavy chains each comprises an amino acid sequence of SEQ ID NO: 126, wherein the two light chains each comprises an amino acid sequence of SEQ ID NO: 129.

In some embodiments, there is provided a multispecific (e.g., bispecific) construct comprising a first antibody moiety that specifically recognizes Sclerostin and a second antibody moiety that specifically recognizes RANKL, wherein the multispecific construct comprises two heavy chains and two light chains, wherein the two heavy chains each comprises an amino acid sequence of SEQ ID NO: 127, wherein the two light chains each comprises an amino acid sequence of SEQ ID NO: 129.

In some embodiments, there is provided a multispecific (e.g., bispecific) construct comprising a first antibody moiety that specifically recognizes Sclerostin and a second antibody moiety that specifically recognizes RANKL, wherein the multispecific construct comprises two heavy chains and two light chains, wherein the two heavy chains each comprises an amino acid sequence of SEQ ID NO: 128, wherein the two light chains each comprises an amino acid sequence of SEQ ID NO: 129.

In some embodiments, there is provided a multispecific (e.g., bispecific) construct comprising a first antibody moiety that specifically recognizes Sclerostin and a second antibody moiety that specifically recognizes RANKL, wherein the multispecific construct comprises two heavy chains and two light chains, wherein the two heavy chains each comprises an amino acid sequence of SEQ ID NO: 133 wherein the two light chains each comprises an amino acid sequence of SEQ ID NO: 130.

In some embodiments, there is provided a multispecific (e.g., bispecific) construct comprising a first antibody moiety that specifically recognizes Sclerostin and a second antibody moiety that specifically recognizes RANKL, wherein the multispecific construct comprises two heavy chains and two light chains, wherein the two heavy chains each comprises an amino acid sequence of SEQ ID NO: 133, wherein the two light chains each comprises an amino acid sequence of SEQ ID NO: 131.

In some embodiments, there is provided a multispecific (e.g., bispecific) construct comprising a first antibody moiety that specifically recognizes Sclerostin and a second antibody moiety that specifically recognizes RANKL, wherein the multispecific construct comprises two heavy chains and two light chains, wherein the two heavy chains each comprises an amino acid sequence of SEQ ID NO: 133, wherein the two light chains each comprises an amino acid sequence of SEQ ID NO: 132.

In some embodiments, there is provided a multispecific (e.g., bispecific) anti-Sclerostin construct comprising a) a first antibody moiety that specifically recognizes Sclerostin comprising a first heavy chain variable region (V_{H-1}) and a first light chain variable region (V_{L-1}), wherein the V_{H-1} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9, and the V_{L-1} comprises the LC-CDR1 comprising the amino acid sequence of SEQ ID NO: 85, the LC-CDR2 comprising the amino acid sequence of SEQ ID NO: 18, and the LC-CDR3 comprising the amino acid sequence of SEQ ID NO: 20, and b) a second antibody moiety that specifically recognizes RANKL comprising a second heavy chain variable region (V_{H-2}) and a second light chain variable region (V_{L-2}), wherein the V_{H-2} comprises the HC-CDR1 comprising the amino acid sequence of SEQ ID NO: 66, the HC-CDR2 comprising the amino acid sequence of SEQ ID NO: 67, and the HC-CDR3 comprising the amino acid sequence of SEQ ID NO: 68, and the V_{L-2} comprises the LC-CDR1 comprising the amino acid sequence of SEQ ID NO: 69, the LC-CDR2 comprising the amino acid sequence of SEQ ID NO: 70, and the LC-CDR3 comprising the amino acid sequence of SEQ ID NO: 71. In some embodiments, the V_{H-1} comprises an amino acid sequence of SEQ ID NO: 87 or 168, and the V_{L-1} comprises an amino acid sequence of SEQ ID NO: 89 or 170. In some embodiments, the V_{H-1} comprises an amino acid sequence of SEQ ID NO: 87, and the V_{L-1} comprises an amino acid sequence of SEQ ID NO: 89. In some embodiments, the V_{H-1} comprises an amino acid sequence of SEQ ID NO: 168, and the V_{L-1} comprises an amino acid sequence of SEQ ID NO: 170. In some embodiments, the V_{H-2} comprises an amino acid sequence of SEQ ID NO: 72, and the V_{L-2} comprises an amino acid sequence of SEQ ID NO: 73. In some embodiments, the first antibody moiety is a full-length antibody comprising two heavy chains and two light chains, and the second antibody moiety is a scFv comprising the V_{H-2} and V_{L-2}. In some embodiments, the V_{H-2} is fused to the N-terminus of the V_{L-2}. In some embodiments, the V_{H-2} is fused to the C-terminus of the V_{L-2} optionally with a single alanine amino acid appended to the C-terminus of V_{H-2}. In some embodiments, the V_{H-2} and the V_{L-2} are fused via a linker (e.g., a GS linker, e.g., (GGGGS)₄). In some embodiments, the V_{H-1} and the V_{L-1} are fused without a linker. In some embodiments, the scFv is converted to a dsscFv with H44-L100 disulfide bond formed by G44C mutation on V_{H-2} and G100C mutation on V_{L-2}. In some embodiments, the second antibody moiety is fused to N-terminus of two heavy chains of the full-length antibody. In some embodiments, the second antibody moiety is fused to C-terminus of two heavy chains of the full-length antibody. In some embodiments, the second antibody moiety is fused to N-terminus of two light chains of the full-length antibody. In some embodiments, the second antibody moiety is fused to C-terminus of two light chains of the full-length antibody. In some embodiments, the second antibody moiety is a full-length antibody comprising two heavy chains and two light chains, and the first antibody moiety is a scFv comprising the V_{H-1} and the V_{L-1}. In some embodiments, the V_{H-1} is fused to the N-terminus of the V_{L-1}. In some embodiments, the V_{H-1} is fused to the C-terminus of the V_{L-1} optionally with a single alanine amino acid appended to the C-terminus of V_{H-1}. In some embodiments, the V_{H-1} and the V_{L-1} are fused via a linker (e.g., a GS linker, e.g., (GGGGS)₄). In some embodiments, the V_{H-1} and the V_{L-1} are fused via a linker without a linker. In some embodiments, the scFv is a dsscFv optionally comprising a) a G44C mutation in the V_{H} or V_{H-2} according to the numbering of SEQ ID NO: 60, and b) a G100C mutation in the V_{L} or V_{L-2} according to the numbering of SEQ ID NO: 61. In some embodiments, the first antibody moiety is fused to N-terminus of one or two heavy chains of the full-length antibody. In some embodiments, the first antibody moiety is fused to C-terminus of one or two heavy chains of the full-length antibody. In some embodiments, the first antibody moiety is fused to N-terminus of one or two light chains of the full-length antibody. In some embodiments, the first antibody moiety is fused to C-terminus of one or two light chains of the full-length antibody. In some embodiments, the first antibody moiety and the second antibody moiety are fused via a linker (e.g., a GS linker, e.g., (GGGGS)₃). In some embodiments, the first antibody moiety and the second antibody moiety are fused without a linker.

In some embodiments, the anti-RANKL antibody moiety and the anti-Sclerostin antibody moiety are fused with each other via a linker such as any of the linkers described herein with any operable form that allows the proper function of the binding moieties.

### Multispecific Constructs Targeting Both Sclerostin and DKK1

In some embodiments, the anti-Sclerostin construct is a multispecific (e.g., bispecific) anti-Sclerostin construct comprising a) an anti-Sclerostin antibody moiety according to any one of the anti-Sclerostin antibody moieties described herein; b) a second antibody moiety specifically recognizing DKK1 (an anti-DKK1 antibody moiety).

DKK1 (Dickkopf-related protein 1) is a member of the dickkopf family of proteins. Members of this family are secreted proteins characterized by two cysteine-rich domains that mediate protein-protein interactions. DKK1 binds to the LRP6 co-receptor and inhibits beta-catenin-dependent Wnt signaling. This gene plays a role in embryonic development and may be important in bone formation in adults.

### Exemplary Anti-DKK1 Antibody Moieties

In some embodiments, the anti-DKK1 antibody moiety (such as an scFv) used in multispecific anti-Sclerostin constructs comprises an antibody moiety comprising a heavy chain variable region (V_{H}) and a light chain variable region (V_{L}), wherein the antibody moiety competes for a binding epitope of DKK1 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 42, the HC-CDR2 comprising the amino acid sequence of SEQ ID NO: 43, and the HC-CDR3 comprising the amino acid sequence of SEQ ID NO: 44, and the V_{L-2} comprises the LC-CDR1 comprising the amino acid sequence of SEQ ID NO: 45, the LC-CDR2 comprising the amino acid sequence of SEQ ID NO: 46, and the LC-CDR3 comprising the amino acid sequence of SEQ ID NO: 47.

In some embodiments, the anti-DKK1 antibody moiety (such as an scFv) used in multispecific anti-Sclerostin constructs comprises an antibody moiety comprising a heavy chain variable region (V_{H}) and a light chain variable region (V_{L}), wherein the antibody moiety competes for a binding epitope of DKK1 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 48, the HC-CDR2 comprising the amino acid sequence of SEQ ID NO: 49, and the HC-CDR3 comprising the amino acid sequence of SEQ ID NO: 50, and the V_{L-2} comprises the LC-CDR1 comprising the amino acid sequence of SEQ ID NO: 51, the LC-CDR2 comprising the amino acid sequence of SEQ ID NO: 52, and the LC-CDR3 comprising the amino acid sequence of SEQ ID NO: 58.

In some embodiments, the anti-DKK1 antibody moiety (such as an scFv) used in multispecific anti-Sclerostin constructs comprises an antibody moiety comprising a heavy chain variable region (V_{H}) and a light chain variable region (V_{L}), wherein the antibody moiety competes for a binding epitope of DKK1 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 54, the HC-CDR2 comprising the amino acid sequence of SEQ ID NO: 55, and the HC-CDR3 comprising the amino acid sequence of SEQ ID NO: 56, and the V_{L-2} comprises the LC-CDR1 comprising the amino acid sequence of SEQ ID NO: 57, the LC-CDR2 comprising the amino acid sequence of SEQ ID NO: 58, and the LC-CDR3 comprising the amino acid sequence of SEQ ID NO: 59.

In some embodiments, the anti-DKK1 antibody moiety (such as an scFv) used in multispecific anti-Sclerostin constructs comprises an antibody moiety comprising a heavy chain variable region (V_{H}) and a light chain variable region (V_{L}), wherein the V_{H} comprises the HC-CDR1 comprising the amino acid sequence of SEQ ID NO: 42, the HC-CDR2 comprising the amino acid sequence of SEQ ID NO: 43, and the HC-CDR3 comprising the amino acid sequence of SEQ ID NO: 44, and the V_{L} comprises the LC-CDR1 comprising the amino acid sequence of SEQ ID NO: 45, the LC-CDR2 comprising the amino acid sequence of SEQ ID NO: 46, and the LC-CDR3 comprising the amino acid sequence of SEQ ID NO: 47. In some embodiments, the V_{H} comprises an amino acid sequence of SEQ ID NO: 60, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 61, or a variant comprising an amino acid sequence having at least about 80% (such as at least about any one of 80%, 85%, 90%, 95%, 96%, 97%, 98%, or 99%) sequence identity.

In some embodiments, the anti-DKK1 antibody moiety (such as an scFv) used in multispecific anti-Sclerostin constructs comprises an antibody moiety comprising a heavy chain variable region (V_{H}) and a light chain variable region (V_{L}), wherein the V_{H} comprises the HC-CDR1 comprising the amino acid sequence of SEQ ID NO: 48, the HC-CDR2 comprising the amino acid sequence of SEQ ID NO: 49, and the HC-CDR3 comprising the amino acid sequence of SEQ ID NO: 50, and the V_{L} comprises the LC-CDR1 comprising the amino acid sequence of SEQ ID NO: 51, the LC-CDR2 comprising the amino acid sequence of SEQ ID NO: 52, and the LC-CDR3 comprising the amino acid sequence of SEQ ID NO: 58. In some embodiments, the V_{H} comprises an amino acid sequence of SEQ ID NO: 62, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 63, or a variant comprising an amino acid sequence having at least about 80% (such as at least about any one of 80%, 85%, 90%, 95%, 96%, 97%, 98%, or 99%) sequence identity.

In some embodiments, the anti-DKK1 antibody moiety (such as an scFv) used in multispecific anti-Sclerostin constructs comprises an antibody moiety comprising a heavy chain variable region (V_{H}) and a light chain variable region (V_{L}), wherein the V_{H} comprises the HC-CDR1 comprising the amino acid sequence of SEQ ID NO: 54, the HC-CDR2 comprising the amino acid sequence of SEQ ID NO: 55, and the HC-CDR3 comprising the amino acid sequence of SEQ ID NO: 56, and the V_{L} comprises the LC-CDR1 comprising the amino acid sequence of SEQ ID NO: 57, the LC-CDR2 comprising the amino acid sequence of SEQ ID NO: 58, and the LC-CDR3 comprising the amino acid sequence of SEQ ID NO: 59. In some embodiments, the V_{H} comprises an amino acid sequence of SEQ ID NO: 64, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 65, or a variant comprising an amino acid sequence having at least about 80% (such as at least about any one of 80%, 85%, 90%, 95%, 96%, 97%, 98%, or 99%) sequence identity.

In some embodiments, the anti-DKK1 moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 60, 62, or 64; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 61, 63, or 65.

In some embodiments, there is provided a multispecific (e.g., bispecific) anti-Sclerostin construct comprising a) an anti-Sclerostin full-length antibody comprising two heavy chains and two light chains, wherein the two heavy chains each comprises a heavy chain variable region (V_{H}) and the two light chains each comprises a light chain variable region (V_{L}), b) an anti-DKK1 antibody moiety (such as any of the antibody moiety described herein) fused to at least both of the heavy chains of the anti-Sclerostin full-length antibody. In some embodiments, the anti-DKK1 antibody moiety is fused to N-terminus of both heavy chains. In some embodiments, the anti-DKK1 antibody moiety is fused to C-terminus of both heavy chains.

In some embodiments, there is provided a multispecific (e.g., bispecific) anti-Sclerostin construct comprising a) an anti-DKK1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-Sclerostin antibody moiety (such as any of the anti-Sclerostin antibody moiety described herein) fused to at least one or both of the heavy chains of the anti-DKK1 full-length antibody. In some embodiments, the anti-Sclerostin antibody moiety is fused to N-terminus of both heavy chains. In some embodiments, the anti-Sclerostin antibody moiety is fused to C-terminus of both heavy chains.

In some embodiments, there is provided a multispecific (e.g., bispecific) anti-Sclerostin construct comprising a) an anti-Sclerostin full-length antibody comprising two heavy chains and two light chains, wherein the two heavy chains each comprises a heavy chain variable region (V_{H}) and the two light chains each comprises a light chain variable region (V_{L}), b) an anti-DKK1 antibody moiety (such as any of the antibody moiety described herein) fused to at least both of the light chains of the anti-Sclerostin full-length antibody. In some embodiments, the anti-DKK1 antibody moiety is fused to N-terminus of both light chains. In some embodiments, the anti-DKK1 antibody moiety is fused to C-terminus of both light chains.

In some embodiments, there is provided a multispecific (e.g., bispecific) anti-Sclerostin construct comprising a) an anti-DKK1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-Sclerostin antibody moiety (such as any of the antibody moiety described herein) fused to at least one or both of the light chains of the anti-DKK1 full-length antibody. In some embodiments, the anti-Sclerostin antibody moiety is fused to N-terminus of both light chains. In some embodiments, the anti-Sclerostin antibody moiety is fused to C-terminus of both light chains.

In some embodiments, the anti-Sclerostin antibody moiety and the anti-DKK1 antibody moiety are fused with each other via a linker such as any of the linkers described herein with any operable form that allows the proper function of the binding moieties. In some embodiments, the anti-Sclerostin antibody moiety and the anti-DKK1 antibody moiety are fused without a linker.

In some embodiments, there is provided a multispecific (e.g., bispecific) anti-Sclerostin construct comprising a) a first antibody moiety that specifically recognizes Sclerostin comprising a first heavy chain variable region (V_{H-1}) and a first light chain variable region (V_{L-1}), wherein the V_{H-1} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9, and the V_{L-1} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of SEQ ID NO: 20, and b) a second antibody moiety that specifically recognizes DKK1 comprising a second heavy chain variable region (V_{H-2}) and a second light chain variable region (V_{L-2}), wherein the V_{H-2} comprises the HC-CDR1 comprising the amino acid sequence of SEQ ID NO: 42, the HC-CDR2 comprising the amino acid sequence of SEQ ID NO: 43, and the HC-CDR3 comprising the amino acid sequence of SEQ ID NO: 44, and the V_{L-2} comprises the LC-CDR1 comprising the amino acid sequence of SEQ ID NO: 45, the LC-CDR2 comprising the amino acid sequence of SEQ ID NO: 46, and the LC-CDR3 comprising the amino acid sequence of SEQ ID NO: 47. In some embodiments, the V_{H-1} comprises an amino acid sequence of SEQ ID NO: 22, and the V_{L-1} comprises an amino acid sequence of SEQ ID NO: 23. In some embodiments, the V_{H-1} comprises an amino acid sequence of SEQ ID NO: 87 or 168, and the V_{L-1} comprises an amino acid sequence of SEQ ID NO: 88 or 169. In some embodiments, the V_{H-1} comprises an amino acid sequence of SEQ ID NO: 87, and the V_{L-1} comprises an amino acid sequence of SEQ ID NO: 88. In some embodiments, the V_{H-1} comprises an amino acid sequence of SEQ ID NO: 168, and the V_{L-1} comprises an amino acid sequence of SEQ ID NO: 169. In some embodiments, the V_{H-2} comprises an amino acid sequence of SEQ ID NO: 60, 164, or 166, and the V_{L-2} comprises an amino acid sequence of SEQ ID NO: 61, 165, or 167. In some embodiments, the V_{H-2} comprises an amino acid sequence of SEQ ID NO: 164, and the V_{L-2} comprises an amino acid sequence of SEQ ID NO: 165. In some embodiments, the V_{H-2} comprises an amino acid sequence of SEQ ID NO: 166, and the V_{L-2} comprises an amino acid sequence of SEQ ID NO: 167. In some embodiments, the V_{H-2} comprises an amino acid sequence of SEQ ID NO: 61, and the V_{L-2} comprises an amino acid sequence of SEQ ID NO: 167. In some embodiments, the first antibody moiety is a full-length antibody comprising two heavy chains and two light chains, and the second antibody moiety is a scFv comprising the V_{H-2} and V_{L-2}. In some embodiments, the V_{H-2} is fused to the N-terminus of the V_{L-2} optionally with a single alanine amino acid appended to the C-terminus of V_{H-2}. In some embodiments, the V_{H-2} is fused to the C-terminus of the V_{L-2}. In some embodiments, the V_{H-2} and the V_{L-2} are fused via a linker (e.g., a GS linker, e.g., (GGGGS)₄). In some embodiments, the V_{H}-1 and the V_{L-1} are fused without a linker. In some embodiments, the scFv is a dsscFv optionally comprising a) a G44C mutation in the V_{H} or V_{H-2} according to the numbering of SEQ ID NO: 60, and b) a G100C mutation in the V_{L} or V_{L-2} according to the numbering of SEQ ID NO: 61. In some embodiments, the second antibody moiety is fused to N-terminus of two heavy chains of the full-length antibody. In some embodiments, the second antibody moiety is fused to C-terminus of two heavy chains of the full-length antibody. In some embodiments, the second antibody moiety is fused to N-terminus of two light chains of the full-length antibody. In some embodiments, the second antibody moiety is fused to C-terminus of two light chains of the full-length antibody. In some embodiments, the second antibody moiety is a full-length antibody comprising two heavy chains and two light chains, and the first antibody moiety is a scFv comprising the V_{H-1} and the V_{L-1}. In some embodiments, the V_{H-1} is fused to the N-terminus of the V_{L-1}. In some embodiments, the V_{H-1} is fused to the C-terminus of the V_{L-1} optionally with a single alanine amino acid appended to the C-terminus of V_{H-1}. In some embodiments, the V_{H-1} and the V_{L-1} are fused via a linker (e.g., a GS linker, e.g., (GGGGS)₄). In some embodiments, the V_{H-1} and the V_{L-1} are fused without a linker. In some embodiments, the scFv is a dsscFv optionally comprising a) a G44C mutation in the V_{H} or V_{H-2} according to the numbering of SEQ ID NO: 60, and b) a G100C mutation in the V_{L} or V_{L-2} according to the numbering of SEQ ID NO: 61. In some embodiments, the first antibody moiety is fused to N-terminus of one or two heavy chains of the full-length antibody. In some embodiments, the first antibody moiety is fused to C-terminus of one or two heavy chains of the full-length antibody. In some embodiments, the first antibody moiety is fused to N-terminus of one or two light chains of the full-length antibody. In some embodiments, the first antibody moiety is fused to C-terminus of one or two light chains of the full-length antibody. In some embodiments, the first antibody moiety and the second antibody moiety are fused via a linker (e.g., a GS linker, e.g., (GGGGS)₃). In some embodiments, the first antibody moiety and the second antibody moiety are fused without a linker.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 91, wherein the two light chains each comprises an amino acid sequence of SEQ ID NO: 93.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 92, wherein the two light chains each comprises an amino acid sequence of SEQ ID NO: 93.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 94, wherein the two light chains each comprises an amino acid sequence of SEQ ID NO: 96.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 95, wherein the two light chains each comprises an amino acid sequence of SEQ ID NO: 96.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 97, wherein the two light chains each comprises an amino acid sequence of SEQ ID NO: 99.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 98, wherein the two light chains each comprises an amino acid sequence of SEQ ID NO: 99.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 100, wherein the two light chains each comprises an amino acid sequence of SEQ ID NO: 102.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 101, wherein the two light chains each comprises an amino acid sequence of SEQ ID NO: 102.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 103, wherein the two light chains each comprises an amino acid sequence of SEQ ID NO: 104.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 103, wherein the two light chains each comprises an amino acid sequence of SEQ ID NO: 105.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 106, wherein the two light chains each comprises an amino acid sequence of SEQ ID NO: 107.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 106, wherein the two light chains each comprises an amino acid sequence of SEQ ID NO: 108.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 109, wherein the two light chains each comprises an amino acid sequence of SEQ ID NO: 110.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 109, wherein the two light chains each comprises an amino acid sequence of SEQ ID NO: 111.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 112, wherein the two light chains each comprises an amino acid sequence of SEQ ID NO: 113.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 112, wherein the two light chains each comprises an amino acid sequence of SEQ ID NO: 114.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 117, wherein the two light chains each comprises an amino acid sequence of SEQ ID NO: 119.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 118, wherein the two light chains each comprises an amino acid sequence of SEQ ID NO: 119.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 123, wherein the two light chains each comprises an amino acid sequence of SEQ ID NO: 125.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 124, wherein the two light chains each comprises an amino acid sequence of SEQ ID NO: 125.

In some embodiments, there is provided a multispecific (e.g., bispecific) anti-Sclerostin construct comprising a) a first antibody moiety that specifically recognizes Sclerostin comprising a first heavy chain variable region (V_{H-1}) and a first light chain variable region (V_{L-1}), wherein the V_{H-1} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9, and the V_{L-1} comprises the LC-CDR1 comprising the amino acid sequence of SEQ ID NO: 85, the LC-CDR2 comprising the amino acid sequence of SEQ ID NO: 18, and the LC-CDR3 comprising the amino acid sequence of SEQ ID NO: 20, and b) a second antibody moiety that specifically recognizes DKK1 comprising a second heavy chain variable region (V_{H-2}) and a second light chain variable region (V_{L-2}), wherein the V_{H-2} comprises the HC-CDR1 comprising the amino acid sequence of SEQ ID NO: 42, the HC-CDR2 comprising the amino acid sequence of SEQ ID NO: 43, and the HC-CDR3 comprising the amino acid sequence of SEQ ID NO: 44, and the V_{L-2} comprises the LC-CDR1 comprising the amino acid sequence of SEQ ID NO: 45, the LC-CDR2 comprising the amino acid sequence of SEQ ID NO: 46, and the LC-CDR3 comprising the amino acid sequence of SEQ ID NO: 47. In some embodiments, the V_{H-1} comprises an amino acid sequence of SEQ ID NO: 87 or 168, and the V_{L-1} comprises an amino acid sequence of SEQ ID NO: 89 or 170. In some embodiments, the V_{H-1} comprises an amino acid sequence of SEQ ID NO: 87, and the V_{L-1} comprises an amino acid sequence of SEQ ID NO: 89. In some embodiments, the V_{H-1} comprises an amino acid sequence of SEQ ID NO: 168, and the V_{L-1} comprises an amino acid sequence of SEQ ID NO: 170. In some embodiments, the V_{H-2} comprises an amino acid sequence of SEQ ID NO: 60, 164, or 166, and the V_{L-2} comprises an amino acid sequence of SEQ ID NO: 61, 165, or 167. In some embodiments, the V_{H-2} comprises an amino acid sequence of SEQ ID NO: 164, and the V_{L-2} comprises an amino acid sequence of SEQ ID NO: 165. In some embodiments, the V_{H-2} comprises an amino acid sequence of SEQ ID NO: 166, and the V_{L-2} comprises an amino acid sequence of SEQ ID NO: 167. In some embodiments, the V_{H-2} comprises an amino acid sequence of SEQ ID NO: 61, and the V_{L-2} comprises an amino acid sequence of SEQ ID NO: 167. In some embodiments, the first antibody moiety is a full-length antibody comprising two heavy chains and two light chains, and the second antibody moiety is a scFv comprising the V_{H-2} and V_{L-2}. In some embodiments, the V_{H-2} is fused to the N-terminus of the V_{L-2}. In some embodiments, the V_{H-2} is fused to the C-terminus of the V_{L-2} optionally with a single alanine amino acid appended to the C-terminus of V_{H-2}. In some embodiments, the V_{H-2} and the V_{L-2} are fused via a linker (e.g., a GS linker, e.g., (GGGGS)₄). In some embodiments, the V_{H-1} and the V_{L-1} are fused without a linker. In some embodiments, the scFv is a dsscFv optionally comprising a) a G44C mutation in the V_{H} or V_{H-2} according to the numbering of SEQ ID NO: 60, and b) a G100C mutation in the V_{L} or V_{L-2} according to the numbering of SEQ ID NO: 61. In some embodiments, the second antibody moiety is fused to N-terminus of two heavy chains of the full-length antibody. In some embodiments, the second antibody moiety is fused to C-terminus of two heavy chains of the full-length antibody. In some embodiments, the second antibody moiety is fused to N-terminus of two light chains of the full-length antibody. In some embodiments, the second antibody moiety is fused to C-terminus of two light chains of the full-length antibody. In some embodiments, the second antibody moiety is a full-length antibody comprising two heavy chains and two light chains, and the first antibody moiety is a scFv comprising the V_{H-1} and the V_{L-1}. In some embodiments, the V_{H-1} is fused to the N-terminus of the V_{L-1}. In some embodiments, the V_{H-1} is fused to the C-terminus of the V_{L-1} optionally with a single alanine amino acid appended to the C-terminus of V_{H-1}. In some embodiments, the V_{H-1} and the V_{L-1} are fused via a linker (e.g., a GS linker, e.g., (GGGGS)₄). In some embodiments, the V_{H-1} and the V_{L-1} are fused without a linker. In some embodiments, the scFv is converted to a dsscFv with H44-L100 disulfide bond formed by G44C mutation on V_{H-1} and G100C mutation on V_{L-1}. In some embodiments, the first antibody moiety is fused to N-terminus of one or two heavy chains of the full-length antibody. In some embodiments, the first antibody moiety is fused to C-terminus of one or two heavy chains of the full-length antibody. In some embodiments, the first antibody moiety is fused to N-terminus of one or two light chains of the full-length antibody. In some embodiments, the first antibody moiety is fused to C-terminus of one or two light chains of the full-length antibody. In some embodiments, the first antibody moiety and the second antibody moiety are fused via a linker (e.g., a GS linker, e.g., (GGGGS)₃). In some embodiments, the first antibody moiety and the second antibody moiety are fused without a linker.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 115, wherein the two light chains each comprises an amino acid sequence of SEQ ID NO: 119.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 116, wherein the two light chains each comprises an amino acid sequence of SEQ ID NO: 119.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 120, wherein the two light chains each comprises an amino acid sequence of SEQ ID NO: 122.

In some embodiments, there is provided a multispecific (e.g., bispecific) construct comprising a first antibody moiety that specifically recognizes Sclerostin and a second antibody moiety that specifically recognizes DKK1, wherein the multispecific construct comprises two heavy chains and two light chains, wherein the two heavy chains each comprises an amino acid sequence of SEQ ID NO: 121, wherein the two light chains each comprises an amino acid sequence of SEQ ID NO: 122.

In some embodiments, there is provided a multispecific (e.g., bispecific) anti-Sclerostin construct comprising a) a first antibody moiety that specifically recognizes Sclerostin comprising a first heavy chain variable region (V_{H-1}) and a first light chain variable region (V_{L-1}), wherein the V_{H-1} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9, and the V_{L-1} comprises the LC-CDR1 comprising the amino acid sequence of SEQ ID NO: 85, the LC-CDR2 comprising the amino acid sequence of SEQ ID NO: 18, and the LC-CDR3 comprising the amino acid sequence of SEQ ID NO: 20, and b) a second antibody moiety that specifically recognizes DKK1 comprising a second heavy chain variable region (V_{H-2}) and a second light chain variable region (V_{L-2}), wherein the V_{H-2} comprises the HC-CDR1 comprising the amino acid sequence of SEQ ID NO: 48, the HC-CDR2 comprising the amino acid sequence of SEQ ID NO: 49, and the HC-CDR3 comprising the amino acid sequence of SEQ ID NO: 50, and the V_{L-2} comprises the LC-CDR1 comprising the amino acid sequence of SEQ ID NO: 51, the LC-CDR2 comprising the amino acid sequence of SEQ ID NO: 52, and the LC-CDR3 comprising the amino acid sequence of SEQ ID NO: 58. In some embodiments, the V_{H-1} comprises an amino acid sequence of SEQ ID NO: 87 or 168, and the V_{L-1} comprises an amino acid sequence of SEQ ID NO: 89 or 170. In some embodiments, the V_{H-1} comprises an amino acid sequence of SEQ ID NO: 87, and the V_{L-1} comprises an amino acid sequence of SEQ ID NO: 89. In some embodiments, the V_{H-1} comprises an amino acid sequence of SEQ ID NO: 168, and the V_{L-1} comprises an amino acid sequence of SEQ ID NO: 170. In some embodiments, the V_{H-2} comprises an amino acid sequence of SEQ ID NO: 62, and the V_{L-2} comprises an amino acid sequence of SEQ ID NO: 63. In some embodiments, the first antibody moiety is a full-length antibody comprising two heavy chains and two light chains, and the second antibody moiety is a scFv comprising the V_{H-2} and V_{L-2}. In some embodiments, the V_{H-2} is fused to the N-terminus of the V_{L-2} optionally with a single alanine amino acid appended to the C-terminus of V_{H-2}. In some embodiments, the V_{H-2} is fused to the C-terminus of the V_{L-2}. In some embodiments, the V_{H-2} and the V_{L-2} are fused via a linker (e.g., a GS linker, e.g., (GGGGS)₄). In some embodiments, the V_{H-1} and the V_{L-1} are fused without a linker. In some embodiments, the scFv is a dsscFv optionally comprising a) a G44C mutation in the V_{H} or V_{H-2} according to the numbering of SEQ ID NO: 60, and b) a G100C mutation in the V_{L} or V_{L-2} according to the numbering of SEQ ID NO: 61. In some embodiments, the second antibody moiety is fused to N-terminus of two heavy chains of the full-length antibody. In some embodiments, the second antibody moiety is fused to C-terminus of two heavy chains of the full-length antibody. In some embodiments, the second antibody moiety is fused to N-terminus of two light chains of the full-length antibody. In some embodiments, the second antibody moiety is fused to C-terminus of two light chains of the full-length antibody. In some embodiments, the second antibody moiety is a full-length antibody comprising two heavy chains and two light chains, and the first antibody moiety is a scFv comprising the V_{H-1} and the V_{L-1}. In some embodiments, the V_{H-1} is fused to the N-terminus of the V_{L-1}. In some embodiments, the V_{H-1} is fused to the C-terminus of the V_{L-1} optionally with a single alanine amino acid appended to the C-terminus of V_{H-1}. In some embodiments, the V_{H-1} and the V_{L-1} are fused via a linker (e.g., a GS linker, e.g., (GGGGS)₄). In some embodiments, the V_{H-1} and the V_{L-1} are fused without a linker. In some embodiments, the scFv is a dsscFv optionally comprising a) a G44C mutation in the V_{H} or V_{H-2} according to the numbering of SEQ ID NO: 60, and b) a G100C mutation in the V_{L} or V_{L-2} according to the numbering of SEQ ID NO: 61. In some embodiments, the first antibody moiety is fused to N-terminus of one or two heavy chains of the full-length antibody. In some embodiments, the first antibody moiety is fused to C-terminus of one or two heavy chains of the full-length antibody. In some embodiments, the first antibody moiety is fused to N-terminus of one or two light chains of the full-length antibody. In some embodiments, the first antibody moiety is fused to C-terminus of one or two light chains of the full-length antibody. In some embodiments, the first antibody moiety and the second antibody moiety are fused via a linker (e.g., a GS linker, e.g., (GGGGS)₃). In some embodiments, the first antibody moiety and the second antibody moiety are fused without a linker.

In some embodiments, there is provided a multispecific (e.g., bispecific) anti-Sclerostin construct comprising a) a first antibody moiety that specifically recognizes Sclerostin comprising a first heavy chain variable region (V_{H-1}) and a first light chain variable region (V_{L-1}), wherein the V_{H-1} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9, and the V_{L-1} comprises the LC-CDR1 comprising the amino acid sequence of SEQ ID NO: 85, the LC-CDR2 comprising the amino acid sequence of SEQ ID NO: 18, and the LC-CDR3 comprising the amino acid sequence of SEQ ID NO: 20, and b) a second antibody moiety that specifically recognizes DKK1 comprising a second heavy chain variable region (V_{H-2}) and a second light chain variable region (V_{L-2}), wherein the V_{H-2} comprises the HC-CDR1 comprising the amino acid sequence of SEQ ID NO: 54, the HC-CDR2 comprising the amino acid sequence of SEQ ID NO: 55, and the HC-CDR3 comprising the amino acid sequence of SEQ ID NO: 56, and the V_{L-2} comprises the LC-CDR1 comprising the amino acid sequence of SEQ ID NO: 57, the LC-CDR2 comprising the amino acid sequence of SEQ ID NO: 58, and the LC-CDR3 comprising the amino acid sequence of SEQ ID NO: 59. In some embodiments, the V_{H-1} comprises an amino acid sequence of SEQ ID NO: 87 or 168, and the V_{L-1} comprises an amino acid sequence of SEQ ID NO: 89 or 170. In some embodiments, the V_{H-1} comprises an amino acid sequence of SEQ ID NO: 87, and the V_{L-1} comprises an amino acid sequence of SEQ ID NO: 89. In some embodiments, the V_{H-1} comprises an amino acid sequence of SEQ ID NO: 168, and the V_{L-1} comprises an amino acid sequence of SEQ ID NO: 170. In some embodiments, the V_{H-2} comprises an amino acid sequence of SEQ ID NO: 64, and the V_{L-2} comprises an amino acid sequence of SEQ ID NO: 65. In some embodiments, the first antibody moiety is a full-length antibody comprising two heavy chains and two light chains, and the second antibody moiety is a scFv comprising the V_{H-2} and V_{L-2}. In some embodiments, the V_{H-2} is fused to the N-terminus of the V_{L-2}. In some embodiments, the V_{H-2} is fused to the C-terminus of the V_{L-2} optionally with a single alanine amino acid appended to the C-terminus of V_{H-2}. In some embodiments, the V_{H-2} and the V_{L-2} are fused via a linker (e.g., a GS linker, e.g., (GGGGS)₄). In some embodiments, the V_{H-1} and the V_{L-1} are fused without a linker. In some embodiments, the scFv is a dsscFv optionally comprising a) a G44C mutation in the V_{H} or V_{H-2} according to the numbering of SEQ ID NO: 60, and b) a G100C mutation in the V_{L} or V_{L-2} according to the numbering of SEQ ID NO: 61. In some embodiments, the second antibody moiety is fused to N-terminus of two heavy chains of the full-length antibody. In some embodiments, the second antibody moiety is fused to C-terminus of two heavy chains of the full-length antibody. In some embodiments, the second antibody moiety is fused to N-terminus of two light chains of the full-length antibody. In some embodiments, the second antibody moiety is fused to C-terminus of two light chains of the full-length antibody. In some embodiments, the second antibody moiety is a full-length antibody comprising two heavy chains and two light chains, and the first antibody moiety is a scFv comprising the V_{H-1} and the V_{L-1}. In some embodiments, the V_{H-1} is fused to the N-terminus of the V_{L-1}. In some embodiments, the V_{H-1} is fused to the C-terminus of the V_{L-1} optionally with a single alanine amino acid appended to the C-terminus of V_{H-2}. In some embodiments, the V_{H-1} and the V_{L-1} are fused via a linker (e.g., a GS linker, e.g., (GGGGS)₄). In some embodiments, the V_{H-1} and the V_{L-1} are fused without a linker. In some embodiments, the scFv is a dsscFv optionally comprising a) a G44C mutation in the V_{H} or V_{H-2} according to the numbering of SEQ ID NO: 60, and b) a G100C mutation in the V_{L} or V_{L-2} according to the numbering of SEQ ID NO: 61. In some embodiments, the first antibody moiety is fused to N-terminus of one or two heavy chains of the full-length antibody. In some embodiments, the first antibody moiety is fused to C-terminus of one or two heavy chains of the full-length antibody. In some embodiments, the first antibody moiety is fused to N-terminus of one or two light chains of the full-length antibody. In some embodiments, the first antibody moiety is fused to C-terminus of one or two light chains of the full-length antibody. In some embodiments, the first antibody moiety and the second antibody moiety are fused via a linker (e.g., a GS linker, e.g., (GGGGS)₃). In some embodiments, the first antibody moiety and the second antibody moiety are fused without a linker.

In some embodiments, there is provided a multispecific (e.g., bispecific) anti-Sclerostin construct comprising a) a first antibody moiety that specifically recognizes Sclerostin comprising a first heavy chain variable region (V_{H-1}) and a first light chain variable region (V_{L-1}), wherein the V_{H-1} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9, and the V_{L-1} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of SEQ ID NO: 20, and b) a second antibody moiety that specifically recognizes DKK1 comprising a second heavy chain variable region (V_{H-2}) and a second light chain variable region (V_{L-2}), wherein the V_{H-2} comprises the HC-CDR1 comprising the amino acid sequence of SEQ ID NO: 48, the HC-CDR2 comprising the amino acid sequence of SEQ ID NO: 49, and the HC-CDR3 comprising the amino acid sequence of SEQ ID NO: 50, and the V_{L-2} comprises the LC-CDR1 comprising the amino acid sequence of SEQ ID NO: 51, the LC-CDR2 comprising the amino acid sequence of SEQ ID NO: 52, and the LC-CDR3 comprising the amino acid sequence of SEQ ID NO: 58. In some embodiments, the V_{H-1} comprises an amino acid sequence of SEQ ID NO: 22, and the V_{L-1} comprises an amino acid sequence of SEQ ID NO: 23. In some embodiments, the V_{H-1} comprises an amino acid sequence of SEQ ID NO: 87 or 168, and the V_{L-1} comprises an amino acid sequence of SEQ ID NO: 88 or 169. In some embodiments, the V_{H-1} comprises an amino acid sequence of SEQ ID NO: 87, and the V_{L-1} comprises an amino acid sequence of SEQ ID NO: 88. In some embodiments, the V_{H-1} comprises an amino acid sequence of SEQ ID NO: 168, and the V_{L-1} comprises an amino acid sequence of SEQ ID NO: 169. In some embodiments, the V_{H-2} comprises an amino acid sequence of SEQ ID NO: 62, and the V_{L-2} comprises an amino acid sequence of SEQ ID NO: 63. In some embodiments, the first antibody moiety is a full-length antibody comprising two heavy chains and two light chains, and the second antibody moiety is a scFv comprising the V_{H-2} and V_{L-2}. In some embodiments, the V_{H-2} is fused to the N-terminus of the V_{L}-2. In some embodiments, the V_{H-2} is fused to the C-terminus of the V_{L-2} optionally with a single alanine amino acid appended to the C-terminus of V_{H-2}. In some embodiments, the V_{H-2} and the V_{L-2} are fused via a linker (e.g., a GS linker, e.g., (GGGGS)₄). In some embodiments, the V_{H-1} and the V_{L-1} are fused without a linker. In some embodiments, the scFv is a dsscFv optionally comprising a) a G44C mutation in the V_{H} or V_{H-2} according to the numbering of SEQ ID NO: 60, and b) a G100C mutation in the V_{L} or V_{L-2} according to the numbering of SEQ ID NO: 61. In some embodiments, the second antibody moiety is fused to N-terminus of two heavy chains of the full-length antibody. In some embodiments, the second antibody moiety is fused to C-terminus of two heavy chains of the full-length antibody. In some embodiments, the second antibody moiety is fused to N-terminus of two light chains of the full-length antibody. In some embodiments, the second antibody moiety is fused to C-terminus of two light chains of the full-length antibody. In some embodiments, the second antibody moiety is a full-length antibody comprising two heavy chains and two light chains, and the first antibody moiety is a scFv comprising the V_{H-1} and the V_{L-1}. In some embodiments, the V_{H-1} is fused to the N-terminus of the V_{L-1}. In some embodiments, the V_{H-1} is fused to the C-terminus of the V_{L-1} optionally with a single alanine amino acid appended to the C-terminus of V_{H-1}. In some embodiments, the V_{H-1} and the V_{L-1} are fused via a linker (e.g., a GS linker, e.g., (GGGGS)₄). In some embodiments, the V_{H-1} and the V_{L-1} are fused without a linker. In some embodiments, the scFv is a dsscFv optionally comprising a) a G44C mutation in the V_{H} or V_{H-2} according to the numbering of SEQ ID NO: 60, and b) a G100C mutation in the V_{L} or V_{L-2} according to the numbering of SEQ ID NO: 61. In some embodiments, the first antibody moiety is fused to N-terminus of one or two heavy chains of the full-length antibody. In some embodiments, the first antibody moiety is fused to C-terminus of one or two heavy chains of the full-length antibody. In some embodiments, the first antibody moiety is fused to N-terminus of one or two light chains of the full-length antibody. In some embodiments, the first antibody moiety is fused to C-terminus of one or two light chains of the full-length antibody. In some embodiments, the first antibody moiety and the second antibody moiety are fused via a linker (e.g., a GS linker, e.g., (GGGGS)₃). In some embodiments, the first antibody moiety and the second antibody moiety are fused without a linker.

In some embodiments, there is provided a multispecific (e.g., bispecific) anti-Sclerostin construct comprising a) a first antibody moiety that specifically recognizes Sclerostin comprising a first heavy chain variable region (V_{H-1}) and a first light chain variable region (V_{L-1}), wherein the V_{H-1} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9, and the V_{L-1} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of SEQ ID NO: 20, and b) a second antibody moiety that specifically recognizes DKK1 comprising a second heavy chain variable region (V_{H-2}) and a second light chain variable region (V_{L-2}), wherein the V_{H-2} comprises the HC-CDR1 comprising the amino acid sequence of SEQ ID NO: 54, the HC-CDR2 comprising the amino acid sequence of SEQ ID NO: 55, and the HC-CDR3 comprising the amino acid sequence of SEQ ID NO: 56, and the V_{L-2} comprises the LC-CDR1 comprising the amino acid sequence of SEQ ID NO: 57, the LC-CDR2 comprising the amino acid sequence of SEQ ID NO: 58, and the LC-CDR3 comprising the amino acid sequence of SEQ ID NO: 59. In some embodiments, the V_{H-1} comprises an amino acid sequence of SEQ ID NO: 22, and the V_{L-1} comprises an amino acid sequence of SEQ ID NO: 23. In some embodiments, the V_{H-1} comprises an amino acid sequence of SEQ ID NO: 87 or 168, and the V_{L-1} comprises an amino acid sequence of SEQ ID NO: 88 or 169. In some embodiments, the V_{H-1} comprises an amino acid sequence of SEQ ID NO: 87, and the V_{L-1} comprises an amino acid sequence of SEQ ID NO: 88. In some embodiments, the V_{H-1} comprises an amino acid sequence of SEQ ID NO: 168, and the V_{L-1} comprises an amino acid sequence of SEQ ID NO: 169. In some embodiments, the V_{H-2} comprises an amino acid sequence of SEQ ID NO: 64, and the V_{L-2} comprises an amino acid sequence of SEQ ID NO: 65. In some embodiments, the first antibody moiety is a full-length antibody comprising two heavy chains and two light chains, and the second antibody moiety is a scFv comprising the V_{H-2} and V_{L-2}. In some embodiments, the V_{H-2} is fused to the N-terminus of the V_{L-2}. In some embodiments, the V_{H-2} is fused to the C-terminus of the V_{L-2} optionally with a single alanine amino acid appended to the C-terminus of V_{H-2}. In some embodiments, the V_{H-2} and the V_{L-2} are fused via a linker (e.g., a GS linker, e.g., (GGGGS)₄). In some embodiments, the V_{H-1} and the V_{L-1} are fused without a linker. In some embodiments, the scFv is a dsscFv optionally comprising a) a G44C mutation in the V_{H} or V_{H-2} according to the numbering of SEQ ID NO: 60, and b) a G100C mutation in the V_{L} or V_{L-2} according to the numbering of SEQ ID NO: 61. In some embodiments, the second antibody moiety is fused to N-terminus of two heavy chains of the full-length antibody. In some embodiments, the second antibody moiety is fused to C-terminus of two heavy chains of the full-length antibody. In some embodiments, the second antibody moiety is fused to N-terminus of two light chains of the full-length antibody. In some embodiments, the second antibody moiety is fused to C-terminus of two light chains of the full-length antibody. In some embodiments, the second antibody moiety is a full-length antibody comprising two heavy chains and two light chains, and the first antibody moiety is a scFv comprising the V_{H-1} and the V_{L-1}. In some embodiments, the V_{H-1} is fused to the N-terminus of the V_{L-1}. In some embodiments, the V_{H-1} is fused to the C-terminus of the V_{L-1} optionally with a single alanine amino acid appended to the C-terminus of V_{H-1}. In some embodiments, the V_{H-1} and the V_{L-1} are fused via a linker (e.g., a GS linker, e.g., (GGGGS)₄). In some embodiments, the V_{H-1} and the V_{L-1} are fused without a linker. In some embodiments, the scFv is a dsscFv optionally comprising a) a G44C mutation in the V_{H} or V_{H-2} according to the numbering of SEQ ID NO: 60, and b) a G100C mutation in the V_{L} or V_{L-2} according to the numbering of SEQ ID NO: 61. In some embodiments, the first antibody moiety is fused to N-terminus of one or two heavy chains of the full-length antibody. In some embodiments, the first antibody moiety is fused to C-terminus of one or two heavy chains of the full-length antibody. In some embodiments, the first antibody moiety is fused to N-terminus of one or two light chains of the full-length antibody. In some embodiments, the first antibody moiety is fused to C-terminus of one or two light chains of the full-length antibody. In some embodiments, the first antibody moiety and the second antibody moiety are fused via a linker (e.g., a GS linker, e.g., (GGGGS)₃). In some embodiments, the first antibody moiety and the second antibody moiety are fused without a linker.

In some embodiments, there is provided a multispecific construct specifically recognizing Sclerostin and DKK1, comprising a first antibody moiety and a second antibody, wherein the first antibody moiety comprises a first heavy chain variable region (V_{H-1}) and a first light chain variable region (V_{L-1}), and wherein the second antibody moiety comprises a second heavy chain variable region (V_{H-2}) and a second light chain variable region (V_{L-2}), wherein the construct comprises: a) a first polypeptide comprising a first light chain comprising, from N-terminus to C-terminus, i) the V_{L-1}, ii) a first light chain constant domain (“first CL domain”); b) a second polypeptide comprising a first heavy chain, from N-terminus to C-terminus, i) the V_{H-1}, ii) a first heavy chain constant domain (“first CH1 domain”), and iii) a first Fc domain; c) a third polypeptide comprising a second heavy chain comprising, from N-terminus to C-terminus, i) the V_{H-2}, ii) a second heavy chain constant domain (“second CH1 domain”), and iii) a second Fc domain; and d) a fourth polypeptide comprising a second light chain, from N-terminus to C-terminus, i) the V_{L-2}, ii) ii) a first light chain constant domain (“the second CL domain”), wherein the first and the second Fc domains form a Fc fragment. In some embodiments, the first antibody moiety specifically recognizes Sclerostin, and the second antibody moiety specifically recognizes DKK1. In some embodiments, the first antibody moiety specifically recognizes DKK1, and the second antibody moiety specifically recognizes Sclerostin.

In some embodiments, one of the first and the second Fc domains comprises a T366W mutation, and optionally a S354C mutation, and wherein the other Fc domain comprises a T366S mutation, a L368A mutation, a Y407V mutation, and optionally a Y349C mutation, wherein numbering is according to the EU index.

In some embodiments, either i) the first CH1 domain and the first CL domain or ii) the second CH1 domain and the second CL domain are selected from the group consisting of: a) a CH1 domain wherein the amino acid at position 141 is substituted for cysteine and the cysteine at position 131 (e.g., in IgG2 or IgG4) or 220 (e.g., in IgG1) is substituted for a non-cysteine amino acid; and a CL domain wherein the amino acid at position 116 is substituted for cysteine and the cysteine at position 214 is substituted for a non-cysteine amino acid; b) a CH1 domain wherein the amino acid at position 168 is substituted for cysteine and the cysteine at position 131 (e.g., in IgG2 or IgG4) or 220 (e.g., in IgG1) is substituted for a non-cysteine amino acid; and a CL domain wherein the amino acid at position 164 is substituted for cysteine and the cysteine at position 214 is substituted for a non-cysteine amino acid; c) a CH1 domain wherein the amino acid at position 126 is 131 or substituted for cysteine and the cysteine at position 220 is substituted for a non-cysteine amino acid; and a CL domain wherein the amino acid at position 121 is substituted for cysteine and the cysteine at position 214 is substituted for a non-cysteine amino acid; d) a CH1 domain wherein the amino acid at position 128 is substituted for cysteine and the cysteine at position 131 (e.g., in IgG2 or IgG4) or 220 (e.g., in IgG1) is substituted for a non-cysteine amino acid; and a CL domain wherein the amino acid at position 118 is substituted for cysteine and the cysteine at position 214 is substituted for a non-cysteine amino acid; e) a CH1 domain wherein the amino acid at position 170 is substituted for cysteine and the cysteine at position 131 (e.g., in IgG2 or IgG4) or 220 (e.g., in IgG1) is substituted for a non-cysteine amino acid; and a CL domain wherein the amino acid at position 176 is substituted for cysteine and the cysteine at position 214 is substituted for a non-cysteine amino acid; f) a CH1 domain wherein the amino acid at position 171 is substituted for cysteine and the cysteine at position 131 (e.g., in IgG2 or IgG4) or 220 (e.g., in IgG1) is substituted for a non-cysteine amino acid; and a CL domain wherein the amino acid at position 162 is substituted for cysteine and the cysteine at position 214 is substituted for a non-cysteine amino acid; g) a CH1 domain wherein the amino acid at position 173 is substituted for cysteine and the cysteine at position 131 (e.g., in IgG2 or IgG4) or 220 (e.g., in IgG1) is substituted for a non-cysteine amino acid; and a CL domain wherein the amino acid at position 160 is substituted for cysteine and the cysteine at position 214 is substituted for a non-cysteine amino acid; wherein numbering is according to the EU index.

In some embodiments, the first heavy chain comprises a modified human IgG4 heavy chain Fc region comprising S228P, T366W, H435R, and Y436F; the second heavy chain comprises a modified human IgG4 heavy chain Fc region comprising F126C, C131S, S228P, T366S, L368A, and Y407V; and the second light chain comprises a modified human Ig kappa light chain constant region comprising S121C and C214S.

In some embodiments, the first heavy chain comprises a modified human IgG4 heavy chain Fc region comprising S228P, T366S, L368A, and Y407V; the second heavy chain comprises a modified human IgG4 heavy chain Fc region comprising F126C, C131S, S228P, T366W, H435R, and Y436F; and the second light chain comprises a modified human Ig kappa light chain constant region comprising S121C and C214S.

In some embodiments, the first heavy chain comprises a modified human IgG4 heavy chain Fc region comprising S228P, T366W, H435R, and Y436F; the second heavy chain comprises a modified human IgG4 heavy chain Fc region comprising L128C, C131S, S228P, T366S, L368A, and Y407V; and the second light chain comprises a modified human Ig kappa light chain constant region comprising F118C and C214S.

In some embodiments, the first heavy chain comprises a modified human IgG4 heavy chain Fc region comprising S228P, T366S, L368A, and Y407V; the second heavy chain comprises a modified human IgG4 heavy chain Fc region comprising L128C, C131S, S228P, T366W, H435R, and Y436F; and the second light chain comprises a modified human Ig kappa light chain constant region comprising F118C and C214S.

In some embodiments, the first heavy chain comprises a modified human IgG4 heavy chain Fc region comprising S228P, T366S, L368A, and Y407V; the second heavy chain comprises a modified human IgG4 heavy chain Fc region comprising F170C, C131S, S228P, T366W, H435R, and Y436F; and the second light chain comprises a modified human Ig kappa light chain constant region comprising S176C and C214S.

In some embodiments, the first heavy chain comprises a modified human IgG4 heavy chain Fc region comprising S228P, T366S, L368A, and Y407V; the second heavy chain comprises a modified human IgG4 heavy chain Fc region comprising V173C, C131S, S228P, T366W, H435R, and Y436F; and the second light chain comprises a modified human Ig kappa light chain constant region comprising Q160C and C214S.

In some embodiments, the first antibody moiety specifically recognizing Sclerostin, and the second antibody moiety specifically recognizing DKK1. In some embodiments, the V_{H-1} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9, and the V_{L-1} comprises the LC-CDR1 comprising the amino acid sequence of SEQ ID NO: 15 or 85, the LC-CDR2 comprising the amino acid sequence of SEQ ID NO: 18, and the LC-CDR3 comprising the amino acid sequence of SEQ ID NO: 20. In some embodiments, the V_{H-1} comprises an amino acid sequence of SEQ ID NO: 22, and the V_{L-1} comprises an amino acid sequence of SEQ ID NO: 23. In some embodiments, the V_{H-1} comprises an amino acid sequence of SEQ ID NO: 87 or 168, and the V_{L-1} comprises an amino acid sequence of SEQ ID NO: 88 or 169. In some embodiments, the V_{H-1} comprises an amino acid sequence of SEQ ID NO: 87, and the V_{L-1} comprises an amino acid sequence of SEQ ID NO: 88. In some embodiments, the V_{H-1} comprises an amino acid sequence of SEQ ID NO: 168, and the V_{L-1} comprises an amino acid sequence of SEQ ID NO: 169. In some embodiments, the V_{H-1} comprises an amino acid sequence of SEQ ID NO: 87 or 168, and the V_{L-1} comprises an amino acid sequence of SEQ ID NO: 89 or 160. In some embodiments, the V_{H-1} comprises an amino acid sequence of SEQ ID NO: 87, and the V_{L-1} comprises an amino acid sequence of SEQ ID NO: 89. In some embodiments, the V_{H-1} comprises an amino acid sequence of SEQ ID NO: 168, and the V_{L-1} comprises an amino acid sequence of SEQ ID NO: 170. In some embodiments, a) the V_{H-2} comprises the HC-CDR1 comprising the amino acid sequence of SEQ ID NO: 42, the HC-CDR2 comprising the amino acid sequence of SEQ ID NO: 43, and the HC-CDR3 comprising the amino acid sequence of SEQ ID NO: 44, and the V_{L-2} comprises the LC-CDR1 comprising the amino acid sequence of SEQ ID NO: 45, the LC-CDR2 comprising the amino acid sequence of SEQ ID NO: 46, and the LC-CDR3 comprising the amino acid sequence of SEQ ID NO: 47; b) the V_{H-2} comprises the HC-CDR1 comprising the amino acid sequence of SEQ ID NO: 48, the HC-CDR2 comprising the amino acid sequence of SEQ ID NO: 49, and the HC-CDR3 comprising the amino acid sequence of SEQ ID NO: 50, and the V_{L-2} comprises the LC-CDR1 comprising the amino acid sequence of SEQ ID NO: 51, the LC-CDR2 comprising the amino acid sequence of SEQ ID NO: 52, and the LC-CDR3 comprising the amino acid sequence of SEQ ID NO: 58; or c) the V_{H-2} comprises the HC-CDR1 comprising the amino acid sequence of SEQ ID NO: 54, the HC-CDR2 comprising the amino acid sequence of SEQ ID NO: 55, and the HC-CDR3 comprising the amino acid sequence of SEQ ID NO: 56, and the V_{L-2} comprises the LC-CDR1 comprising the amino acid sequence of SEQ ID NO: 57, the LC-CDR2 comprising the amino acid sequence of SEQ ID NO: 58, and the LC-CDR3 comprising the amino acid sequence of SEQ ID NO: 59.

In some embodiments, the first antibody moiety specifically recognizing DKK1, and the second antibody moiety specifically recognizing Sclerostin. In some embodiments, the V_{H-2} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9, and the V_{L-2} comprises the LC-CDR1 comprising the amino acid sequence of SEQ ID NO: 15 or 85, the LC-CDR2 comprising the amino acid sequence of SEQ ID NO: 18, and the LC-CDR3 comprising the amino acid sequence of SEQ ID NO: 20. In some embodiments, the V_{H-2} comprises an amino acid sequence of SEQ ID NO: 22, and the V_{L-2} comprises an amino acid sequence of SEQ ID NO: 23. In some embodiments, the V_{H-2} comprises an amino acid sequence of SEQ ID NO: 87 or 168, and the V_{L-2} comprises an amino acid sequence of SEQ ID NO: 88 or 169. In some embodiments, the V_{H-2} comprises an amino acid sequence of SEQ ID NO: 87, and the V_{L-2} comprises an amino acid sequence of SEQ ID NO: 88. In some embodiments, the V_{H-2} comprises an amino acid sequence of SEQ ID NO: 168, and the V_{L-2} comprises an amino acid sequence of SEQ ID NO: 169. In some embodiments, the V_{H-2} comprises an amino acid sequence of SEQ ID NO: 87 or 169, and the V_{L-2} comprises an amino acid sequence of SEQ ID NO: 89 or 170. In some embodiments, the V_{H-2} comprises an amino acid sequence of SEQ ID NO: 87, and the V_{L-2} comprises an amino acid sequence of SEQ ID NO: 89. In some embodiments, the V_{H-2} comprises an amino acid sequence of SEQ ID NO: 168, and the V_{L-2} comprises an amino acid sequence of SEQ ID NO: 170. In some embodiments, a) the V_{H-1} comprises the HC-CDR1 comprising the amino acid sequence of SEQ ID NO: 42, the HC-CDR2 comprising the amino acid sequence of SEQ ID NO: 43, and the HC-CDR3 comprising the amino acid sequence of SEQ ID NO: 44, and the V_{L-1} comprises the LC-CDR1 comprising the amino acid sequence of SEQ ID NO: 45, the LC-CDR2 comprising the amino acid sequence of SEQ ID NO: 46, and the LC-CDR3 comprising the amino acid sequence of SEQ ID NO: 47; b) the V_{H-1} comprises the HC-CDR1 comprising the amino acid sequence of SEQ ID NO: 48, the HC-CDR2 comprising the amino acid sequence of SEQ ID NO: 49, and the HC-CDR3 comprising the amino acid sequence of SEQ ID NO: 50, and the V_{L-1} comprises the LC-CDR1 comprising the amino acid sequence of SEQ ID NO: 51, the LC-CDR2 comprising the amino acid sequence of SEQ ID NO: 52, and the LC-CDR3 comprising the amino acid sequence of SEQ ID NO: 58; or c) the V_{H-1} comprises the HC-CDR1 comprising the amino acid sequence of SEQ ID NO: 54, the HC-CDR2 comprising the amino acid sequence of SEQ ID NO: 55, and the HC-CDR3 comprising the amino acid sequence of SEQ ID NO: 56, and the V_{L-1} comprises the LC-CDR1 comprising the amino acid sequence of SEQ ID NO: 57, the LC-CDR2 comprising the amino acid sequence of SEQ ID NO: 58, and the LC-CDR3 comprising the amino acid sequence of SEQ ID NO: 59.

In some embodiments, there is provided a multispecific construct comprising a first antibody moiety that specifically recognizes Sclerostin and a second antibody moiety that specifically recognizes DKK1, wherein the first antibody moiety is an anti-Sclerostin full-length antibody comprising two heavy chains and two light chains, wherein the two heavy chains each comprises a first heavy chain variable region (V_{H-1}), wherein the two light chains each comprises a first light chain variable region (V_{L-1}), and wherein the second antibody moiety comprises an anti-DKK1 single chain Fv fragment (scFv) comprising a second heavy chain variable region (V_{H-2}) and a second light chain variable region (V_{L-2}), wherein the second antibody moiety is fused to C-terminus of both heavy chains of the anti-Sclerostin full-length antibody. In some embodiments, the V_{H-1} comprises an amino acid sequence of SEQ ID NO: 22, or a variant comprising an amino acid sequence having at least about 80% (such as at least about any one of 80%, 85%, 90%, 95%, 96%, 97%, 98%, or 99%) sequence identity; and the V_{L-1} comprises an amino acid sequence of SEQ ID NO: 23, or a variant comprising an amino acid sequence having at least about 80% (such as at least about any one of 80%, 85%, 90%, 95%, 96%, 97%, 98%, or 99%) sequence identity. In some embodiments, the V_{H-2} comprises the HC-CDR1 comprising the amino acid sequence of SEQ ID NO: 42, the HC-CDR2 comprising the amino acid sequence of SEQ ID NO: 43, and the HC-CDR3 comprising the amino acid sequence of SEQ ID NO: 44, and the V_{L-2} comprises the LC-CDR1 comprising the amino acid sequence of SEQ ID NO: 45, the LC-CDR2 comprising the amino acid sequence of SEQ ID NO: 46, and the LC-CDR3 comprising the amino acid sequence of SEQ ID NO: 47. In some embodiments, the V_{H-2} comprises an amino acid sequence of SEQ ID NO: 60, or a variant comprising an amino acid sequence having at least about 80% (such as at least about any one of 80%, 85%, 90%, 95%, 96%, 97%, 98%, or 99%) sequence identity; and the V_{L-1} comprises an amino acid sequence of SEQ ID NO: 61, or a variant comprising an amino acid sequence having at least about 80% (such as at least about any one of 80%, 85%, 90%, 95%, 96%, 97%, 98%, or 99%) sequence identity.

In some embodiments, there is provided a multispecific construct specifically recognizing Sclerostin and DKK1, comprising a first antibody moiety and a second antibody, wherein the first antibody moiety comprises a first heavy chain variable region (V_{H-1}) and a first light chain variable region (V_{L-1}), and wherein the second antibody moiety comprises a second heavy chain variable region (V_{H-2}) and a second light chain variable region (V_{L-2}), wherein the construct comprises: a) a first polypeptide comprising a first light chain comprising, from N-terminus to C-terminus, i) the V_{L-1}, ii) a first light chain constant domain (“first CL domain”); b) a second polypeptide comprising a first heavy chain, from N-terminus to C-terminus, i) the V_{H-1}, ii) a first heavy chain constant domain (“first CH1 domain”), and iii) a first Fc domain; c) a third polypeptide comprising a second heavy chain comprising, from N-terminus to C-terminus, i) the V_{H-2}, ii) a second heavy chain constant domain (“second CH1 domain”), and iii) a second Fc domain; and d) a fourth polypeptide comprising a second light chain, from N-terminus to C-terminus, i) the V_{L-2}, ii) ii) a first light chain constant domain (“the second CL domain”), wherein the first and the second Fc domains form a Fc fragment, wherein the first antibody moiety specifically recognizes Sclerostin, and wherein the second antibody moiety specifically recognizes DKK1. In some embodiments, the V_{H-1} comprises an amino acid sequence of SEQ ID NO: 22, or a variant comprising an amino acid sequence having at least about 80% (such as at least about any one of 80%, 85%, 90%, 95%, 96%, 97%, 98%, or 99%) sequence identity; and the V_{L-1} comprises an amino acid sequence of SEQ ID NO: 23, or a variant comprising an amino acid sequence having at least about 80% (such as at least about any one of 80%, 85%, 90%, 95%, 96%, 97%, 98%, or 99%) sequence identity. In some embodiments, the V_{H-2} comprises the HC-CDR1 comprising the amino acid sequence of SEQ ID NO: 42, the HC-CDR2 comprising the amino acid sequence of SEQ ID NO: 43, and the HC-CDR3 comprising the amino acid sequence of SEQ ID NO: 44, and the V_{L-2} comprises the LC-CDR1 comprising the amino acid sequence of SEQ ID NO: 45, the LC-CDR2 comprising the amino acid sequence of SEQ ID NO: 46, and the LC-CDR3 comprising the amino acid sequence of SEQ ID NO: 47. In some embodiments, the V_{H-2} comprises an amino acid sequence of SEQ ID NO: 60, or a variant comprising an amino acid sequence having at least about 80% (such as at least about any one of 80%, 85%, 90%, 95%, 96%, 97%, 98%, or 99%) sequence identity; and the V_{L-1} comprises an amino acid sequence of SEQ ID NO: 61, or a variant comprising an amino acid sequence having at least about 80% (such as at least about any one of 80%, 85%, 90%, 95%, 96%, 97%, 98%, or 99%) sequence identity.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 134; b) a first light chain comprising a first light chain variable region (V_{L-1}) and a first light chain constant region, wherein the first light chain comprises an amino acid sequence of SEQ ID NO: 135; c) a second heavy chain comprising a second variable heavy chain variable region (V_{H-2}) and a second heavy chain constant region, wherein the second heavy chain comprises the amino acid sequence of SEQ ID NO: 136; and d) a second light chain comprising a second light chain variable region (V_{L-2}) and a second light chain constant region, wherein the second light chain comprises the amino acid sequence of SEQ ID NO: 137, wherein the V_{H-1} and the V_{L-1} comprises a first antibody moiety specifically recognizing Sclerostin, wherein the V_{H-2} and the V_{L-2} comprises a second antibody moiety specifically recognizing DKK1.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 134; b) a first light chain comprising a first light chain variable region (V_{L-1}) and a first light chain constant region, wherein the first light chain comprises an amino acid sequence of SEQ ID NO: 135; c) a second heavy chain comprising a second variable heavy chain variable region (V_{H-2}) and a second heavy chain constant region, wherein the second heavy chain comprises the amino acid sequence of SEQ ID NO: 138; and d) a second light chain comprising a second light chain variable region (V_{L-2}) and a second light chain constant region, wherein the second light chain comprises the amino acid sequence of SEQ ID NO: 139, wherein the V_{H-1} and the V_{L-1} comprises a first antibody moiety specifically recognizing Sclerostin, wherein the V_{H-2} and the V_{L-2} comprises a second antibody moiety specifically recognizing DKK1.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 134; b) a first light chain comprising a first light chain variable region (V_{L-1}) and a first light chain constant region, wherein the first light chain comprises an amino acid sequence of SEQ ID NO: 135; c) a second heavy chain comprising a second variable heavy chain variable region (V_{H-2}) and a second heavy chain constant region, wherein the second heavy chain comprises the amino acid sequence of SEQ ID NO: 140; and d) a second light chain comprising a second light chain variable region (V_{L-2}) and a second light chain constant region, wherein the second light chain comprises the amino acid sequence of SEQ ID NO: 141, wherein the V_{H-1} and the V_{L-1} comprises a first antibody moiety specifically recognizing Sclerostin, wherein the V_{H-2} and the V_{L-2} comprises a second antibody moiety specifically recognizing DKK1.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 134; b) a first light chain comprising a first light chain variable region (V_{L-1}) and a first light chain constant region, wherein the first light chain comprises an amino acid sequence of SEQ ID NO: 135; c) a second heavy chain comprising a second variable heavy chain variable region (V_{H-2}) and a second heavy chain constant region, wherein the second heavy chain comprises the amino acid sequence of SEQ ID NO: 142; and d) a second light chain comprising a second light chain variable region (V_{L-2}) and a second light chain constant region, wherein the second light chain comprises the amino acid sequence of SEQ ID NO: 143, wherein the V_{H-1} and the V_{L-1} comprises a first antibody moiety specifically recognizing Sclerostin, wherein the V_{H-2} and the V_{L-2} comprises a second antibody moiety specifically recognizing DKK1.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 134; b) a first light chain comprising a first light chain variable region (V_{L-1}) and a first light chain constant region, wherein the first light chain comprises an amino acid sequence of SEQ ID NO: 135; c) a second heavy chain comprising a second variable heavy chain variable region (V_{H-2}) and a second heavy chain constant region, wherein the second heavy chain comprises the amino acid sequence of SEQ ID NO: 144; and d) a second light chain comprising a second light chain variable region (V_{L-2}) and a second light chain constant region, wherein the second light chain comprises the amino acid sequence of SEQ ID NO: 145, wherein the V_{H-1} and the V_{L-1} comprises a first antibody moiety specifically recognizing Sclerostin, wherein the V_{H-2} and the V_{L-2} comprises a second antibody moiety specifically recognizing DKK1.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 146; b) a first light chain comprising a first light chain variable region (V_{L-1}) and a first light chain constant region, wherein the first light chain comprises an amino acid sequence of SEQ ID NO: 147; c) a second heavy chain comprising a second variable heavy chain variable region (V_{H-2}) and a second heavy chain constant region, wherein the second heavy chain comprises the amino acid sequence of SEQ ID NO: 148; and d) a second light chain comprising a second light chain variable region (V_{L-2}) and a second light chain constant region, wherein the second light chain comprises the amino acid sequence of SEQ ID NO: 149, wherein the V_{H-1} and the V_{L-1} comprises a first antibody moiety specifically recognizing Sclerostin, wherein the V_{H-2} and the V_{L-2} comprises a second antibody moiety specifically recognizing DKK1.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 146; b) a first light chain comprising a first light chain variable region (V_{L-1}) and a first light chain constant region, wherein the first light chain comprises an amino acid sequence of SEQ ID NO: 147; c) a second heavy chain comprising a second variable heavy chain variable region (V_{H-2}) and a second heavy chain constant region, wherein the second heavy chain comprises the amino acid sequence of SEQ ID NO: 150; and d) a second light chain comprising a second light chain variable region (V_{L-2}) and a second light chain constant region, wherein the second light chain comprises the amino acid sequence of SEQ ID NO: 151, wherein the V_{H-1} and the V_{L-1} comprises a first antibody moiety specifically recognizing Sclerostin, wherein the V_{H-2} and the V_{L-2} comprises a second antibody moiety specifically recognizing DKK1.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 146; b) a first light chain comprising a first light chain variable region (V_{L-1}) and a first light chain constant region, wherein the first light chain comprises an amino acid sequence of SEQ ID NO: 147; c) a second heavy chain comprising a second variable heavy chain variable region (V_{H-2}) and a second heavy chain constant region, wherein the second heavy chain comprises the amino acid sequence of SEQ ID NO: 152; and d) a second light chain comprising a second light chain variable region (V_{L-2}) and a second light chain constant region, wherein the second light chain comprises the amino acid sequence of SEQ ID NO: 153, wherein the V_{H-1} and the V_{L-1} comprises a first antibody moiety specifically recognizing Sclerostin, wherein the V_{H-2} and the V_{L-2} comprises a second antibody moiety specifically recognizing DKK1.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 146; b) a first light chain comprising a first light chain variable region (V_{L-1}) and a first light chain constant region, wherein the first light chain comprises an amino acid sequence of SEQ ID NO: 147; c) a second heavy chain comprising a second variable heavy chain variable region (V_{H-2}) and a second heavy chain constant region, wherein the second heavy chain comprises the amino acid sequence of SEQ ID NO: 154; and d) a second light chain comprising a second light chain variable region (V_{L-2}) and a second light chain constant region, wherein the second light chain comprises the amino acid sequence of SEQ ID NO: 155, wherein the V_{H-1} and the V_{L-1} comprises a first antibody moiety specifically recognizing Sclerostin, wherein the V_{H-2} and the V_{L-2} comprises a second antibody moiety specifically recognizing DKK1.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 146; b) a first light chain comprising a first light chain variable region (V_{L-1}) and a first light chain constant region, wherein the first light chain comprises an amino acid sequence of SEQ ID NO: 147; c) a second heavy chain comprising a second variable heavy chain variable region (V_{H-2}) and a second heavy chain constant region, wherein the second heavy chain comprises the amino acid sequence of SEQ ID NO: 156; and d) a second light chain comprising a second light chain variable region (V_{L-2}) and a second light chain constant region, wherein the second light chain comprises the amino acid sequence of SEQ ID NO: 157, wherein the V_{H-1} and the V_{L-1} comprises a first antibody moiety specifically recognizing Sclerostin, wherein the V_{H-2} and the V_{L-2} comprises a second antibody moiety specifically recognizing RANKL.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 146; b) a first light chain comprising a first light chain variable region (V_{L-1}) and a first light chain constant region, wherein the first light chain comprises an amino acid sequence of SEQ ID NO: 147; c) a second heavy chain comprising a second variable heavy chain variable region (V_{H-2}) and a second heavy chain constant region, wherein the second heavy chain comprises the amino acid sequence of SEQ ID NO: 158; and d) a second light chain comprising a second light chain variable region (V_{L-2}) and a second light chain constant region, wherein the second light chain comprises the amino acid sequence of SEQ ID NO: 159, wherein the V_{H-1} and the V_{L-1} comprises a first antibody moiety specifically recognizing Sclerostin, wherein the V_{H-2} and the V_{L-2} comprises a second antibody moiety specifically recognizing RANKL.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 146; b) a first light chain comprising a first light chain variable region (V_{L-1}) and a first light chain constant region, wherein the first light chain comprises an amino acid sequence of SEQ ID NO: 147; c) a second heavy chain comprising a second variable heavy chain variable region (V_{H-2}) and a second heavy chain constant region, wherein the second heavy chain comprises the amino acid sequence of SEQ ID NO: 160; and d) a second light chain comprising a second light chain variable region (V_{L-2}) and a second light chain constant region, wherein the second light chain comprises the amino acid sequence of SEQ ID NO: 161, wherein the V_{H-1} and the V_{L-1} comprises a first antibody moiety specifically recognizing Sclerostin, wherein the V_{H-2} and the V_{L-2} comprises a second antibody moiety specifically recognizing RANKL.

In some embodiments, there is provided a multispecific (e.g., bispecific) construct, comprising a) a first heavy chain comprising a first variable heavy chain variable region (V_{H-1}) and a first heavy chain constant region, wherein the first heavy chain comprises an amino acid sequence of SEQ ID NO: 146; b) a first light chain comprising a first light chain variable region (V_{L-1}) and a first light chain constant region, wherein the first light chain comprises an amino acid sequence of SEQ ID NO: 147; c) a second heavy chain comprising a second variable heavy chain variable region (V_{H-2}) and a second heavy chain constant region, wherein the second heavy chain comprises the amino acid sequence of SEQ ID NO: 162; and d) a second light chain comprising a second light chain variable region (V_{L-2}) and a second light chain constant region, wherein the second light chain comprises the amino acid sequence of SEQ ID NO: 163, wherein the V_{H-1} and the V_{L-1} comprises a first antibody moiety specifically recognizing Sclerostin, wherein the V_{H-2} and the V_{L-2} comprises a second antibody moiety specifically recognizing RANKL.

### Anti-Sclerostin Fusion Proteins or Antibody-Drug Conjugate

The anti-Sclerostin constructs in some embodiments is a fusion protein or an antibody-drug conjugate that comprises an anti-Sclerostin antibody moiety (e.g., an anti-Sclerostin scFv) and a second moiety.

In some embodiments, the second moiety comprises a half-life extending moiety. In some embodiments, the half-life extending moiety is an albumin binding moiety (e.g., an albumin binding antibody moiety).

In some embodiments, the second moiety comprises an agent selected from the group consisting of a parathyroid hormone (PTH), a selective estrogen receptor modulator (SERM), a bisphosphonate, a prostaglandin E (PGE) receptor agonist, VEGF, TGFβ, growth factor (myostatin) and calcitonin.

In some embodiments, the anti-Sclerostin antibody moiety and the second moiety are fused via a linker (such as any of the linkers described in the “Linkers” section). In some embodiments, the anti-Sclerostin antibody moiety and the second moiety are fused without a linker.

### Linkers

In some embodiments, the anti-Sclerostin constructs described herein comprise one or more linkers between two moieties (e.g., the anti-Sclerostin antibody moiety and the half-life extending moiety, the anti-Sclerostin antibody moiety and the second binding moiety in the multispecific constructs described above). The length, the degree of flexibility and/or other properties of the linker(s) used in the anti-Sclerostin constructs may have some influence on properties, including but not limited to the affinity, specificity or avidity for one or more particular antigens or epitopes. For example, longer linkers may be selected to ensure that two adjacent domains do not sterically interfere with one another. In some embodiment, a linker (such as peptide linker) comprises flexible residues (such as glycine and serine) so that the adjacent domains are free to move relative to each other. For example, a glycine-serine doublet can be a suitable peptide linker. In some embodiments, the linker is a non-peptide linker. In some embodiments, the linker is a peptide linker. In some embodiments, the linker is a non-cleavable linker. In some embodiments, the linker is a cleavable linker.

Other linker considerations include the effect on physical or pharmacokinetic properties of the resulting compound, such as solubility, lipophilicity, hydrophilicity, hydrophobicity, stability (more or less stable as well as planned degradation), rigidity, flexibility, immunogenicity, modulation of antibody binding, the ability to be incorporated into a micelle or liposome, and the like.

### Peptide Linkers

The peptide linker may have a naturally occurring sequence, or a non-naturally occurring sequence. For example, a sequence derived from the hinge region of heavy chain only antibodies may be used as the linker. See, for example, WO1996/34103.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acid to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

An essential technical feature of such peptide linker is that said peptide linker does not comprise any polymerization activity. The characteristics of a peptide linker, which comprise the absence of the promotion of secondary structures, are known in the art and described, e.g., in Dall'Acqua et al. (Biochem. (1998) 37, 9266-9273), Cheadle et al. (Mol Immunol (1992) 29, 21-30) and Raag and Whitlow (FASEB (1995) 9(1), 73-80). A particularly preferred amino acid in context of the “peptide linker” is Gly. Furthermore, peptide linkers that also do not promote any secondary structures are preferred. The linkage of the domains to each other can be provided by, e.g., genetic engineering. Methods for preparing fused and operatively linked bispecific single chain constructs and expressing them in mammalian cells or bacteria are well-known in the art (e.g. WO 99/54440, Ausubel, Current Protocols in Molecular Biology, Green Publishing Associates and Wiley Interscience, N. Y. 1989 and 1994 or Sambrook et al., Molecular Cloning: A Laboratory Manual, Cold Spring Harbor Laboratory Press, Cold Spring Harbor, N. Y., 2001).

The peptide linker can be a stable linker, which is not cleavable by proteases, especially by Matrix metalloproteinases (MMPs).

The linker can also be a flexible linker. Exemplary flexible linkers include glycine polymers (G)ₙ (SEQ ID NO: 78), glycine-serine polymers (including, for example, (GS)ₙ (SEQ ID NO: 79), (GSGGS)ₙ (SEQ ID NO: 80), (GGGGS)ₙ (SEQ ID NO: 81), and (GGGS)ₙ (SEQ ID NO: 82), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (See Scheraga, Rev. Computational Chem. 11 173-142 (1992)). The ordinarily skilled artisan will recognize that design of an antibody fusion protein can include linkers that are all or partially flexible, such that the linker can include a flexible linker portion as well as one or more portions that confer less flexible structure to provide a desired antibody fusion protein structure.

Furthermore, exemplary linkers also include the amino acid sequence of such as (GGGGS)ₙ (SEQ ID NO: 81), wherein n is an integer between 1 and 8, e.g. (GGGGS)₃ (SEQ ID NO: 76), (GGGGS)₄ (SEQ ID NO: 77), or (GGGGS)₆ (SEQ ID NO: 83). In some embodiments, the peptide linker comprises the amino acid sequence of (GSTSGSGKPGSGEGS)ₙ (SEQ ID NO: 84), wherein n is an integer between 1 and 3.

### Non-Peptide Linkers

Coupling of two moieties may be accomplished by any chemical reaction that will bind the two molecules so long as both components retain their respective activities, e.g., binding to Sclerostin and a second agent in an anti-Sclerostin multispecific antibody, respectively. This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents may be useful in coupling protein molecules in this context. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (See Killen and Lindstrom, Jour. Immun. 133:1335-2549 (1984); Jansen et al., Immunological Reviews 62:185-216 (1982); and Vitetta et al., Science 238:1098 (1987)).

Linkers that can be applied in the present application are described in the literature (see, for example, Ramakrishnan, S. et al., Cancer Res. 44:201-208 (1984) describing use of MBS (M-maleimidobenzoyl-N-hydroxysuccinimide ester). In some embodiments, non-peptide linkers used herein include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus may lead to bispecific antibodies with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form antibody fusion protein with increased stability. Disulfide linkages, are in general, less stable than other linkages because the disulfide linkage is cleaved in vitro, resulting in less antibody fusion protein available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

### III. Methods of Preparation

In some embodiments, there is provided a method of preparing an anti-Sclerostin construct or antibody moiety that specifically binds to Sclerostin and a composition such as polynucleotide, nucleic acid construct, vector, host cell, or culture medium that is produced during the preparation of the anti-Sclerostin construct or antibody moiety. The anti-Sclerostin construct or antibody moiety or composition described herein may be prepared by a number of processes as generally described below and more specifically in the Examples.

### Antibody Expression and Production

The antibodies (including anti-Sclerostin monoclonal antibodies, anti-Sclerostin bispecific antibodies, and anti-Sclerostin antibody moieties) described herein can be prepared using any known methods in the art, including those described below and in the Examples.

### Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or a llama, is immunized as hereinabove described to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986). Also See Example 1 for immunization in Camels.

The immunizing agent will typically include the antigenic protein or a fusion variant thereof. An immunizing agent may be comprised of purified full-length or truncated Sclerostin polypeptides, or variants or fragments (i.e., peptides) thereof. Such peptides may be generated by proteolytic cleavage of a larger polypeptide, by recombinant molecular methodologies, or may be chemically synthesized by methods as described herein and known in the art. Based on crystallographic structure of human Sclerostin in complex with LRP6 (Kim, J. 2020, Nat Commun 11: 5357-5357), the loop tip containing the ‘IGRGKWWR’ motif (SEQ ID NO: 186) was found to be the main binding determinant for binding of Sclerostin to the first propeller of LRP6. Peptides useful as immunizing agent typically may have an amino acid sequence of at least 7 consecutive amino acids from a Sclerostin amino acid sequence covering the ‘IGRGKWWR’ motif (SEQ ID NO: 186) on the second loop such as those described herein, and preferably have at least 8, 9, 10, 11, 12, 14, 15, 16, 18, 19, 20, 21, 22, 23, or 24 consecutive amino acids comprising the ‘IGRGKWWR’ motif (SEQ ID NO: 186). Certain other preferred peptide agents comprise at least 8 but no more than 12 or more consecutive amino acids of the Sclerostin sequence covering the ‘IGRGKWWR’ motif (SEQ ID NO: 186), and other preferred peptide agent comprises 24 consecutive amino acids of the second loop of Sclerostin polypeptide. Other preferred peptide agents comprise any whole integer number of amino acids between and including 8 and 24 consecutive amino acids covering ‘IGRGKWWR’ motif (SEQ ID NO: 186) in the second loop thereof, or polypeptide comprising full-length Sclerostin sequence. Within one embodiment the immunizing agent is full-length human Sclerostin, or preferably a portion of the second loop thereof, e.g., synthesized peptides within the second loop as described herein covering amino acids 110-133, or more preferably 119-130, or more preferably 116-126, or more preferably 118-125, to generated antibodies that specifically recognize the ‘IGRGKWWR’ motif (SEQ ID NO: 186) of the second loop on Sclerostin. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof, e.g., X63-Ag8-653) available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined ‘by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen. Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980). To select the monoclonal antibodies that specifically neutralize Sclerostin's ability to inhibit Wnt activity, the resulting hybridomas can be screened in order to determine the presence of antibodies which are reactive with both full-length human Sclerostin and peptides from the second loop containing the ‘IGRGKWWR’ motif (SEQ ID NO: 186) using ELISA or BLI approach. Hybridomas that produce monoclonal antibodies that specifically bind to both full-length Sclerostin and the peptides containing the ‘IGRGKWWR’ motif (SEQ ID NO: 186) thereof are preferred as described in Example 3 and 4. And monoclonal antibodies that were able to block, impair, or inhibit the binding of full-length human Sclerostin to a low-density lipoprotein receptor-related protein family member, e.g., LRP5 or LRP6, are selected using HEK293/TCF/LEF/Wnt1 reporter gene assay (Hannoush R N., 2008, PLoS ONE, 3: e3498) as described in Example 7.

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose™, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Nucleic Acid Molecules Encoding Antibody Moieties

In some embodiments, there is provided a polynucleotide encoding any one of the anti-Sclerostin constructs or antibody moieties described herein. In some embodiments, there is provided a polynucleotide prepared using any one of the methods as described herein. In some embodiments, a nucleic acid molecule comprises a polynucleotide that encodes a heavy chain or a light chain of an antibody moiety (e.g., anti-Sclerostin antibody moiety). In some embodiments, a nucleic acid molecule comprises both a polynucleotide that encodes a heavy chain and a polynucleotide that encodes a light chain, of an antibody moiety (e.g., anti-Sclerostin antibody moiety). In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is a scFv, a single polynucleotide encodes a single polypeptide comprising both a heavy chain and a light chain linked together.

In some embodiments, a polynucleotide encoding a heavy chain or light chain of an antibody moiety (e.g., anti-Sclerostin antibody moiety) comprises a nucleotide sequence that encodes a leader sequence, which, when translated, is located at the N terminus of the heavy chain or light chain. As discussed above, the leader sequence may be the native heavy or light chain leader sequence, or may be another heterologous leader sequence.

In some embodiments, the polynucleotide is a DNA. In some embodiments, the polynucleotide is an RNA. In some embodiments, the RNA is an mRNA.

Nucleic acid molecules may be constructed using recombinant DNA techniques conventional in the art. In some embodiments, a nucleic acid molecule is an expression vector that is suitable for expression in a selected host cell.

### Nucleic Acid Construct

In some embodiments, there is provided a nucleic acid construct comprising any one of the polynucleotides described herein. In some embodiments, there is provided a nucleic acid construct prepared using any method described herein.

In some embodiments, the nucleic acid construct further comprises a promoter operably linked to the polynucleotide. In some embodiments, the polynucleotide corresponds to a gene, wherein the promoter is a wild-type promoter for the gene.

### Vectors

In some embodiments, there is provided a vector comprising any polynucleotides that encode the heavy chains and/or light chains of any one of the antibody moieties described herein (e.g., anti-Sclerostin antibody moieties) or nucleic acid construct described herein. In some embodiments, there is provided a vector prepared using any method described herein. Vectors comprising polynucleotides that encode any of anti-Sclerostin constructs such as antibodies, scFvs, fusion proteins or other forms of constructs described herein (e.g., anti-Sclerostin scFv) are also provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is a scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NS0 cells. Exemplary such vectors are described, e.g., in Running Deer et al., Biotechnol. Prog. 20:880-889 (2004).

### Host Cells

In some embodiments, there is provided a host cell comprising any polypeptide, nucleic acid construct and/or vector described herein. In some embodiments, there is provided a host cell prepared using any method described herein. In some embodiments, the host cell is capable of producing any of antibody moieties described herein under a fermentation condition.

In some embodiments, the antibody moieties described herein (e.g., anti-Sclerostin antibody moieties) may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells (Crucell); and NS0 cells. In some embodiments, the antibody moieties described herein (e.g., anti-Sclerostin antibody moieties) may be expressed in yeast. See, e.g., U.S. Publication No. US 2006/0270045 A1. In some embodiments, a particular eukaryotic host cell is selected based on its ability to make desired post-translational modifications to the heavy chains and/or light chains of the antibody moiety. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Non-limiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

The present application also provides host cells comprising any of the polynucleotides or vectors described herein. In some embodiments, the invention provides a host cell comprising an anti-Sclerostin antibody. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

In some embodiments, the antibody moiety is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### Culture Medium

In some embodiments, there is provided a culture medium comprising any antibody moiety, polynucleotide, nucleic acid construct, vector, and/or host cell described herein. In some embodiments, there is provided a culture medium prepared using any method described herein.

In some embodiments, the medium comprises hypoxanthine, aminopterin, and/or thymidine (e.g., HAT medium). In some embodiments, the medium does not comprise serum. In some embodiments, the medium comprises serum. In some embodiments, the medium is a D-MEM or RPMI-1640 medium.

### Purification of Antibody Moieties

The anti-Sclerostin constructs (e.g., anti-Sclerostin monoclonal antibodies or multispecific antibodies) may be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A, Protein G, Protein A/G, or an antibody affinity column may be used to bind the constant region and to purify an anti-Sclerostin construct comprising an Fc fragment. Hydrophobic interactive chromatography, for example, a butyl or phenyl column, may also suitable for purifying some polypeptides such as antibodies. Ion exchange chromatography (e.g. anion exchange chromatography and/or cation exchange chromatography) may also suitable for purifying some polypeptides such as antibodies. Mixed-mode chromatography (e.g. reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

### IV. Methods of Treatments

In some embodiments, there is provided a method of treating a disease or condition in an individual, comprising administering to the individual an effective mount of the anti-Sclerostin construct or pharmaceutical composition described herein. In some embodiments, the disease or condition is a bone-related disorder or cartilage related disorder, a bone marrow or haemotological disorder, a musculoskeletal rare disease, a muscle-related disorder, or a cancer.

The methods described herein are applicable to any bone-related disease or condition. In some embodiments, the bone-related disorder is osteogenesis imperfecta, osteoporosis or osteopenia (in men and/or women), osteonecrosis, delay bone healing, non-union bone fractures, multiple myeloma, multiple myeloma related bone disorders, primary bone tumor, bone metastasis of malignancies, inflammatory or infectious bone disease, osteomalacia, hypercalcemia, Paget's disease, immobilization-induced bone loss, glucocorticoid-induced bone loss, inflammation-induced bone loss including arthritis-induced bone loss, spaceflight osteoporosis/osteopenia and bone loss caused by reduced gravity or other disease or condition associated with a) bone loss of either quantity or quality or both and/or b) abnormality of bone structure and quality. In some embodiments, the bone-related disorder is osteoporosis or osteopenia. In some embodiments, the bone-related disorder is osteogenesis imperfecta. In some embodiments, the bone-related disorder is multiple myeloma and multiple myeloma related bone disorders.

In some embodiments, the disease or condition is a cartilage disorder. In some embodiments, the cartilage disorder is chondromatosis, chondrodysplasia, achondroplasia, epiphyseal dysplasia, chondrodystrophic myotonia, juxtacortical chondroma, tear of cartilage of knee, osteofibrous dysplasia, osteoarthritis, osteogenesis imperfecta, hypophosphatemic rickets or osteochondrodystrophy.

In some embodiments, the disease or condition is a muscle-related disorder. In some embodiments, the muscle-related disorder is sarcopenia and cancer sarcopenia.

In some embodiments, the disease or condition is a cancer (e.g., a hematological malignancy, e.g., multiple myeloma).

In some embodiments, there is provided a method of facilitation of heal after bone or joint surgeries in an individual, comprising administering to the individual an effective mount of the anti-Sclerostin construct (such as any of the anti-Sclerostin constructs described herein).

In some embodiments, there is provided a method of treating a disease or condition (e.g., a bone-related disease) in an individual, comprising administering to the individual an effective mount of an anti-Sclerostin construct comprising an antibody moiety comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 9, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 15 or 85, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs.

In some embodiments, there is provided a method of treating a disease or condition (e.g., a bone-related disease) in an individual, comprising administering to the individual an effective mount of an multispecific construct comprising a first antibody moiety that specifically recognizes Sclerostin and a second antibody moiety specifically recognizes RANKL. In some embodiments, the first antibody moiety comprises a first heavy chain variable region (V_{H-1}) and a first light chain variable region (V_{L-1}), wherein the V_{H-1} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 9, or a variant thereof comprising up to 5, 4, 3, 2, or 1 amino acid substitutions in the HC-CDRs, and the V_{L-1} comprises i) the LC-CDR1 comprising the amino acid sequence of SEQ ID NO: 15 or 85, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs. In some embodiments, the second antibody moiety comprising a second heavy chain variable region (V_{H-2}) and a second light chain variable region (V_{L-2}), wherein the V_{H-2} comprises the HC-CDR1 comprising the amino acid sequence of SEQ ID NO: 66, the HC-CDR2 comprising the amino acid sequence of SEQ ID NO: 67, and the HC-CDR3 comprising the amino acid sequence of SEQ ID NO: 68, and the V_{L-2} comprises the LC-CDR1 comprising the amino acid sequence of SEQ ID NO: 69, the LC-CDR2 comprising the amino acid sequence of SEQ ID NO: 70, and the LC-CDR3 comprising the amino acid sequence of SEQ ID NO: 71.

In some embodiments, there is provided a method of treating a disease or condition (e.g., a bone-related disease) in an individual, comprising administering to the individual an effective mount of an multispecific construct comprising a first antibody moiety that specifically recognizes Sclerostin and a second antibody moiety specifically recognizes DKK1. In some embodiments, the first antibody moiety comprises a first heavy chain variable region (V_{H-1}) and a first light chain variable region (V_{L-1}), wherein the V_{H-1} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 9, or a variant thereof comprising up to 5, 4, 3, 2, or 1 amino acid substitutions in the HC-CDRs, and the V_{L-1} comprises i) the LC-CDR1 comprising the amino acid sequence of SEQ ID NO: 15 or 85, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs. In some embodiments, the second antibody moiety comprising a second heavy chain variable region (V_{H-2}) and a second light chain variable region (V_{L-2}), wherein: a) the V_{H-2} comprises the HC-CDR1 comprising the amino acid sequence of SEQ ID NO: 42, the HC-CDR2 comprising the amino acid sequence of SEQ ID NO: 43, and the HC-CDR3 comprising the amino acid sequence of SEQ ID NO: 44, and the V_{L-2} comprises the LC-CDR1 comprising the amino acid sequence of SEQ ID NO: 45, the LC-CDR2 comprising the amino acid sequence of SEQ ID NO: 46, and the LC-CDR3 comprising the amino acid sequence of SEQ ID NO: 47; b) the V_{H-2} comprises the HC-CDR1 comprising the amino acid sequence of SEQ ID NO: 48, the HC-CDR2 comprising the amino acid sequence of SEQ ID NO: 49, and the HC-CDR3 comprising the amino acid sequence of SEQ ID NO: 50, and the V_{L-2} comprises the LC-CDR1 comprising the amino acid sequence of SEQ ID NO: 51, the LC-CDR2 comprising the amino acid sequence of SEQ ID NO: 52, and the LC-CDR3 comprising the amino acid sequence of SEQ ID NO: 58; or c) the V_{H-2} comprises the HC-CDR1 comprising the amino acid sequence of SEQ ID NO: 54, the HC-CDR2 comprising the amino acid sequence of SEQ ID NO: 55, and the HC-CDR3 comprising the amino acid sequence of SEQ ID NO: 56, and the V_{L-2} comprises the LC-CDR1 comprising the amino acid sequence of SEQ ID NO: 57, the LC-CDR2 comprising the amino acid sequence of SEQ ID NO: 58, and the LC-CDR3 comprising the amino acid sequence of SEQ ID NO: 59.

In some embodiments, the subject is a mammal (such as a human).

### Dosing and Method of Administering the Anti-Sclerostin Construct

The dosing regimen of the anti-Sclerostin construct (such as the specific dosages and frequencies) used for treating a disease or disorder as described herein administered into the individual may vary with the particular anti-Sclerostin construct (such as anti-Sclerostin monoclonal or multispecific antibodies, such as anti-Sclerostin fusion proteins), the mode of administration, and the type of disease or condition being treated.

In some embodiments of any of the above aspects, the effective amount of an anti-Sclerostin construct (such as anti-Sclerostin monoclonal or multispecific antibodies) is in the range of about 0.001 μg/kg to about 500 mg/kg of total body weight, for example, about 0.005 μg/kg to about 100 mg/kg, about 0.01 μg/kg to about 50 mg/kg, or about 0.01 μg/kg to about 5 mg/kg.

In some embodiments, the treatment comprises more than one administration of the anti-Sclerostin constructs (such as about two, three, four, five, six, seven, eight, night, or ten administrations of anti-Sclerostin constructs). In some embodiments, the anti-Sclerostin construct is administered at a frequency of about daily, weekly, two times per week, once a month, once every three months, once every six months, or once a year.

The anti-Sclerostin construct can be administered to an individual (such as human) via various routes, including, for example, intravenous, intra-articular, intra-arterial, intraperitoneal, intrapulmonary, oral, inhalation, intravesicular, intramuscular, intra-tracheal, subcutaneous, intraocular, intrathecal, transmucosal, and transdermal. In some embodiments, the anti-Sclerostin construct is included in a pharmaceutical composition while administered into the individual. In some embodiments, sustained continuous release formulation of the composition may be used. In some embodiments, the composition is administered intravenously. In some embodiments, the composition is administered intraperitoneally. In some embodiments, the composition is administered intravenously. In some embodiments, the composition is administered intraperitoneally. In some embodiments, the composition is administered intramuscularly. In some embodiments, the composition is administered subcutaneously. In some embodiments, the composition is administered intravenously. In some embodiments, the composition is administered orally.

### Combination Therapy

This application also provides methods of administering an anti-Sclerostin construct into an individual for treating a disease or condition (such as a bone-related disease), wherein the method further comprises administering a second agent or therapy. In some embodiments, the second agent or therapy is a standard or commonly used agent or therapy for treating the disease or condition.

In some embodiments, there is provided a method of treating a disease or condition (e.g., a bone-related disease) in an individual, comprising administering to the individual a) an effective mount of an anti-Sclerostin construct (such as any of the anti-Sclerostin constructs described herein); and b) a second therapy or agent. In some embodiments, the second therapy or agent is an anti-DKK1 antibody or an anti-RANKL antibody. In some embodiments, the second agent or therapy comprises an agent selected from the group consisting of a parathyroid hormone (PTH), a selective estrogen receptor modulator (SERM), a bisphosphonate, a prostaglandin E (PGE) receptor agonist, VEGF, and TGFβ, growth factor (myostatin) and calcitonin.

In some embodiments, there is provided a method of treating a disease or condition (e.g., a bone-related disease) in an individual, comprising administering to the individual a) an effective mount of an anti-Sclerostin construct (such as any of the anti-Sclerostin constructs described herein); and b) an anti-DKK1 antibody. In some embodiments, the anti-Sclerostin construct comprises an antibody moiety comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 9, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 15 or 85, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs. In some embodiments, the anti-DKK1 antibody comprises a second heavy chain variable region (V_{H-2}) and a second light chain variable region (V_{L-2}), wherein the V_{H-2} comprises the HC-CDR1 comprising the amino acid sequence of SEQ ID NO: 66, the HC-CDR2 comprising the amino acid sequence of SEQ ID NO: 67, and the HC-CDR3 comprising the amino acid sequence of SEQ ID NO: 68, and the V_{L-2} comprises the LC-CDR1 comprising the amino acid sequence of SEQ ID NO: 69, the LC-CDR2 comprising the amino acid sequence of SEQ ID NO: 70, and the LC-CDR3 comprising the amino acid sequence of SEQ ID NO: 71.

In some embodiments, there is provided a method of treating a disease or condition (e.g., a bone-related disease) in an individual, comprising administering to the individual a) an effective mount of an anti-Sclerostin construct (such as any of the anti-Sclerostin constructs described herein); and b) an anti-RANKL antibody. In some embodiments, the anti-Sclerostin construct comprises an antibody moiety comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 5, and iii) the HC-CDR3 comprising the amino acid sequence of SEQ ID NO: 9, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 15 or 85, ii) the LC-CDR2 comprising the amino acid sequence of SEQ ID NO: 18, and iii) the LC-CDR3 comprising the amino acid sequence of SEQ ID NO: 20, or a variant thereof comprising up to 5, 4, 3, 2, or 1 amino acid substitutions in the LC-CDRs. In some embodiments, the anti-RANKL antibody comprises a second heavy chain variable region (V_{H-2}) and a second light chain variable region (V_{L-2}), wherein: a) the V_{H-2} comprises the HC-CDR1 comprising the amino acid sequence of SEQ ID NO: 42, the HC-CDR2 comprising the amino acid sequence of SEQ ID NO: 43, and the HC-CDR3 comprising the amino acid sequence of SEQ ID NO: 44, and the V_{L-2} comprises the LC-CDR1 comprising the amino acid sequence of SEQ ID NO: 45, the LC-CDR2 comprising the amino acid sequence of SEQ ID NO: 46, and the LC-CDR3 comprising the amino acid sequence of SEQ ID NO: 47; b) the V_{H-2} comprises the HC-CDR1 comprising the amino acid sequence of SEQ ID NO: 48, the HC-CDR2 comprising the amino acid sequence of SEQ ID NO: 49, and the HC-CDR3 comprising the amino acid sequence of SEQ ID NO: 50, and the V_{L-2} comprises the LC-CDR1 comprising the amino acid sequence of SEQ ID NO: 51, the LC-CDR2 comprising the amino acid sequence of SEQ ID NO: 52, and the LC-CDR3 comprising the amino acid sequence of SEQ ID NO: 58; or c) the V_{H-2} comprises the HC-CDR1 comprising the amino acid sequence of SEQ ID NO: 54, the HC-CDR2 comprising the amino acid sequence of SEQ ID NO: 55, and the HC-CDR3 comprising the amino acid sequence of SEQ ID NO: 56, and the V_{L-2} comprises the LC-CDR1 comprising the amino acid sequence of SEQ ID NO: 57, the LC-CDR2 comprising the amino acid sequence of SEQ ID NO: 58, and the LC-CDR3 comprising the amino acid sequence of SEQ ID NO: 59.

In some embodiments, the anti-Sclerostin construct and the second agent or therapy are administered simultaneously. In some embodiments, the anti-Sclerostin construct and the second agent or therapy are administered concurrently. In some embodiments, the anti-Sclerostin construct and the second agent or therapy are administered sequentially.

### V. Compositions, Kits and Articles of Manufacture

Also provided herein are compositions (such as formulations, such as pharmaceutical compositions) comprising any one of the anti-Sclerostin construct or anti-Sclerostin antibody moiety described herein, nucleic acid encoding the antibody moieties, vector comprising the nucleic acid encoding the antibody moieties, or host cells comprising the nucleic acid or vector.

In some embodiments, there is provided a pharmaceutical composition comprising an anti-Sclerostin construct (such as any of the anti-Sclerostin constructs described herein) and a pharmaceutically acceptable carrier. In some embodiments, the composition further comprises an agent selected from the group consisting of a parathyroid hormone (PTH), a selective estrogen receptor modulator (SERM), VEGF, TGFβ, growth factor (myostatin) and calcitonin.

Suitable formulations of the anti-Sclerostin construct described herein can be obtained by mixing the anti-Sclerostin construct or anti-Sclerostin antibody moiety having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ PLURONICS™ or polyethylene glycol (PEG). Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be imaged, diagnosed, or treated herein.

The formulations to be used for in vivo administration must be sterile. This is readily accomplished by, e.g., filtration through sterile filtration membranes.

Also provided are kits comprising any one of the anti-Sclerostin construct or anti-Sclerostin antibody moiety described herein. The kits may be useful for any of the methods of modulating cell composition or treatment described herein.

In some embodiments, there is provided a kit comprising an anti-Sclerostin construct specifically binding to Sclerostin.

In some embodiments, the kit further comprises a device capable of delivering the anti-Sclerostin construct into an individual. One type of device, for applications such as parenteral delivery, is a syringe that is used to inject the composition into the body of a subject. Inhalation devices may also be used for certain applications.

In some embodiments, the kit further comprises a therapeutic agent for treating a disease or condition, e.g., a bone-related disease, e.g., osteogenesis imperfecta, osteopetrosis, or a disease or condition associated with bone loss.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information.

The present application thus also provides articles of manufacture. The article of manufacture can comprise a container and a label or package insert on or associated with the container. Suitable containers include vials (such as sealed vials), bottles, jars, flexible packaging, and the like. Generally, the container holds a composition, and may have a sterile access port (for example the container may be an intravenous solution bag or a vial having a stopper pierceable by a hypodermic injection needle). The label or package insert indicates that the composition is used for imaging, diagnosing, or treating a particular condition in an individual. The label or package insert will further comprise instructions for administering the composition to the individual and for imaging the individual. The label may indicate directions for reconstitution and/or use. The container holding the composition may be a multi-use vial, which allows for repeat administrations (e.g. from 2-6 administrations) of the reconstituted formulation. Package insert refers to instructions customarily included in commercial packages of diagnostic products that contain information about the indications, usage, dosage, administration, contraindications and/or warnings concerning the use of such diagnostic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the compositions and instructions for use, packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXEMPLARY EMBODIMENTS

Embodiment 1. An anti-Sclerostin construct comprising an antibody moiety that specifically recognizes Sclerostin, wherein the antibody moiety binds to an epitope on Sclerostin, wherein the epitope comprises the amino acid sequence set forth in SEQ ID NO: 186.

Embodiment 2. An anti-Sclerostin construct comprising an antibody moiety that specifically recognizes Sclerostin, wherein the antibody moiety comprises a heavy chain variable region (V_{H}) and a light chain variable region (V_{L}), wherein:
the V_{H} comprises a HC-CDR1 comprising the amino acid sequence of any one of SEQ ID NOs: 1-4 and 12, a HC-CDR2 comprising the amino acid sequence of any one of SEQ ID NOs: 5-8 and 13, and a HC-CDR3 comprising the amino acid sequence of any one of SEQ ID NOs: 9-11 and 14, or a variant thereof comprising up to 5, 4, 3, 2, or 1 amino acid substitutions in the HC-CDRs; and
the V_{L} comprises a LC-CDR1 comprising the amino acid sequence of any one of SEQ ID NOs: 15-17, 85, and 86, a LC-CDR2 comprising the amino acid sequence of any one of SEQ ID NOs: 18 and 19, and a LC-CDR3 comprising the amino acid sequence of any one of SEQ ID NOs: 20 and 21, or a variant thereof comprising up to 5, 4, 3, 2, or 1 amino acid substitutions in the LC-CDRs.

Embodiment 3. The anti-Sclerostin construct of embodiment 1 or embodiment 2, wherein:
a) the V_{H} comprises the HC-CDR1 comprising the amino acid sequence of SEQ ID NO: 12, the HC-CDR2 comprising the amino acid sequence of SEQ ID NO: 13, and the HC-CDR3 comprising the amino acid sequence of SEQ ID NO: 14; and the V_{L} comprises the LC-CDR1 comprising the amino acid sequence of SEQ ID NO: 15 or 16, the LC-CDR2 comprising the amino acid sequence of SEQ ID NO: 18, and the LC-CDR3 comprising the amino acid sequence of any one of SEQ ID NOs: 20, or
b) the V_{H} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9; and the V_{L} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of any one of SEQ ID NOs: 20.

Embodiment 4. The anti-Sclerostin construct of embodiment 1 or embodiment 2, wherein the V_{H} comprises the HC-CDR1 comprising the amino acid sequence of SEQ ID NO: 1, the HC-CDR2 comprising the amino acid sequence of SEQ ID NO: 6, and the HC-CDR3 comprising the amino acid sequence of SEQ ID NO: 10; and the V_{L} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of any one of SEQ ID NOs: 20.

Embodiment 5. The anti-Sclerostin construct of embodiment 1 or embodiment 2, wherein the V_{H} comprises the HC-CDR1 comprising the amino acid sequence of SEQ ID NO: 2, the HC-CDR2 comprising the amino acid sequence of SEQ ID NO: 7, and the HC-CDR3 comprising the amino acid sequence of SEQ ID NO: 10; and the V_{L} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of any one of SEQ ID NOs: 20.

Embodiment 6. The anti-Sclerostin construct of embodiment 1 or embodiment 2, wherein the V_{H} comprises the HC-CDR1 comprising the amino acid sequence of SEQ ID NO: 2, the HC-CDR2 comprising the amino acid sequence of SEQ ID NO: 6, and the HC-CDR3 comprising the amino acid sequence of SEQ ID NO: 10; and the V_{L} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of any one of SEQ ID NOs: 20.

Embodiment 7. The anti-Sclerostin construct of embodiment 1 or embodiment 2, wherein the V_{H} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 10; and the V_{L} comprises the LC-CDR1 comprising the amino acid sequence of SEQ ID NO: 16, the LC-CDR2 comprising the amino acid sequence of SEQ ID NO: 18, and the LC-CDR3 comprising the amino acid sequence of any one of SEQ ID NOs: 20.

Embodiment 8. The anti-Sclerostin construct of embodiment 1 or embodiment 2, wherein the V_{H} comprises the HC-CDR1 comprising the amino acid sequence of SEQ ID NO: 3, the HC-CDR2 comprising the amino acid sequence of SEQ ID NO: 7, and the HC-CDR3 comprising the amino acid sequence of SEQ ID NO: 10; and the V_{L} comprises the LC-CDR1 comprising the amino acid sequence of SEQ ID NO: 15, the LC-CDR2 comprising the amino acid sequence of SEQ ID NO: 18, and the LC-CDR3 comprising the amino acid sequence of any one of SEQ ID NOs: 20.

Embodiment 9. The anti-Sclerostin construct of embodiment 1 or embodiment 2, wherein the V_{H} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 10; and the V_{L} comprises the LC-CDR1 comprising the amino acid sequence of SEQ ID NO: 16, the LC-CDR2 comprising the amino acid sequence of SEQ ID NO: 18, and the LC-CDR3 comprising the amino acid sequence of any one of SEQ ID NOs: 20.

Embodiment 10. The anti-Sclerostin construct of embodiment 1 or embodiment 2, wherein the V_{H} comprises the HC-CDR1 comprising the amino acid sequence of SEQ ID NO: 4, the HC-CDR2 comprising the amino acid sequence of SEQ ID NO: 8, and the HC-CDR3 comprising the amino acid sequence of SEQ ID NO: 11; and the V_{L} comprises the LC-CDR1 comprising the amino acid sequence of SEQ ID NO: 17, the LC-CDR2 comprising the amino acid sequence of SEQ ID NO: 19, and the LC-CDR3 comprising the amino acid sequence of any one of SEQ ID NOs: 21.

Embodiment 11. The anti-Sclerostin construct of embodiment 1 or embodiment 2, wherein the V_{H} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9; and the V_{L} comprises the LC-CDR1 comprising the amino acid sequence of SEQ ID NO: 85, the LC-CDR2 comprising the amino acid sequence of SEQ ID NO: 18, and the LC-CDR3 comprising the amino acid sequence of any one of SEQ ID NOs: 20.

Embodiment 12. The anti-Sclerostin construct of embodiment 1 or embodiment 2, wherein the V_{H} comprises the HC-CDR1 comprising the amino acid sequence of SEQ ID NO: 1, the HC-CDR2 comprising the amino acid sequence of SEQ ID NO: 5, and the HC-CDR3 comprising the amino acid sequence of SEQ ID NO: 9; and the V_{L} comprises the LC-CDR1 comprising the amino acid sequence of SEQ ID NO: 86, the LC-CDR2 comprising the amino acid sequence of SEQ ID NO: 18, and the LC-CDR3 comprising the amino acid sequence of any one of SEQ ID NOs: 20.

Embodiment 13. An anti-Sclerostin construct comprising an antibody moiety that specifically recognizes Sclerostin, wherein the antibody moiety comprises a heavy chain variable region (V_{H}) and a light chain variable region (V_{L}), wherein:
a) a HC-CDR1, a HC-CDR2, and a HC-CDR3, respectively comprising the amino acid sequences of a CDR1, a CDR2, and a CDR3 within the V_{H} having the sequence set forth in SEQ ID NO: 22, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 23;
b) a HC-CDR1, a HC-CDR2, and a HC-CDR3, respectively comprising the amino acid sequences of a CDR1, a CDR2, and a CDR3 within the V_{H} having the sequence set forth in SEQ ID NO: 24, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 25;
c) a HC-CDR1, a HC-CDR2, and a HC-CDR3, respectively comprising the amino acid sequences of a CDR1, a CDR2, and a CDR3 within the V_{H} having the sequence set forth in SEQ ID NO: 26, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 27;
d) a HC-CDR1, a HC-CDR2, and a HC-CDR3, respectively comprising the amino acid sequences of a CDR1, a CDR2, and a CDR3 within the V_{H} having the sequence set forth in SEQ ID NO: 28, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 29;
e) a HC-CDR1, a HC-CDR2, and a HC-CDR3, respectively comprising the amino acid sequences of a CDR1, a CDR2, and a CDR3 within the V_{H} having the sequence set forth in SEQ ID NO: 30, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 31;
f) a HC-CDR1, a HC-CDR2, and a HC-CDR3, respectively comprising the amino acid sequences of a CDR1, a CDR2, and a CDR3 within the V_{H} having the sequence set forth in SEQ ID NO: 32, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 33;
g) a HC-CDR1, a HC-CDR2, and a HC-CDR3, respectively comprising the amino acid sequences of a CDR1, a CDR2, and a CDR3 within the V_{H} having the sequence set forth in SEQ ID NO: 34, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 35;
h) a HC-CDR1, a HC-CDR2, and a HC-CDR3, respectively comprising the amino acid sequences of a CDR1, a CDR2, and a CDR3 within the V_{H} having the sequence set forth in SEQ ID NO: 36, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 37;
i) a HC-CDR1, a HC-CDR2, and a HC-CDR3, respectively comprising the amino acid sequences of a CDR1, a CDR2, and a CDR3 within the V_{H} having the sequence set forth in SEQ ID NO: 38, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 39;
j) a HC-CDR1, a HC-CDR2, and a HC-CDR3, respectively comprising the amino acid sequences of a CDR1, a CDR2, and a CDR3 within the V_{H} having the sequence set forth in SEQ ID NO: 40, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 41;
k) a HC-CDR1, a HC-CDR2, and a HC-CDR3, respectively comprising the amino acid sequences of a CDR1, a CDR2, and a CDR3 within the V_{H} having the sequence set forth in SEQ ID NO: 87, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 88;
l) a HC-CDR1, a HC-CDR2, and a HC-CDR3, respectively comprising the amino acid sequences of a CDR1, a CDR2, and a CDR3 within the V_{H} having the sequence set forth in SEQ ID NO: 87, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 89; or
m) a HC-CDR1, a HC-CDR2, and a HC-CDR3, respectively comprising the amino acid sequences of a CDR1, a CDR2, and a CDR3 within the V_{H} having the sequence set forth in SEQ ID NO: 87, and a LC-CDR1, a LC-CDR2, and a LC-CDR3, respectively comprising the amino acid sequences of a CDR1, a CDR2, and a CDR3 within the V_{L} having the sequence set forth in SEQ ID NO: 90.

Embodiment 14. The anti-Sclerostin construct of any one of embodiments 1-13, wherein the V_{H} comprises an amino acid sequence of any one of SEQ ID NOs: 22, 24, 26, 28, 30, 32, 34, 36, 38, 40, and 87, or a variant comprising an amino acid sequence having at least about 80% sequence identity; and/or wherein the V_{L} comprises an amino acid sequence of any one of SEQ ID NOs: 23, 25, 27, 29, 31, 33, 35, 37, 39, 41, and 88-90 or a variant comprising an amino acid sequence having at least about 80% sequence identity.

Embodiment 15. The anti-Sclerostin construct of embodiment 14, wherein:
a) the V_{H} comprises an amino acid sequence of SEQ ID NO: 22, or a variant comprising an amino acid sequence having at least about 80% sequence identity; and the V_{L} comprises an amino acid sequence of SEQ ID NO: 23, or a variant comprising an amino acid sequence having at least about 80% sequence identity,
b) the V_{H} comprises an amino acid sequence of SEQ ID NO: 24, or a variant comprising an amino acid sequence having at least about 80% sequence identity; and the V_{L} comprises an amino acid sequence of SEQ ID NO: 25, or a variant comprising an amino acid sequence having at least about 80% sequence identity,
c) the V_{H} comprises an amino acid sequence of SEQ ID NO: 26, or a variant comprising an amino acid sequence having at least about 80% sequence identity; and the V_{L} comprises an amino acid sequence of SEQ ID NO: 27, or a variant comprising an amino acid sequence having at least about 80% sequence identity,
d) the V_{H} comprises an amino acid sequence of SEQ ID NO: 28, or a variant comprising an amino acid sequence having at least about 80% sequence identity; and the V_{L} comprises an amino acid sequence of SEQ ID NO: 29, or a variant comprising an amino acid sequence having at least about 80% sequence identity,
e) the V_{H} comprises an amino acid sequence of SEQ ID NO: 30, or a variant comprising an amino acid sequence having at least about 80% sequence identity; and the V_{L} comprises an amino acid sequence of SEQ ID NO: 31, or a variant comprising an amino acid sequence having at least about 80% sequence identity,
f) the V_{H} comprises an amino acid sequence of SEQ ID NO: 32, or a variant comprising an amino acid sequence having at least about 80% sequence identity; and the V_{L} comprises an amino acid sequence of SEQ ID NO: 33, or a variant comprising an amino acid sequence having at least about 80% sequence identity,
g) the V_{H} comprises an amino acid sequence of SEQ ID NO: 34, or a variant comprising an amino acid sequence having at least about 80% sequence identity; and the V_{L} comprises an amino acid sequence of SEQ ID NO: 35, or a variant comprising an amino acid sequence having at least about 80% sequence identity,
h) the V_{H} comprises an amino acid sequence of SEQ ID NO: 36, or a variant comprising an amino acid sequence having at least about 80% sequence identity; and the V_{L} comprises an amino acid sequence of SEQ ID NO: 37, or a variant comprising an amino acid sequence having at least about 80% sequence identity,
i) the V_{H} comprises an amino acid sequence of SEQ ID NO: 38, or a variant comprising an amino acid sequence having at least about 80% sequence identity; and the V_{L} comprises an amino acid sequence of SEQ ID NO: 39, or a variant comprising an amino acid sequence having at least about 80% sequence identity,
j) the V_{H} comprises an amino acid sequence of SEQ ID NO: 40, or a variant comprising an amino acid sequence having at least about 80% sequence identity; and the V_{L} comprises an amino acid sequence of SEQ ID NO: 41, or a variant comprising an amino acid sequence having at least about 80% sequence identity,
k) the V_{H} comprises an amino acid sequence of SEQ ID NO: 87, or a variant comprising an amino acid sequence having at least about 80% sequence identity; and the V_{L} comprises an amino acid sequence of SEQ ID NO: 88, or a variant comprising an amino acid sequence having at least about 80% sequence identity,
l) the V_{H} comprises an amino acid sequence of SEQ ID NO: 87, or a variant comprising an amino acid sequence having at least about 80% sequence identity; and the V_{L} comprises an amino acid sequence of SEQ ID NO: 89, or a variant comprising an amino acid sequence having at least about 80% sequence identity, or
m) the V_{H} comprises an amino acid sequence of SEQ ID NO: 87, or a variant comprising an amino acid sequence having at least about 80% sequence identity; and the V_{L} comprises an amino acid sequence of SEQ ID NO: 90, or a variant comprising an amino acid sequence having at least about 80% sequence identity.

Embodiment 16. An anti-Sclerostin construct comprising an antibody moiety that specifically recognizes Sclerostin, wherein the antibody moiety is a humanized antibody moiety derived from an anti-Sclerostin antibody comprising a heavy chain variable region (V_{H}) and a second light chain variable region (V_{L}), wherein:
a) the V_{H} comprises a HC-CDR1 comprising the amino acid sequence of SEQ ID NO: 1, a HC-CDR2 comprising the amino acid sequence of SEQ ID NO: 5, and a HC-CDR3 comprising the amino acid sequence of SEQ ID NO: 9, or a variant thereof comprising up to 5, 4, 3, 2, or 1 amino acid substitutions in the HC-CDRs; and the V_{L} comprises a LC-CDR1 comprising the amino acid sequence of SEQ ID NO: 15, a LC-CDR2 comprising the amino acid sequence of SEQ ID NO: 18, and a LC-CDR3 comprising the amino acid sequence of any one of SEQ ID NOs: 20, or a variant thereof comprising up to 5, 4, 3, 2, or 1 amino acid substitutions in the LC-CDRs;
b) the V_{H} comprises a HC-CDR1 comprising the amino acid sequence of SEQ ID NO: 1, a HC-CDR2 comprising the amino acid sequence of SEQ ID NO: 6, and a HC-CDR3 comprising the amino acid sequence of SEQ ID NO: 10, or a variant thereof comprising up to 5, 4, 3, 2, or 1 amino acid substitutions in the HC-CDRs; and the V_{L} comprises a LC-CDR1 comprising the amino acid sequence of SEQ ID NO: 15, a LC-CDR2 comprising the amino acid sequence of SEQ ID NO: 18, and a LC-CDR3 comprising the amino acid sequence of any one of SEQ ID NOs: 20, or a variant thereof comprising up to 5, 4, 3, 2, or 1 amino acid substitutions in the LC-CDRs;
c) the V_{H} comprises a HC-CDR1 comprising the amino acid sequence of SEQ ID NO: 2, a HC-CDR2 comprising the amino acid sequence of SEQ ID NO: 7, and a HC-CDR3 comprising the amino acid sequence of SEQ ID NO: 10, or a variant thereof comprising up to 5, 4, 3, 2, or 1 amino acid substitutions in the HC-CDRs; and the V_{L} comprises a LC-CDR1 comprising the amino acid sequence of SEQ ID NO: 15, a LC-CDR2 comprising the amino acid sequence of SEQ ID NO: 18, and a LC-CDR3 comprising the amino acid sequence of any one of SEQ ID NOs: 20, or a variant thereof comprising up to 5, 4, 3, 2, or 1 amino acid substitutions in the LC-CDRs;
d) the V_{H} comprises a HC-CDR1 comprising the amino acid sequence of SEQ ID NO: 3, a HC-CDR2 comprising the amino acid sequence of SEQ ID NO: 6, and a HC-CDR3 comprising the amino acid sequence of SEQ ID NO: 10, or a variant thereof comprising up to 5, 4, 3, 2, or 1 amino acid substitutions in the HC-CDRs; and the V_{L} comprises a LC-CDR1 comprising the amino acid sequence of SEQ ID NO: 15, a LC-CDR2 comprising the amino acid sequence of SEQ ID NO: 18, and a LC-CDR3 comprising the amino acid sequence of any one of SEQ ID NOs: 20, or a variant thereof comprising up to 5, 4, 3, 2, or 1 amino acid substitutions in the LC-CDRs;
e) the V_{H} comprises a HC-CDR1 comprising the amino acid sequence of SEQ ID NO: 199, a HC-CDR2 comprising the amino acid sequence of SEQ ID NO: 5, and a HC-CDR3 comprising the amino acid sequence of SEQ ID NO: 10, or a variant thereof comprising up to 5, 4, 3, 2, or 1 amino acid substitutions in the HC-CDRs; and the V_{L} comprises a LC-CDR1 comprising the amino acid sequence of SEQ ID NO: 16, a LC-CDR2 comprising the amino acid sequence of SEQ ID NO: 18, and a LC-CDR3 comprising the amino acid sequence of any one of SEQ ID NOs: 20, or a variant thereof comprising up to 5, 4, 3, 2, or 1 amino acid substitutions in the LC-CDRs;
f) the V_{H} comprises a HC-CDR1 comprising the amino acid sequence of SEQ ID NO: 3, a HC-CDR2 comprising the amino acid sequence of SEQ ID NO: 200, and a HC-CDR3 comprising the amino acid sequence of SEQ ID NO: 10, or a variant thereof comprising up to 5, 4, 3, 2, or 1 amino acid substitutions in the HC-CDRs; and the V_{L} comprises a LC-CDR1 comprising the amino acid sequence of SEQ ID NO: 15, a LC-CDR2 comprising the amino acid sequence of SEQ ID NO: 18, and a LC-CDR3 comprising the amino acid sequence of any one of SEQ ID NOs: 20, or a variant thereof comprising up to 5, 4, 3, 2, or 1 amino acid substitutions in the LC-CDRs;
g) the V_{H} comprises a HC-CDR1 comprising the amino acid sequence of SEQ ID NO: 199, a HC-CDR2 comprising the amino acid sequence of SEQ ID NO: 5, and a HC-CDR3 comprising the amino acid sequence of SEQ ID NO: 10, or a variant thereof comprising up to 5, 4, 3, 2, or 1 amino acid substitutions in the HC-CDRs; and the V_{L} comprises a LC-CDR1 comprising the amino acid sequence of SEQ ID NO: 16, a LC-CDR2 comprising the amino acid sequence of SEQ ID NO: 18, and a LC-CDR3 comprising the amino acid sequence of any one of SEQ ID NOs: 20, or a variant thereof comprising up to 5, 4, 3, 2, or 1 amino acid substitutions in the LC-CDRs; or
h) the V_{H} comprises a HC-CDR1 comprising the amino acid sequence of SEQ ID NO: 4, a HC-CDR2 comprising the amino acid sequence of SEQ ID NO: 8, and a HC-CDR3 comprising the amino acid sequence of SEQ ID NO: 11, or a variant thereof comprising up to 5, 4, 3, 2, or 1 amino acid substitutions in the HC-CDRs; and the V_{L} comprises a LC-CDR1 comprising the amino acid sequence of SEQ ID NO: 17, a LC-CDR2 comprising the amino acid sequence of SEQ ID NO: 19, and a LC-CDR3 comprising the amino acid sequence of any one of SEQ ID NOs: 21, or a variant thereof comprising up to 5, 4, 3, 2, or 1 amino acid substitutions in the LC-CDRs.

Embodiment 17. The anti-Sclerostin construct of any one of embodiments 1-16, wherein the antibody moiety is an antibody or antigen-binding fragment thereof selected from the group consisting of a full-length antibody, a bispecific antibody, a single-chain Fv (scFv) fragment, a Fab fragment, a Fab′ fragment, a F(ab′)2, an Fv fragment, a disulfide stabilized Fv fragment (dsFv), a disulfide stabilized scFv (dsscFv), a (dsFv)2, a Fv-Fc fusion, a scFv-Fc fusion, a scFv-Fv fusion, a diabody, a tribody, and a tetrabody.

Embodiment 18. The anti-Sclerostin construct of any one of embodiments 1-17, wherein the construct is a full-length antibody comprising an Fc fragment.

Embodiment 19. The anti-Sclerostin construct of embodiment 17, wherein the antibody moiety is a scFv fragment.

Embodiment 20. The anti-Sclerostin construct of any one of embodiments 1-19, wherein the Sclerostin is a human Sclerostin.

Embodiment 21. The anti-Sclerostin construct of any one of embodiments 1-17 and 19-20, wherein the anti-Sclerostin construct further comprises a second moiety.

Embodiment 22. The anti-Sclerostin construct of embodiment 21, wherein the second moiety comprises a second antibody moiety that specifically recognizes an antigen.

Embodiment 23. The anti-Sclerostin construct of embodiment 22, wherein the second antibody moiety comprises a second heavy chain variable region (V_{H-2}) and a light chain variable region (V_{L-2}).

Embodiment 24. The anti-Sclerostin construct of embodiment 22 or embodiment 23, wherein the antigen is DKK1.

Embodiment 25. The anti-Sclerostin construct of embodiment 24, wherein the DKK1 is a human DKK1.

Embodiment 26. The anti-Sclerostin construct of embodiment 24 or embodiment 25, wherein the second antibody moiety competes for a binding epitope of DKK1 with a third antibody moiety comprising a third heavy chain variable region (V_{H-3}) and a third light chain variable region (V_{L-3}), wherein:
a) the V_{H-3} comprising a HC-CDR1 comprising the amino acid sequence of SEQ ID NO: 42, a HC-CDR2 comprising the amino acid sequence of SEQ ID NO: 43, and a HC-CDR3 comprising the amino acid sequence of SEQ ID NO: 44, and the V_{L-3} comprises a LC-CDR1 comprising the amino acid sequence of SEQ ID NO: 45, a LC-CDR2 comprising the amino acid sequence of SEQ ID NO: 46, and a LC-CDR3 comprising the amino acid sequence of SEQ ID NO: 47;
b) the V_{H-3} comprises a HC-CDR1 comprising the amino acid sequence of SEQ ID NO: 48, a HC-CDR2 comprising the amino acid sequence of SEQ ID NO: 49, and a HC-CDR3 comprising the amino acid sequence of SEQ ID NO: 50, and the V_{L-3} comprises a LC-CDR1 comprising the amino acid sequence of SEQ ID NO: 51, a LC-CDR2 comprising the amino acid sequence of SEQ ID NO: 52, and a LC-CDR3 comprising the amino acid sequence of SEQ ID NO: 58; or
c) the V_{H-3} comprises a HC-CDR1 comprising the amino acid sequence of SEQ ID NO: 54, a HC-CDR2 comprising the amino acid sequence of SEQ ID NO: 55, and a HC-CDR3 comprising the amino acid sequence of SEQ ID NO: 56, and the V_{L-3} comprises a LC-CDR1 comprising the amino acid sequence of SEQ ID NO: 57, a LC-CDR2 comprising the amino acid sequence of SEQ ID NO: 58, and a LC-CDR3 comprising the amino acid sequence of SEQ ID NO: 59.

Embodiment 27. The anti-Sclerostin construct of embodiment 26, wherein V_{H-2} comprises the HC-CDR1 comprising the amino acid sequence of SEQ ID NO: 42, the HC-CDR2 comprising the amino acid sequence of SEQ ID NO: 43, and the HC-CDR3 comprising the amino acid sequence of SEQ ID NO: 44, and the V_{L-2} comprises the LC-CDR1 comprising the amino acid sequence of SEQ ID NO: 45, the LC-CDR2 comprising the amino acid sequence of SEQ ID NO: 46, and the LC-CDR3 comprising the amino acid sequence of SEQ ID NO: 47.

Embodiment 28. The anti-Sclerostin construct of embodiment 26, wherein the V_{H-2} comprises the HC-CDR1 comprising the amino acid sequence of SEQ ID NO: 48, the HC-CDR2 comprising the amino acid sequence of SEQ ID NO: 49, and the HC-CDR3 comprising the amino acid sequence of SEQ ID NO: 50, and the V_{L-2} comprises the LC-CDR1 comprising the amino acid sequence of SEQ ID NO: 51, the LC-CDR2 comprising the amino acid sequence of SEQ ID NO: 52, and the LC-CDR3 comprising the amino acid sequence of SEQ ID NO: 58.

Embodiment 29. The anti-Sclerostin construct of embodiment 26, wherein the V_{H-2} comprising the HC-CDR1 comprising the amino acid sequence of SEQ ID NO: 54, the HC-CDR2 comprising the amino acid sequence of SEQ ID NO: 55, and the HC-CDR3 comprising the amino acid sequence of SEQ ID NO: 56, and the V_{L-2} comprises the LC-CDR1 comprising the amino acid sequence of SEQ ID NO: 57, the LC-CDR2 comprising the amino acid sequence of SEQ ID NO: 58, and the LC-CDR3 comprising the amino acid sequence of SEQ ID NO: 59.

Embodiment 30. The anti-Sclerostin construct of embodiment 22 or embodiment 23, wherein the antigen is RANKL.

Embodiment 31. The anti-Sclerostin construct of embodiment 30, wherein the antigen is human RANKL.

Embodiment 32. The anti-Sclerostin construct of embodiment 30 or embodiment 31, wherein the second antibody moiety competes for a binding epitope of RANKL with a third antibody moiety comprising a third heavy chain variable region (V_{H-3}) and a third light chain variable region (V_{L-3}), wherein the V_{H-3} comprising a HC-CDR1 comprising the amino acid sequence of SEQ ID NO: 66, a HC-CDR2 comprising the amino acid sequence of SEQ ID NO: 67, and a HC-CDR3 comprising the amino acid sequence of SEQ ID NO: 68, and the V_{L-3} comprises a LC-CDR1 comprising the amino acid sequence of SEQ ID NO: 69, a LC-CDR2 comprising the amino acid sequence of SEQ ID NO: 70, and a LC-CDR3 comprising the amino acid sequence of SEQ ID NO: 71.

Embodiment 33. The anti-Sclerostin construct of embodiment 31, wherein the V_{H-2} comprises the HC-CDR1 comprising the amino acid sequence of SEQ ID NO: 66, the HC-CDR2 comprising the amino acid sequence of SEQ ID NO: 67, and the HC-CDR3 comprising the amino acid sequence of SEQ ID NO: 68, and the V_{L-2} comprises the LC-CDR1 comprising the amino acid sequence of SEQ ID NO: 69, the LC-CDR2 comprising the amino acid sequence of SEQ ID NO: 70, and the LC-CDR3 comprising the amino acid sequence of SEQ ID NO: 71.

Embodiment 34. The anti-Sclerostin construct of any one of embodiments 22-33, wherein the second antibody moiety is a full-length antibody, a Fab, a Fab′, a (Fab′)2, an Fv, a single chain Fv (scFv) fragment, an scFv-scFv, a minibody, a diabody, or an sdAb.

Embodiment 35. The anti-Sclerostin construct of embodiment 34, wherein the second antibody moiety is a full-length antibody comprising two heavy chains, two light chains and a Fc fragment, and wherein the anti-Sclerostin antibody moiety is a single chain Fv (scFv) fragment comprising the V_{H} fused with the V_{L}.

Embodiment 36. The anti-Sclerostin construct of embodiment 35, wherein the anti-Sclerostin antibody moiety is fused to one or both of the heavy chains of the full-length antibody.

Embodiment 37. The anti-Sclerostin construct of any one of embodiments 34-36, wherein the anti-Sclerostin antibody moiety is fused to one or both of the light chains of the full-length antibody.

Embodiment 38. The anti-Sclerostin construct of embodiment 36 or 37, wherein the anti-Sclerostin antibody moiety is fused to N-terminus of the one or both of the heavy chains or light chains of the full-length antibody.

Embodiment 39. The anti-Sclerostin construct of any one of embodiments 36-38, wherein the anti-Sclerostin antibody moiety is fused to C-terminus of the one or both of the heavy chains or light chains of the full-length antibody.

Embodiment 40. The anti-Sclerostin construct of any one of embodiments 36-39, wherein the anti-Sclerostin antibody moiety is fused to the full-length antibody via a first linker.

Embodiment 41. The anti-Sclerostin construct of any one of embodiments 36-39, wherein the anti-Sclerostin antibody moiety is fused to the full-length antibody without a linker.

Embodiment 42. The anti-Sclerostin construct of embodiment 40, wherein the first linker is a GS linker selected from the group consisting of SEQ ID NOs: 74-84.

Embodiment 43. The anti-Sclerostin construct of any one of embodiments 35-42, wherein the V_{H} is fused with the V_{L} via a second linker.

Embodiment 44. The anti-Sclerostin construct of embodiment 43, wherein the scFv fragment comprises, from N-terminus to C-terminus, the V_{H}, the second linker, and the V_{L}.

Embodiment 45. The anti-Sclerostin construct of embodiment 43, wherein the scFv fragment comprises, from N-terminus to C-terminus, the V_{L}, the second linker, and the V_{H}, and optionally a C-terminal alanine residue.

Embodiment 46. The anti-Sclerostin construct of any one of embodiments 43-45, wherein the second linker comprises an amino acid sequence of SEQ ID NO: 76 or 77.

Embodiment 47. The anti-Sclerostin construct of any one of embodiments 23-33, wherein the second antibody moiety is a scFv fragment comprising the V_{H-2} and the V_{L-2}, and wherein the anti-Sclerostin antibody moiety is a full-length antibody comprising two heavy chains, two light chains and a Fc fragment.

Embodiment 48. The anti-Sclerostin construct of embodiment 46, wherein the second antibody moiety is fused to both of the heavy chains of the full-length antibody.

Embodiment 49. The anti-Sclerostin construct of embodiment 47 or 48, wherein the second antibody moiety is fused to both of the light chains of the full-length antibody.

Embodiment 50. The anti-Sclerostin construct of embodiment 48 or 49, wherein the antibody moiety is fused to N-terminus of both of the heavy chains or light chains of the full-length antibody.

Embodiment 51. The anti-Sclerostin construct of any one of embodiments 47-50, wherein the antibody moiety is fused to C-terminus of both of the heavy chains or light chains of the full-length antibody.

Embodiment 52. The anti-Sclerostin construct of any one of embodiments 47-51, wherein the antibody moiety is fused to the full-length antibody via a first linker.

Embodiment 53. The anti-Sclerostin construct of any one of embodiments 47-51, wherein the antibody moiety is fused to the full-length antibody without a linker.

Embodiment 54. The anti-Sclerostin construct of embodiment 52, wherein the first linker is a GS first linker selected from the group consisting of SEQ ID NOs: 74-84.

Embodiment 55. The anti-Sclerostin construct of any one of embodiments 47-54, wherein the V_{H-2} is fused with the V_{L-2} via a second linker.

Embodiment 56. The anti-Sclerostin construct of any one of embodiments 47-54, wherein the V_{H-2} is fused with the V_{L-2} without a linker.

Embodiment 57. The anti-Sclerostin construct of embodiment 55, wherein the scFv fragment comprises, from N-terminus to C-terminus, the V_{H-2}, the second linker, and the V_{L-2}.

Embodiment 58. The anti-Sclerostin construct of embodiment 55, wherein the scFv fragment comprises, from N-terminus to C-terminus, the V_{L-2}, the second linker, and the V_{H-2}, and optionally a C-terminal alanine residue.

Embodiment 59. The anti-Sclerostin construct of any one of embodiments 55 and 57-58, wherein the second linker comprises an amino acid sequence of SEQ ID NO: 76 or 77.

Embodiment 60. The anti-Sclerostin construct of any one of embodiments 35-59, wherein the scFv is a disulfide stabilized scFv (“dsscFv”).

Embodiment 61. The anti-Sclerostin construct of embodiment 60, wherein the dsscFv comprises a) a G44C mutation in the V_{H} or V_{H-2} according to the numbering of SEQ ID NO: 60, and b) a G100C mutation in the V_{L} or V_{L-2} according to the numbering of SEQ ID NO: 61.

Embodiment 62. The anti-Sclerostin construct of any one of embodiments 35-61, wherein the construct comprises:
1) two heavy chains fused with the anti-Sclerostin or second antibody moiety each comprising the amino acid sequence of SEQ ID NO: 91, and two light chains each comprising the amino acid sequence of SEQ ID NO: 93;
2) two heavy chains fused with the anti-Sclerostin or second antibody moiety each comprising the amino acid sequence of SEQ ID NO: 92, and two light chains each comprising the amino acid sequence of SEQ ID NO: 93;
3) two heavy chains fused with the anti-Sclerostin or second antibody moiety each comprising the amino acid sequence of SEQ ID NO: 94, and two light chains each comprising the amino acid sequence of SEQ ID NO: 96;
4) two heavy chains fused with the anti-Sclerostin or second antibody moiety each comprising the amino acid sequence of SEQ ID NO: 95, and two light chains each comprising the amino acid sequence of SEQ ID NO: 96;
5) two heavy chains fused with the anti-Sclerostin or second antibody moiety each comprising the amino acid sequence of SEQ ID NO: 97, and two light chains each comprising the amino acid sequence of SEQ ID NO: 99;
6) two heavy chains fused with the anti-Sclerostin or second antibody moiety each comprising the amino acid sequence of SEQ ID NO: 98, and two light chains each comprising the amino acid sequence of SEQ ID NO: 99;
7) two heavy chains fused with the anti-Sclerostin or second antibody moiety each comprising the amino acid sequence of SEQ ID NO: 100, and two light chains each comprising the amino acid sequence of SEQ ID NO: 102;
8) two heavy chains fused with the anti-Sclerostin or second antibody moiety each comprising the amino acid sequence of SEQ ID NO: 101, and two light chains each comprising the amino acid sequence of SEQ ID NO: 102;
9) two heavy chains each comprising the amino acid sequence of SEQ ID NO:103, and two light chains fused with the anti-Sclerostin or second antibody moiety each comprising the amino acid sequence of SEQ ID NO: 104;
10) two heavy chains each comprising the amino acid sequence of SEQ ID NO: 103, and two light chains fused with the anti-Sclerostin or second antibody moiety each comprising the amino acid sequence of SEQ ID NO: 105;
11) two heavy chains each comprising the amino acid sequence of SEQ ID NO: 106, and two light chains fused with the anti-Sclerostin or second antibody moiety each comprising the amino acid sequence of SEQ ID NO: 107;
12) two heavy chains each comprising the amino acid sequence of SEQ ID NO: 106, and two light chains fused with the anti-Sclerostin or second antibody moiety each comprising the amino acid sequence of SEQ ID NO: 108;
13) two heavy chains each comprising the amino acid sequence of SEQ ID NO: 109, and two light chains fused with the anti-Sclerostin or second antibody moiety each comprising the amino acid sequence of SEQ ID NO: 110;
14) two heavy chains each comprising the amino acid sequence of SEQ ID NO: 109, and two light chains fused with the anti-Sclerostin or second antibody moiety each comprising the amino acid sequence of SEQ ID NO: 111;
15) two heavy chains each comprising the amino acid sequence of SEQ ID NO: 112, and two light chains fused with the anti-Sclerostin or second antibody moiety each comprising the amino acid sequence of SEQ ID NO: 113;
16) two heavy chains each comprising the amino acid sequence of SEQ ID NO: 112, and two light chains fused with the anti-Sclerostin or second antibody moiety each comprising the amino acid sequence of SEQ ID NO: 114;
17) two heavy chains fused with the anti-Sclerostin or second antibody moiety each comprising the amino acid sequence of SEQ ID NO: 115, and two light chains each comprising the amino acid sequence of SEQ ID NO: 119;
18) two heavy chains fused with the anti-Sclerostin or second antibody moiety each comprising the amino acid sequence of SEQ ID NO: 116, and two light chains each comprising the amino acid sequence of SEQ ID NO: 119;
19) two heavy chains fused with the anti-Sclerostin or second antibody moiety each comprising the amino acid sequence of SEQ ID NO: 117, and two light chains each comprising the amino acid sequence of SEQ ID NO: 119;
20) two heavy chains fused with the anti-Sclerostin or second antibody moiety each comprising the amino acid sequence of SEQ ID NO: 118, and two light chains each comprising the amino acid sequence of SEQ ID NO: 119;
21) two heavy chains fused with the anti-Sclerostin or second antibody moiety each comprising the amino acid sequence of SEQ ID NO: 120, and two light chains each comprising the amino acid sequence of SEQ ID NO: 122;
22) two heavy chains fused with the anti-Sclerostin or second antibody moiety each comprising the amino acid sequence of SEQ ID NO: 121, and two light chains each comprising the amino acid sequence of SEQ ID NO: 122;
23) two heavy chains fused with the anti-Sclerostin or second antibody moiety each comprising the amino acid sequence of SEQ ID NO: 123, and two light chains each comprising the amino acid sequence of SEQ ID NO: 125;
24) two heavy chains fused with the anti-Sclerostin or second antibody moiety each comprising the amino acid sequence of SEQ ID NO: 124, and two light chains each comprising the amino acid sequence of SEQ ID NO: 125;
25) two heavy chains fused with the anti-Sclerostin or second antibody moiety each comprising the amino acid sequence of SEQ ID NO: 126, and two light chains each comprising the amino acid sequence of SEQ ID NO: 129;
26) two heavy chains fused with the anti-Sclerostin or second antibody moiety each comprising the amino acid sequence of SEQ ID NO: 127, and two light chains each comprising the amino acid sequence of SEQ ID NO: 129;
27) two heavy chains fused with the anti-Sclerostin or second antibody moiety each comprising the amino acid sequence of SEQ ID NO: 128, and two light chains each comprising the amino acid sequence of SEQ ID NO: 129;
28) two heavy chains each comprising the amino acid sequence of SEQ ID NO: 133, and two light chains fused with the anti-Sclerostin or second antibody moiety each comprising the amino acid sequence of SEQ ID NO: 130;
29) two heavy chains each comprising the amino acid sequence of SEQ ID NO: 133, and two light chains fused with the anti-Sclerostin or second antibody moiety each comprising the amino acid sequence of SEQ ID NO: 131;
30) two heavy chains each comprising the amino acid sequence of SEQ ID NO: 133, and two light chains fused with the anti-Sclerostin or second antibody moiety each comprising the amino acid sequence of SEQ ID NO: 132;
31) two heavy chains fused with the anti-Sclerostin or second antibody moiety each comprising the amino acid sequence of SEQ ID NO: 177, and two light chains each comprising the amino acid sequence of SEQ ID NO: 125;
32) two heavy chains fused with the anti-Sclerostin or second antibody moiety each comprising the amino acid sequence of SEQ ID NO: 178, and two light chains each comprising the amino acid sequence of SEQ ID NO: 125;
33) two heavy chains fused with the anti-Sclerostin or second antibody moiety each comprising the amino acid sequence of SEQ ID NO: 178, and two light chains each comprising the amino acid sequence of SEQ ID NO: 122;
34) two heavy chains fused with the anti-Sclerostin or second antibody moiety each comprising the amino acid sequence of SEQ ID NO: 179, and two light chains each comprising the amino acid sequence of SEQ ID NO: 122; or
35) two heavy chains fused with the anti-Sclerostin or second antibody moiety each comprising the amino acid sequence of SEQ ID NO: 180, and two light chains each comprising the amino acid sequence of SEQ ID NO: 122.

Embodiment 63. The anti-Sclerostin construct of any one of embodiments 23-33, wherein the construct comprises:
a) a first polypeptide comprising a first light chain comprising, from N-terminus to C-terminus, i) the V_{L}, ii) a first light chain constant domain (“first CL domain”);
b) a second polypeptide comprising a first heavy chain comprising, from N-terminus to C-terminus, i) the V_{H}, ii) a first heavy chain constant domain (“first CH1 domain”), and iii) a first Fc domain;
c) a third polypeptide comprising a second heavy chain comprising, from N-terminus to C-terminus, i) the V_{H-2}, ii) a second heavy chain constant domain (“second CH1 domain”), and iii) a second Fc domain; and
d) a fourth polypeptide comprising a second light chain comprising, from N-terminus to C-terminus, i) the V_{L-2}, ii) ii) a second light chain constant domain (“second CL domain”), wherein the first and the second Fc domains form an Fc fragment.

Embodiment 64. The anti-Sclerostin construct of embodiment 63, wherein one of the first and the second Fc domains comprises a T366W mutation, and optionally a S354C mutation, and wherein the other Fc domain comprises a T366S mutation, a L368A mutation, a Y407V mutation, and optionally a Y349C mutation, wherein numbering is according to the EU index.

Embodiment 65. The anti-Sclerostin construct of embodiment 63 or embodiment 64, wherein either i) the first CH1 domain and the first CL domain or ii) the second CH1 domain and the second CL domain are selected from the group consisting of:
a) a CH1 domain wherein the amino acid at position 141 is substituted for cysteine and the cysteine at position 131 (e.g., in IgG2 or IgG4) or 220 (e.g., in IgG1) is substituted for a non-cysteine amino acid; and a CL domain wherein the amino acid at position 116 is substituted for cysteine and the cysteine at position 214 is substituted for a non-cysteine amino acid;
b) a CH1 domain wherein the amino acid at position 168 is substituted for cysteine and the cysteine at position 131 (e.g., in IgG2 or IgG4) or 220 (e.g., in IgG1) is substituted for a non-cysteine amino acid; and a CL domain wherein the amino acid at position 164 is substituted for cysteine and the cysteine at position 214 is substituted for a non-cysteine amino acid;
c) a CH1 domain wherein the amino acid at position 126 is substituted for cysteine and the cysteine at position 131 (e.g., in IgG2 or IgG4) or 220 (e.g., in IgG1) is substituted for a non-cysteine amino acid; and a CL domain wherein the amino acid at position 121 is substituted for cysteine and the cysteine at position 214 is substituted for a non-cysteine amino acid;
d) a CH1 domain wherein the amino acid at position 128 is substituted for cysteine and the cysteine at position 131 (e.g., in IgG2 or IgG4) or 220 (e.g., in IgG1) is substituted for a non-cysteine amino acid; and a CL domain wherein the amino acid at position 118 is substituted for cysteine and the cysteine at position 214 is substituted for a non-cysteine amino acid;
e) a CH1 domain wherein the amino acid at position 170 is substituted for cysteine and the cysteine at position 131 (e.g., in IgG2 or IgG4) or 220 (e.g., in IgG1) is substituted for a non-cysteine amino acid; and a CL domain wherein the amino acid at position 176 is substituted for cysteine and the cysteine at position 214 is substituted for a non-cysteine amino acid;
f) a CH1 domain wherein the amino acid at position 171 is substituted for cysteine and the cysteine at position 131 (e.g., in IgG2 or IgG4) or 220 (e.g., in IgG1) is substituted for a non-cysteine amino acid; and a CL domain wherein the amino acid at position 162 is substituted for cysteine and the cysteine at position 214 is substituted for a non-cysteine amino acid;
g) a CH1 domain wherein the amino acid at position 173 is substituted for cysteine and the cysteine at position 131 (e.g., in IgG2 or IgG4) or 220 (e.g., in IgG1) is substituted for a non-cysteine amino acid; and a CL domain wherein the amino acid at position 160 is substituted for cysteine and the cysteine at position 214 is substituted for a non-cysteine amino acid;
wherein numbering is according to the EU index.

Embodiment 66. The anti-Sclerostin construct of any of embodiments 63-65, comprising:
1) the first polypeptide comprises the amino acid sequence of SEQ ID NO: 135, the second polypeptide comprises the amino acid sequence of SEQ ID NO: 134, the third polypeptide comprises the amino acid sequence of SEQ ID NO: 136, and the fourth polypeptide comprises the amino acid sequence of SEQ ID NO: 137;
2) the first polypeptide comprises the amino acid sequence of SEQ ID NO: 135, the second polypeptide comprises the amino acid sequence of SEQ ID NO: 134, the third polypeptide comprises the amino acid sequence of SEQ ID NO: 138, and the fourth polypeptide comprises the amino acid sequence of SEQ ID NO: 139;
3) the first polypeptide comprises the amino acid sequence of SEQ ID NO: 135, the second polypeptide comprises the amino acid sequence of SEQ ID NO: 134, the third polypeptide comprises the amino acid sequence of SEQ ID NO: 140, and the fourth polypeptide comprises the amino acid sequence of SEQ ID NO: 141;
4) the first polypeptide comprises the amino acid sequence of SEQ ID NO: 135, the second polypeptide comprises the amino acid sequence of SEQ ID NO: 134, the third polypeptide comprises the amino acid sequence of SEQ ID NO: 142, and the fourth polypeptide comprises the amino acid sequence of SEQ ID NO: 143;
5) the first polypeptide comprises the amino acid sequence of SEQ ID NO: 135, the second polypeptide comprises the amino acid sequence of SEQ ID NO: 134, the third polypeptide comprises the amino acid sequence of SEQ ID NO: 144, and the fourth polypeptide comprises the amino acid sequence of SEQ ID NO: 145;
6) the first polypeptide comprises the amino acid sequence of SEQ ID NO: 147, the second polypeptide comprises the amino acid sequence of SEQ ID NO: 146, the third polypeptide comprises the amino acid sequence of SEQ ID NO: 148, and the fourth polypeptide comprises the amino acid sequence of SEQ ID NO: 149;
7) the first polypeptide comprises the amino acid sequence of SEQ ID NO: 147, the second polypeptide comprises the amino acid sequence of SEQ ID NO: 146, the third polypeptide comprises the amino acid sequence of SEQ ID NO: 150, and the fourth polypeptide comprises the amino acid sequence of SEQ ID NO: 151;
8) the first polypeptide comprises the amino acid sequence of SEQ ID NO: 147, the second polypeptide comprises the amino acid sequence of SEQ ID NO: 146, the third polypeptide comprises the amino acid sequence of SEQ ID NO: 152, and the fourth polypeptide comprises the amino acid sequence of SEQ ID NO: 153;
9) the first polypeptide comprises the amino acid sequence of SEQ ID NO: 147, the second polypeptide comprises the amino acid sequence of SEQ ID NO: 146, the third polypeptide comprises the amino acid sequence of SEQ ID NO: 154, and the fourth polypeptide comprises the amino acid sequence of SEQ ID NO: 155;
10) the first polypeptide comprises the amino acid sequence of SEQ ID NO: 147, the second polypeptide comprises the amino acid sequence of SEQ ID NO: 146, the third polypeptide comprises the amino acid sequence of SEQ ID NO: 156, and the fourth polypeptide comprises the amino acid sequence of SEQ ID NO: 157;
11) the first polypeptide comprises the amino acid sequence of SEQ ID NO: 147, the second polypeptide comprises the amino acid sequence of SEQ ID NO: 146, the third polypeptide comprises the amino acid sequence of SEQ ID NO: 158, and the fourth polypeptide comprises the amino acid sequence of SEQ ID NO: 159;
12) the first polypeptide comprises the amino acid sequence of SEQ ID NO: 147, the second polypeptide comprises the amino acid sequence of SEQ ID NO: 146, the third polypeptide comprises the amino acid sequence of SEQ ID NO: 160, and the fourth polypeptide comprises the amino acid sequence of SEQ ID NO: 161;
13) the first polypeptide comprises the amino acid sequence of SEQ ID NO: 147, the second polypeptide comprises the amino acid sequence of SEQ ID NO: 146, the third polypeptide comprises the amino acid sequence of SEQ ID NO: 162, and the fourth polypeptide comprises the amino acid sequence of SEQ ID NO: 163;
14) the first polypeptide comprises the amino acid sequence of SEQ ID NO: 147, the second polypeptide comprises the amino acid sequence of SEQ ID NO: 171, the third polypeptide comprises the amino acid sequence of SEQ ID NO: 172, and the fourth polypeptide comprises the amino acid sequence of SEQ ID NO: 163.
15) the first polypeptide comprises the amino acid sequence of SEQ ID NO: 147, the second polypeptide comprises the amino acid sequence of SEQ ID NO: 173, the third polypeptide comprises the amino acid sequence of SEQ ID NO: 174, and the fourth polypeptide comprises the amino acid sequence of SEQ ID NO: 163.
16) the first polypeptide comprises the amino acid sequence of SEQ ID NO: 147, the second polypeptide comprises the amino acid sequence of SEQ ID NO: 175, the third polypeptide comprises the amino acid sequence of SEQ ID NO: 176, and the fourth polypeptide comprises the amino acid sequence of SEQ ID NO: 163. Embodiment 67. The anti-Sclerostin construct of embodiment 21, wherein the second moiety comprises a half-life extending moiety.

Embodiment 68. The anti-Sclerostin construct of embodiment 67, wherein the half-life extending moiety is an Fc fragment.

Embodiment 69. The anti-Sclerostin construct of any one of embodiments 18, and 35-66 and 68, wherein the Fc fragment is selected from the group consisting of Fc fragments form IgG, IgA, IgD, IgE, IgM, and combinations and hybrids thereof.

Embodiment 70. The anti-Sclerostin construct of embodiment 69, wherein the Fc fragment is selected from the group consisting of Fc fragments from IgG1, IgG2, IgG3, IgG4, and combinations and hybrids thereof.

Embodiment 71. The anti-Sclerostin construct of embodiment 69 or embodiment 70, wherein the Fc fragment comprises a H435R mutation and a Y436F mutation.

Embodiment 72. The anti-Sclerostin construct of any of embodiments 69-71, wherein the Fc fragment has a reduced effector function as compared to the corresponding wildtype Fc fragment.

Embodiment 73. The anti-Sclerostin construct of any of embodiments 69-71, wherein the Fc fragment has: a) an enhanced effector function as compared to the corresponding wildtype Fc fragment, and/or b) an enhanced FcRn binding affinity as compared to the corresponding wildtype Fc fragment.

Embodiment 74. The anti-Sclerostin construct of embodiment 21, wherein the construct is an antibody-drug conjugate or antibody fusion protein.

Embodiment 75. The anti-Sclerostin construct of embodiment 74, wherein the second moiety comprises an agent or agents selected from the group consisting of a parathyroid hormone (PTH), a selective estrogen receptor modulator (SERM), a bisphosphonate, a prostaglandin E (PGE) receptor agonist, VEGF, TGFβ, growth factor (myostatin), calcitonin and combinations thereof.

Embodiment 76. An anti-Sclerostin construct that specifically binds to Sclerostin competitively with the anti-Sclerostin construct of any one of embodiments 1-75.

Embodiment 77. A pharmaceutical composition comprising the anti-Sclerostin construct of any one of embodiments 1-76 and a pharmaceutically acceptable carrier.

Embodiment 78. The pharmaceutical composition of embodiment 77, wherein the composition further comprises an agent or agents selected from the group consisting of a parathyroid hormone (PTH), a selective estrogen receptor modulator (SERM), VEGF, and TGFβ, growth factor (myostatin), calcitonin and combinations thereof.

Embodiment 79. An isolated nucleic acid encoding the anti-Sclerostin construct of any one of embodiments 1-76.

Embodiment 80. A vector comprising the isolated nucleic acid of embodiment 79.

Embodiment 81. An isolated host cell comprising the isolated nucleic acid of embodiment 79, or the vector of embodiment 80.

Embodiment 82. A method of producing an anti-Sclerostin construct comprising:
a) culturing the isolated host cell of embodiment 81 under conditions effective to express the anti-Sclerostin construct or a portion thereof, and
b) obtaining the expressed anti-Sclerostin construct or a portion thereof from the host cell.

Embodiment 83. A method of treating and/or preventing a disease or condition in an individual, comprising administering to the individual an effective mount of the anti-Sclerostin construct of any one of embodiments 1-76, or the pharmaceutical composition of embodiment 77 or embodiment 78.

Embodiment 84. The method of embodiment 83, wherein the disease or condition is a bone-related disorder or cartilage-related disorder, a bone marrow or haemotological disorder, a musculoskeletal rare disease, a muscle-related disorder, or a cancer.

Embodiment 85. The method of embodiment 84, wherein the bone-related disorder is osteogenesis imperfecta, osteoporosis or osteopenia (in men and women), osteonecrosis, delay bone healing, non-union bone fractures, multiple myeloma, multiple myeloma related bone disorders, primary bone tumor, bone metastasis of malignancies, inflammatory or infectious bone disease, osteomalacia, hypercalcemia, Paget's disease, immobilization-induced bone loss, glucocorticoid-induced bone loss, inflammation-induced bone loss including arthritis-induced bone loss, spaceflight osteoporosis/osteopenia and bone loss caused by reduced gravity or other disease or condition associated with a) bone loss of either quantity or quality or both and/or b) abnormality of bone structure and quality.

Embodiment 86. The method of embodiment 85, wherein the bone-related disorder is osteoporosis or osteopenia.

Embodiment 87. The method of embodiment 85, wherein the bone-related disorder is osteogenesis imperfecta.

Embodiment 88. The method of embodiment 85, wherein the bone-related disorder is multiple myeloma and multiple myeloma related bone disorders.

Embodiment 89. The method of embodiment 84, wherein the cartilage disorder is chondromatosis, chondrodysplasia, achondroplasia, epiphyseal dysplasia, chondrodystrophic myotonia, juxtacortical chondroma, tear of cartilage of knee, osteofibrous dysplasia, osteoarthritis, osteogenesis imperfecta, hypophosphatemic rickets or osteochondrodystrophy.

Embodiment 90. The method of embodiment 84, wherein the muscle-related disorder is sarcopenia and cancer sarcopenia.

Embodiment 91. A method of facilitation of heal after bone or joint surgeries in an individual, comprising administering to the individual an effective mount of the anti-Sclerostin construct of any one of embodiments 1-76, and/or the pharmaceutical composition of embodiment 77 or embodiment 78.

Embodiment 92. The method of any one of embodiments 83-91, wherein the anti-Sclerostin construct is administered by subcutaneous injection, intravenous injection, intramuscular injection or administered orally or parenterally into the individual.

Embodiment 93. The method of any one of embodiments 83-92, wherein the method further comprises administering a second agent or therapy.

Embodiment 94. The method of embodiment 93, wherein the second agent or therapy comprises an anti-DKK1 antibody.

Embodiment 95. The method of embodiment 93, wherein the second agent or therapy comprises an anti-RANKL antibody.

Embodiment 96. The method of embodiment 93, wherein the second agent or therapy comprises an agent selected from the group consisting of a parathyroid hormone (PTH), a selective estrogen receptor modulator (SERM), a bisphosphonate, a prostaglandin E (PGE) receptor agonist, VEGF, TGFβ, growth factor (myostatin) and calcitonin.

Embodiment 97. The method of any one of embodiments 83-96, wherein the individual is a human.

### EXAMPLES

The examples below are intended to be purely exemplary of the application and should therefore not be considered to limit the application in any way. The following examples and detailed description are offered by way of illustration and not by way of limitation.

### Example 1. Generation of Anti-Sclerostin Monoclonal Antibodies

The anti-human Sclerostin monoclonal antibody was produced by immunizing mice. Experimental BALB/c mice (18-20 g, 7-8 weeks of age, Beijing Vital River Laboratory Animal Technology Co., Ltd.) and C57 mice (18-20 g, 7-8 weeks of age, Beijing Vital River Laboratory Animal Technology Co., Ltd.) were used. The animals were kept in the SPF level laboratory for 1 week, with 12/12 hours light/dark cycle, at a temperature of 20-25° C., and humidity of 40-60%. Human SOST recombinant protein with a His tag (HST-H5245, Acro Biosystems) was used as an immunogen. A proprietary adjuvant (Lot No 20200120) was used for emulsification, and the antigen was added with equal volume of adjuvant to reach total volume of 300 ul. The antigen/adjuvant solution was emulsified with electro blender and mixed with multi-dimension rotator. After sanitation with 75% alcohol, the antigen/adjuvant solution was injected subcutaneously. The antigen was emulsified and inoculated on days 0, 14, 28. A booster immunization was performed by intraperitoneal (IP) injection of antigen solution formulated with saline at 50 μg/mouse 3 days prior to splenocyte fusion. A blood titer test was performed on days 22, 36, and 45. After the third immunization, two mice with highest blood titer tending to platform were selected for splenocyte fusion. Hybridoma cells were obtained by fusing splenocyte with myeloma Sp2/0 cells by using a conventional fusion procedure with Electro Cell Manipulator (BTX, ECM2001). The binding activity of mouse serum to human Sclerostin was measured by the ELISA method of Example 3, and the monoclonal hybridoma cell strains with good binding activities in vitro were selected. The results are shown in Table 5.

### Example 2. Cloning and Sequencing of Anti-Sclerostin Monoclonal Antibodies

The process of hybridoma sequencing was performed as follows. Total RNA was isolated from the hybridoma cells following the technical manual of TRIzol® Reagent. Total RNA was then reverse-transcribed into cDNA using either isotype-specific anti-sense primers or universal primers following the technical manual of PrimeScript™ 1st Strand cDNA Synthesis Kit. Antibody fragments of heavy chain and light chain were amplified according to the standard operating procedure (SOP) of rapid amplification of cDNA ends (RACE). Amplified antibody fragments were cloned into a standard cloning vector separately. Colony PCR was performed to screen for clones with inserts of correct sizes. The consensus sequence was provided.

### Sequence Analysis

DNA sequence data from all constructs were analyzed and consensus sequences for heavy and light chain were determined. Tables 3 and 4 list V_{H} and V_{L} CDRs of various antibodies and consensus sequences.

TABLE 3
V_{H} CDRs of various antibodies and consensus sequences.

| | HC-CDR1 | HC-CDR2 | HC-CDR3 |
|---|---|---|---|
| 93B1B7 | DYEIH | AIDPETGGTAYNQKFKG | YDYVTY |
| | (SEQ ID NO: 1) | (SEQ ID NO: 5) | (SEQ ID NO: 9) |
| 94B12D3 | DYEIH | AIDPETGGTAYNQKFKA | YDYVSY |
| | (SEQ ID NO: 1) | (SEQ ID NO: 6) | (SEQ ID NO: 10) |
| 71G6G8 | DYEIH | AIDPETGGTAYNQKFKG | YDYVTY |
| | (SEQ ID NO: 1) | (SEQ ID NO: 5) | (SEQ ID NO: 9) |
| 56E5C10 | DFEMH | AIDPETGGTAYNQKFTA | YDYVSY |
| | (SEQ ID NO: 2) | (SEQ ID NO: 7) | (SEQ ID NO: 10) |
| 91F6D10 | DYEMH | AIDPETGGTAYNQKFKA | YDYVSY |
| | (SEQ ID NO: 3) | (SEQ ID NO: 6) | (SEQ ID NO: 10) |
| 51E8D4 | DFEIH | AIDPETGGTAYNQKFKG | YDYVSY |
| | (SEQ ID NO: | (SEQ ID NO: 5) | (SEQ ID NO: 10) |
| | 199) | | |
| 97C11D7 | DYEMH | AIDPETGGSANNQKFKA | YDYVSY |
| | (SEQ ID NO: 3) | (SEQ ID NO: 200) | (SEQ ID NO: 10) |
| 81B2B6 | DYEMH | AIDPETGGTAYNQKFKA | YDYVSY |
| | (SEQ ID NO: 3) | (SEQ ID NO: 6) | (SEQ ID NO: 10) |
| 84F2D5 | DFEIH | AIDPETGGTAYNQKFKG | YDYVSY |
| | (SEQ ID NO: | (SEQ ID NO: 5) | (SEQ ID NO: 10) |
| | 199) | | |
| 65B12C9 | SYWMH | MIHPNSGSSNYNEKFKS | DYDDEGFAY |
| | (SEQ ID NO: 4) | (SEQ ID NO: 8) | (SEQ ID NO: 11) |
| Consensus | DX₁EX₂H | AIDPETGGX₃AX₄NQKFX₅X₆ | YDYVX₇Y |
| sequence | X₁ = Y or F, | X₃ = T or S, | X₇ = T or S |
| | X₂ = M or I. | X₄ = Y or N, X₅ = K | (SEQ ID NO: 14) |
| | (SEQ ID | or T, X₆ = A, G, or S. | |
| | NO: 12) | (SEQ ID NO: 13) | |

TABLE 4
V_{L} CDRs of various antibodies.

| | LC-CDR1 | LC-CDR2 | LC-CDR3 |
|---|---|---|---|
| 93B1B7 | KSSQSLLYSDGRTYLN | LVSKLDS | WQGTHLPHT |
| | (SEQ ID NO: 15) | (SEQ ID NO: 18) | (SEQ ID NO: 20) |
| 94B12D3 | KSSQSLLYSDGRTYLN | LVSKLDS | WQGTHLPHT |
| | (SEQ ID NO: 15) | (SEQ ID NO: 18) | (SEQ ID NO: 20) |
| 71G6G8 | KSSQSLLYSDGRTYLN | LVSKLDS | WQGTHLPHT |
| | (SEQ ID NO: 15) | (SEQ ID NO: 18) | (SEQ ID NO: 20) |
| 56E5C10 | KSSQSLLYSDGRTYLN | LVSKLDS | WQGTHLPHT |
| | (SEQ ID NO: 15) | (SEQ ID NO: 18) | (SEQ ID NO: 20) |
| 91F6D10 | KSSQSLLYSDGRTYLN | LVSKLDS | WQGTHLPHT |
| | (SEQ ID NO: 15) | (SEQ ID NO: 18) | (SEQ ID NO: 20) |
| 51E8D4 | KSSQSLLYSDGKTYLN | LVSKLDS | WQGTHLPHT |
| | (SEQ ID NO: 16) | (SEQ ID NO: 18) | (SEQ ID NO: 20) |
| 97C11D7 | KSSQSLLYSDGRTYLN | LVSKLDS | WQGTHLPHT |
| | (SEQ ID NO: 15) | (SEQ ID NO: 18) | (SEQ ID NO: 20) |
| 81B2B6 | KSSQSLLYSDGRTYLN | LVSKLDS | WQGTHLPHT |
| | (SEQ ID NO: 15) | (SEQ ID NO: 18) | (SEQ ID NO: 20) |
| 84F2D5 | KSSQSLLYSDGKTYLN | LVSKLDS | WQGTHLPHT |
| | (SEQ ID NO: 16) | (SEQ ID NO: 18) | (SEQ ID NO: 20) |
| 65B12C9 | KASQSVSNDVA | YASNRCT | QQDYSSPWT |
| | (SEQ ID NO: 17) | (SEQ ID NO: 19) | (SEQ ID NO: 21) |

### Example 3. Binding of Anti-Sclerostin Antibodies to Sclerostin as Measured by ELISA

The ability of antibodies to specifically bind Sclerostin was determined by an ELISA capture assay. Plates were coated, in a 96-well half-area, with 100 μl/well of mouse His-tagged human Sclerostin (NCBI® reference No. is NP_079513.1) [0.5 μg/mg], in coating buffer (PBS, pH 7.4), and incubated at RT for one hour, or 4° C. overnight. The plates were washed once with 100 μl/well of washing solution (PBS containing 0.2% Tween™20, BIO-RAD). Plates were then incubated in blocking solution (PBS containing 1% BSA, 1% goat serum and 0.5% Tween™20; 100 μl/well) for one hour at room temperature (RT). Anti-Sclerostin antibodies at various concentrations (1000, 333.3, 111.1, 37.0, 12.3, 4.1, 1.4, 0.5, 0.15, 0.05 ng/ml), a positive control antibody Romosozumab or non-Sclerostin related IgG (human or rat IgG, negative control), were then added to each well in 20 μl/well of blocking solution, and plates were incubated at RT for one hour. NeutrAvidin™ HRP (Pierce, Catalog #31001) diluted in blocking solution (1:50,000 dilution) was added at to each well (20 ml/well), and the plates were incubated at RT for one hour prior to three washes with 100 ml/well of washing solution. SuperSignal ELISA Femto (ThermoFisher Scientific, Catalog #37074) working solution was added to the plates (20 ml/well), and the signal was read using a luminometer at 425 nm.

As shown in Table 5 below, the tested anti-Sclerostin antibodies exhibited comparable or more advantageous EC₅₀ value as compared to that of Romosozumab. Specifically, 93B1B7 exhibited more advantageous EC₅₀ value as compared to that of Romosozumab.

TABLE 5
The ELISA activity of anti-Sclerostin murine antibodies.

| Antibodies | EC₅₀ (nM) |
|---|---|
| 93B1B7 | 0.04 |
| 94B12D3 | 0.04 |
| 71G6G8 | 0.04 |
| 56E5C10 | 0.09 |
| 91F6D10 | 0.06 |
| 51E8D4 | 0.07 |
| 97C11D7 | 0.04 |
| 81B2B6 | 0.01 |
| 84F2D5 | 0.02 |
| 65B12C9 | 0.04 |
| Romosozumab | 0.11 |

### Example 4. Binding Epitopes Identification for Anti-Sclerostin Antibody

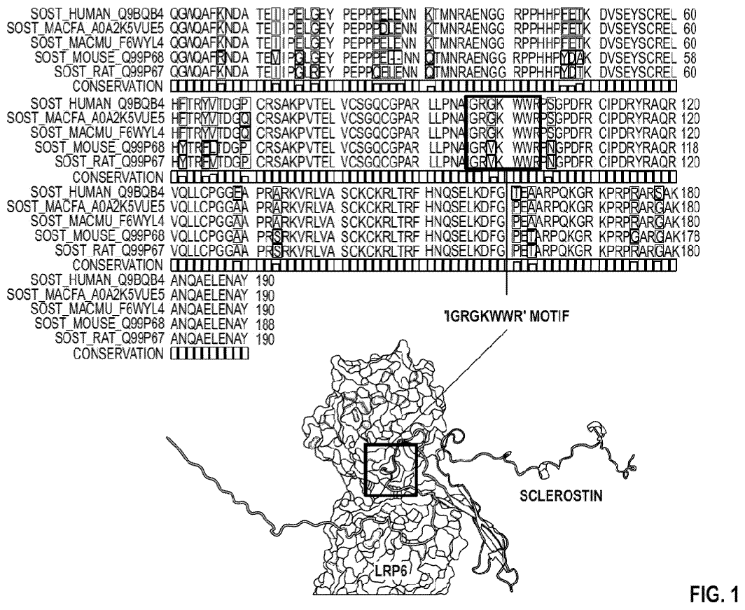

Targeting the correct epitope is a critical step in selection of a monoclonal antibody to achieve the desired mechanism of action (Wilson, P C, 2012, Nat. Rev. Immunol. 12, 709-719). The synthetic peptide-based antibody production against B cell epitopes has been used broadly in developing therapeutic antibodies and vaccines for various diseases in the known art (Ben-Yedidia T., 1997, Curr Opin Bio technol 8:442-448; Bijker M S, 2007, Expert Rev Vaccines 6:591-603), with the specificity advantage of obtained antibodies being restricted to defined epitopes. The second loop on Sclerostin has been proven by NMR and crystallographic structures to interact directly with low-density lipoprotein receptor-related protein family member, e.g., LRP5 or LRP6, and blocks the interaction between LRPs and Wnt family members. Based on crystallographic structure of human Sclerostin in complex with LRP6 (Kim, J. 2020, Nat. Commun. 11: 5357-5357), the loop tip containing the ‘IGRGKWWR’ motif (SEQ ID NO: 186) as shown in FIG. 1 was found to be the main binding determinant for binding of Sclerostin to the first propeller of LRP6.

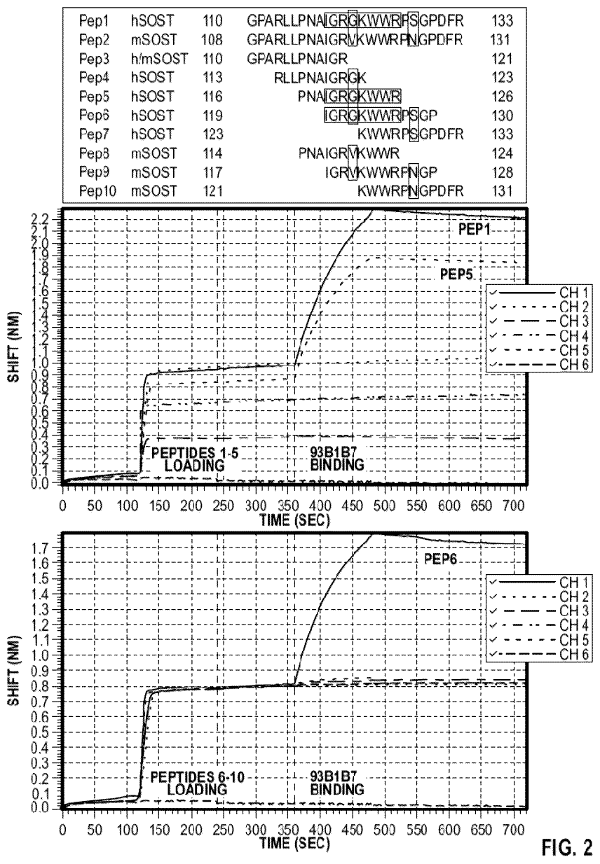

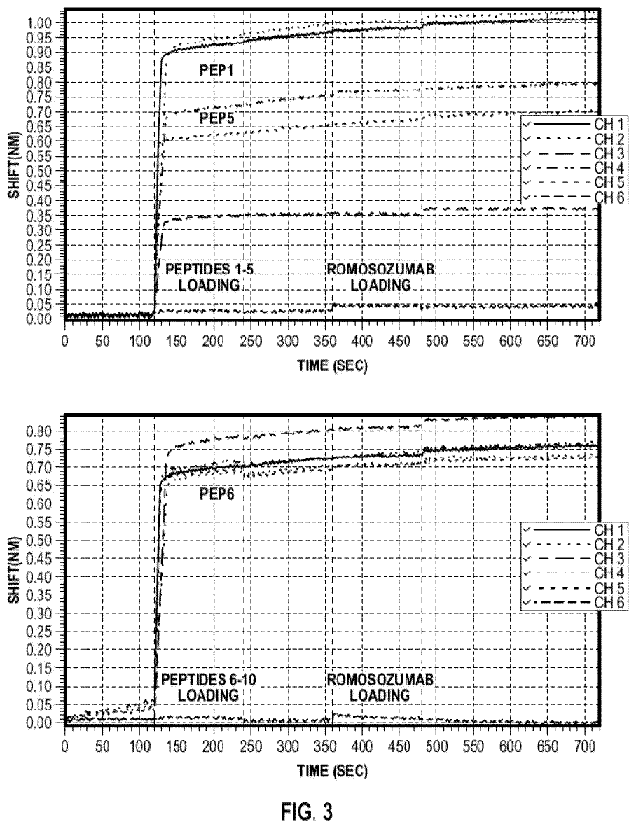

Antibody 93B1B7's binding to chemically synthesized peptides comprising the sequences listed in FIG. 2 from the second loop of human and mouse sclerostin was tested with bio-layer interferometry approach (Gator®, Probe Life). The peptides were biotinylated (NHS-Biotin Reagents, Thermo, #21343) and immobilized on Streptavidin biosensor, using a 0.5 μg/ml solution. 5 μg/ml mAb 93B1B7 in kinetics buffer (PBS, pH 7.4, 0.05% Tween™-20, 0.2% BSA) was used as the analytes and binding sensorgrams are shown in FIG. 1. Pep1 (SEQ ID NO:185), pep5 (SEQ ID NO:187) and (pep6 SEQ ID NO:188) that have the same ‘IGRGKWWR’ (SEQ ID NO:186) motif on human Sclerostin were recognized by 93B1B7 with high affinity. In contrast, these peptides cannot be recognized by Romosozumab as show in FIG. 3.

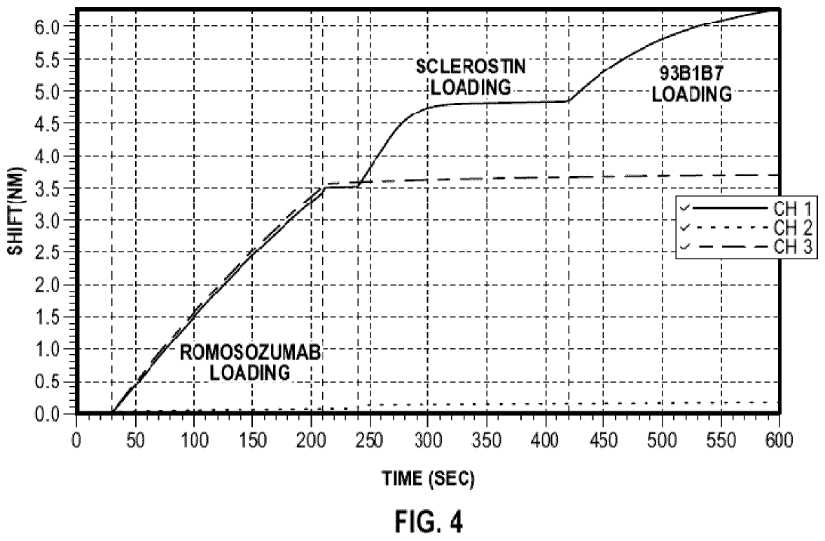

To further demonstrate that the binding epitope of 93B1B7 is completely different from that of Romosozumab, 5 μg/ml Romosozumab was immobilized on anti-hFc probe, and 2 μg/ml human sclerostin and 5 μg/ml 93B1B7 in kinetics buffer (PBS, pH 7.4, 0.05% Tween™-20, 0.2% BSA) were sequentially added as analytes. The binding sensorgram is shown in FIG. 4 which indicating Romosozumab and 93B1B7 can bind sclerostin simultaneously by occupying different binding sites and thus do not cross-block each other. Therefore, the binding epitope (‘IGRGKWWR’ motif, SEQ ID NO: 186) are unique to the antibodies herein and do not overlap with that of Romosozumab.

The majority of anti-sclerostin antibodies discovered here antibodies have very similar binding activities and CDR sequences (see Tables 3 and 4), so we believe that they share the same specificity and binding epitopes. Thus, these antibodies discovered herein were further confirmed that they bind to a specific epitope on Sclerostin that has the ‘IGRGKWWR’ motif (SEQ ID NO: 186). This enables them to completely prevent the binding between Sclerostin and LRP6, thereby resulting in stronger Sclerostin neutralization than other anti-Sclerostin antibodies such as Romosozumab. This is consistent with the observation that the antibodies disclosed herein are more advantageous over Romosozumab in sclerostin binding and neutralizing activities as disclosed in the examples below.

### Example 5. Humanization of Anti-Sclerostin Antibodies

Antibody 93B1B7 was humanized by grafting the complementarity determining regions (CDRs) onto human germline frameworks. The light chain germline acceptor sequence chosen was the human VK2 A19 V-region and VK1 O12 V-region plus JK4 J-region (V-BASE). The heavy chain germline acceptor sequence chosen was the human VH1 1-18 V-region plus JH4 J-region (V BASE). The CDR's grafted from the donor to the acceptor sequence are as defined by Kabat (Kabat et al. 1987), with the exception of CDR-H1 and H2 where the combined Chothia/Kabat definition is used. Genes encoding a full-length IgG4P antibody encompassing initial V-region sequences were constructed by an automated synthesis approach (Vanzyme Inc.), and modified to generate the final grafted versions by oligonucleotide directed mutagenesis. In order to retain full activity and maintain high thermostability, donor residues at positions 93 (Tyrosine) and 94 (Serine) of the humanized heavy chain (Kabat numbering) were retained, and acceptor residue at position 43 (Glutamine) was changed to Lysine which is more populated at this position. Similarly, donor residues at position 46 (Arginine) of the humanized light chains (Kabat numbering) were retained. In addition, one potential aspartate isomerization site in the light chain CDR Li was removed by mutating Glycerin residue at position 30 to Serine. Humanized anti-Sclerostin antibodies hAb-1, hAb-2 and hAb-3 were generated. See Table 6 for their CDR, V_{H} and V_{L} sequences.

TABLE 6
CDR, V_{H} and V_{L} sequences of exemplary
humanized anti-Sclerostin antibodies

| | hAb-1 | hAb-2 | hAb-3 |
|---|---|---|---|
| HC-CDR1 | DYEIH | DYEIH | DYEIH |
| | (SEQ ID NO: 1) | (SEQ ID NO: 1) | (SEQ ID NO: 1) |
| HC-CDR2 | AIDPETGGT | AIDPETGGT | AIDPETGGT |
| | AYNQKFKG | AYNQKFKG | AYNQKFKG |
| | (SEQ ID NO: 5) | (SEQ ID NO: 5) | (SEQ ID NO: 5) |
| HC-CDR3 | YDYVTY | YDYVTY | YDYVTY |
| | (SEQ ID NO: 9) | (SEQ ID NO: 9) | (SEQ ID NO: 9) |
| LC-CDR1 | KSSQSLLYS | RSSQSLLYSD | RSSQSLLYSDSRTYLN |
| | DGRTYLN | GRTYLN | (SEQ ID NO: 86) |
| | (SEQ ID NO: 15) | (SEQ ID NO: 85) | |
| LC-CDR2 | LVSKLDS | LVSKLDS | LVSKLDS |
| | (SEQ ID NO: 18) | (SEQ ID NO: 18) | (SEQ ID NO: 18) |
| LC-CDR3 | WQGTHLPHT | WQGTHLPHT | WQGTHLPHT |
| | (SEQ ID NO: 20) | (SEQ ID NO: 20) | (SEQ ID NO: 20) |
| V_{H} | SEQ ID NO: 87 | SEQ ID NO: 87 | SEQ ID NO: 87 |
| V_{L} | SEQ ID NO: 88 | SEQ ID NO: 89 | SEQ ID NO: 90 |

### Example 6. Binding Kinetics of Humanized Anti-Sclerostin Antibodies for Human and Cynomolgus Monkey Sclerostin Measured by Bio-Layer Interferometry (BLI) Assay

The binding affinity of humanized anti-Sclerostin antibodies to human and cynomolgus monkey Sclerostin were determined with bio-layer interferometry using a Gator® instrument (Probe Life). Antibodies were immobilized on an anti-hFc biosensor, using a 5 μg solution. Serial dilutions of human Sclerostin and cynomolgus monkey Sclerostin from 4 μg/ml by two folds in kinetics buffer (PBS, pH 7.4, 0.05% Tween™-20, 0.2% BSA, 10 μM biotin) were used as the analytes. Affinity (K_{D}) and kinetic parameters (Kₒₙ and K_{off}) were calculated from a global fit (1:1) of the data using the Gator® software.

The results were shown in Table 7 as below. The humanized antibodies hAb-1 and hAb-2 show higher affinities for human and Cynomolgus monkey Sclerostins as compared to reference Romosozumab.

TABLE 7
The binding kinetics of humanized antibody against Sclerostin of different species.

| | Human | | | Cynomolgus Monkey | | |
|---|---|---|---|---|---|---|
| Antibodies | Kₒₙ (1/Ms) | K_{off} (1/s) | K_{D} (M) | Kₒₙ (1/Ms) | K_{off} (1/s) | K_{D} (M) |
| 93B1B7 | 1.0 × 10⁶ | 8.4 × 10⁻⁵ | 5.1 × 10⁻¹¹ | 2.2 × 10⁶ | 6.5 × 10⁻⁵ | 1.8 × 10⁻¹¹ |
| hAb-1 | 7.3 × 10⁵ | 3.0 × 10⁻⁴ | 4.1 × 10⁻¹⁰ | 1.28 × 10⁶ | 2.29 × 10⁻⁴ | 1.8 × 10⁻¹⁰ |
| hAb-2 | 7.8 × 10⁵ | 2.0 × 10⁻⁴ | 2.4 × 10⁻¹⁰ | 1.54 × 10⁶ | 8.06 × 10⁻⁵ | 5.5 × 10⁻¹¹ |
| Romosozumab | 1.4 × 10⁶ | 2.8 × 10⁻⁴ | 7.8 × 10⁻¹⁰ | 7.55 × 10⁵ | 2.39 × 10⁻⁴ | 3.5 × 10⁻¹⁰ |

### Example 7. Sclerostin-Neutralization Activity of Anti-Sclerostin Antibodies in HEK293/TCF/LEF/Wnt1 Cells

The antibodies were capable of neutralizing Sclerostin and blocking Wnt1 induced TCF/LEF luciferase activity, as determined in an HEK293 based assay.

The Wnt proteins are a family of secreted glycoproteins that are critical regulators of osteoblast differentiation and activity in both mice and humans. Wnt signals are transduced by a family of seven-pass transmembrane G-protein coupled receptors of the frizzled (Fzd) family, and a co-receptor of the arrow/LRP family, such as LRP5 and LRP6, or a RYK or ROR transmembrane tyrosine kinase. The binding of a given Wnt to a Fzd receptor and coreceptor activates multiple distinct intracellular signaling cascades, historically divided into the canonical β-catenin-dependent pathway and noncanonical β-catenin-independent pathways. B-catenin is an important transcriptional coactivator that regulates gene transcription in response to Wnt signaling. The binding of Wnt to a Fzd receptor complex results in phosphorylation of the LRP5/6 complex, and subsequently stabilizes β-catenin in cytoplasm. Stabilized β-catenin is then translocated into the nucleus and interacts with the Lymphoid-enhancing factor/T-cell factor (LEF/TCF) family of high mobility group (HMG)-type transcription factors, to stimulate expression of target genes.

In the study of Wnt signaling pathway antagonists and its antibody blockades, the cell line, HEK293/TCF/LEF/Wnt1, was obtained from Askgene (Askgene, CA). The inhibitory activities of Wnt signaling antagonists, such as Sclerostin and DKK1, were analyzed using this HEK293 cell line expressing a TCF/LEF firefly luciferase reporter gene. Addition of Sclerostin or DKK1 antagonizes the Wnt1 signal, resulting in diminished luciferase activity. Conversely, addition of Sclerostin or DKK1 antibody will block the diminished signal and restore the luciferase activities. Upon arrival, cells were cultured in RPMI1640 culture medium with L-glutamine (Life Technologies, CA), containing 10% fetal bovine serum (FBS), non-essential amino acids, sodium pyruvate, 2-merceptoethanol, 1% penicillin/streptomycin, 400 μg/ml G418, and 2 μg/ml puromycin. Cells were harvested and plated into a white 96-well plate, at a concentration of 30,000 cells/well.

When testing the inhibitory effect of the antagonist, the culture medium was removed and 50 μl of assay medium (culture medium without G418 and puromycin) containing 10 mM LiCl, was added into each well. The tested Sclerostin and DKK1 proteins were serial diluted, and equal amount of antagonist was added into the assay plate and incubated for 6 hours at 37° C. with 5% CO₂. Then, 100 μl/well of One-Step firefly luciferase (Pierce, CA) reagent was added, and the luciferase activity was measured using a BioTek® Gen5 luminescence microplate reader (Winooski, VT). When testing the neutralization activities of the antibodies, a serial dilution of the test antibodies and control antibodies was made in assay medium. A fixed amount of either Sclerostin or DKK1 protein was added and incubated for 15 minutes. Then, 50 μl of the medium was transferred into assay plate, incubated for 6 hours at 37° C. with 5% CO₂, and analyzed using BioTek® Gen5 luminescence microplate reader.

Results were shown in Table 8 below. As shown, all murine anti-Sclerostin antibodies except 65B12C9 exhibited comparable or more advantageous EC₅₀ value as compared to that of Romosozumab. The three humanized antibodies, hAb-1, hAb-2 and hAb-3 exhibited more advantageous EC₅₀ value than that of Romosozumab.

TABLE 8
The Sclerostin-neutralisation activity for murine and
humanized antibodies in HEK293/TCF/LEF/Wnt1 cells

| Antibodies | EC₅₀ (nM) |
|---|---|
| 93B1B7 | 57.9 |
| 94B12D3 | 62.8 |
| 71G6G8 | 67.41 |
| 56E5C10 | 64.2 |
| 91F6D10 | 60.58 |
| 51E8D4 | 63.35 |
| 97C11D7 | 55.36 |
| 81B2B6 | 59.61 |
| 84F2D5 | 59.42 |
| 65B12C9 | 152.5 |
| hAb-1 | 13.89 |
| hAb-2 | 14.9 |
| hAb-3 | 15.13 |
| Romosozumab | 42.31 |

### Example 8: Solubility and Stability of Humanized Anti-Sclerostin Antibodies

The humanized anti-human Sclerostin_antibody hAb-1 and hAb-2 described in this invention and the reference antibody Romosozumab were accessed for precipitation at increasing concentration. The starting concentration was 10 mg/mi in PBS buffer at pH 7.4. The cells were centrifuged at 4 degrees 4000 rpm in an ultrafiltration concentrating tube (Millipore), and the centrifugation was stopped every 5-10 minutes and the antibody concentration was sampled until the antibody concentration reached 30 mg/ml and 90 mg/ml. The samples were stored at 4C for 14 days and visually observed for precipitation. The antibodies described in this invention remained clear solution at as high as 90 mg/ml, which reference antibody Romosozumab became precipitated and turbid, indicating hAb-1 and hAb-2 are more stable and soluble than Romosozumab in PBS buffer at pH7.4. This result suggests that the antibodies described in this invent can be stable in high concentration formulations like 90 mg/ml.

| Antibody | Concentration (mg/ml) | Day 14 |
|---|---|---|
| hAb-1 | 10 | Clear solution |
| | 30 | Clear solution |
| | 90 | Clear solution |
| hAb-2 | 10 | Clear solution |
| | 30 | Clear solution |
| | 90 | Clear solution |
| Romosozumab | 10 | Clear solution |
| | 30 | Clear solution |
| | 90 | Sedimentation and turbid |
| | | solution observed |

### Example 9. Generation of Bispecific Antibodies

Bispecific antibodies that target both Sclerostin and DKK1 or RANKL were generated. See Tables 9 and 10 for sequences of the exemplary antibodies that target DKK1 or RANKL.

TABLE 9
CDR Sequences of exemplary anti-DKK1 antibodies

| | 11H10-V_{H}44V_{L}32 | | |
|---|---|---|---|
| | (11H10) | RH2-18 | 71G6G8 |
| HC-CDR1 | GFTFSDYAMA | GYTFTDYYIH | GFTFSSYAIS |
| | (SEQ ID NO: 42) | (SEQ ID NO: 48) | (SEQ ID NO: 54) |
| HC-CDR2 | THIYDGSSTYYR | WIHSNSGAT | SVSGTGLGFQTY |
| | DSVKG | TYAQKFQA | YPDSVKG |
| | (SEQ ID NO: 43) | (SEQ ID NO: 49) | (SEQ ID NO: 55) |
| HC-CDR3 | GLGIATDYFDY | EDY | SLENYAFDY |
| | (SEQ ID NO: 44) | (SEQ ID NO: 50) | (SEQ ID NO: 56) |
| LC-CDR1 | LASEDIYSDLA | TGSSNIGAGYDVH | RASESVDDFGISFIN |
| | (SEQ ID NO: 45) | (SEQ ID NO: 51) | (SEQ ID NO: 57) |
| LC-CDR2 | NANSLQN | GYSNRPS | AGSKQGS |
| | (SEQ ID NO: 46) | (SEQ ID NO: 52) | (SEQ ID NO: 58) |
| LC-CDR3 | QQYNNYPPT | AGSKQGS | QQLKEVPPT |
| | (SEQ ID NO: 47) | (SEQ ID NO: 58) | (SEQ ID NO: 59) |

TABLE 10
CDR Sequences of exemplary
anti-RANKL antibody

| | Denosumab |
|---|---|
| HC-CDR1 | GFTFSSYAMS (SEQ ID NO: 66) |
| HC-CDR2 | GITGSGGSTYYADSVKG (SEQ ID NO: 67) |
| HC-CDR3 | DPGTTVIMSWFDP (SEQ ID NO: 68) |
| LC-CDR1 | RASQSVRGRYLA (SEQ ID NO: 69) |
| LC-CDR2 | GASSRAT (SEQ ID NO: 70) |
| LC-CDR3 | QQYGSSPRT (SEQ ID NO: 71) |

Specifically, the following Sclerostin/DKK1 and Sclerostin/RANKL pairings were generated: hAb-1×11H10-V_{H}44V_{L}32, hAb-2×11H10-V_{H}44V_{L}32, hAb-1×Denosumab, hAb-2×Denosumab.

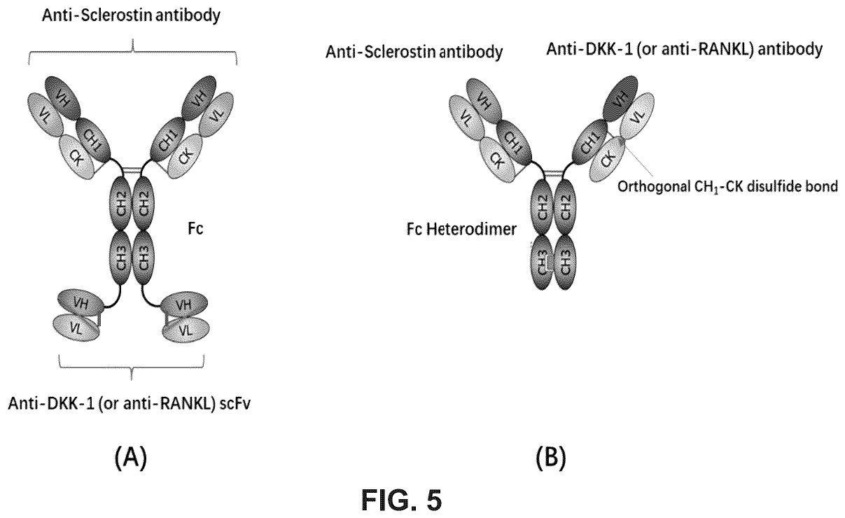

In one embodiment (see (A) of FIG. 5), anti-DKK1 11H10-V_{H}44V_{L}32 (or anti-RANKL Denosumab) Fv domain was transformed into scFvs with two different orientations of V_{H} and V_{L}, linked by a (GGGGS)₄ linker. There are mutations like G100C in V_{H} and G44C in V_{L} to facilitate correct assembly of scFvs. Two anti-DKK1 (or anti-RANKL) scFvs are linked to each of two IgG heavy chain C-termini of anti-Sclerostin humanized antibodies respectively by one GGGGSGGGGSGGGGS (SEQ ID NO: 76) linker to form bispecific of IgG-scFv.

In another embodiment (see (B) of FIG. 5), anti-Sclerostin antibodies and anti-DKK 11H10-V_{H}44V_{L}32 (or anti-RANKL Denosumab) were transformed into ‘Knob-into-Hole’ bispecific antibodies with asymmetric IgG structure. The CL domain of anti-DKK1 11-H10-V_{H}44V_{L}32 (or anti-RANKL Denosumab) contains mutations of S176C and C214S, and the CH1 domain contains mutations like F170C and C131S to form orthogonal CH₁-CK disulfide bond to enable light chains correct paring.

Table 11 below lists format and sequences of various anti-Sclerostin bispecific antibodies.

TABLE 11
Anti-Sclerostin bispecific antibodies

| | | Heavy chain (from | Light chain (from N- |
|---|---|---|---|
| Bispecific | | N-terminus to C- | terminus to C- |
| antibodies | Format | terminus) | terminus) |
| BAP0017 | Anti-Sclerostin scFv (V_{H}- | 11H10 V_{H}2-CH1- | 11H10 V_{L}2-CL |
| | V_{L}) fused to C-terminus of | CH2-CH3-L1- | (SEQ ID NO: 93) |
| | heavy chains of anti-DKK1 | 93B1B7 V_{H}-L2- | |
| | full length antibody | 93B1B7 V_{L} | |
| | | (SEQ ID NO: 91) | |
| BAP0018 | Anti-Sclerostin scFv (V_{L}- | 11H10 V_{H}2-CH1- | 11H10 V_{L}2-CL |
| | V_{H}) fused to C-terminus of | CH2-CH3-L1- | (SEQ ID NO: 93) |
| | heavy chains of anti-DKK1 | 93B1B7 V_{L}-L2- | |
| | full length antibody | 93B1B7 V_{H} | |
| | | (SEQ ID NO: 92) | |
| BAP0019 | Anti-DKK1 scFv (V_{H}-V_{L}) | 93B1B7 V_{H}-CH1- | 93B1B7 V_{L}-CL |
| | fused to C-terminus of | CH2-CH3-L1- | (SEQ ID NO: 96) |
| | heavy chains of anti- | 11H10 V_{H}2-L2- | |
| | Sclerostin full length | 11H10 V_{L}2 | |
| | antibody | (SEQ ID NO: 94) | |
| BAP0020 | Anti-DKK1 scFv (V_{L}-V_{H}) | 93B1B7 V_{H}-CH1- | 93B1B7 V_{L}-CL |
| | fused to C-terminus of | CH2-CH3-L1- | (SEQ ID NO: 96) |
| | heavy chains of anti- | 11H10 V_{L}2-L2- | |
| | Sclerostin full length | 11H10 V_{H}2 | |
| | antibody | (SEQ ID NO: 95) | |
| BAP0021 | Anti-Sclerostin scFv (V_{H}- | 93B1B7 V_{H}-L1- | 11H10 V_{L}2-CL |
| | V_{L}) fused to N-terminus of | 93B1B7 V_{L}-L2- | (SEQ ID NO: 99) |
| | heavy chains of anti-DKK1 | 11H10 V_{H}2-CH1- | |
| | full length antibody | CH2-CH3 | |
| | | (SEQ ID NO: 97) | |
| BAP0022 | Anti-Sclerostin scFv (V_{L}- | 93B1B7 V_{L}-L1- | 11H10 V_{L}2-CL |
| | V_{H}) fused to N-terminus of | 93B1B7 V_{H}-L2- | (SEQ ID NO: 99) |
| | heavy chains of anti-DKK1 | 11H10 V_{H}2-CH1- | |
| | full length antibody | CH2-CH3 | |
| | | (SEQ ID NO: 98) | |
| BAP0023 | Anti-DKK1 scFv (V_{H}-V_{L}) | 11H10 V_{H}2-L1- | 93B1B7 V_{L}-CL |
| | fused to N-terminus of | 11H10 V_{L}2-L2- | (SEQ ID NO: 102) |
| | heavy chains of anti- | 93B1B7 V_{H}-CH1- | |
| | Sclerostin full length | CH2-CH3 | |
| | antibody | (SEQ ID NO: 100) | |
| BAP0024 | Anti-DKK1 scFv (V_{L}-V_{H}) | 11H10 V_{L}2-L1- | 93B1B7 V_{L}-CL |
| | fused to N-terminus of | 11H10 V_{H}2-L2- | (SEQ ID NO: 102) |
| | heavy chains of anti- | 93B1B7 V_{H}-CH1- | |
| | Sclerostin full length | CH2-CH3 | |
| | antibody | (SEQ ID NO: 101) | |
| BAP0025 | Anti-Sclerostin scFv (V_{H}- | 11H10 V_{H}2-CH1- | 93B1B7 V_{H}-L1- |
| | V_{L}) fused to N-terminus of | CH2-CH3 | 93B1B7 V_{L}-L2- |
| | light chains of anti-DKK1 | (SEQ ID NO: 103) | 11H10 V_{L}2-CL |
| | full length antibody | | (SEQ ID NO: 104) |
| BAP0026 | Anti-Sclerostin scFv (V_{L}- | 11H10 V_{H}2-CH1- | 93B1B7 V_{L}-L1- |
| | V_{H}) fused to N-terminus of | CH2-CH3 | 93B1B7 V_{H}-L2- |
| | light chains of anti-DKK1 | (SEQ ID NO: 103) | 11H10 V_{L}2-CL |
| | full length antibody | | (SEQ ID NO: 105) |
| BAP0027 | Anti-DKK1 scFv (V_{H}-V_{L}) | 93B1B7 V_{H}-CH1- | 11H10 V_{H}2-L1- |
| | fused to N-terminus of light | CH2-CH3 | 11H10 V_{L}2-L2- |
| | chains of anti-Sclerostin full | (SEQ ID NO: 106) | 93B1B7 V_{L}-CL |
| | length antibody | | (SEQ ID NO: 107) |
| BAP0028 | Anti-DKK1 scFv (V_{L}-V_{H}) | 93B1B7 V_{H}-CH1- | 11H10 V_{L}2-L1- |
| | fused to N-terminus of light | CH2-CH3 | 11H10 V_{H}2-L2- |
| | chains of anti-Sclerostin full | (SEQ ID NO: 106) | 93B1B7 V_{L}-CL |
| | length antibody | | (SEQ ID NO: 108) |
| BAP0029 | Anti-Sclerostin scFv (V_{H}- | 11H10 V_{H}2-CH1- | 11H10 V_{L}2-CL-L1- |
| | V_{L}) fused to C-terminus of | CH2-CH3 | 93B1B7 V_{H}-L2- |
| | light chains of anti-DKK1 | (SEQ ID NO: 109) | 93B1B7 V_{L} |
| | full length antibody | | (SEQ ID NO: 110) |
| BAP0030 | Anti-Sclerostin scFv (V_{L}- | 11H10 V_{H}2-CH1- | 11H10 V_{L}2-CL-L1- |
| | V_{H}) fused to C-terminus of | CH2-CH3 | 93B1B7 V_{L}-L2- |
| | light chains of anti-DKK1 | (SEQ ID NO: 109) | 93B1B7 V_{H} |
| | full length antibody | | (SEQ ID NO: 111) |
| BAP0031 | Anti-DKK1 scFv (V_{H}-V_{L}) | 93B1B7 V_{H}-CH1- | 93B1B7 V_{L}-CL-L1- |
| | fused to C-terminus of light | CH2-CH3 | 11H10 V_{H}2-L2- |
| | chains of anti-Sclerostin full | (SEQ ID NO: 112) | 11H10 V_{L}2 |
| | length antibody | | (SEQ ID NO: 113) |
| BAP0032 | Anti-DKK1 scFv (V_{L}-V_{H}) | 93B1B7 V_{H}- CH1- | 93B1B7 V_{L}-CL-L1- |
| | fused to C-terminus of light | CH2-CH3 | 11H10 V_{L}2-L2- |
| | chains of anti-Sclerostin full | (SEQ ID NO: 112) | 11H10 V_{H}2 |
| | length antibody | | (SEQ ID NO: 114) |
| BAP0033 | Anti-Sclerostin scFv (V_{L}- | 11H10 V_{H}-CH1- | 11H10 V_{L}-CL |
| | V_{H}) fused to C-terminus of | CH2-CH3-L1- | (SEQ ID NO: 119) |
| | heavy chains of anti-DKK1 | hAb2 V_{L}2-L2-hAb | |
| | full length antibody | V_{H}2 | |
| | | (SEQ ID NO: 115) | |
| BAP0034 | Anti-Sclerostin scFv (V_{H}- | 11H10 V_{H}-CH1- | 11H10 V_{L}3-CL |
| | V_{L}) fused to C-terminus of | CH2-CH3-L1-hAb | (SEQ ID NO: 119) |
| | heavy chains of anti-DKK1 | V_{H}2-L2-hAb2 V_{L}2 | |
| | full length antibody | (SEQ ID NO: 116) | |
| BAP0035 | Anti-Sclerostin scFv (V_{L}- | 11H10 V_{H}-CH1- | 11H10 V_{L}3-CL |
| | V_{H}) fused to C-terminus of | CH2-CH3-L1- | (SEQ ID NO: 119) |
| | heavy chains of anti-DKK1 | hAb1 V_{L}2-L2-hAb | |
| | full length antibody | V_{H}2 | |
| | | (SEQ ID NO: 117) | |
| BAP0036 | Anti-Sclerostin scFv (V_{H}- | 11H10 V_{H}-CH1- | 11H10 V_{L}3-CL |
| | V_{L}) fused to C-terminus of | CH2-CH3-L1-hAb | (SEQ ID NO: 119) |
| | heavy chains of anti-DKK1 | V_{H}2-L2-hAb1 V_{L}2 | |
| | full length antibody | (SEQ ID NO: 118) | |
| BAP0037 | Anti-DKK1 scFv (V_{H}-V_{L}) | hAb V_{H}-CH1-CH2- | hAb-2 V_{L}-CL |
| | fused to C-terminus of | CH3-L1-11H10 | (SEQ ID NO: 122) |
| | heavy chains of anti- | V_{H}3-L2-11H10 V_{L}3 | |
| | Sclerostin full length | (SEQ ID NO: 120) | |
| | antibody | | |
| BAP0038 | Anti-DKK1 scFv (V_{L}-V_{H}) | hAb V_{H}-CH1-CH2- | hAb-2 V_{L}-CL |
| | fused to C-terminus of | CH3-L1-11H10 | (SEQ ID NO: 122) |
| | heavy chains of anti- | V_{L}3-L2-11H10 V_{H}3 | |
| | Sclerostin full length | (SEQ ID NO: 121) | |
| | antibody | | |
| BAP0039 | Anti-DKK1 scFv (V_{H}-V_{L}) | hAb V_{H}-CH1-CH2- | hAb-1 V_{L}-CL |
| | fused to C-terminus of | CH3-L1-11H10 | (SEQ ID NO: 125) |
| | heavy chains of anti- | V_{H}3-L2-11H10 V_{L}3 | |
| | Sclerostin full length | (SEQ ID NO: 123) | |
| | antibody | | |
| BAP0040 | Anti-DKK1 scFv (V_{L}-V_{H}) | hAb V_{H}-CH1-CH2- | hAb-1 V_{L}-CL |
| | fused to C-terminus of | CH3-L1-11H10 | (SEQ ID NO: 125) |
| | heavy chains of anti- | V_{L}3-L2-11H10 V_{H}3 | |
| | Sclerostin full length | (SEQ ID NO: 124) | |
| | antibody | | |
| BAP0050 | Anti-Sclerostin scFv (V_{L}- | Denosumab V_{H} - | Denosumab V_{L}-CL |
| | V_{H}) fused to C-terminus of | CH1-CH2-CH3- | (SEQ ID NO: 129) |
| | heavy chains of anti- | L1- hAb-2 V_{L}2 - | |
| | RANKL full length | L2-hAb V_{H}2 | |
| | antibody | (SEQ ID NO: 126) | |
| BAP0051 | Anti-Sclerostin scFv (V_{L}- | hAb-2 V_{L}2 -L1- | Denosumab V_{L}-CL |
| | V_{H}) fused to N-terminus of | hAb V_{H}2-L2- | (SEQ ID NO: 129) |
| | heavy chains of anti- | Denosumab V_{H} - | |
| | RANKL full length | CH1-CH2-CH3 | |
| | antibody | (SEQ ID NO: 127) | |
| BAP0052 | Anti-Sclerostin scFv (V_{L}- | Denosumab V_{H} - | Denosumab V_{L}-CL |
| | V_{H}) fused to C-terminus of | CH1-CH2-CH3- | (SEQ ID NO: 129) |
| | heavy chains of anti- | L1- hAb-2 V_{L}2 - | |
| | RANKL full length | L2-hAb V_{H}2 | |
| | antibody | (SEQ ID NO: 128) | |
| BAP0053 | Anti-Sclerostin scFv (V_{L}- | Denosumab V_{H} - | Denosumab V_{L}-CL- |
| | V_{H}) fused to C-terminus of | CH1-CH2-CH3 | L1- hAb-2 V_{L}2 -L2- |
| | light chains of anti-RANKL | (SEQ ID NO: 133) | hAb V_{H}2 |
| | full length antibody | | (SEQ ID NO: 130) |
| BAP0054 | Anti-Sclerostin scFv (V_{L}- | Denosumab V_{H} - | hAb-2 V_{L}2 -L1-hAb |
| | V_{H}) fused to N-terminus of | CH1-CH2-CH3 | V_{H}2-L2- Denosumab |
| | light chains of anti-RANKL | (SEQ ID NO: 133) | V_{L}-CL |
| | full length antibody | | (SEQ ID NO: 131) |
| BAP0055 | Anti-Sclerostin scFv (V_{L}- | Denosumab V_{H} - | Denosumab V_{L}-CL- |
| | V_{H}) fused to C-terminus of | CH1-CH2-CH3 | L1- hAb-1 V_{L}2 -L2- |
| | light chains of anti-RANKL | (SEQ ID NO: 133) | hAb V_{H}2 |
| | full length antibody | | (SEQ ID NO: 132) |
| BAP0041 | A full length antibody | 1^{st} heavy chain: | 1^{st} light chain: |
| | having a) first pair of V_{H}-V_{L} | 93B1B7 V_{H}-CH1- | 93B1B7 V_{L}-CL |
| | (a first Fab) recognizing | CH2-CH3 | (SEQ ID NO: 135) |
| | Sclerostin and b) second | (SEQ ID NO: 134) | 2^{nd} light chain: |
| | pair of V_{H}-V_{L} (a second | 2^{nd} heavy chain: | 11H10 V_{L}2-CL |
| | Fab) recognizing DKK1 | 11H10 V_{H}2-CH1- | (SEQ ID NO: 137) |
| | | CH2-CH3 | |
| | | (SEQ ID NO: 136) | |
| BAP0042 | A full length antibody | 1^{st} heavy chain: | 1^{st} light chain: |
| | having a) first pair of V_{H}-V_{L} | 93B1B7 V_{H}-CH1- | 93B1B7 V_{L}-CL |
| | (a first Fab) recognizing | CH2-CH3 | (SEQ ID NO: 135) |
| | Sclerostin and b) second | (SEQ ID NO: 134) | 2^{nd} light chain: |
| | pair of V_{H}-V_{L} (a second | 2^{nd} heavy chain: | 11H10 V_{L}2-CL |
| | Fab) recognizing DKK1 | 11H10 V_{H}2-CH1- | (SEQ ID NO: 139) |
| | | CH2-CH3 | |
| | | (SEQ ID NO: 138) | |
| BAP0043 | A full length antibody | 1^{st} heavy chain: | 1^{st} light chain: |
| | having a) first pair of V_{H}-V_{L} | 93B1B7 V_{H}-CH1- | 93B1B7 V_{L}-CL |
| | (a first Fab) recognizing | CH2-CH3 | (SEQ ID NO: 135) |
| | Sclerostin and b) second | (SEQ ID NO: 134) | 2^{nd} light chain: |
| | pair of V_{H}-V_{L} (a second | 2^{nd} heavy chain: | 11H10 V_{L}2-CL |
| | Fab) recognizing DKK1 | 11H10 V_{H}2-CH1- | (SEQ ID NO: 141) |
| | | CH2-CH3 | |
| | | (SEQ ID NO: 140) | |
| BAP0044 | A full length antibody | 1^{st} heavy chain: | 1^{st} light chain: |
| | having a) first pair of V_{H}-V_{L} | 93B1B7 V_{H}-CH1- | 93B1B7 V_{L}-CL |
| | (a first Fab) recognizing | CH2-CH3 | (SEQ ID NO: 135) |
| | Sclerostin and b) second | (SEQ ID NO: 134) | 2^{nd} light chain: |
| | pair of V_{H}-V_{L} (a second | 2^{nd} heavy chain: | 11H10 V_{L}2-CL |
| | Fab) recognizing DKK1 | 11H10 V_{H}2-CH1- | (SEQ ID NO: 143) |
| | | CH2-CH3 | |
| | | (SEQ ID NO: 142) | |
| BAP0045 | A full length antibody | 1^{st} heavy chain: | 1^{st} light chain: |
| | having a) first pair of V_{H}-V_{L} | 93B1B7 V_{H}-CH1- | 93B1B7 V_{L}-CL |
| | (a first Fab) recognizing | CH2-CH3 | (SEQ ID NO: 135) |
| | Sclerostin and b) second | (SEQ ID NO: 134) | 2^{nd} light chain: |
| | pair of V_{H}-V_{L} (a second | 2^{nd} heavy chain: | 11H10 V_{L}2-CL |
| | Fab) recognizing DKK1 | 11H10 V_{H}2-CH1- | (SEQ ID NO: 145) |
| | | CH2-CH3 | |
| | | (SEQ ID NO: 144) | |
| BAP0061 | A full length antibody | 1^{st} heavy chain: | 1^{st} light chain: |
| | having a) first pair of V_{H}-V_{L} | hAb V_{H}-CH1-CH2- | hAb-2 V_{L}-CL |
| | (a first Fab) recognizing | CH3 | (SEQ ID NO: 147) |
| | Sclerostin and b) second | (SEQ ID NO: 146) | 2^{nd} light chain: |
| | pair of V_{H}-V_{L} (a second | 2^{nd} heavy chain: | 11H10 V_{L}-CL |
| | Fab) recognizing DKK1 | 11H10 V_{H}-CH1- | SEQ ID NO: 149) |
| | | CH2-CH3 | |
| | | (SEQ ID NO: 148) | |
| BAP0062 | A full length antibody | 1^{st} heavy chain: | 1^{st} light chain: |
| | having a) first pair of V_{H}-V_{L} | hAb V_{H}-CH1-CH2- | hAb-2 V_{L}-CL |
| | (a first Fab) recognizing | CH3 | (SEQ ID NO: 147) |
| | Sclerostin and b) second | (SEQ ID NO: 146) | 2^{nd} light chain: |
| | pair of V_{H}-V_{L} (a second | 2^{nd} heavy chain: | 11H10 V_{L}-CL |
| | Fab) recognizing DKK1 | 11H10 V_{H}-CH1- | SEQ ID NO: 151) |
| | | CH2-CH3 | |
| | | (SEQ ID NO: 150) | |
| BAP0063 | A full length antibody | 1^{st} heavy chain: | 1^{st} light chain: |
| | having a) first pair of V_{H}-V_{L} | hAb V_{H}-CH1-CH2- | hAb-2 V_{L}-CL |
| | (a first Fab) recognizing | CH3 | (SEQ ID NO: 147) |
| | Sclerostin and b) second | (SEQ ID NO: 146) | 2^{nd} light chain: |
| | pair of V_{H}-V_{L} (a second | 2^{nd} heavy chain: | 11H10 V_{L}-CL |
| | Fab) recognizing DKK1 | 11H10 V_{H}-CH1- | SEQ ID NO: 153) |
| | | CH2-CH3 | |
| | | (SEQ ID NO: 152) | |
| BAP0064 | A full length antibody | 1^{st} heavy chain: | 1^{st} light chain: |
| | having a) first pair of V_{H}-V_{L} | hAb V_{H}-CH1-CH2- | hAb-2 V_{L}-CL |
| | (a first Fab) recognizing | CH3 | (SEQ ID NO: 147) |
| | Sclerostin and b) second | (SEQ ID NO: 146) | 2^{nd} light chain: |
| | pair of V_{H}-V_{L} (a second | 2^{nd} heavy chain: | 11H10 V_{L}-CL |
| | Fab) recognizing DKK1 | 11H10 V_{H}-CH1- | SEQ ID NO: 155) |
| | | CH2-CH3 | |
| | | (SEQ ID NO: 154) | |
| BAP0065 | A full length antibody | 1^{st} heavy chain: | 1^{st} light chain: hAb-2 |
| | having a) first pair of V_{H}-V_{L} | hAb V_{H}-CH1-CH2- | V_{L}-CL |
| | (a first Fab) recognizing | CH3 | (SEQ ID NO: 147) |
| | Sclerostin and b) second | (SEQ ID NO: 146) | 2^{nd} light chain: |
| | pair of V_{H}-V_{L} (a second | 2^{nd} heavy chain: | Denosumab V_{L}-CL |
| | Fab) recognizing RANKL | Denosumab V_{H}- | SEQ ID NO: 157) |
| | | CH1-CH2-CH3 | |
| | | (SEQ ID NO: 156) | |
| BAP0066 | A full length antibody | 1^{st} heavy chain: | 1^{st} light chain: hAb-2 |
| | having a) first pair of V_{H}-V_{L} | hAb V_{H}-CH1-CH2- | V_{L}-CL |
| | (a first Fab) recognizing | CH3 | (SEQ ID NO: 147) |
| | Sclerostin and b) second | (SEQ ID NO: 146) | 2^{nd} light chain: |
| | pair of V_{H}-V_{L} (a second | 2^{nd} heavy chain: | Denosumab V_{L}-CL |
| | Fab) recognizing RANKL | Denosumab V_{H}- | SEQ ID NO: 159) |
| | | CH1-CH2-CH3 | |
| | | (SEQ ID NO: 158) | |
| BAP0067 | A full length antibody | 1^{st} heavy chain: | 1^{st} light chain: hAb-2 |
| | having a) first pair of V_{H}-V_{L} | hAb V_{H}-CH1-CH2- | V_{L}-CL |
| | (a first Fab) recognizing | CH3 | (SEQ ID NO: 147) |
| | Sclerostin and b) second | (SEQ ID NO: 146) | 2^{nd} light chain: |
| | pair of V_{H}-V_{L} (a second | 2^{nd} heavy chain: | Denosumab V_{L}-CL |
| | Fab) recognizing RANKL | Denosumab V_{H}- | SEQ ID NO: 163) |
| | | CH1-CH2-CH3 | |
| | | (SEQ ID NO: 162) | |
| BAP0068 | A full length antibody | 1^{st} heavy chain: | 1^{st} light chain: hAb-2 |
| | having a) first pair of V_{H}-V_{L} | hAb V_{H}-CH1-CH2- | V_{L}-CL |
| | (a first Fab) recognizing | CH3 | (SEQ ID NO: 147) |
| | Sclerostin and b) second | (SEQ ID NO: 146) | 2^{nd} light chain: |
| | pair of V_{H}-V_{L} (a second | 2^{nd} heavy chain: | Denosumab V_{L}-CL |
| | Fab) recognizing RANKL | Denosumab V_{H}- | SEQ ID NO: 161) |
| | | CH1-CH2-CH3 | |
| | | (SEQ ID NO: 160) | |
| BAP0069 | Anti-RANKL scFv (VL- | hAb V_{H}-CH1-CH2- | hAb-1 V_{L}-CL |
| | VH) fused to C-terminus of | CH3-L1- | (SEQ ID NO: 125) |
| | heavy chains of anti- | Denosumab V_{L}-L2- | |
| | Sclerostin full length | Denosumab V_{H} | |
| | antibody | (SEQ ID NO: 177) | |
| BAP0070 | Anti-RANKL scFv (VH- | hAb V_{H}-CH1-CH2- | hAb-1 V_{L}-CL |
| | VL) fused to C-terminus of | CH3-L1- | (SEQ ID NO: 125) |
| | heavy chains of anti- | Denosumab V_{H}-L2- | |
| | Sclerostin full length | Denosumab V_{L} | |
| | antibody | (SEQ ID NO: 178) | |
| BAP0071 | Anti-RANKL scFv (VH- | hAb V_{H}-CH1-CH2- | hAb-2 V_{L}-CL |
| | VL) fused to C-terminus of | CH3-L1- | (SEQ ID NO: 122) |
| | heavy chains of anti- | Denosumab V_{H}-L2- | |
| | Sclerostin full length | Denosumab V_{L} | |
| | antibody | (SEQ ID NO: 178) | |
| BAP0072 | Anti-RANKL scFv (VH- | hAb V_{H}-CH1-CH2- | hAb-2 V_{L}-CL |
| | VL) fused to C-terminus of | CH3-L1- | (SEQ ID NO: 122) |
| | heavy chains of anti- | Denosumab V_{H}-L2- | |
| | Sclerostin full length | Denosumab V_{L} | |
| | antibody | (SEQ ID NO: 179) | |
| BAP0073 | Anti-RANKL scFv (VH- | hAb V_{H}-CH1-CH2- | hAb-2 V_{L}-CL |
| | VL) fused to C-terminus of | CH3-L1- | (SEQ ID NO: 122) |
| | heavy chains of anti- | Denosumab V_{H}-L2- | |
| | Sclerostin full length | Denosumab V_{L} | |
| | antibody | (SEQ ID NO: 180) | |
| BAP0074 | A full length antibody | 1^{st} heavy chain: | 1^{st} light chain: hAb-2 |
| | having a) first pair of V_{H}-V_{L} | hAb V_{H}-CH1-CH2- | V_{L}-CL |
| | (a first Fab) recognizing | CH3 | (SEQ ID NO: 147) |
| | Sclerostin and b) second | (SEQ ID NO: 171) | 2^{nd} light chain: |
| | pair of V_{H}-V_{L} (a second | 2^{nd} heavy chain: | Denosumab V_{L}-CL |
| | Fab) recognizing RANKL | Denosumab V_{H}- | SEQ ID NO: 163) |
| | | CH1-CH2-CH3 | |
| | | (SEQ ID NO: 172) | |
| BAP0075 | A full length antibody | 1^{st} heavy chain: | 1^{st} light chain: hAb-2 |
| | having a) first pair of V_{H}-V_{L} | hAb V_{H}-CH1-CH2- | V_{L}-CL |
| | (a first Fab) recognizing | CH3 | (SEQ ID NO: 147) |
| | Sclerostin and b) second | (SEQ ID NO: 173) | 2^{nd} light chain: |
| | pair of V_{H}-V_{L} (a second | 2^{nd} heavy chain: | Denosumab V_{L}-CL |
| | Fab) recognizing RANKL | Denosumab V_{H}- | SEQ ID NO: 163) |
| | | CH1-CH2-CH3 | |
| | | (SEQ ID NO: 174) | |
| BAP0076 | A full length antibody | 1^{st} heavy chain: | 1^{st} light chain: hAb-2 |
| | having a) first pair of V_{H}-V_{L} | hAb V_{H}-CH1-CH2- | V_{L}-CL |
| | (a first Fab) recognizing | CH3 | (SEQ ID NO: 147) |
| | Sclerostin and b) second | (SEQ ID NO: 175) | 2^{nd} light chain: |
| | pair of V_{H}-V_{L} (a second | 2^{nd} heavy chain: | Denosumab V_{L}-CL |
| | Fab) recognizing RANKL | Denosumab V_{H}- | SEQ ID NO: 163) |
| | | CH1-CH2-CH3 | |
| | | (SEQ ID NO: 176) | |

Bispecific molecules were transiently expressed in adherence-adapted 293 6E cells in 96-well plates. Adherent 293 6E cells were seeded in Poly-D-Lysine coated 96-well tissue culture plates at 5E4 cells per well in Serum-free KOP293 medium (Zhuhai Kairui Biotech #K03252) at 25 μg/ml, and 500 FBS 24 hours prior to the transfection and incubated overnight at 37° C. in 5% CO₂. On the day of transfection, 100 ng (40 ng/μl) of each corresponding HC and LC DNA of the binding molecules were mixed together. Serum-free media KOP293 25 μl/well was added to the DNA mixtures. After incubation at RT for 15-30 min, the transfection mixtures were added to the culture plates which were seeded the day before and mixed with gentle rocking motions. The culture plates were put back into the 37° C., 500 CO₂ incubator overnight. The next day, media and transfection mixtures were aspirated and replaced with 130 μl of serum free media containing 0.5% Tryptone. The plates were incubated for another 6 days. Conditioned media (CM) was harvested on day 7 after transfection. The plates were spun at 1000 rpm for 5 min to pellet any cell debris. Supernatants were carefully transferred into sterile polypropylene blocks.

Antibodies were purified by protein A affinity chromatography, and buffer exchanged in PBS (pH 7.2). The concentrations of purified antibodies were determined by reading the absorbance at 280 nm using the theoretically determined extinction coefficient for that protein.

### Example 10. The Binding Kinetics and Affinities of Anti-Sclerostin×Anti-DKK1 and Anti-Sclerostin×Anti-RANKL Bispecific Antibodies

Binding kinetics and affinities of anti-Sclerostin×anti-DKK1 and anti-Sclerostin×anti-RANKL bispecific antibodies were measured with Gator® (Probe Life) bio-layer interferometry (BLI). Antibodies were immobilized on an anti-hFc biosensor, using a 5 μg/ml solution. Serial dilutions of sclerostin (from 1 μg/ml), DKK1 (from 2 μg/ml), or RANKL (from 4 μg/ml) by two folds in kinetics buffer (PBS, pH 7.4, 0.05% Tween™-20, 0.2% BSA) were used as the analytes. Affinity (K_{D}) and kinetic parameters (Kₒₙ and K_{off}) were calculated from a global fit (1:1) of the data using the Gator® software. The results are summarized in Table 12.

TABLE 12
Summary of binding kinetics of the bispecific
antibodies to human Sclerostin, DKK1 and RANKL

| Antibody | Antigen | k_{off} (1/s) | kₒₙ (1/Ms) | K_{D} (M)^{b} |
|---|---|---|---|---|
| BAP0037 | hSclerostin | LOD^{a} | 1.88 × 10⁶ | Estimated 10⁻¹¹ |
| | hDKK1 | 3.01 × 10⁻⁴ | 2.97 × 10⁵ | 1.01 × 10⁻⁹ |
| BAP0039 | hSclerostin | 9.74 × 10⁻⁵ | 1.88 × 10⁶ | 5.2 × 10⁻¹¹ |
| | hDKK1 | 4.32 × 10⁻⁵ | 3.09 × 10⁵ | 1.4 × 10⁻¹⁰ |
| BAP0061 | hSclerostin | LOD | 2.62 × 10⁶ | Estimated 10⁻¹¹ |
| | hDKK1 | LOD | 3.30 × 10⁵ | Estimated 10⁻¹¹ |
| BAP0063 | hSclerostin | LOD | 2.59 × 10⁶ | Estimated 10⁻¹¹ |
| | hDKK1 | LOD | 1.29 × 10⁵ | Estimated 10⁻¹¹ |
| BAP0069 | hSclerostin | LOD | 7.57 × 10⁵ | Estimated 10⁻¹¹ |
| | hRANKL | LOD | 8.67 × 10⁴ | Estimated 10⁻¹¹ |
| BAP0075 | hSclerostin | LOD | 5.78 × 10⁵ | Estimated 10⁻¹¹ |
| | hRANKL | LOD | 1.45 × 10⁵ | Estimated 10⁻¹¹ |

^{a}Limit of detection, which indicates the off-rate is very slow and beyond the quantification limit of Gator
^{b}The affinity K_{D} values are calculated by K_{off}/Kₒₙ, in the case that K_{off} is LOD, the K_{D} values are estimated around 10 pM, which is the detection limit of Gator

### Example 11. Simultaneous Binding of Human Sclerostin and DKK1 to bsAbs Using BLI

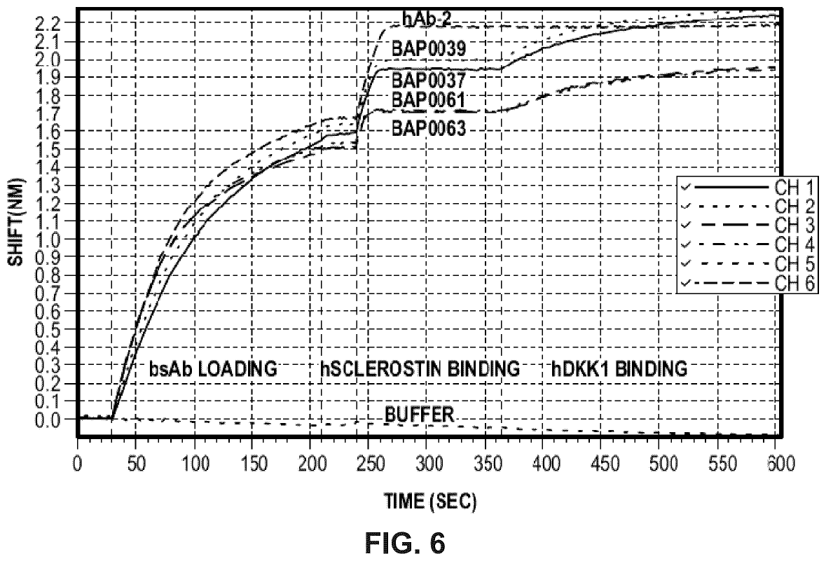

The simultaneous binding of human Sclerostin and DKK1 to bsAbs was confirmed using BLI. The bsAb constructs (5 μg/ml) were captured to the anti-hFc HFC probe, then sequentially adding hSclerostin (2 μg/ml) and hDKK1 (2 μg/ml) in kinetics buffer (PBS, pH 7.4, 0.05% Tween™-20, 0.2% BSA). Two incremental binding responses were observed for four bsAbs but not for blank buffer or anti-Sclerostin mAb as shown in FIG. 6, indicating that the bispecific antibodies can bind Sclerostin and DKK1 at the same time and the binding of one target protein does not block that of the other target.

### Example 12. Sclerostin/DKK1-Neutralisation Activity of Bispecific Antibodies on HEK293/TCF/LEF/Wnt1 Cells

The WNT signaling assays were performed as described in Example 7, where 1 μg/ml human Sclerostin and DKK1 were added separately. All bsAbs were able to fully neutralize Wnt-antagonizing effect from both sclerostin and DKK1 in a dose dependent manner. The EC₅₀ activities as shown in Table 13.

TABLE 13
Sclerostin and DKK1 neutralization activity in
HEK293/TCF/LEF/Wnt1 reporter gene assay for anti-
Sclerostin × anti-DKK1 bispecific antibodies

EC₅₀ (nM)

| Antibody | Sclerostin | DKK1 |
|---|---|---|
| BAP0037 | 23.63 | 24.13 |
| BAP0039 | 26.66 | 20.87 |
| BAP0061 | 63.83 | 41.39 |
| BAP0063 | 65.44 | 44.72 |

### Example 13. Pharmacokinetics Study of Bispecific Antibodies

Naive male cynomolgus monkeys were purchased from Guangzhou Huazhen Laboratory Animal Co., Ltd. The animals weighed between 2.7 kg and 3.5 kg and were 3-5 years old at the initiation of dosing. The animals were randomly assigned to 4 dose groups (n=3 per group). On the day of dosing, 30 mg/kg of antibodies were administered subcutaneously. Blood samples were collected on days 1 (pre-dose, 12 and 24 hr after dosing), 2, 3, 5, 7, 10, 14, 18, 21, 28 after dosing. The PK sampling scheme used in this study was based on known PK properties of other human IgG therapeutic mAbs in cynomolgus monkeys.

The assay for the determination of bsAbs in cynomolgus monkey serum employed a qualified fit-for-purpose sandwich ELISA method. CORNING® High binding 96 well polystyrene Microplates were coated with a specific human IgG-heavy and light chain monkey-adsorbed Antibody. Standard, quality control and unknown samples were loaded into the wells, and any human antibody present was bound by the immobilized antibody. After washing away any unbound substances, a human IgG-heavy and light chain monkey-adsorbed Antibody Conjugate HRP and TMB Substrate Solution for ELISA were added to the wells. Only the samples that contain human bsAbs bound to both the capture and detection antibody produced absorption signal at 450 nm. The signal produced was proportional to the amount of human bsAb present. The lowest quantifiable concentration (LLOQ) of antibodies in a sample was 0.1 μg/ml.

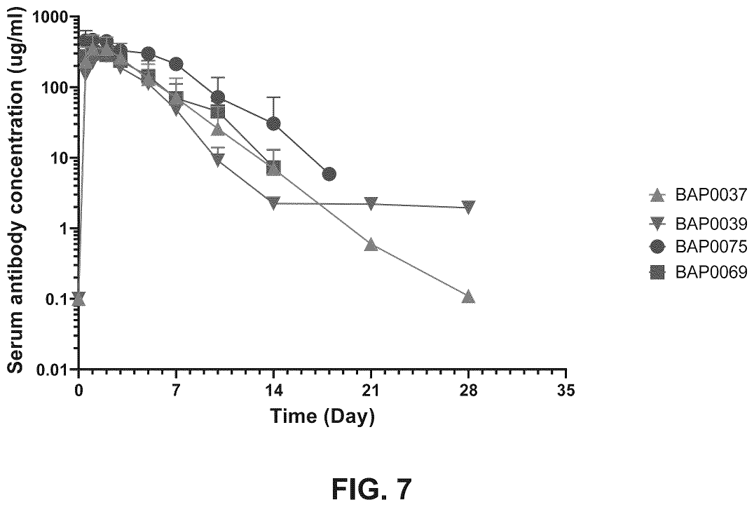

The serum concentrations of the bispecific antibodies in cynomolgus monkeys are shown in FIG. 7. Data are presented as mean±S.E. n=3. The half-lives were calculated to range from 17 to 286 hours.

### Example 14. Pharmacodynamics Study of Bispecific Antibodies

The bone formation biomarker procollagen type IN-terminal propeptide (P1NP) as well as bone resorption biomarker C-terminal end of the telopeptide of type I collagen (CTX-1) are two widely accepted specific bone homeostatic indicators. To test the pharmacodynamic effects of the bispecific antibodies, normal male cynomolgus monkeys were subjected subcutaneous injection of different bispecific antibodies described above at 30 mg/kg. The blood samples were obtained on day 3 after the injection. Serum samples were then separated by centrifugation. Serum P1NP and CTX-1 levels were measured using P1NP Test Kit (PC-007, Guangzhou Phicon Biotech Co. Guangzhou, China) and Serum CrossLaps® ELISA kit (AC-021F1R, Immunodiagnostic Systems, UK) respectively following the manufacturer's instruction. The results showed that the administration of bispecific antibody caused a dramatic increase in serum P1NP levels only 3 days after the injection. When compared to the pre-dose baseline, serum P1NP levels at day 3 were increased between 104.50±8.5% and 212.75±20.6% for different bispecific antibodies (Table 14). Moreover, the serum CTX-1 levels decreased between 46.73±2.5% and 90.11±6.8% for different bispecific antibodies (Table 14) compared to the baseline in response to bispecific antibody injection on day 3. This result indicated that the bispecific antibodies could robustly increase bone formation and decrease bone resorption simultaneously, suggesting their profound therapeutic efficacy to treat diseases or conditions associated with low bone mass or poor bone quality such as osteopenia or osteoporosis in both men and women, osteogenesis imperfecta, multiple myeloma bone diseases. Data was shown in Table 14.

TABLE 14
The serum concentrations of P1NP and CTX-1 in
cynomolgus monkeys 3 days after injection

% Change from pre-dose (mean ± S.E. n = 3)

| Antibody | Serum P1NP | Serum CTX-1 |
|---|---|---|
| BAP0037 | 212.75 ± 20.6 | −90.11 ± 6.8 |
| BAP0039 | 127.92 ± 18.8 | −75.03 ± 10.1 |
| BAP0061 | 180.39 ± 23.8 | −46.73 ± 2.5 |
| BAP0063 | 117.12 ± 16.0 | −68.81 ± 12.5 |
| BAP0069 | 104.50 ± 8.5 | −75.07 ± 2.9 |

Furthermore, when using the bispecific antibody to treat sham operated (Sham) or long-term ovariectomized (OVX) female cynomolgus monkeys, a very well validated postmenopausal osteoporosis animal model, single injection of 10 mg/kg of the bispecific antibody induced a significant increase in serum P1NP and a reduction in serum CTX-1 levels. Compared with pre-dose baseline value, serum P1NP level increases 241.36±60.9% in OVX monkeys while it increases 182.67±60.1% in Sham monkeys at 14 days post-dosing respectively. Simultaneously, serum CTX-1 decreased −52.12±5.9% in OVX monkeys while it decreases −17.43±8.5% in Sham monkeys at 14 days post-dosing respectively. These data provided further proof of therapeutic efficacy of increasing bone formation and decreasing bone resorption in age-related or postmenopausal osteoporosis.

### Example 15. Bispecific Antibodies are Able to Prevent Bone Fractures in Osteogenesis Imperfecta (OI)

The multiscale hierarchical structure of bone is naturally optimized to resist fractures. Enhanced bone formation and reduced bone absorption are two important process to keep the bone strength strong and healthy. In osteogenesis imperfecta (OI) or brittle bone disease, genetic mutations affect the quality and/or the quantity of collagen and dramatically increased bone fracture risk. Clinically, low bone mass and strength, extreme bone fragility and skeletal deformities, as well as frequent fractures becomes the hallmark of the disease. The treatment to reduce the fracture rates in OI is the ultimate goal for clinical improvement. Therefore, we investigated the bispecific antibodies in osteogenesis imperfecta (OI) mouse model (oim/oim) where a spontaneous mutation in the pro-a2 chain of type I collagen causes poor bone quality and lower bone mass resulted in frequent brittle bone fractures. At age of 4 weeks-old, the homozygous oim/oim mouse started to have fractures in their long bones. After 6 weeks of subcutaneous administration of either individual or bispecific antibodies (25 mg/kg, once per week for each antibody), the number of newly occurred fractures were reduced dramatically less than that in vehicle treated animals at 10 weeks-old. In vehicle treated control group, from age of 4 weeks to 10 weeks (6 weeks observation period), the number of fractures was increased by 2.27±0.7 per mouse, counting on X-ray images. Treatment of either sclerostin antibody or DKK1 antibody alone caused a reduction in number of fractures per mouse (0.75±0.2, 1.44±0.4, respectively) as compared with vehicle treatment controls. More dramatically, oim/oim mice treated with the bispecific antibody completely prevent the newly occurred fractures (0.00±0.5 new fracture per mouse from age 4 weeks to 10 weeks), much more superior than either of the mono-therapeutic alone. These results suggest that the bispecific antibodies achieved a synergistic bone anabolic effect by preventing further fractures when neutralizing Sclerostin and DKK1 at the same time. Given that both sclerostin and DKK1 binds to Wnt receptor LRP5/6 and act as antagonists to block the anabolic property of the signaling pathway, it is noticeable if blocking either one of the binding site with specific antibody would cause a up regulation of other antagonists by compensatory mechanism, as demonstrated by the observed up regulation of DKK1 after the treatment with anti-sclerostin antibody in osteoporosis patients (Holdsworth G, 2018, Bone 107, 93-103) Therefore, the above results from current study strongly suggested the synergistic effects on bone by preventing further fractures. This promising result clearly suggested the bispecific antibody is capable to reduce the unmet need of OI patients and to reduce the burden of repeated fractures.

### Example 16. Bispecific Antibodies Suppress In Vitro Growth of RPMI8226 Multiple Myeloma Cell

The bispecific antibodies are also evaluated in RPMI8226 human multiple myeloma (MM) cells. As indicated in numerous scientific reports demonstrated that DKK1 expression levels are increased in MM patients and are associated with the severity of the disease (Fulciniti M. et al. Blood, 2009; 114(2): 371-379., Feng Y et al. Cancer Biomarkers. 2018; 1: 1-7). Furthermore, studies have shown that about 80-90% of MM patients develop MM related bone disorder (MMBD). The major clinical expression of MMBD is osteolytic lesions and fractures due to increased activities of bone resorption and suppression of bone formation. Therefore, the nature of the bispecific antibodies will benefit MMBD patients by providing bone resorptive inhibition as well as by inhibiting DKK1 expression that leads to increase bone formation. Our preliminary studies demonstrated that in vitro cell culture of MM cells (RPMI 8226) subjected to the molecule showed a decrease in cell surface marker CD138 levels, suggesting the cells might be undergone apoptosis process. Furthermore, CCK8 analysis of tumor growth showed a dose dependent reduction in tumor cell numbers. When administration of the bispecific antibody at 100 μg/ml to the RPMI8226 MM cells for 48 hours, the viable cell numbers dropped about 28%. This evidence indicated that the bispecific antibody could cause the RPMI8226 MM cells apoptosis and arrested the tumor cell growth. The results suggested the therapeutic application to treat MMBD and tumor metastatic related bone disorders by not only preventing bone disorders (as data shown in Example 14 and 15) but also directly inhibiting tumor growth.

SEQUENCE TABLE

| | | |
|---|---|---|
| SEQ ID | | |
| NO. | Description | Nucleotide or Amino Acid Sequence |

Exemplary anti-Sclerostin mAb sequences

| | | |
|---|---|---|
| 1. | HC-CDR1 | DYEIH |
| 2. | HC-CDR1 | DFEMH |
| 3. | HC-CDR1 | DYEMH |
| 4. | HC-CDR1 | SYWMH |
| 5. | HC-CDR2 | AIDPETGGTAYNQKFKG |
| 6. | HC-CDR2 | AIDPETGGTAYNQKFKA |
| 7. | HC-CDR2 | AIDPETGGTAYNQKFTA |
| 8. | HC-CDR2 | MIHPNSGSSNYNEKFKS |
| 9. | HC-CDR3 | YDYVTY |
| 10. | HC-CDR3 | YDYVSY |
| 11. | HC-CDR3 | DYDDEGFAY |
| 12. | HC-CDR1 | DX₁EX₂H |
| | consensus | X₁ = Y or F, X₂ = M or I. |
| | sequence | |
| 13. | HC-CDR2 | AIDPETGGX₁AX₂NQKFX₃X₄ |
| | consensus | X₁ = T or S, X₂ = Y or N, |
| | sequence | X₃ = K or T, X₄ = A, G, or S. |
| 14. | HC-CDR3 | YDYVX₁Y |
| | consensus | X₁ = T or S |
| | sequence | |
| 15 | LC-CDR1 | KSSQSLLYSDGRTYLN |
| 16. | LC-CDR1 | KSSQSLLYSDGKTYLN |
| 17. | LC-CDR1 | KASQSVSNDVA |
| 18. | LC-CDR2 | LVSKLDS |
| 19. | LC-CDR2 | YASNRCT |
| 20. | LC-CDR3 | WQGTHLPHT |
| 21. | LC-CDR3 | QQDYSSPWT |
| 22. | 93B1B7 VH | QAQLQQSGAELVRPGASVTLSCKASGYTFTDYEIHWM |
| | | KQTPVHGLEWIGAIDPETGGTAYNQKFKGKAILTADRS |
| | | SSTAYLELRSLTSEDSAVYYCYSYDYVTYWGQGTLVT |
| | | VSA |
| 23. | 93B1B7 VL | DVVMTQTPLTWSITIGQPASISCKSSQSLLYSDGRTYLN |
| | | WLLQRPGQSPKRLIYLVSKLDSGVPDRFSGSGSGTDFTL |
| | | KISRVEAEDLGFYYCWQGTHLPHTFGAGTKLELK |
| 24. | 94B12D3 V_{H} | QVQLQQSGAELVRPGASVTLSCKASGYTFTDYEIHWVK |
| | | QTPVHGLEWIGAIDPETGGTAYNQKFKAKAILTADRSSS |
| | | TAYMELRSLTSEDSAVYYSFSYDYVSYWGQGTLVTVS |
| | | A |
| 25. | 94B12D3 VL | DVVMTQTPLTLSVTIGQPASISCKSSQSLLYSDGRTYLN |
| | | WLLQRPGQSPKRLIYLVSKLDSGVPDRFAGSGSGTDFSL |
| | | KISRVEAEDLGVYYCWQGTHLPHTFGAGTKLELK |
| 26. | 71G6G8 VH | QAQLQQSGAELVRPGASVTLSCKASGYTFTDYEIHWVK |
| | | QTPVHGLEWIGAIDPETGGTAYNQKFKGKAILTADRSSS |
| | | TAYLELRSLTSEDSAVYYCFSYDYVTYWGQGTLVTVT |
| | | A |
| 27. | 71G6G8 VL | DVVMTQTPLTWSITIGQPASISCKSSQSLLYSDGRTYLN |
| | | WLLQRPGQSPKRLIYLVSKLDSGVPDRFTGSGSGTDFTL |
| | | KISRVEAEDLGFYYCWQGTHLPHTFGAGTKLELK |
| 28. | 56E5C10 VH | QVQLQQSGAELVRPGASVTLSCKASGYTFTDFEMHWV |
| | | KQTPVHGLEWIGAIDPETGGTAYNQKFTAKAILTADRS |
| | | SSTAYMELRSLTSEDSAVYYCFSYDYVSYWGQGTLVT |
| | | VSA |
| 29. | 56E5C10 VL | DVMMTQTPLTLSVTIGQPASISCKSSQSLLYSDGRTYLN |
| | | WLLQRPGQSPKRLIYLVSKLDSGVPDRFTGSGSGTDFTL |
| | | KISRVEAEDLGVYYCWQGTHLPHTFGAGTKLELK |
| 30. | 91F6D10 VH | QVQLQQSGAELVRPGASVTLSCKASGYTFTDYEMHWV |
| | | KQTPVHGLEWIGAIDPETGGTAYNQKFKAKAILTADRS |
| | | SSTAYMELRSLTSEDSAVYYCFSYDYVSYWGQGTLVT |
| | | VSA |
| 31. | 91F6D10 VL | DVVMTQTPLTLSVTIGQPASISCKSSQSLLYSDGRTYLN |
| | | WLLQRPGQSPKRLIYLVSKLDSGVPDRFTGSGSGTDFTL |
| | | KISRVEAEDLGVYYCWQGTHLPHTFGVGTKLELK |
| 32. | 51E8D4 VH | QVQLQQSGAELVRPGASVTLSCKASGYTFTDFEIHWMK |
| | | QTPVPGLEWIGAIDPETGGTAYNQKFKGKALLTADKSS |
| | | STAYMDLRSLTSEDSAVYFCFSYDYVSYWGQGTLVTV |
| | | SA |
| 33. | 51E8D4 VL | DVVMTQTPLTLSVTFGQPASISCKSSQSLLYSDGKTYLN |
| | | WLLQRPGQSPKRLIYLVSKLDSGVPDRFTGSGSGTAFTL |
| | | KISRVEAEDLGVYYCWQGTHLPHTFGAGTKLELR |
| 34. | 97C11D7 VH | QVQLQQSGAELVRPGASVTLSCKASGYTFTDYEMHWV |
| | | KQTPVHGLEWIGAIDPETGGSANNQKFKAKAILTADRS |
| | | SSTAYMELRSLTSEDSAVYYCFSYDYVSYWGQGTLVT |
| | | VSA |
| 35. | 97C11D7 VL | DVVMTQTPLTLSVTIGQPASISCKSSQSLLYSDGRTYLN |
| | | WLLQRPGQSPKRLIYLVSKLDSGVPDRFTGSGSGTDFTL |
| | | KIGRVEAEDLGVYYCWQGTHLPHTFGAGTKLELK |
| 36. | 81B2B6 VH | QVQLQQSGAELVRPGASVTLSCKASGYTFTDYEMHWV |
| | | KQTPVHGLEWIGAIDPETGGTAYNQKFKAKAILTADRS |
| | | SSTAYMELRSLTSEDSAVYYCFSYDYVSYWGQGTLVT |
| | | VSA |
| 37. | 81B2B6 VL | DVVMTQTPLTLSVTLGQPASISCKSSQSLLYSDGRTYLN |
| | | WLLQRPGQSPKRLIYLVSKLDSGVPDRFTGSGSGTDFTL |
| | | KISRVEAEDLGLYYCWQGTHLPHTFGAGTKLELK |
| 38. | 84F2D5 VH | QVQLQQSGAELVRPGASVTLSCKASGYTFTDFEIHWLK |
| | | QTPVPGLEWIGAIDPETGGTAYNQKFKGKALLTADKSS |
| | | STAYMELRSLTSEDSAVYYCFSYDYVSYWGQGTLVTV |
| | | SA |
| 39. | 84F2D5 VL | DVVMTQTPLTLSVTIGQPASISCKSSQSLLYSDGKTYLN |
| | | WLLQRPGQSPKRLIYLVSKLDSGVPDRFTGSGSGTAFTL |
| | | KISRVEAEDLGVYYCWQGTHLPHTFGAGTKLELR |
| 40. | 65B12C9 VH | QVQLQQPGAELVKPGASVKLSCKASGYTFTSYWMHW |
| | | VKQRPGQGLEWIGMIHPNSGSSNYNEKFKSKATLTVDK |
| | | SSSTAYMQLSSLTSEDSAVYYCANDYDDEGFAYWGQG |
| | | TLVTVSA |
| 41. | 65B12C9 VL | SIVMTQTPKFLLVSAGDRVTITCKASQSVSNDVAWYQQ |
| | | KPGQSPKLLIYYASNRCTGVPDRFTGSGYGTDFTFTIST |
| | | VQAEDLAVYFCQQDYSSPWTFGGGTKLEIK |

Exemplary anti-DKK1 sequences

| | | |
|---|---|---|
| 42. | 11H10- | GFTFSDYAMA |
| | VH44VL32 | |
| | HC-CDR1 | |
| 43. | 11H10- | THIYDGSSTYYRDSVKG |
| | VH44VL32 | |
| | HC-CDR2 | |
| 44. | 11H10- | GLGIATDYFDY |
| | VH44VL32 | |
| | HC-CDR3 | |
| 45. | 11H10- | LASEDIYSDLA |
| | VH44VL32 | |
| | LC-CDR1 | |
| 46. | 11H10- | NANSLQN |
| | VH44VL32 | |
| | LC-CDR2 | |
| 47. | 11H10- | QQYNNYPPT |
| | VH44VL32 | |
| | LC-CDR3 | |
| 48. | RH2-18 HC- | GYTFTDYYIH |
| | CDR1 | |
| 49. | RH2-18 HC- | WIHSNSGATTYAQKFQA |
| | CDR2 | |
| 50. | RH2-18 HC- | EDY |
| | CDR3 | |
| 51. | RH2-18 LC- | TGSSNIGAGYDVH |
| | CDR1 | |
| 52. | RH2-18 LC- | GYSNRPS |
| | CDR2 | |
| 53. | RH2-18 LC- | QSYDNSLSSYV |
| | CDR3 | |
| 54. | 71G6G8 HC- | GFTFSSYAIS |
| | CDR1 | |
| 55. | 71G6G8 HC- | SVSGTGLGFQTYYPDSVKG |
| | CDR2 | |
| 56. | 71G6G8 HC- | SLENYAFDY |
| | CDR3 | |
| 57. | 71G6G8 LC- | RASESVDDFGISFIN |
| | CDR1 | |
| 58. | 71G6G8 LC- | AGSKQGS |
| | CDR2 | |
| 59. | 71G6G8 LC- | QQLKEVPPT |
| | CDR3 | |
| 60. | 11H10- | EVQLVESGGGVVQPGRSLRLSCAASGFTFSDYAMAWV |
| | VH44VL32 | RQAPGKGLEWVATIIYDGSSTYYRDSVKGRFTISRDNS |
| | VH (11H10 | KNTLYLQMNSLRAEDTAVYYCATGLGIATDYFDYWG |
| | VH) | QGTLVTVSS |
| 61. | 11H10- | DIRMTQSPFSLSASVGDRVTITCLASEDIYSDLAWYQQK |
| | VH44VL32 | PAKAPKLFIYNANSLQNGVPSRFSGSGSGTDYTLTISSL |
| | VL (11H10 | QPEDFATYYCQQYNNYPPTFGGGTKVEIK |
| | VL) | |
| 62. | RH2-18 VH | EVQLVQSGAEVKKPGASVKVSCKASGYTFTDYYIHWV |
| | | RQAPGQGLEWMGWIHSNSGATTYAQKFQARVTMSRD |
| | | TSSSTAYMELSRLESDDTAMYFCSREDYWGQGTLVTV |
| | | SS |
| 63. | RH2-18 VL | QSVLTQPPSVSGAPGQRVTISCTGSSNIGAGYDVHWYQ |
| | | QLPGTAPKLLIYGYSNRPSGVPDRFSGSKSGASASLAIT |
| | | GLRPDDEADYYCQSYDNSLSSYVFGGGTQLTVL |
| 64. | 71G6G8 VH | EVQLVESGGGLVQPGGSLRLSCAASGFTFSSYAISWVR |
| | | QAPGKGLEWVASVSGTGLGFQTYYPDSVKGRFTISRDN |
| | | AKNSLYLQMNSLRAEDTAVYYCATSLENYAFDYWGQ |
| | | GTTVTVSS |
| 65. | 71G6G8 VL | EIVLTQSPATLSLSPGERATLSCRASESVDDFGISFINWY |
| | | QQKPGQAPRLLIYAGSKQGSGIPARFSGSGSGTDFTLTIS |
| | | SLEPEDFAVYYCQQLKEVPPTFGGGTKVEIK |

Exemplary anti-RANKL sequences

| | | |
|---|---|---|
| 66. | Denosumab | GFTFSSYAMS |
| | HC-CDR1 | |
| 67. | Denosumab | GITGSGGSTYYADSVKG |
| | HC-CDR2 | |
| 68. | Denosumab | DPGTTVIMSWFDP |
| | HC-CDR3 | |
| 69. | Denosumab | RASQSVRGRYLA |
| | LC-CDR1 | |
| 70. | Denosumab | GASSRAT |
| | LC-CDR2 | |
| 71. | Denosumab | QQYGSSPRT |
| | LC-CDR3 | |
| 72. | Denosumab | EVQLLESGGGLVQPGGSLRLSCAASGFTFSSYAMSWVR |
| | VH | QAPGKGLEWVSGITGSGGSTYYADSVKGRFTISRDNSK |
| | | NTLYLQMNSLRAEDTAVYYCAKDPGTTVIMSWFDPW |
| | | GQGTLVTVSS |
| 73. | Denosumab | EIVLTQSPGTLSLSPGERATLSCRASQSVRGRYLAWYQ |
| | VL | QKPGQAPRLLIYGASSRATGIPDRFSGSGSGTDFTLTISR |
| | | LEPEDFAVFYCQQYGSSPRTFGQGTKVEIK |

Exemplary linker sequences

| | | |
|---|---|---|
| 74. | Linker | GGGGS |
| 75. | Linker | GGGGSGGGGS |
| 76. | Linker | GGGGSGGGGSGGGGS |
| 77. | Linker | GGGGSGGGGSGGGGSGGGGS |
| 78. | Linker | (G)ₙ, n > = 1 |
| 79. | Linker | (GS)ₙ, 8 > = n > = 1 |
| 80. | Linker | (GSGGS)ₙ, 8 > = n > = 1 |
| 81. | Linker | (GGGGS)ₙ, 8 > = n > = 1 |
| 82. | Linker | (GGGS)ₙ, 8 > = n > = 1 |
| 83. | Linker | (GGGGS)₆ |
| 84. | Linker | (GSTSGSGKPGSGEGS)ₙ |
| | | 3 > = n > = 1 |

Exemplary humanized anti-sclerostin antibody sequences

| | | |
|---|---|---|
| 85. | hAb-2 LC- | RSSQSLLYSDGRTYLN |
| | CDR1 | |
| 86. | hAb-3 LC- | RSSQSLLYSDSRTYLN |
| | CDR1 | |
| 87. | hAb-1/hAb-2/ | EVQLVQSGAEVKKPGASVKVSCKASGYTFTDYEIHWV |
| | hAb-3 VH | RQAPGKGLEWMGAIDPETGGTAYNQKFKGRVTMTTDT |
| | (hAb VH) | STSTAYMELRSLRSDDTAVYYCYSYDYVTYWGQGTTV |
| | | TVSS |
| 88. | hAb-1 VL | DVVMTQSPLSLSVSPGERASLSCKSSQSLLYSDGRTYLN |
| | | WYLQKPGQSPQRLIYLVSKLDSGVPDRFSGSGSGTDFTL |
| | | KISRVQSEDVGVYYCWQGTHLPHTFGGGTKVEIK |
| 89. | hAb-2 VL | DIQMTQSPSSLSASVGDRVTITCRSSQSLLYSDGRTYLN |
| | | WYQQKPGKSPKRLIYLVSKLDSGVPDRFSGSGSGTDFT |
| | | LTISSLQPEDFATYYCWQGTHLPHTFGGGTKVEIK |
| 90. | hAb-3 VL | DIQMTQSPSSLSASVGDRVTITCRSSQSLLYSDSRTYLN |
| | | WLQQKPGKSPKRLIYLVSKLDSGVPDRFSGSGSGTDFTL |
| | | TISSLQPEDFATYYCWQGTHLPHTFGGGTKVEIK |

Exemplary bispecific anti-sclerostin antibody sequences

| | | |
|---|---|---|
| 91. | BAP0017 | EVQLVESGGGLVQPANSLKLSCAASGFTFSDYAMAWV |
| | | RQSPKKGLEWVATIIYDGSSTYYRDSVKGRFTISRDNAK |
| | | STLYLQMDSLRSEDTATYYCATGLGIATDYFDYWGQG |
| | | VLVTVSSAETTAPSVYPLAPGTALKSNSMVTLGCLVKG |
| | | YFPEPVTVTWNSGALSSGVHTFPAVLQSGLYTLTSSVT |
| | | VPSSTWPSQTVTCNVAHPASSTKVDKKIVPRNCGGDCK |
| | | PCICTGSEVSSVFIFPPKPKDVLTITLTPKVTCVVVDISQD |
| | | DPEVHFSWFVDDVEVHTAQTRPPEEQFNSTFRSVSELPI |
| | | LHQDWLNGRTFRCKVTSAAFPSPIEKTISKPEGRTQVPH |
| | | VYTMSPTKEEMTQNEVSITCMVKGFYPPDIYVEWQMN |
| | | GQPQENYKNTPPTMDTDGSYFLYSKLNVKKEKWQQG |
| | | NTFTCSVLHEGLHNHHTEKSLSHSPGKGGGGSGGGGSG |
| | | GGGSQAQLQQSGAELVRPGASVTLSCKASGYTFTDYEI |
| | | HWMKQTPVHGLEWIGAIDPETGGTAYNQKFKGKAILT |
| | | ADRSSSTAYLELRSLTSEDSAVYYCYSYDYVTYWGQG |
| | | TLVTVSAGGGGSGGGGSGGGGSGGGGSDVVMTQTPLT |
| | | WSITIGQPASISCKSSQSLLYSDGRTYLNWLLQRPGQSP |
| | | KRLIYLVSKLDSGVPDRFSGSGSGTDFTLKISRVEAEDL |
| | | GFYYCWQGTHLPHTFGAGTKLELK |
| 92. | heavy chain | EVQLVESGGGLVQPANSLKLSCAASGFTFSDYAMAWV |
| | BAP0018 | RQSPKKGLEWVATIIYDGSSTYYRDSVKGRFTISRDNAK |
| | heavy chain | STLYLQMDSLRSEDTATYYCATGLGIATDYFDYWGQG |
| | | VLVTVSSAETTAPSVYPLAPGTALKSNSMVTLGCLVKG |
| | | YFPEPVTVTWNSGALSSGVHTFPAVLQSGLYTLTSSVT |
| | | VPSSTWPSQTVTCNVAHPASSTKVDKKIVPRNCGGDCK |
| | | PCICTGSEVSSVFIFPPKPKDVLTITLTPKVTCVVVDISQD |
| | | DPEVHFSWFVDDVEVHTAQTRPPEEQFNSTFRSVSELPI |
| | | LHQDWLNGRTFRCKVTSAAFPSPIEKTISKPEGRTQVPH |
| | | VYTMSPTKEEMTQNEVSITCMVKGFYPPDIYVEWQMN |
| | | GQPQENYKNTPPTMDTDGSYFLYSKLNVKKEKWQQG |
| | | NTFTCSVLHEGLHNHHTEKSLSHSPGKGGGGSGGGGSG |
| | | GGGSDVVMTQTPLTWSITIGQPASISCKSSQSLLYSDGR |
| | | TYLNWLLQRPGQSPKRLIYLVSKLDSGVPDRFSGSGSGT |
| | | DFTLKISRVEAEDLGFYYCWQGTHLPHTFGAGTKLELK |
| | | GGGGSGGGGSGGGGSGGGGSQAQLQQSGAELVRPGAS |
| | | VTLSCKASGYTFTDYEIHWMKQTPVHGLEWIGAIDPET |
| | | GGTAYNQKFKGKAILTADRSSSTAYLELRSLTSEDSAV |
| | | YYCYSYDYVTYWGQGTLVTVSA |
| 93. | BAP0017/ | DIRMTQSPASLSASLGETVNIECLASEDIYSDLAWYQQK |
| | BAP0018 light | PGKSPQLLIYNANSLQNGVPSRFSGSGSGTQYSLKINSL |
| | chain | QSEDVATYFCQQYNNYPPTFGGGTKLELKRADAAPTVS |
| | | IFPPSTEQLATGGASVVCLMNNFYPRDISVKWKIDGTER |
| | | RDGVLDSVTDQDSKDSTYSMSSTLSLTKADYESHNLYT |
| | | CEVVHKTSSSPVVKSFNRNEC |
| 94. | BAP0019 | QAQLQQSGAELVRPGASVTLSCKASGYTFTDYEIHWM |
| | heavy chain | KQTPVHGLEWIGAIDPETGGTAYNQKFKGKAILTADRS |
| | | SSTAYLELRSLTSEDSAVYYCYSYDYVTYWGQGTLVT |
| | | VSAAKTTPPSVYPLAPGSAAQTNSMVTLGCLVKGYFPE |
| | | PVTVTWNSGSLSSGVHTFPAVLQSDLYTLSSSVTVPSSP |
| | | RPSETVTCNVAHPASSTKVDKKIVPRDCGCKPCICTVPE |
| | | VSSVFIFPPKPKDVLTITLTPKVTCVVVDISKDDPEVQFS |
| | | WFVDDVEVHTAQTQPREEQFNSTFRSVSELPIMHQDWL |
| | | NGKEFKCRVNSAAFPAPIEKTISKTKGRPKAPQVYTIPPP |
| | | KEQMAKDKVSLTCMITDFFPEDITVEWQWNGQPAENY |
| | | KNTQPIMNTNGSYFVYSKLNVQKSNWEAGNTFTCSVL |
| | | HEGLHNHHTEKSLSHSPGKGGGGSGGGGSGGGGSEVQ |
| | | LVESGGGLVQPANSLKLSCAASGFTFSDYAMAWVRQS |
| | | PKKGLEWVATIIYDGSSTYYRDSVKGRFTISRDNAKSTL |
| | | YLQMDSLRSEDTATYYCATGLGIATDYFDYWGQGVLV |
| | | TVSSGGGGSGGGGSGGGGSGGGGSDIRMTQSPASLSAS |
| | | LGETVNIECLASEDIYSDLAWYQQKPGKSPQLLIYNANS |
| | | LQNGVPSRFSGSGSGTQYSLKINSLQSEDVATYFCQQY |
| | | NNYPPTFGGGTKLELK |
| 95. | BAP0020 | QAQLQQSGAELVRPGASVTLSCKASGYTFTDYEIHWM |
| | heavy chain | KQTPVHGLEWIGAIDPETGGTAYNQKFKGKAILTADRS |
| | | SSTAYLELRSLTSEDSAVYYCYSYDYVTYWGQGTLVT |
| | | VSAAKTTPPSVYPLAPGSAAQTNSMVTLGCLVKGYFPE |
| | | PVTVTWNSGSLSSGVHTFPAVLQSDLYTLSSSVTVPSSP |
| | | RPSETVTCNVAHPASSTKVDKKIVPRDCGCKPCICTVPE |
| | | VSSVFIFPPKPKDVLTITLTPKVTCVVVDISKDDPEVQFS |
| | | WFVDDVEVHTAQTQPREEQFNSTFRSVSELPIMHQDWL |
| | | NGKEFKCRVNSAAFPAPIEKTISKTKGRPKAPQVYTIPPP |
| | | KEQMAKDKVSLTCMITDFFPEDITVEWQWNGQPAENY |
| | | KNTQPIMNTNGSYFVYSKLNVQKSNWEAGNTFTCSVL |
| | | HEGLHNHHTEKSLSHSPGKGGGGSGGGGSGGGGSDIR |
| | | MTQSPASLSASLGETVNIECLASEDIYSDLAWYQQKPG |
| | | KSPQLLIYNANSLQNGVPSRFSGSGSGTQYSLKINSLQSE |
| | | DVATYFCQQYNNYPPTFGGGTKLELKGGGGSGGGGSG |
| | | GGGSGGGGSEVQLVESGGGLVQPANSLKLSCAASGFTF |
| | | SDYAMAWVRQSPKKGLEWVATIIYDGSSTYYRDSVKG |
| | | RFTISRDNAKSTLYLQMDSLRSEDTATYYCATGLGIATD |
| | | YFDYWGQGVLVTVSS |
| 96. | BAP0019/ | DVVMTQTPLTWSITIGQPASISCKSSQSLLYSDGRTYLN |
| | BAP0020 light | WLLQRPGQSPKRLIYLVSKLDSGVPDRFSGSGSGTDFTL |
| | chain | KISRVEAEDLGFYYCWQGTHLPHTFGAGTKLELKRAD |
| | | AAPTVSIFPPSSEQLTSGGASVVCFLNNFYPKDINVKWK |
| | | IDGSERQNGVLNSWTDQDSKDSTYSMSSTLTLTKDEYE |
| | | RHNSYTCEATHKTSTSPIVKSFNRNEC |
| 97. | BAP0021 | QAQLQQSGAELVRPGASVTLSCKASGYTFTDYEIHWM |
| | heavy chain | KQTPVHGLEWIGAIDPETGGTAYNQKFKGKAILTADRS |
| | | SSTAYLELRSLTSEDSAVYYCYSYDYVTYWGQGTLVT |
| | | VSAGGGGSGGGGSGGGGSGGGGSDVVMTQTPLTWSIT |
| | | IGQPASISCKSSQSLLYSDGRTYLNWLLQRPGQSPKRLIY |
| | | LVSKLDSGVPDRFSGSGSGTDFTLKISRVEAEDLGFYYC |
| | | WQGTHLPHTFGAGTKLELKGGGGSGGGGSGGGGSEVQ |
| | | LVESGGGLVQPANSLKLSCAASGFTFSDYAMAWVRQS |
| | | PKKGLEWVATIIYDGSSTYYRDSVKGRFTISRDNAKSTL |
| | | YLQMDSLRSEDTATYYCATGLGIATDYFDYWGQGVLV |
| | | TVSSAETTAPSVYPLAPGTALKSNSMVTLGCLVKGYFP |
| | | EPVTVTWNSGALSSGVHTFPAVLQSGLYTLTSSVTVPSS |
| | | TWPSQTVTCNVAHPASSTKVDKKIVPRNCGGDCKPCIC |
| | | TGSEVSSVFIFPPKPKDVLTITLTPKVTCVVVDISQDDPE |
| | | VHFSWFVDDVEVHTAQTRPPEEQFNSTFRSVSELPILHQ |
| | | DWLNGRTFRCKVTSAAFPSPIEKTISKPEGRTQVPHVYT |
| | | MSPTKEEMTQNEVSITCMVKGFYPPDIYVEWQMNGQP |
| | | QENYKNTPPTMDTDGSYFLYSKLNVKKEKWQQGNTFT |
| | | CSVLHEGLHNHHTEKSLSHSPGK |
| 98. | BAP0022 | DVVMTQTPLTWSITIGQPASISCKSSQSLLYSDGRTYLN |
| | heavy chain | WLLQRPGQSPKRLIYLVSKLDSGVPDRFSGSGSGTDFTL |
| | | KISRVEAEDLGFYYCWQGTHLPHTFGAGTKLELKGGG |
| | | GSGGGGSGGGGSGGGGSQAQLQQSGAELVRPGASVTL |
| | | SCKASGYTFTDYEIHWMKQTPVHGLEWIGAIDPETGGT |
| | | AYNQKFKGKAILTADRSSSTAYLELRSLTSEDSAVYYC |
| | | YSYDYVTYWGQGTLVTVSAGGGGSGGGGSGGGGSEV |
| | | QLVESGGGLVQPANSLKLSCAASGFTFSDYAMAWVRQ |
| | | SPKKGLEWVATIIYDGSSTYYRDSVKGRFTISRDNAKST |
| | | LYLQMDSLRSEDTATYYCATGLGIATDYFDYWGQGVL |
| | | VTVSSAETTAPSVYPLAPGTALKSNSMVTLGCLVKGYF |
| | | PEPVTVTWNSGALSSGVHTFPAVLQSGLYTLTSSVTVPS |
| | | STWPSQTVTCNVAHPASSTKVDKKIVPRNCGGDCKPCI |
| | | CTGSEVSSVFIFPPKPKDVLTITLTPKVTCVVVDISQDDP |
| | | EVHFSWFVDDVEVHTAQTRPPEEQFNSTFRSVSELPILH |
| | | QDWLNGRTFRCKVTSAAFPSPIEKTISKPEGRTQVPHVY |
| | | TMSPTKEEMTQNEVSITCMVKGFYPPDIYVEWQMNGQ |
| | | PQENYKNTPPTMDTDGSYFLYSKLNVKKEKWQQGNTF |
| | | TCSVLHEGLHNHHTEKSLSHSPGK |
| 99. | BAP0021/ | DIRMTQSPASLSASLGETVNIECLASEDIYSDLAWYQQK |
| | BAP0022 light | PGKSPQLLIYNANSLQNGVPSRFSGSGSGTQYSLKINSL |
| | chain | QSEDVATYFCQQYNNYPPTFGGGTKLELKRADAAPTVS |
| | | IFPPSTEQLATGGASVVCLMNNFYPRDISVKWKIDGTER |
| | | RDGVLDSVTDQDSKDSTYSMSSTLSLTKADYESHNLYT |
| | | CEVVHKTSSSPVVKSFNRNEC |
| 100. | BAP0023 | EVQLVESGGGLVQPANSLKLSCAASGFTFSDYAMAWV |
| | heavy chain | RQSPKKGLEWVATIIYDGSSTYYRDSVKGRFTISRDNAK |
| | | STLYLQMDSLRSEDTATYYCATGLGIATDYFDYWGQG |
| | | VLVTVSSGGGGSGGGGSGGGGSGGGGSDIRMTQSPASL |
| | | SASLGETVNIECLASEDIYSDLAWYQQKPGKSPQLLIYN |
| | | ANSLQNGVPSRFSGSGSGTQYSLKINSLQSEDVATYFCQ |
| | | QYNNYPPTFGGGTKLELKGGGGSGGGGSGGGGSQAQL |
| | | QQSGAELVRPGASVTLSCKASGYTFTDYEIHWMKQTPV |
| | | HGLEWIGAIDPETGGTAYNQKFKGKAILTADRSSSTAY |
| | | LELRSLTSEDSAVYYCYSYDYVTYWGQGTLVTVSAAK |
| | | TTPPSVYPLAPGSAAQTNSMVTLGCLVKGYFPEPVTVT |
| | | WNSGSLSSGVHTFPAVLQSDLYTLSSSVTVPSSPRPSET |
| | | VTCNVAHPASSTKVDKKIVPRDCGCKPCICTVPEVSSVF |
| | | IFPPKPKDVLTITLTPKVTCVVVDISKDDPEVQFSWFVD |
| | | DVEVHTAQTQPREEQFNSTFRSVSELPIMHQDWLNGKE |
| | | FKCRVNSAAFPAPIEKTISKTKGRPKAPQVYTIPPPKEQ |
| | | MAKDKVSLTCMITDFFPEDITVEWQWNGQPAENYKNT |
| | | QPIMNTNGSYFVYSKLNVQKSNWEAGNTFTCSVLHEG |
| | | LHNHHTEKSLSHSPGK |
| 101. | BAP0024 | DIRMTQSPASLSASLGETVNIECLASEDIYSDLAWYQQK |
| | heavy chain | PGKSPQLLIYNANSLQNGVPSRFSGSGSGTQYSLKINSL |
| | | QSEDVATYFCQQYNNYPPTFGGGTKLELKGGGGSGGG |
| | | GSGGGGSGGGGSEVQLVESGGGLVQPANSLKLSCAAS |
| | | GFTFSDYAMAWVRQSPKKGLEWVATIIYDGSSTYYRDS |
| | | VKGRFTISRDNAKSTLYLQMDSLRSEDTATYYCATGLG |
| | | IATDYFDYWGQGVLVTVSSGGGGSGGGGSGGGGSQAQ |
| | | LQQSGAELVRPGASVTLSCKASGYTFTDYEIHWMKQTP |
| | | VHGLEWIGAIDPETGGTAYNQKFKGKAILTADRSSSTA |
| | | YLELRSLTSEDSAVYYCYSYDYVTYWGQGTLVTVSAA |
| | | KTTPPSVYPLAPGSAAQTNSMVTLGCLVKGYFPEPVTV |
| | | TWNSGSLSSGVHTFPAVLQSDLYTLSSSVTVPSSPRPSET |
| | | VTCNVAHPASSTKVDKKIVPRDCGCKPCICTVPEVSSVF |
| | | IFPPKPKDVLTITLTPKVTCVVVDISKDDPEVQFSWFVD |
| | | DVEVHTAQTQPREEQFNSTFRSVSELPIMHQDWLNGKE |
| | | FKCRVNSAAFPAPIEKTISKTKGRPKAPQVYTIPPPKEQ |
| | | MAKDKVSLTCMITDFFPEDITVEWQWNGQPAENYKNT |
| | | QPIMNTNGSYFVYSKLNVQKSNWEAGNTFTCSVLHEG |
| | | LHNHHTEKSLSHSPGK |
| 102. | BAP0023/ | DVVMTQTPLTWSITIGQPASISCKSSQSLLYSDGRTYLN |
| | BAP0024 light | WLLQRPGQSPKRLIYLVSKLDSGVPDRFSGSGSGTDFTL |
| | chain | KISRVEAEDLGFYYCWQGTHLPHTFGAGTKLELKRAD |
| | | AAPTVSIFPPSSEQLTSGGASVVCFLNNFYPKDINVKWK |
| | | IDGSERQNGVLNSWTDQDSKDSTYSMSSTLTLTKDEYE |
| | | RHNSYTCEATHKTSTSPIVKSFNRNEC |
| 103. | BAP0025/ | EVQLVESGGGLVQPANSLKLSCAASGFTFSDYAMAWV |
| | BAP0026 heavy | RQSPKKGLEWVATIIYDGSSTYYRDSVKGRFTISRDNAK |
| | chain | STLYLQMDSLRSEDTATYYCATGLGIATDYFDYWGQG |
| | | VLVTVSSAETTAPSVYPLAPGTALKSNSMVTLGCLVKG |
| | | YFPEPVTVTWNSGALSSGVHTFPAVLQSGLYTLTSSVT |
| | | VPSSTWPSQTVTCNVAHPASSTKVDKKIVPRNCGGDCK |
| | | PCICTGSEVSSVFIFPPKPKDVLTITLTPKVTCVVVDISQD |
| | | DPEVHFSWFVDDVEVHTAQTRPPEEQFNSTFRSVSELPI |
| | | LHQDWLNGRTFRCKVTSAAFPSPIEKTISKPEGRTQVPH |
| | | VYTMSPTKEEMTQNEVSITCMVKGFYPPDIYVEWQMN |
| | | GQPQENYKNTPPTMDTDGSYFLYSKLNVKKEKWQQG |
| | | NTFTCSVLHEGLHNHHTEKSLSHSPGK |
| 104. | BAP0025 | QAQLQQSGAELVRPGASVTLSCKASGYTFTDYEIHWM |
| | light chain | KQTPVHGLEWIGAIDPETGGTAYNQKFKGKAILTADRS |
| | | SSTAYLELRSLTSEDSAVYYCYSYDYVTYWGQGTLVT |
| | | VSAGGGGSGGGGSGGGGSGGGGSDVVMTQTPLTWSIT |
| | | IGQPASISCKSSQSLLYSDGRTYLNWLLQRPGQSPKRLIY |
| | | LVSKLDSGVPDRFSGSGSGTDFTLKISRVEAEDLGFYYC |
| | | WQGTHLPHTFGAGTKLELKGGGGSGGGGSGGGGSDIR |
| | | MTQSPASLSASLGETVNIECLASEDIYSDLAWYQQKPG |
| | | KSPQLLIYNANSLQNGVPSRFSGSGSGTQYSLKINSLQSE |
| | | DVATYFCQQYNNYPPTFGGGTKLELKRADAAPTVSIFP |
| | | PSTEQLATGGASVVCLMNNFYPRDISVKWKIDGTERRD |
| | | GVLDSVTDQDSKDSTYSMSSTLSLTKADYESHNLYTCE |
| | | VVHKTSSSPVVKSFNRNEC |
| 105. | BAP0026 | DVVMTQTPLTWSITIGQPASISCKSSQSLLYSDGRTYLN |
| | light chain | WLLQRPGQSPKRLIYLVSKLDSGVPDRFSGSGSGTDFTL |
| | | KISRVEAEDLGFYYCWQGTHLPHTFGAGTKLELKGGG |
| | | GSGGGGSGGGGSGGGGSQAQLQQSGAELVRPGASVTL |
| | | SCKASGYTFTDYEIHWMKQTPVHGLEWIGAIDPETGGT |
| | | AYNQKFKGKAILTADRSSSTAYLELRSLTSEDSAVYYC |
| | | YSYDYVTYWGQGTLVTVSAGGGGSGGGGSGGGGSDIR |
| | | MTQSPASLSASLGETVNIECLASEDIYSDLAWYQQKPG |
| | | KSPQLLIYNANSLQNGVPSRFSGSGSGTQYSLKINSLQSE |
| | | DVATYFCQQYNNYPPTFGGGTKLELKRADAAPTVSIFP |
| | | PSTEQLATGGASVVCLMNNFYPRDISVKWKIDGTERRD |
| | | GVLDSVTDQDSKDSTYSMSSTLSLTKADYESHNLYTCE |
| | | VVHKTSSSPVVKSFNRNEC |
| 106. | BAP0027/ | QAQLQQSGAELVRPGASVTLSCKASGYTFTDYEIHWM |
| | BAP0028 heavy | KQTPVHGLEWIGAIDPETGGTAYNQKFKGKAILTADRS |
| | chain | SSTAYLELRSLTSEDSAVYYCYSYDYVTYWGQGTLVT |
| | | VSAAKTTPPSVYPLAPGSAAQTNSMVTLGCLVKGYFPE |
| | | PVTVTWNSGSLSSGVHTFPAVLQSDLYTLSSSVTVPSSP |
| | | RPSETVTCNVAHPASSTKVDKKIVPRDCGCKPCICTVPE |
| | | VSSVFIFPPKPKDVLTITLTPKVTCVVVDISKDDPEVQFS |
| | | WFVDDVEVHTAQTQPREEQFNSTFRSVSELPIMHQDWL |
| | | NGKEFKCRVNSAAFPAPIEKTISKTKGRPKAPQVYTIPPP |
| | | KEQMAKDKVSLTCMITDFFPEDITVEWQWNGQPAENY |
| | | KNTQPIMNTNGSYFVYSKLNVQKSNWEAGNTFTCSVL |
| | | HEGLHNHHTEKSLSHSPGK |
| 107. | BAP0027 | EVQLVESGGGLVQPANSLKLSCAASGFTFSDYAMAWV |
| | light chain | RQSPKKGLEWVATIIYDGSSTYYRDSVKGRFTISRDNAK |
| | | STLYLQMDSLRSEDTATYYCATGLGIATDYFDYWGQG |
| | | VLVTVSSGGGGSGGGGSGGGGSGGGGSDIRMTQSPASL |
| | | SASLGETVNIECLASEDIYSDLAWYQQKPGKSPQLLIYN |
| | | ANSLQNGVPSRFSGSGSGTQYSLKINSLQSEDVATYFCQ |
| | | QYNNYPPTFGGGTKLELKGGGGSGGGGSGGGGSDVV |
| | | MTQTPLTWSITIGQPASISCKSSQSLLYSDGRTYLNWLL |
| | | QRPGQSPKRLIYLVSKLDSGVPDRFSGSGSGTDFTLKISR |
| | | VEAEDLGFYYCWQGTHLPHTFGAGTKLELKRADAAPT |
| | | VSIFPPSSEQLTSGGASVVCFLNNFYPKDINVKWKIDGS |
| | | ERQNGVLNSWTDQDSKDSTYSMSSTLTLTKDEYERHN |
| | | SYTCEATHKTSTSPIVKSFNRNEC |
| 108. | BAP0028 | DIRMTQSPASLSASLGETVNIECLASEDIYSDLAWYQQK |
| | light chain | PGKSPQLLIYNANSLQNGVPSRFSGSGSGTQYSLKINSL |
| | | QSEDVATYFCQQYNNYPPTFGGGTKLELKGGGGSGGG |
| | | GSGGGGSGGGGSEVQLVESGGGLVQPANSLKLSCAAS |
| | | GFTFSDYAMAWVRQSPKKGLEWVATIIYDGSSTYYRDS |
| | | VKGRFTISRDNAKSTLYLQMDSLRSEDTATYYCATGLG |
| | | IATDYFDYWGQGVLVTVSSGGGGSGGGGSGGGGSDVV |
| | | MTQTPLTWSITIGQPASISCKSSQSLLYSDGRTYLNWLL |
| | | QRPGQSPKRLIYLVSKLDSGVPDRFSGSGSGTDFTLKISR |
| | | VEAEDLGFYYCWQGTHLPHTFGAGTKLELKRADAAPT |
| | | VSIFPPSSEQLTSGGASVVCFLNNFYPKDINVKWKIDGS |
| | | ERQNGVLNSWTDQDSKDSTYSMSSTLTLTKDEYERHN |
| | | SYTCEATHKTSTSPIVKSFNRNEC |
| 109. | BAP0029/ | EVQLVESGGGLVQPANSLKLSCAASGFTFSDYAMAWV |
| | BAP0030 heavy | RQSPKKGLEWVATIIYDGSSTYYRDSVKGRFTISRDNAK |
| | chain | STLYLQMDSLRSEDTATYYCATGLGIATDYFDYWGQG |
| | | VLVTVSSAETTAPSVYPLAPGTALKSNSMVTLGCLVKG |
| | | YFPEPVTVTWNSGALSSGVHTFPAVLQSGLYTLTSSVT |
| | | VPSSTWPSQTVTCNVAHPASSTKVDKKIVPRNCGGDCK |
| | | PCICTGSEVSSVFIFPPKPKDVLTITLTPKVTCVVVDISQD |
| | | DPEVHFSWFVDDVEVHTAQTRPPEEQFNSTFRSVSELPI |
| | | LHQDWLNGRTFRCKVTSAAFPSPIEKTISKPEGRTQVPH |
| | | VYTMSPTKEEMTQNEVSITCMVKGFYPPDIYVEWQMN |
| | | GQPQENYKNTPPTMDTDGSYFLYSKLNVKKEKWQQG |
| | | NTFTCSVLHEGLHNHHTEKSLSHSPGK |
| 110. | BAP0029 | DIRMTQSPASLSASLGETVNIECLASEDIYSDLAWYQQK |
| | light chain | PGKSPQLLIYNANSLQNGVPSRFSGSGSGTQYSLKINSL |
| | | QSEDVATYFCQQYNNYPPTFGGGTKLELKRADAAPTVS |
| | | IFPPSTEQLATGGASVVCLMNNFYPRDISVKWKIDGTER |
| | | RDGVLDSVTDQDSKDSTYSMSSTLSLTKADYESHNLYT |
| | | CEVVHKTSSSPVVKSFNRNECGGGGSGGGGSGGGGSQ |
| | | AQLQQSGAELVRPGASVTLSCKASGYTFTDYEIHWMK |
| | | QTPVHGLEWIGAIDPETGGTAYNQKFKGKAILTADRSSS |
| | | TAYLELRSLTSEDSAVYYCYSYDYVTYWGQGTLVTVS |
| | | AGGGGSGGGGSGGGGSGGGGSDVVMTQTPLTWSITIG |
| | | QPASISCKSSQSLLYSDGRTYLNWLLQRPGQSPKRLIYL |
| | | VSKLDSGVPDRFSGSGSGTDFTLKISRVEAEDLGFYYC |
| | | WQGTHLPHTFGAGTKLELK |
| 111. | BAP0030 | DIRMTQSPASLSASLGETVNIECLASEDIYSDLAWYQQK |
| | light chain | PGKSPQLLIYNANSLQNGVPSRFSGSGSGTQYSLKINSL |
| | | QSEDVATYFCQQYNNYPPTFGGGTKLELKRADAAPTVS |
| | | IFPPSTEQLATGGASVVCLMNNFYPRDISVKWKIDGTER |
| | | RDGVLDSVTDQDSKDSTYSMSSTLSLTKADYESHNLYT |
| | | CEVVHKTSSSPVVKSFNRNECGGGGSGGGGSGGGGSD |
| | | VVMTQTPLTWSITIGQPASISCKSSQSLLYSDGRTYLNW |
| | | LLQRPGQSPKRLIYLVSKLDSGVPDRFSGSGSGTDFTLKI |
| | | SRVEAEDLGFYYCWQGTHLPHTFGAGTKLELKGGGGS |
| | | GGGGSGGGGSGGGGSQAQLQQSGAELVRPGASVTLSC |
| | | KASGYTFTDYEIHWMKQTPVHGLEWIGAIDPETGGTAY |
| | | NQKFKGKAILTADRSSSTAYLELRSLTSEDSAVYYCYSY |
| | | DYVTYWGQGTLVTVSA |
| 112. | BAP0031/ | QAQLQQSGAELVRPGASVTLSCKASGYTFTDYEIHWM |
| | BAP0032 heavy | KQTPVHGLEWIGAIDPETGGTAYNQKFKGKAILTADRS |
| | chain | SSTAYLELRSLTSEDSAVYYCYSYDYVTYWGQGTLVT |
| | | VSAAKTTPPSVYPLAPGSAAQTNSMVTLGCLVKGYFPE |
| | | PVTVTWNSGSLSSGVHTFPAVLQSDLYTLSSSVTVPSSP |
| | | RPSETVTCNVAHPASSTKVDKKIVPRDCGCKPCICTVPE |
| | | VSSVFIFPPKPKDVLTITLTPKVTCVVVDISKDDPEVQFS |
| | | WFVDDVEVHTAQTQPREEQFNSTFRSVSELPIMHQDWL |
| | | NGKEFKCRVNSAAFPAPIEKTISKTKGRPKAPQVYTIPPP |
| | | KEQMAKDKVSLTCMITDFFPEDITVEWQWNGQPAENY |
| | | KNTQPIMNTNGSYFVYSKLNVQKSNWEAGNTFTCSVL |
| | | HEGLHNHHTEKSLSHSPGK |
| 113. | BAP0031 | DVVMTQTPLTWSITIGQPASISCKSSQSLLYSDGRTYLN |
| | light chain | WLLQRPGQSPKRLIYLVSKLDSGVPDRFSGSGSGTDFTL |
| | | KISRVEAEDLGFYYCWQGTHLPHTFGAGTKLELKRAD |
| | | AAPTVSIFPPSSEQLTSGGASVVCFLNNFYPKDINVKWK |
| | | IDGSERQNGVLNSWTDQDSKDSTYSMSSTLTLTKDEYE |
| | | RHNSYTCEATHKTSTSPIVKSFNRNECGGGGSGGGGSG |
| | | GGGSEVQLVESGGGLVQPANSLKLSCAASGFTFSDYAM |
| | | AWVRQSPKKGLEWVATIIYDGSSTYYRDSVKGRFTISR |
| | | DNAKSTLYLQMDSLRSEDTATYYCATGLGIATDYFDY |
| | | WGQGVLVTVSSGGGGSGGGGSGGGGSGGGGSDIRMT |
| | | QSPASLSASLGETVNIECLASEDIYSDLAWYQQKPGKSP |
| | | QLLIYNANSLQNGVPSRFSGSGSGTQYSLKINSLQSEDV |
| | | ATYFCQQYNNYPPTFGGGTKLELK |
| 114. | BAP0032 | DVVMTQTPLTWSITIGQPASISCKSSQSLLYSDGRTYLN |
| | light chain | WLLQRPGQSPKRLIYLVSKLDSGVPDRFSGSGSGTDFTL |
| | | KISRVEAEDLGFYYCWQGTHLPHTFGAGTKLELKRAD |
| | | AAPTVSIFPPSSEQLTSGGASVVCFLNNFYPKDINVKWK |
| | | IDGSERQNGVLNSWTDQDSKDSTYSMSSTLTLTKDEYE |
| | | RHNSYTCEATHKTSTSPIVKSFNRNECGGGGSGGGGSG |
| | | GGGSDIRMTQSPASLSASLGETVNIECLASEDIYSDLAW |
| | | YQQKPGKSPQLLIYNANSLQNGVPSRFSGSGSGTQYSL |
| | | KINSLQSEDVATYFCQQYNNYPPTFGGGTKLELKGGGG |
| | | SGGGGSGGGGSGGGGSEVQLVESGGGLVQPANSLKLS |
| | | CAASGFTFSDYAMAWVRQSPKKGLEWVATIIYDGSSTY |
| | | YRDSVKGRFTISRDNAKSTLYLQMDSLRSEDTATYYCA |
| | | TGLGIATDYFDYWGQGVLVTVSA |
| 115. | BAP0033 | EVQLVESGGGVVQPGRSLRLSCAASGFTFSDYAMAWV |
| | heavy chain | RQAPGKGLEWVATIIYDGSSTYYRDSVKGRFTISRDNSK |
| | | NTLYLQMNSLRAEDTAVYYCATGLGIATDYFDYWGQG |
| | | TLVTVSSASTKGPSVFPLAPCSRSTSESTAALGCLVKDY |
| | | FPEPVTVSWNSGALTSGVHTFPAVLQSSGLYSLSSVVTV |
| | | PSSSLGTKTYTCNVDHKPSNTKVDKRVESKYGPPCPPCP |
| | | APEFLGGPSVFLFPPKPKDTLMISRTPEVTCVVVDVSQE |
| | | DPEVQFNWYVDGVEVHNAKTKPREEQFNSTYRVVSVL |
| | | TVLHQDWLNGKEYKCKVSNKGLPSSIEKTISKAKGQPR |
| | | EPQVYTLPPSQEEMTKNQVSLTCLVKGFYPSDIAVEWE |
| | | SNGQPENNYKTTPPVLDSDGSFFLYSRLTVDKSRWQEG |
| | | NVFSCSVMHEALHNHYTQKSLSLSLGKGGGGSGGGGS |
| | | GGGGSDIQMTQSPSSLSASVGDRVTITCRSSQSLLYSDG |
| | | RTYLNWYQQKPGKSPKRLIYLVSKLDSGVPDRFSGSGS |
| | | GTDFTLTISSLQPEDFATYYCWQGTHLPHTFGCGTKVEI |
| | | KGGGGSGGGGSGGGGSGGGGSEVQLVQSGAEVKKPG |
| | | ASVKVSCKASGYTFTDYEIHWVRQAPGKCLEWMGAID |
| | | PETGGTAYNQKFKGRVTMTTDTSTSTAYMELRSLRSDD |
| | | TAVYYCYSYDYVTYWGQGTTVTVSA |
| 116. | BAP0034 | EVQLVESGGGVVQPGRSLRLSCAASGFTFSDYAMAWV |
| | heavy chain | RQAPGKGLEWVATIIYDGSSTYYRDSVKGRFTISRDNSK |
| | | NTLYLQMNSLRAEDTAVYYCATGLGIATDYFDYWGQG |
| | | TLVTVSSASTKGPSVFPLAPCSRSTSESTAALGCLVKDY |
| | | FPEPVTVSWNSGALTSGVHTFPAVLQSSGLYSLSSVVTV |
| | | PSSSLGTKTYTCNVDHKPSNTKVDKRVESKYGPPCPPCP |
| | | APEFLGGPSVFLFPPKPKDTLMISRTPEVTCVVVDVSQE |
| | | DPEVQFNWYVDGVEVHNAKTKPREEQFNSTYRVVSVL |
| | | TVLHQDWLNGKEYKCKVSNKGLPSSIEKTISKAKGQPR |
| | | EPQVYTLPPSQEEMTKNQVSLTCLVKGFYPSDIAVEWE |
| | | SNGQPENNYKTTPPVLDSDGSFFLYSRLTVDKSRWQEG |
| | | NVFSCSVMHEALHNHYTQKSLSLSLGKGGGGSGGGGS |
| | | GGGGSEVQLVQSGAEVKKPGASVKVSCKASGYTFTDY |
| | | EIHWVRQAPGKCLEWMGAIDPETGGTAYNQKFKGRVT |
| | | MTTDTSTSTAYMELRSLRSDDTAVYYCYSYDYVTYWG |
| | | QGTTVTVSSGGGGSGGGGSGGGGSGGGGSDIQMTQSPS |
| | | SLSASVGDRVTITCRSSQSLLYSDGRTYLNWYQQKPGK |
| | | SPKRLIYLVSKLDSGVPDRFSGSGSGTDFTLTISSLQPED |
| | | FATYYCWQGTHLPHTFGCGTKVEIK |
| 117. | BAP0035 | EVQLVESGGGVVQPGRSLRLSCAASGFTFSDYAMAWV |
| | heavy chain | RQAPGKGLEWVATIIYDGSSTYYRDSVKGRFTISRDNSK |
| | | NTLYLQMNSLRAEDTAVYYCATGLGIATDYFDYWGQG |
| | | TLVTVSSASTKGPSVFPLAPCSRSTSESTAALGCLVKDY |
| | | FPEPVTVSWNSGALTSGVHTFPAVLQSSGLYSLSSVVTV |
| | | PSSSLGTKTYTCNVDHKPSNTKVDKRVESKYGPPCPPCP |
| | | APEFLGGPSVFLFPPKPKDTLMISRTPEVTCVVVDVSQE |
| | | DPEVQFNWYVDGVEVHNAKTKPREEQFNSTYRVVSVL |
| | | TVLHQDWLNGKEYKCKVSNKGLPSSIEKTISKAKGQPR |
| | | EPQVYTLPPSQEEMTKNQVSLTCLVKGFYPSDIAVEWE |
| | | SNGQPENNYKTTPPVLDSDGSFFLYSRLTVDKSRWQEG |
| | | NVFSCSVMHEALHNHYTQKSLSLSLGKGGGGSGGGGS |
| | | GGGGSDVVMTQSPLSLSVSPGERASLSCKSSQSLLYSDG |
| | | RTYLNWYLQKPGQSPQRLIYLVSKLDSGVPDRFSGSGS |
| | | GTDFTLKISRVQSEDVGVYYCWQGTHLPHTFGCGTKVE |
| | | IKGGGGSGGGGSGGGGSGGGGSEVQLVQSGAEVKKPG |
| | | ASVKVSCKASGYTFTDYEIHWVRQAPGKCLEWMGAID |
| | | PETGGTAYNQKFKGRVTMTTDTSTSTAYMELRSLRSDD |
| | | TAVYYCYSYDYVTYWGQGTTVTVSA |
| 118. | BAP0036 | EVQLVESGGGVVQPGRSLRLSCAASGFTFSDYAMAWV |
| | heavy chain | RQAPGKGLEWVATIIYDGSSTYYRDSVKGRFTISRDNSK |
| | | NTLYLQMNSLRAEDTAVYYCATGLGIATDYFDYWGQG |
| | | TLVTVSSASTKGPSVFPLAPCSRSTSESTAALGCLVKDY |
| | | FPEPVTVSWNSGALTSGVHTFPAVLQSSGLYSLSSVVTV |
| | | PSSSLGTKTYTCNVDHKPSNTKVDKRVESKYGPPCPPCP |
| | | APEFLGGPSVFLFPPKPKDTLMISRTPEVTCVVVDVSQE |
| | | DPEVQFNWYVDGVEVHNAKTKPREEQFNSTYRVVSVL |
| | | TVLHQDWLNGKEYKCKVSNKGLPSSIEKTISKAKGQPR |
| | | EPQVYTLPPSQEEMTKNQVSLTCLVKGFYPSDIAVEWE |
| | | SNGQPENNYKTTPPVLDSDGSFFLYSRLTVDKSRWQEG |
| | | NVFSCSVMHEALHNHYTQKSLSLSLGKGGGGSGGGGS |
| | | GGGGSEVQLVQSGAEVKKPGASVKVSCKASGYTFTDY |
| | | EIHWVRQAPGKCLEWMGAIDPETGGTAYNQKFKGRVT |
| | | MTTDTSTSTAYMELRSLRSDDTAVYYCYSYDYVTYWG |
| | | QGTTVTVSSGGGGSGGGGSGGGGSGGGGSDVVMTQSP |
| | | LSLSVSPGERASLSCKSSQSLLYSDGRTYLNWYLQKPG |
| | | QSPQRLIYLVSKLDSGVPDRFSGSGSGTDFTLKISRVQSE |
| | | DVGVYYCWQGTHLPHTFGCGTKVEIK |
| 119. | BAP0033/ | DIRMTQSPFSLSASVGDRVTITCLASEDIYSDLAWYQQK |
| | BAP0034/ | PAKAPKLFIYNANSLQNGVPSRFSGSGSGTDYTLTISSLQ |
| | BAP0035/BAP0036 | PEDFATYYCQQYNNYPPTFGGGTKVEIKRTVAAPSVFIF |
| | light chain | PPSDEQLKSGTASVVCLLNNFYPREAKVQWKVDNALQ |
| | | SGNSQESVTEQDSKDSTYSLSSTLTLSKADYEKHKVYA |
| | | CEVTHQGLSSPVTKSFNRGEC |
| 120. | BAP0037 | EVQLVQSGAEVKKPGASVKVSCKASGYTFTDYEIHWV |
| | heavy chain | RQAPGKGLEWMGAIDPETGGTAYNQKFKGRVTMTTDT |
| | | STSTAYMELRSLRSDDTAVYYCYSYDYVTYWGQGTTV |
| | | TVSSASTKGPSVFPLAPCSRSTSESTAALGCLVKDYFPEP |
| | | VTVSWNSGALTSGVHTFPAVLQSSGLYSLSSVVTVPSSS |
| | | LGTKTYTCNVDHKPSNTKVDKRVESKYGPPCPPCPAPE |
| | | FLGGPSVFLFPPKPKDTLMISRTPEVTCVVVDVSQEDPE |
| | | VQFNWYVDGVEVHNAKTKPREEQFNSTYRVVSVLTVL |
| | | HQDWLNGKEYKCKVSNKGLPSSIEKTISKAKGQPREPQ |
| | | VYTLPPSQEEMTKNQVSLTCLVKGFYPSDIAVEWESNG |
| | | QPENNYKTTPPVLDSDGSFFLYSRLTVDKSRWQEGNVF |
| | | SCSVMHEALHNHYTQKSLSLSLGKGGGGSGGGGSGGG |
| | | GSEVQLVESGGGVVQPGRSLRLSCAASGFTFSDYAMA |
| | | WVRQAPGKCLEWVATIIYDGSSTYYRDSVKGRFTISRD |
| | | NSKNTLYLQMNSLRAEDTAVYYCATGLGIATDYFDYW |
| | | GQGTLVTVSSGGGGSGGGGSGGGGSGGGGSDIRMTQS |
| | | PFSLSASVGDRVTITCLASEDIYSDLAWYQQKPAKAPKL |
| | | FIYNANSLQNGVPSRFSGSGSGTDYTLTISSLQPEDFATY |
| | | YCQQYNNYPPTFGCGTKVEIK |
| 121. | BAP0038 | EVQLVQSGAEVKKPGASVKVSCKASGYTFTDYEIHWV |
| | heavy chain | RQAPGKGLEWMGAIDPETGGTAYNQKFKGRVTMTTDT |
| | | STSTAYMELRSLRSDDTAVYYCYSYDYVTYWGQGTTV |
| | | TVSSASTKGPSVFPLAPCSRSTSESTAALGCLVKDYFPEP |
| | | VTVSWNSGALTSGVHTFPAVLQSSGLYSLSSVVTVPSSS |
| | | LGTKTYTCNVDHKPSNTKVDKRVESKYGPPCPPCPAPE |
| | | FLGGPSVFLFPPKPKDTLMISRTPEVTCVVVDVSQEDPE |
| | | VQFNWYVDGVEVHNAKTKPREEQFNSTYRVVSVLTVL |
| | | HQDWLNGKEYKCKVSNKGLPSSIEKTISKAKGQPREPQ |
| | | VYTLPPSQEEMTKNQVSLTCLVKGFYPSDIAVEWESNG |
| | | QPENNYKTTPPVLDSDGSFFLYSRLTVDKSRWQEGNVF |
| | | SCSVMHEALHNHYTQKSLSLSLGKGGGGSGGGGSGGG |
| | | GSDIRMTQSPFSLSASVGDRVTITCLASEDIYSDLAWYQ |
| | | QKPAKAPKLFIYNANSLQNGVPSRFSGSGSGTDYTLTIS |
| | | SLQPEDFATYYCQQYNNYPPTFGCGTKVEIKGGGGSGG |
| | | GGSGGGGSGGGGSEVQLVESGGGVVQPGRSLRLSCAA |
| | | SGFTFSDYAMAWVRQAPGKCLEWVATIIYDGSSTYYR |
| | | DSVKGRFTISRDNSKNTLYLQMNSLRAEDTAVYYCATG |
| | | LGIATDYFDYWGQGTLVTVSA |
| 122. | BAP0037/ | DIQMTQSPSSLSASVGDRVTITCRSSQSLLYSDGRTYLN |
| | BAP0038/ | WYQQKPGKSPKRLIYLVSKLDSGVPDRFSGSGSGTDFT |
| | BAP0071/ | LTISSLQPEDFATYYCWQGTHLPHTFGGGTKVEIKRTVA |
| | BAP0072/BAP0073 | APSVFIFPPSDEQLKSGTASVVCLLNNFYPREAKVQWK |
| | light chain | VDNALQSGNSQESVTEQDSKDSTYSLSSTLTLSKADYE |
| | | KHKVYACEVTHQGLSSPVTKSFNRGEC |
| 123. | BAP0039 | EVQLVQSGAEVKKPGASVKVSCKASGYTFTDYEIHWV |
| | heavy chain | RQAPGKGLEWMGAIDPETGGTAYNQKFKGRVTMTTDT |
| | | STSTAYMELRSLRSDDTAVYYCYSYDYVTYWGQGTTV |
| | | TVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPE |
| | | FLGGPSVFLFPPKPKDTLMISRTPEVTCVVVDVSQEDPE |
| | | VQFNWYVDGVEVHNAKTKPREEQFNSTYRVVSVLTVL |
| | | HQDWLNGKEYKCKVSNKGLPSSIEKTISKAKGQPREPQ |
| | | VYTLPPSQEEMTKNQVSLTCLVKGFYPSDIAVEWESNG |
| | | QPENNYKTTPPVLDSDGSFFLYSRLTVDKSRWQEGNVF |
| | | SCSVMHEALHNHYTQKSLSLSLGKGGGGSGGGGSGGG |
| | | GSEVQLVESGGGVVQPGRSLRLSCAASGFTFSDYAMA |
| | | WVRQAPGKCLEWVATIIYDGSSTYYRDSVKGRFTISRD |
| | | NSKNTLYLQMNSLRAEDTAVYYCATGLGIATDYFDYW |
| | | GQGTLVTVSSGGGGSGGGGSGGGGSGGGGSDIRMTQS |
| | | PFSLSASVGDRVTITCLASEDIYSDLAWYQQKPAKAPKL |
| | | FIYNANSLQNGVPSRFSGSGSGTDYTLTISSLQPEDFATY |
| | | YCQQYNNYPPTFGCGTKVEIK |
| 124. | BAP0040 | EVQLVQSGAEVKKPGASVKVSCKASGYTFTDYEIHWV |
| | heavy chain | RQAPGKGLEWMGAIDPETGGTAYNQKFKGRVTMTTDT |
| | | STSTAYMELRSLRSDDTAVYYCYSYDYVTYWGQGTTV |
| | | TVSSASTKGPSVFPLAPCSRSTSESTAALGCLVKDYFPEP |
| | | VTVSWNSGALTSGVHTFPAVLQSSGLYSLSSVVTVPSSS |
| | | LGTKTYTCNVDHKPSNTKVDKRVESKYGPPCPPCPAPE |
| | | FLGGPSVFLFPPKPKDTLMISRTPEVTCVVVDVSQEDPE |
| | | VQFNWYVDGVEVHNAKTKPREEQFNSTYRVVSVLTVL |
| | | HQDWLNGKEYKCKVSNKGLPSSIEKTISKAKGQPREPQ |
| | | VYTLPPSQEEMTKNQVSLTCLVKGFYPSDIAVEWESNG |
| | | QPENNYKTTPPVLDSDGSFFLYSRLTVDKSRWQEGNVF |
| | | SCSVMHEALHNHYTQKSLSLSLGKGGGGSGGGGSGGG |
| | | GSDIRMTQSPFSLSASVGDRVTITCLASEDIYSDLAWYQ |
| | | QKPAKAPKLFIYNANSLQNGVPSRFSGSGSGTDYTLTIS |
| | | SLQPEDFATYYCQQYNNYPPTFGCGTKVEIKGGGGSGG |
| | | GGSGGGGSGGGGSEVQLVESGGGVVQPGRSLRLSCAA |
| | | SGFTFSDYAMAWVRQAPGKCLEWVATIIYDGSSTYYR |
| | | DSVKGRFTISRDNSKNTLYLQMNSLRAEDTAVYYCATG |
| | | LGIATDYFDYWGQGTLVTVSA |
| 125. | BAP0039/ | DVVMTQSPLSLSVSPGERASLSCKSSQSLLYSDGRTYLN |
| | BAP0040/ | WYLQKPGQSPQRLIYLVSKLDSGVPDRFSGSGSGTDFTL |
| | BAP0069/BAP0070 | KISRVQSEDVGVYYCWQGTHLPHTFGGGTKVEIKRTVA |
| | light chain | APSVFIFPPSDEQLKSGTASVVCLLNNFYPREAKVQWK |
| | | VDNALQSGNSQESVTEQDSKDSTYSLSSTLTLSKADYE |
| | | KHKVYACEVTHQGLSSPVTKSFNRGEC |
| 126. | BAP0050 | EVQLLESGGGLVQPGGSLRLSCAASGFTFSSYAMSWVR |
| | heavy chain | QAPGKGLEWVSGITGSGGSTYYADSVKGRFTISRDNSK |
| | | NTLYLQMNSLRAEDTAVYYCAKDPGTTVIMSWFDPWG |
| | | QGTLVTVSSASTKGPSVFPLAPCSRSTSESTAALGCLVK |
| | | DYFPEPVTVSWNSGALTSGVHTFPAVLQSSGLYSLSSV |
| | | VTVPSSNFGTQTYTCNVDHKPSNTKVDKTVERKCCVEC |
| | | PPCPAPPVAGPSVFLFPPKPKDTLMISRTPEVTCVVVDV |
| | | SHEDPEVQFNWYVDGVEVHNAKTKPREEQFNSTFRVV |
| | | SVLTVVHQDWLNGKEYKCKVSNKGLPAPIEKTISKTKG |
| | | QPREPQVYTLPPSREEMTKNQVSLTCLVKGFYPSDIAVE |
| | | WESNGQPENNYKTTPPMLDSDGSFFLYSKLTVDKSRW |
| | | QQGNVFSCSVMHEALHNHYTQKSLSLSPGKGGGGSGG |
| | | GGSGGGGSDIQMTQSPSSLSASVGDRVTITCRSSQSLLY |
| | | SDGRTYLNWYQQKPGKSPKRLIYLVSKLDSGVPDRFSG |
| | | SGSGTDFTLTISSLQPEDFATYYCWQGTHLPHTFGCGTK |
| | | VEIKGGGGSGGGGSGGGGSGGGGSEVQLVQSGAEVKK |
| | | PGASVKVSCKASGYTFTDYEIHWVRQAPGKCLEWMGA |
| | | IDPETGGTAYNQKFKGRVTMTTDTSTSTAYMELRSLRS |
| | | DDTAVYYCYSYDYVTYWGQGTTVTVSA |
| 127. | BAP0051 | DIQMTQSPSSLSASVGDRVTITCRSSQSLLYSDGRTYLN |
| | heavy chain | WYQQKPGKSPKRLIYLVSKLDSGVPDRFSGSGSGTDFT |
| | | LTISSLQPEDFATYYCWQGTHLPHTFGCGTKVEIKGGG |
| | | GSGGGGSGGGGSGGGGSEVQLVQSGAEVKKPGASVKV |
| | | SCKASGYTFTDYEIHWVRQAPGKCLEWMGAIDPETGG |
| | | TAYNQKFKGRVTMTTDTSTSTAYMELRSLRSDDTAVY |
| | | YCYSYDYVTYWGQGTTVTVSSGGGGSGGGGSGGGGS |
| | | EVQLLESGGGLVQPGGSLRLSCAASGFTFSSYAMSWVR |
| | | QAPGKGLEWVSGITGSGGSTYYADSVKGRFTISRDNSK |
| | | NTLYLQMNSLRAEDTAVYYCAKDPGTTVIMSWFDPWG |
| | | QGTLVTVSSASTKGPSVFPLAPCSRSTSESTAALGCLVK |
| | | DYFPEPVTVSWNSGALTSGVHTFPAVLQSSGLYSLSSV |
| | | VTVPSSNFGTQTYTCNVDHKPSNTKVDKTVERKCCVEC |
| | | PPCPAPPVAGPSVFLFPPKPKDTLMISRTPEVTCVVVDV |
| | | SHEDPEVQFNWYVDGVEVHNAKTKPREEQFNSTFRVV |
| | | SVLTVVHQDWLNGKEYKCKVSNKGLPAPIEKTISKTKG |
| | | QPREPQVYTLPPSREEMTKNQVSLTCLVKGFYPSDIAVE |
| | | WESNGQPENNYKTTPPMLDSDGSFFLYSKLTVDKSRW |
| | | QQGNVFSCSVMHEALHNHYTQKSLSLSPGK |
| 128. | BAP0052 | EVQLLESGGGLVQPGGSLRLSCAASGFTFSSYAMSWVR |
| | heavy chain | QAPGKGLEWVSGITGSGGSTYYADSVKGRFTISRDNSK |
| | | NTLYLQMNSLRAEDTAVYYCAKDPGTTVIMSWFDPWG |
| | | QGTLVTVSSASTKGPSVFPLAPCSRSTSESTAALGCLVK |
| | | DYFPEPVTVSWNSGALTSGVHTFPAVLQSSGLYSLSSV |
| | | VTVPSSNFGTQTYTCNVDHKPSNTKVDKTVERKCCVEC |
| | | PPCPAPPVAGPSVFLFPPKPKDTLMISRTPEVTCVVVDV |
| | | SHEDPEVQFNWYVDGVEVHNAKTKPREEQFNSTFRVV |
| | | SVLTVVHQDWLNGKEYKCKVSNKGLPAPIEKTISKTKG |
| | | QPREPQVYTLPPSREEMTKNQVSLTCLVKGFYPSDIAVE |
| | | WESNGQPENNYKTTPPMLDSDGSFFLYSKLTVDKSRW |
| | | QQGNVFSCSVMHEALHNHYTQKSLSLSPGKGGGGSGG |
| | | GGSGGGGSDVVMTQSPLSLSVSPGERASLSCKSSQSLLY |
| | | SDGRTYLNWYLQKPGQSPQRLIYLVSKLDSGVPDRFSG |
| | | SGSGTDFTLKISRVQSEDVGVYYCWQGTHLPHTFGCGT |
| | | KVEIKGGGGSGGGGSGGGGSGGGGSEVQLVQSGAEVK |
| | | KPGASVKVSCKASGYTFTDYEIHWVRQAPGKCLEWMG |
| | | AIDPETGGTAYNQKFKGRVTMTTDTSTSTAYMELRSLR |
| | | SDDTAVYYCYSYDYVTYWGQGTTVTVSA |
| 129. | BAP0050/ | EIVLTQSPGTLSLSPGERATLSCRASQSVRGRYLAWYQQ |
| | BAP0051/BAP0052 | KPGQAPRLLIYGASSRATGIPDRFSGSGSGTDFTLTISRL |
| | light chain | EPEDFAVFYCQQYGSSPRTFGQGTKVEIKRTVAAPSVFI |
| | | FPPSDEQLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGEC |
| 130. | BAP0053 | EIVLTQSPGTLSLSPGERATLSCRASQSVRGRYLAWYQQ |
| | light chain | KPGQAPRLLIYGASSRATGIPDRFSGSGSGTDFTLTISRL |
| | | EPEDFAVFYCQQYGSSPRTFGQGTKVEIKRTVAAPSVFI |
| | | FPPSDEQLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGECGGGGSGGGGSGGGGS |
| | | DIQMTQSPSSLSASVGDRVTITCRSSQSLLYSDGRTYLN |
| | | WYQQKPGKSPKRLIYLVSKLDSGVPDRFSGSGSGTDFT |
| | | LTISSLQPEDFATYYCWQGTHLPHTFGCGTKVEIKGGG |
| | | GSGGGGSGGGGSGGGGSEVQLVQSGAEVKKPGASVKV |
| | | SCKASGYTFTDYEIHWVRQAPGKCLEWMGAIDPETGG |
| | | TAYNQKFKGRVTMTTDTSTSTAYMELRSLRSDDTAVY |
| | | YCYSYDYVTYWGQGTTVTVSA |
| 131. | BAP0054 | DIQMTQSPSSLSASVGDRVTITCRSSQSLLYSDGRTYLN |
| | light chain | WYQQKPGKSPKRLIYLVSKLDSGVPDRFSGSGSGTDFT |
| | | LTISSLQPEDFATYYCWQGTHLPHTFGCGTKVEIKGGG |
| | | GSGGGGSGGGGSGGGGSEVQLVQSGAEVKKPGASVKV |
| | | SCKASGYTFTDYEIHWVRQAPGKCLEWMGAIDPETGG |
| | | TAYNQKFKGRVTMTTDTSTSTAYMELRSLRSDDTAVY |
| | | YCYSYDYVTYWGQGTTVTVSSGGGGSGGGGSGGGGS |
| | | EIVLTQSPGTLSLSPGERATLSCRASQSVRGRYLAWYQQ |
| | | KPGQAPRLLIYGASSRATGIPDRFSGSGSGTDFTLTISRL |
| | | EPEDFAVFYCQQYGSSPRTFGQGTKVEIKRTVAAPSVFI |
| | | FPPSDEQLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGEC |
| 132. | BAP0055 | EIVLTQSPGTLSLSPGERATLSCRASQSVRGRYLAWYQQ |
| | light chain | KPGQAPRLLIYGASSRATGIPDRFSGSGSGTDFTLTISRL |
| | | EPEDFAVFYCQQYGSSPRTFGQGTKVEIKRTVAAPSVFI |
| | | FPPSDEQLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGECGGGGSGGGGSGGGGS |
| | | DVVMTQSPLSLSVSPGERASLSCKSSQSLLYSDGRTYLN |
| | | WYLQKPGQSPQRLIYLVSKLDSGVPDRFSGSGSGTDFTL |
| | | KISRVQSEDVGVYYCWQGTHLPHTFGCGTKVEIKGGG |
| | | GSGGGGSGGGGSGGGGSEVQLVQSGAEVKKPGASVKV |
| | | SCKASGYTFTDYEIHWVRQAPGKCLEWMGAIDPETGG |
| | | TAYNQKFKGRVTMTTDTSTSTAYMELRSLRSDDTAVY |
| | | YCYSYDYVTYWGQGTTVTVSA |
| 133. | BAP0053/ | EVQLLESGGGLVQPGGSLRLSCAASGFTFSSYAMSWVR |
| | PBA0054/ | QAPGKGLEWVSGITGSGGSTYYADSVKGRFTISRDNSK |
| | BAP0055 heavy | NTLYLQMNSLRAEDTAVYYCAKDPGTTVIMSWFDPWG |
| | chain | QGTLVTVSSASTKGPSVFPLAPCSRSTSESTAALGCLVK |
| | | DYFPEPVTVSWNSGALTSGVHTFPAVLQSSGLYSLSSV |
| | | VTVPSSNFGTQTYTCNVDHKPSNTKVDKTVERKCCVEC |
| | | PPCPAPPVAGPSVFLFPPKPKDTLMISRTPEVTCVVVDV |
| | | SHEDPEVQFNWYVDGVEVHNAKTKPREEQFNSTFRVV |
| | | SVLTVVHQDWLNGKEYKCKVSNKGLPAPIEKTISKTKG |
| | | QPREPQVYTLPPSREEMTKNQVSLTCLVKGFYPSDIAVE |
| | | WESNGQPENNYKTTPPMLDSDGSFFLYSKLTVDKSRW |
| | | QQGNVFSCSVMHEALHNHYTQKSLSLSPGK |
| 134. | BAP0041/ | QAQLQQSGAELVRPGASVTLSCKASGYTFTDYEIHWM |
| | BAP0042/ | KQTPVHGLEWIGAIDPETGGTAYNQKFKGKAILTADRS |
| | BAP0043/ | SSTAYLELRSLTSEDSAVYYCYSYDYVTYWGQGTLVT |
| | BAP0044/BAP0045 | VSSASTKGPSVFPLAPCSRSTSESTAALGCLVKDYFPEP |
| | heavy chain | VTVSWNSGALTSGVHTFPAVLQSSGLYSLSSVVTVPSSS |
| | #1 | LGTKTYTCNVDHKPSNTKVDKRVESKYGPPCPPCPAPE |
| | | FLGGPSVFLFPPKPKDTLMISRTPEVTCVVVDVSQEDPE |
| | | VQFNWYVDGVEVHNAKTKPREEQFNSTYRVVSVLTVL |
| | | HQDWLNGKEYKCKVSNKGLPSSIEKTISKAKGQPREPQ |
| | | VYTLPPSQEEMTKNQVSLSCAVKGFYPSDIAVEWESNG |
| | | QPENNYKTTPPVLDSDGSFFLVSRLTVDKSRWQEGNVF |
| | | SCSVMHEALHNRFTQKSLSLSLGK |
| 135. | BAP0041/ | DVVMTQTPLTWSITIGQPASISCKSSQSLLYSDGRTYLN |
| | BAP0042/ | WLLQRPGQSPKRLIYLVSKLDSGVPDRFSGSGSGTDFTL |
| | BAP0043/ | KISRVEAEDLGFYYCWQGTHLPHTFGAGTKLELKRTVA |
| | BAP0044/BAP0045 | APSVFIFPPSDEQLKSGTASVVCLLNNFYPREAKVQWK |
| | light chain #1 | VDNALQSGNSQESVTEQDSKDSTYSLSSTLTLSKADYE |
| | | KHKVYACEVTHQGLSSPVTKSFNRGEC |
| 136. | BAP0041 | EVQLVESGGGLVQPANSLKLSCAASGFTFSDYAMAWV |
| | heavy chain | RQSPKKGLEWVATIIYDGSSTYYRDSVKGRFTISRDNAK |
| | #2 | STLYLQMDSLRSEDTATYYCATGLGIATDYFDYWGQG |
| | | VLVTVSSASTKGPSVCPLAPSSRSTSESTAALGCLVKDY |
| | | FPEPVTVSWNSGALTSGVHTFPAVLQSSGLYSLSSVVTV |
| | | PSSSLGTKTYTCNVDHKPSNTKVDKRVESKYGPPCPPCP |
| | | APEFLGGPSVFLFPPKPKDTLMISRTPEVTCVVVDVSQE |
| | | DPEVQFNWYVDGVEVHNAKTKPREEQFNSTYRVVSVL |
| | | TVLHQDWLNGKEYKCKVSNKGLPSSIEKTISKAKGQPR |
| | | EPQVYTLPPSQEEMTKNQVSLWCLVKGFYPSDIAVEWE |
| | | SNGQPENNYKTTPPVLDSDGSFFLYSRLTVDKSRWQEG |
| | | NVFSCSVMHEALHNHYTQKSLSLSLGK |
| 137. | BAP0041 | DIRMTQSPASLSASLGETVNIECLASEDIYSDLAWYQQK |
| | light chain #2 | PGKSPQLLIYNANSLQNGVPSRFSGSGSGTQYSLKINSL |
| | | QSEDVATYFCQQYNNYPPTFGGGTKLELKRTVAAPSVF |
| | | IFPPCDEQLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGES |
| 138. | BAP0042 | EVQLVESGGGLVQPANSLKLSCAASGFTFSDYAMAWV |
| | heavy chain | RQSPKKGLEWVATIIYDGSSTYYRDSVKGRFTISRDNAK |
| | #2 | STLYLQMDSLRSEDTATYYCATGLGIATDYFDYWGQG |
| | | VLVTVSSASTKGPSVFPCAPSSRSTSESTAALGCLVKDY |
| | | FPEPVTVSWNSGALTSGVHTFPAVLQSSGLYSLSSVVTV |
| | | PSSSLGTKTYTCNVDHKPSNTKVDKRVESKYGPPCPPCP |
| | | APEFLGGPSVFLFPPKPKDTLMISRTPEVTCVVVDVSQE |
| | | DPEVQFNWYVDGVEVHNAKTKPREEQFNSTYRVVSVL |
| | | TVLHQDWLNGKEYKCKVSNKGLPSSIEKTISKAKGQPR |
| | | EPQVYTLPPSQEEMTKNQVSLWCLVKGFYPSDIAVEWE |
| | | SNGQPENNYKTTPPVLDSDGSFFLYSRLTVDKSRWQEG |
| | | NVFSCSVMHEALHNHYTQKSLSLSLGK |
| 139. | BAP0042 | DIRMTQSPASLSASLGETVNIECLASEDIYSDLAWYQQK |
| | light chain #2 | PGKSPQLLIYNANSLQNGVPSRFSGSGSGTQYSLKINSL |
| | | QSEDVATYFCQQYNNYPPTFGGGTKLELKRTVAAPSVF |
| | | ICPPSDEQLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGES |
| 140. | BAP0043 | EVQLVESGGGLVQPANSLKLSCAASGFTFSDYAMAWV |
| | heavy chain | RQSPKKGLEWVATIIYDGSSTYYRDSVKGRFTISRDNAK |
| | #2 | STLYLQMDSLRSEDTATYYCATGLGIATDYFDYWGQG |
| | | VLVTVSSASTKGPSVFPLAPSSRSTSESTAALGCLVKDY |
| | | FPEPVTVSWNSGALTSGVHTCPAVLQSSGLYSLSSVVT |
| | | VPSSSLGTKTYTCNVDHKPSNTKVDKRVESKYGPPCPP |
| | | CPAPEFLGGPSVFLFPPKPKDTLMISRTPEVTCVVVDVS |
| | | QEDPEVQFNWYVDGVEVHNAKTKPREEQFNSTYRVVS |
| | | VLTVLHQDWLNGKEYKCKVSNKGLPSSIEKTISKAKGQ |
| | | PREPQVYTLPPSQEEMTKNQVSLWCLVKGFYPSDIAVE |
| | | WESNGQPENNYKTTPPVLDSDGSFFLYSRLTVDKSRWQ |
| | | EGNVFSCSVMHEALHNHYTQKSLSLSLGK |
| 141. | BAP0043 | DIRMTQSPASLSASLGETVNIECLASEDIYSDLAWYQQK |
| | light chain #2 | PGKSPQLLIYNANSLQNGVPSRFSGSGSGTQYSLKINSL |
| | | QSEDVATYFCQQYNNYPPTFGGGTKLELKRTVAAPSVF |
| | | IFPPSDEQLKSGTASVVCLLNNFYPREAKVQWKVDNAL |
| | | QSGNSQESVTEQDSKDSTYSLCSTLTLSKADYEKHKVY |
| | | ACEVTHQGLSSPVTKSFNRGES |
| 142. | BAP0044 | EVQLVESGGGLVQPANSLKLSCAASGFTFSDYAMAWV |
| | heavy chain | RQSPKKGLEWVATIIYDGSSTYYRDSVKGRFTISRDNAK |
| | #2 | STLYLQMDSLRSEDTATYYCATGLGIATDYFDYWGQG |
| | | VLVTVSSASTKGPSVFPLAPSSRSTSESTAALGCLVKDY |
| | | FPEPVTVSWNSGALTSGVHTFPACLQSSGLYSLSSVVTV |
| | | PSSSLGTKTYTCNVDHKPSNTKVDKRVESKYGPPCPPCP |
| | | APEFLGGPSVFLFPPKPKDTLMISRTPEVTCVVVDVSQE |
| | | DPEVQFNWYVDGVEVHNAKTKPREEQFNSTYRVVSVL |
| | | TVLHQDWLNGKEYKCKVSNKGLPSSIEKTISKAKGQPR |
| | | EPQVYTLPPSQEEMTKNQVSLWCLVKGFYPSDIAVEWE |
| | | SNGQPENNYKTTPPVLDSDGSFFLYSRLTVDKSRWQEG |
| | | NVFSCSVMHEALHNHYTQKSLSLSLGK |
| 143. | BAP0044 | DIRMTQSPASLSASLGETVNIECLASEDIYSDLAWYQQK |
| | light chain #2 | PGKSPQLLIYNANSLQNGVPSRFSGSGSGTQYSLKINSL |
| | | QSEDVATYFCQQYNNYPPTFGGGTKLELKRTVAAPSVF |
| | | IFPPSDEQLKSGTASVVCLLNNFYPREAKVQWKVDNAL |
| | | QSGNSCESVTEQDSKDSTYSLSSTLTLSKADYEKHKVY |
| | | ACEVTHQGLSSPVTKSFNRGES |
| 144. | BAP0045 | EVQLVESGGGLVQPANSLKLSCAASGFTFSDYAMAWV |
| | heavy chain | RQSPKKGLEWVATIIYDGSSTYYRDSVKGRFTISRDNAK |
| | #2 | STLYLQMDSLRSEDTATYYCATGLGIATDYFDYWGQG |
| | | VLVTVSSASTKGPSVFPLAPSSRSTSESTAALGCLVKDY |
| | | FPEPVTVSWNSGALTSGVHTFPACLQSSGLYSLSSVVTV |
| | | PSSSLGTKTYTCNVDHKPSNTKVDKRVESKYGPPCPPCP |
| | | APEFLGGPSVFLFPPKPKDTLMISRTPEVTCVVVDVSQE |
| | | DPEVQFNWYVDGVEVHNAKTKPREEQFNSTYRVVSVL |
| | | TVLHQDWLNGKEYKCKVSNKGLPSSIEKTISKAKGQPR |
| | | EPQVYTLPPSQEEMTKNQVSLWCLVKGFYPSDIAVEWE |
| | | SNGQPENNYKTTPPVLDSDGSFFLYSRLTVDKSRWQEG |
| | | NVFSCSVMHEALHNHYTQKSLSLSLGK |
| 145. | BAP0045 | DIRMTQSPASLSASLGETVNIECLASEDIYSDLAWYQQK |
| | light chain #2 | PGKSPQLLIYNANSLQNGVPSRFSGSGSGTQYSLKINSL |
| | | QSEDVATYFCQQYNNYPPTFGGGTKLELKRTVAAPSVF |
| | | IFPPSDEQLKSGTASVVCLLNNFYPREAKVQWKVDNAL |
| | | QSGNSCESVTEQDSKDSTYSLSSTLTLSKADYEKHKVY |
| | | ACEVTHQGLSSPVTKSFNRGES |
| 146. | BAP0061/ | EVQLVQSGAEVKKPGASVKVSCKASGYTFTDYEIHWV |
| | BAP0062/ | RQAPGKGLEWMGAIDPETGGTAYNQKFKGRVTMTTDT |
| | BAP0063/ | STSTAYMELRSLRSDDTAVYYCYSYDYVTYWGQGTTV |
| | BAP0064/ | TVSSASTKGPSVFPLAPCSRSTSESTAALGCLVKDYFPEP |
| | BAP0065/ | VTVSWNSGALTSGVHTFPAVLQSSGLYSLSSVVTVPSSS |
| | BAP0066/ | LGTKTYTCNVDHKPSNTKVDKRVESKYGPPCPPCPAPE |
| | BAP0067/ | FLGGPSVFLFPPKPKDTLMISRTPEVTCVVVDVSQEDPE |
| | BAP0068 | VQFNWYVDGVEVHNAKTKPREEQFNSTYRVVSVLTVL |
| | heavy chain #1 | HQDWLNGKEYKCKVSNKGLPSSIEKTISKAKGQPREPQ |
| | | VYTLPPSQEEMTKNQVSLSCAVKGFYPSDIAVEWESNG |
| | | QPENNYKTTPPVLDSDGSFFLVSRLTVDKSRWQEGNVF |
| | | SCSVMHEALHNRFTQKSLSLSLGK |
| 147. | BAP0061/ | DIQMTQSPSSLSASVGDRVTITCRSSQSLLYSDGRTYLN |
| | BAP0062/ | WYQQKPGKSPKRLIYLVSKLDSGVPDRFSGSGSGTDFT |
| | BAP0063/ | LTISSLQPEDFATYYCWQGTHLPHTFGGGTKVEIKRTVA |
| | BAP0064/ | APSVFIFPPSDEQLKSGTASVVCLLNNFYPREAKVQWK |
| | BAP0065/ | VDNALQSGNSQESVTEQDSKDSTYSLSSTLTLSKADYE |
| | BAP0066/ | KHKVYACEVTHQGLSSPVTKSFNRGEC |
| | BAP0067/ | |
| | BAP0068/ | |
| | BAP0074/ | |
| | PBA0075/ | |
| | BAP0076 | |
| | light chain | |
| | #1 | |
| 148. | BAP0061 | EVQLVESGGGVVQPGRSLRLSCAASGFTFSDYAMAWV |
| | heavy chain | RQAPGKGLEWVATIIYDGSSTYYRDSVKGRFTISRDNSK |
| | #2 | NTLYLQMNSLRAEDTAVYYCATGLGIATDYFDYWGQG |
| | | TLVTVSSASTKGPSVFPCAPSSRSTSESTAALGCLVKDY |
| | | FPEPVTVSWNSGALTSGVHTFPAVLQSSGLYSLSSVVTV |
| | | PSSSLGTKTYTCNVDHKPSNTKVDKRVESKYGPPCPPCP |
| | | APEFLGGPSVFLFPPKPKDTLMISRTPEVTCVVVDVSQE |
| | | DPEVQFNWYVDGVEVHNAKTKPREEQFNSTYRVVSVL |
| | | TVLHQDWLNGKEYKCKVSNKGLPSSIEKTISKAKGQPR |
| | | EPQVYTLPPSQEEMTKNQVSLWCLVKGFYPSDIAVEWE |
| | | SNGQPENNYKTTPPVLDSDGSFFLYSRLTVDKSRWQEG |
| | | NVFSCSVMHEALHNHYTQKSLSLSLGK |
| 149. | BAP0061 | DIRMTQSPFSLSASVGDRVTITCLASEDIYSDLAWYQQK |
| | light chain #2 | PAKAPKLFIYNANSLQNGVPSRFSGSGSGTDYTLTISSLQ |
| | | PEDFATYYCQQYNNYPPTFGGGTKVEIKRTVAAPSVFIC |
| | | PPSDEQLKSGTASVVCLLNNFYPREAKVQWKVDNALQ |
| | | SGNSQESVTEQDSKDSTYSLSSTLTLSKADYEKHKVYA |
| | | CEVTHQGLSSPVTKSFNRGES |
| 150. | BAP0062 | EVQLVESGGGVVQPGRSLRLSCAASGFTFSDYAMAWV |
| | heavy chain | RQAPGKGLEWVATIIYDGSSTYYRDSVKGRFTISRDNSK |
| | #2 | NTLYLQMNSLRAEDTAVYYCATGLGIATDYFDYWGQG |
| | | TLVTVSSASTKGPSVFPLAPSSRSTSESTAALGCLVKDY |
| | | FPEPVTVSWNSGALTSGVHTCPAVLQSSGLYSLSSVVT |
| | | VPSSSLGTKTYTCNVDHKPSNTKVDKRVESKYGPPCPP |
| | | CPAPEFLGGPSVFLFPPKPKDTLMISRTPEVTCVVVDVS |
| | | QEDPEVQFNWYVDGVEVHNAKTKPREEQFNSTYRVVS |
| | | VLTVLHQDWLNGKEYKCKVSNKGLPSSIEKTISKAKGQ |
| | | PREPQVYTLPPSQEEMTKNQVSLWCLVKGFYPSDIAVE |
| | | WESNGQPENNYKTTPPVLDSDGSFFLYSRLTVDKSRWQ |
| | | EGNVFSCSVMHEALHNHYTQKSLSLSLGK |
| 151. | BAP0062 | DIRMTQSPFSLSASVGDRVTITCLASEDIYSDLAWYQQK |
| | light chain #2 | PAKAPKLFIYNANSLQNGVPSRFSGSGSGTDYTLTISSLQ |
| | | PEDFATYYCQQYNNYPPTFGGGTKVEIKRTVAAPSVFIF |
| | | PPSDEQLKSGTASVVCLLNNFYPREAKVQWKVDNALQ |
| | | SGNSQESVTEQDSKDSTYSLCSTLTLSKADYEKHKVYA |
| | | CEVTHQGLSSPVTKSFNRGES |
| 152. | BAP0063 | EVQLVESGGGVVQPGRSLRLSCAASGFTFSDYAMAWV |
| | heavy chain | RQAPGKGLEWVATIIYDGSSTYYRDSVKGRFTISRDNSK |
| | #2 | NTLYLQMNSLRAEDTAVYYCATGLGIATDYFDYWGQG |
| | | TLVTVSSASTKGPSVFPLAPSSRSTSESTAALGCLVKDY |
| | | FPEPVTVSWNSGALTSGVHTFPACLQSSGLYSLSSVVTV |
| | | PSSSLGTKTYTCNVDHKPSNTKVDKRVESKYGPPCPPCP |
| | | APEFLGGPSVFLFPPKPKDTLMISRTPEVTCVVVDVSQE |
| | | DPEVQFNWYVDGVEVHNAKTKPREEQFNSTYRVVSVL |
| | | TVLHQDWLNGKEYKCKVSNKGLPSSIEKTISKAKGQPR |
| | | EPQVYTLPPSQEEMTKNQVSLWCLVKGFYPSDIAVEWE |
| | | SNGQPENNYKTTPPVLDSDGSFFLYSRLTVDKSRWQEG |
| | | NVFSCSVMHEALHNHYTQKSLSLSLGK |
| 153. | BAP0063 | DIRMTQSPFSLSASVGDRVTITCLASEDIYSDLAWYQQK |
| | light chain #2 | PAKAPKLFIYNANSLQNGVPSRFSGSGSGTDYTLTISSLQ |
| | | PEDFATYYCQQYNNYPPTFGGGTKVEIKRTVAAPSVFIF |
| | | PPSDEQLKSGTASVVCLLNNFYPREAKVQWKVDNALQ |
| | | SGNSCESVTEQDSKDSTYSLSSTLTLSKADYEKHKVYA |
| | | CEVTHQGLSSPVTKSFNRGES |
| 154. | BAP0064 | EVQLVESGGGVVQPGRSLRLSCAASGFTFSDYAMAWV |
| | heavy chain | RQAPGKGLEWVATIIYDGSSTYYRDSVKGRFTISRDNSK |
| | #2 | NTLYLQMNSLRAEDTAVYYCATGLGIATDYFDYWGQG |
| | | TLVTVSSASTKGPSVCPLAPSSRSTSESTAALGCLVKDY |
| | | FPEPVTVSWNSGALTSGVHTFPAVLQSSGLYSLSSVVTV |
| | | PSSSLGTKTYTCNVDHKPSNTKVDKRVESKYGPPCPPCP |
| | | APEFLGGPSVFLFPPKPKDTLMISRTPEVTCVVVDVSQE |
| | | DPEVQFNWYVDGVEVHNAKTKPREEQFNSTYRVVSVL |
| | | TVLHQDWLNGKEYKCKVSNKGLPSSIEKTISKAKGQPR |
| | | EPQVYTLPPSQEEMTKNQVSLWCLVKGFYPSDIAVEWE |
| | | SNGQPENNYKTTPPVLDSDGSFFLYSRLTVDKSRWQEG |
| | | NVFSCSVMHEALHNHYTQKSLSLSLGK |
| 155. | BAP0064 | DIRMTQSPFSLSASVGDRVTITCLASEDIYSDLAWYQQK |
| | light chain #2 | PAKAPKLFIYNANSLQNGVPSRFSGSGSGTDYTLTISSLQ |
| | | PEDFATYYCQQYNNYPPTFGGGTKVEIKRTVAAPSVFIC |
| | | PPSDEQLKSGTASVVCLLNNFYPREAKVQWKVDNALQ |
| | | SGNSQESVTEQDSKDSTYSLSSTLTLSKADYEKHKVYA |
| | | CEVTHQGLSSPVTKSFNRGES |
| 156. | BAP0065 | EVQLLESGGGLVQPGGSLRLSCAASGFTFSSYAMSWVR |
| | heavy chain | QAPGKGLEWVSGITGSGGSTYYADSVKGRFTISRDNSK |
| | #2 | NTLYLQMNSLRAEDTAVYYCAKDPGTTVIMSWFDPWG |
| | | QGTLVTVSSASTKGPSVFPCAPSSRSTSESTAALGCLVK |
| | | DYFPEPVTVSWNSGALTSGVHTFPAVLQSSGLYSLSSV |
| | | VTVPSSSLGTKTYTCNVDHKPSNTKVDKRVESKYGPPC |
| | | PPCPAPEFLGGPSVFLFPPKPKDTLMISRTPEVTCVVVDV |
| | | SQEDPEVQFNWYVDGVEVHNAKTKPREEQFNSTYRVV |
| | | SVLTVLHQDWLNGKEYKCKVSNKGLPSSIEKTISKAKG |
| | | QPREPQVYTLPPSQEEMTKNQVSLWCLVKGFYPSDIAV |
| | | EWESNGQPENNYKTTPPVLDSDGSFFLYSRLTVDKSRW |
| | | QEGNVFSCSVMHEALHNHYTQKSLSLSLGK |
| 157. | BAP0065 | EIVLTQSPGTLSLSPGERATLSCRASQSVRGRYLAWYQQ |
| | light chain #2 | KPGQAPRLLIYGASSRATGIPDRFSGSGSGTDFTLTISRL |
| | | EPEDFAVFYCQQYGSSPRTFGQGTKVEIKRTVAAPSVFI |
| | | CPPSDEQLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGES |
| 158. | BAP0066 | EVQLLESGGGLVQPGGSLRLSCAASGFTFSSYAMSWVR |
| | heavy chain | QAPGKGLEWVSGITGSGGSTYYADSVKGRFTISRDNSK |
| | #2 | NTLYLQMNSLRAEDTAVYYCAKDPGTTVIMSWFDPWG |
| | | QGTLVTVSSASTKGPSVFPLAPSSRSTSESTAALGCLVK |
| | | DYFPEPVTVSWNSGALTSGVHTCPAVLQSSGLYSLSSV |
| | | VTVPSSSLGTKTYTCNVDHKPSNTKVDKRVESKYGPPC |
| | | PPCPAPEFLGGPSVFLFPPKPKDTLMISRTPEVTCVVVDV |
| | | SQEDPEVQFNWYVDGVEVHNAKTKPREEQFNSTYRVV |
| | | SVLTVLHQDWLNGKEYKCKVSNKGLPSSIEKTISKAKG |
| | | QPREPQVYTLPPSQEEMTKNQVSLWCLVKGFYPSDIAV |
| | | EWESNGQPENNYKTTPPVLDSDGSFFLYSRLTVDKSRW |
| | | QEGNVFSCSVMHEALHNHYTQKSLSLSLGK |
| 159. | BAP0066 | EIVLTQSPGTLSLSPGERATLSCRASQSVRGRYLAWYQQ |
| | light chain #2 | KPGQAPRLLIYGASSRATGIPDRFSGSGSGTDFTLTISRL |
| | | EPEDFAVFYCQQYGSSPRTFGQGTKVEIKRTVAAPSVFI |
| | | FPPSDEQLKSGTASVVCLLNNFYPREAKVQWKVDNAL |
| | | QSGNSQESVTEQDSKDSTYSLCSTLTLSKADYEKHKVY |
| | | ACEVTHQGLSSPVTKSFNRGES |
| 160. | BAP0067 | EVQLLESGGGLVQPGGSLRLSCAASGFTFSSYAMSWVR |
| | heavy chain | QAPGKGLEWVSGITGSGGSTYYADSVKGRFTISRDNSK |
| | #2 | NTLYLQMNSLRAEDTAVYYCAKDPGTTVIMSWFDPWG |
| | | QGTLVTVSSASTKGPSVCPLAPSSRSTSESTAALGCLVK |
| | | DYFPEPVTVSWNSGALTSGVHTFPAVLQSSGLYSLSSV |
| | | VTVPSSSLGTKTYTCNVDHKPSNTKVDKRVESKYGPPC |
| | | PPCPAPEFLGGPSVFLFPPKPKDTLMISRTPEVTCVVVDV |
| | | SQEDPEVQFNWYVDGVEVHNAKTKPREEQFNSTYRVV |
| | | SVLTVLHQDWLNGKEYKCKVSNKGLPSSIEKTISKAKG |
| | | QPREPQVYTLPPSQEEMTKNQVSLWCLVKGFYPSDIAV |
| | | EWESNGQPENNYKTTPPVLDSDGSFFLYSRLTVDKSRW |
| | | QEGNVFSCSVMHEALHNHYTQKSLSLSLGK |
| 161. | BAP0067 | EIVLTQSPGTLSLSPGERATLSCRASQSVRGRYLAWYQQ |
| | light chain #2 | KPGQAPRLLIYGASSRATGIPDRFSGSGSGTDFTLTISRL |
| | | EPEDFAVFYCQQYGSSPRTFGQGTKVEIKRTVAAPSVFI |
| | | CPPSDEQLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGES |
| 162. | BAP0068 | EVQLLESGGGLVQPGGSLRLSCAASGFTFSSYAMSWVR |
| | heavy chain | QAPGKGLEWVSGITGSGGSTYYADSVKGRFTISRDNSK |
| | #2 | NTLYLQMNSLRAEDTAVYYCAKDPGTTVIMSWFDPWG |
| | | QGTLVTVSSASTKGPSVFPLAPSSRSTSESTAALGCLVK |
| | | DYFPEPVTVSWNSGALTSGVHTFPACLQSSGLYSLSSVV |
| | | TVPSSSLGTKTYTCNVDHKPSNTKVDKRVESKYGPPCP |
| | | PCPAPEFLGGPSVFLFPPKPKDTLMISRTPEVTCVVVDVS |
| | | QEDPEVQFNWYVDGVEVHNAKTKPREEQFNSTYRVVS |
| | | VLTVLHQDWLNGKEYKCKVSNKGLPSSIEKTISKAKGQ |
| | | PREPQVYTLPPSQEEMTKNQVSLWCLVKGFYPSDIAVE |
| | | WESNGQPENNYKTTPPVLDSDGSFFLYSRLTVDKSRWQ |
| | | EGNVFSCSVMHEALHNHYTQKSLSLSLGK |
| 163. | BAP0068/ | EIVLTQSPGTLSLSPGERATLSCRASQSVRGRYLAWYQQ |
| | BAP0074/ | KPGQAPRLLIYGASSRATGIPDRFSGSGSGTDFTLTISRL |
| | 7BAP005/BAP0076 | EPEDFAVFYCQQYGSSPRTFGQGTKVEIKRTVAAPSVFI |
| | light chain #2 | FPPSDEQLKSGTASVVCLLNNFYPREAKVQWKVDNAL |
| | | QSGNSCESVTEQDSKDSTYSLSSTLTLSKADYEKHKVY |
| | | ACEVTHQGLSSPVTKSFNRGES |
| 171. | BAP0074 | EVQLVQSGAEVKKPGASVKVSCKASGYTFTDYEIHWV |
| | heavy chain | RQAPGKGLEWMGAIDPETGGTAYNQKFKGRVTMTTDT |
| | #1 | STSTAYMELRSLRSDDTAVYYCYSYDYVTYWGQGTTV |
| | | TVSSASTKGPSVFPLAPCSRSTSESTAALGCLVKDYFPEP |
| | | VTVSWNSGALTSGVHTFPAVLQSSGLYSLSSVVTVPSS |
| | | NFGTQTYTCNVDHKPSNTKVDKTVERKCCVECPPCPAP |
| | | PVAGPSVFLFPPKPKDTLMISRTPEVTCVVVDVSHEDPE |
| | | VQFNWYVDGVEVHNAKTKPREEQFNSTFRVVSVLTVV |
| | | HQDWLNGKEYKCKVSNKGLPAPIEKTISKTKGQPREPQ |
| | | VYTLPPCREEMTKNQVSLWCLVKGFYPSDIAVEWESN |
| | | GQPENNYKTTPPMLDSDGSFFLYSKLTVDKSRWQQGN |
| | | VFSCSVMHEALHNHYTQKSLSLSPGK |
| 172. | BAP0074 | EVQLLESGGGLVQPGGSLRLSCAASGFTFSSYAMSWVR |
| | heavy chain | QAPGKGLEWVSGITGSGGSTYYADSVKGRFTISRDNSK |
| | #2 | NTLYLQMNSLRAEDTAVYYCAKDPGTTVIMSWFDPWG |
| | | QGTLVTVSSASTKGPSVFPLAPSSRSTSESTAALGCLVK |
| | | DYFPEPVTVSWNSGALTSGVHTFPACLQSSGLYSLSSVV |
| | | TVPSSNFGTQTYTCNVDHKPSNTKVDKTVERKCCVECP |
| | | PCPAPPVAGPSVFLFPPKPKDTLMISRTPEVTCVVVDVS |
| | | HEDPEVQFNWYVDGVEVHNAKTKPREEQFNSTFRVVS |
| | | VLTVVHQDWLNGKEYKCKVSNKGLPAPIEKTISKTKGQ |
| | | PREPQVCTLPPSREEMTKNQVSLSCAVKGFYPSDIAVE |
| | | WESNGQPENNYKTTPPMLDSDGSFFLVSKLTVDKSRW |
| | | QQGNVFSCSVMHEALHNRFTQKSLSLSPGK |
| 173. | BAP0075 | EVQLVQSGAEVKKPGASVKVSCKASGYTFTDYEIHWV |
| | heavy chain | RQAPGKGLEWMGAIDPETGGTAYNQKFKGRVTMTTDT |
| | #1 | STSTAYMELRSLRSDDTAVYYCYSYDYVTYWGQGTTV |
| | | TVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKKVEPKSCDKTHTCPPC |
| | | PAPEAAGGPSVFLFPPKPKDTLMISRTPEVTCVVVDVSH |
| | | EDPEVKFNWYVDGVEVHNAKTKPREEQYNSTYRVVSV |
| | | LTVLHQDWLNGKEYKCKVSNKALPAPIEKTISKAKGQP |
| | | REPQVCTLPPSRDELTKNQVSLSCAVKGFYPSDIAVEWE |
| | | SNGQPENNYKTTPPVLDSDGSFFLVSKLTVDKSRWQQG |
| | | NVFSCSVMHEALHNRFTQKSLSLSPGK |
| 174. | BAP0075 | EVQLLESGGGLVQPGGSLRLSCAASGFTFSSYAMSWVR |
| | heavy chain | QAPGKGLEWVSGITGSGGSTYYADSVKGRFTISRDNSK |
| | #2 | NTLYLQMNSLRAEDTAVYYCAKDPGTTVIMSWFDPWG |
| | | QGTLVTVSSASTKGPSVFPLAPSSKSTSGGTAALGCLVK |
| | | DYFPEPVTVSWNSGALTSGVHTFPACLQSSGLYSLSSVV |
| | | TVPSSSLGTQTYICNVNHKPSNTKVDKKVEPKSSDKTH |
| | | TCPPCPAPEAAGGPSVFLFPPKPKDTLMISRTPEVTCVV |
| | | VDVSHEDPEVKFNWYVDGVEVHNAKTKPREEQYNSTY |
| | | RVVSVLTVLHQDWLNGKEYKCKVSNKALPAPIEKTISK |
| | | AKGQPREPQVYTLPPCRDELTKNQVSLWCLVKGFYPSD |
| | | IAVEWESNGQPENNYKTTPPVLDSDGSFFLYSKLTVDK |
| | | SRWQQGNVFSCSVMHEALHNHYTQKSLSLSPGK |
| 175. | BAP0076 | EVQLVQSGAEVKKPGASVKVSCKASGYTFTDYEIHWV |
| | heavy chain | RQAPGKGLEWMGAIDPETGGTAYNQKFKGRVTMTTDT |
| | #1 | STSTAYMELRSLRSDDTAVYYCYSYDYVTYWGQGTTV |
| | | TVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKKVEPKSCDKTHTCPPC |
| | | PAPEAAGGPSVFLFPPKPKDTLMISRTPEVTCVVVDVSH |
| | | EDPEVKFNWYVDGVEVHNAKTKPREEQYNSTYRVVSV |
| | | LTVLHQDWLNGKEYKCKVSNKALPAPIEKTISKAKGQP |
| | | REPQVCTLPPSRDELTKNQVSLSCAVKGFYPSDIAVEWE |
| | | SNGQPENNYKTTPPVLDSDGSFFLVSKLTVDKSRWQQG |
| | | NVFSCSVLHEALHARFTQKSLSLSPGK |
| 176. | BAP0076 | EVQLLESGGGLVQPGGSLRLSCAASGFTFSSYAMSWVR |
| | heavy chain | QAPGKGLEWVSGITGSGGSTYYADSVKGRFTISRDNSK |
| | #2 | NTLYLQMNSLRAEDTAVYYCAKDPGTTVIMSWFDPWG |
| | | QGTLVTVSSASTKGPSVFPLAPSSKSTSGGTAALGCLVK |
| | | DYFPEPVTVSWNSGALTSGVHTFPACLQSSGLYSLSSVV |
| | | TVPSSSLGTQTYICNVNHKPSNTKVDKKVEPKSSDKTH |
| | | TCPPCPAPEAAGGPSVFLFPPKPKDTLMISRTPEVTCVV |
| | | VDVSHEDPEVKFNWYVDGVEVHNAKTKPREEQYNSTY |
| | | RVVSVLTVLHQDWLNGKEYKCKVSNKALPAPIEKTISK |
| | | AKGQPREPQVYTLPPCRDELTKNQVSLWCLVKGFYPSD |
| | | IAVEWESNGQPENNYKTTPPVLDSDGSFFLYSKLTVDK |
| | | SRWQQGNVFSCSVLHEALHAHYTQKSLSLSPGK |
| 177. | BAP0069 | EVQLVQSGAEVKKPGASVKVSCKASGYTFTDYEIHWV |
| | heavy chain | RQAPGKGLEWMGAIDPETGGTAYNQKFKGRVTMTTDT |
| | | STSTAYMELRSLRSDDTAVYYCYSYDYVTYWGQGTTV |
| | | TVSSASTKGPSVFPLAPCSRSTSESTAALGCLVKDYFPEP |
| | | VTVSWNSGALTSGVHTFPAVLQSSGLYSLSSVVTVPSSS |
| | | LGTKTYTCNVDHKPSNTKVDKRVESKYGPPCPPCPAPE |
| | | AAGGPSVFLFPPKPKDTLMISRTPEVTCVVVDVSQEDPE |
| | | VQFNWYVDGVEVHNAKTKPREEQFNSTYRVVSVLTVL |
| | | HQDWLNGKEYKCKVSNKGLPSSIEKTISKAKGQPREPQ |
| | | VYTLPPSQEEMTKNQVSLTCLVKGFYPSDIAVEWESNG |
| | | QPENNYKTTPPVLDSDGSFFLYSKLTVDKSRWQEGNVF |
| | | SCSVMHEALHNHYTQKSLSLSPGKGGGGSGGGGSGGG |
| | | GSEIVLTQSPGTLSLSPGERATLSCRASQSVRGRYLAWY |
| | | QQKPGQAPRLLIYGASSRATGIPDRFSGSGSGTDFTLTIS |
| | | RLEPEDFAVFYCQQYGSSPRTFGCGTKVEIKGGGGSGG |
| | | GGSGGGGSGGGGSEVQLLESGGGLVQPGGSLRLSCAAS |
| | | GFTFSSYAMSWVRQAPGKCLEWVSGITGSGGSTYYAD |
| | | SVKGRFTISRDNSKNTLYLQMNSLRAEDTAVYYCAKDP |
| | | GTTVIMSWFDPWGQGTLVTVSA |
| 178. | BAP0070/ | EVQLVQSGAEVKKPGASVKVSCKASGYTFTDYEIHWV |
| | BAP0071 heavy | RQAPGKGLEWMGAIDPETGGTAYNQKFKGRVTMTTDT |
| | chain | STSTAYMELRSLRSDDTAVYYCYSYDYVTYWGQGTTV |
| | | TVSSASTKGPSVFPLAPCSRSTSESTAALGCLVKDYFPEP |
| | | VTVSWNSGALTSGVHTFPAVLQSSGLYSLSSVVTVPSSS |
| | | LGTKTYTCNVDHKPSNTKVDKRVESKYGPPCPPCPAPE |
| | | AAGGPSVFLFPPKPKDTLMISRTPEVTCVVVDVSQEDPE |
| | | VQFNWYVDGVEVHNAKTKPREEQFNSTYRVVSVLTVL |
| | | HQDWLNGKEYKCKVSNKGLPSSIEKTISKAKGQPREPQ |
| | | VYTLPPSQEEMTKNQVSLTCLVKGFYPSDIAVEWESNG |
| | | QPENNYKTTPPVLDSDGSFFLYSKLTVDKSRWQEGNVF |
| | | SCSVMHEALHNHYTQKSLSLSPGKGGGGSGGGGSGGG |
| | | GSEVQLLESGGGLVQPGGSLRLSCAASGFTFSSYAMSW |
| | | VRQAPGKCLEWVSGITGSGGSTYYADSVKGRFTISRDN |
| | | SKNTLYLQMNSLRAEDTAVYYCAKDPGTTVIMSWFDP |
| | | WGQGTLVTVSSGGGGSGGGGSGGGGSGGGGSEIVLTQ |
| | | SPGTLSLSPGERATLSCRASQSVRGRYLAWYQQKPGQA |
| | | PRLLIYGASSRATGIPDRFSGSGSGTDFTLTISRLEPEDFA |
| | | VFYCQQYGSSPRTFGCGTKVEIK |
| 179. | BAP0072 | EVQLVQSGAEVKKPGASVKVSCKASGYTFTDYEIHWV |
| | heavy chain | RQAPGKGLEWMGAIDPETGGTAYNQKFKGRVTMTTDT |
| | | STSTAYMELRSLRSDDTAVYYCYSYDYVTYWGQGTTV |
| | | TVSSASTKGPSVFPLAPCSRSTSESTAALGCLVKDYFPEP |
| | | VTVSWNSGALTSGVHTFPAVLQSSGLYSLSSVVTVPSS |
| | | NFGTQTYTCNVDHKPSNTKVDKTVERKCCVECPPCPAP |
| | | PVAGPSVFLFPPKPKDTLMISRTPEVTCVVVDVSHEDPE |
| | | VQFNWYVDGVEVHNAKTKPREEQFNSTFRVVSVLTVV |
| | | HQDWLNGKEYKCKVSNKGLPAPIEKTISKTKGQPREPQ |
| | | VYTLPPSREEMTKNQVSLTCLVKGFYPSDIAVEWESNG |
| | | QPENNYKTTPPMLDSDGSFFLYSKLTVDKSRWQQGNV |
| | | FSCSVMHEALHNHYTQKSLSLSPGKGGGGSGGGGSGG |
| | | GGSEVQLLESGGGLVQPGGSLRLSCAASGFTFSSYAMS |
| | | WVRQAPGKCLEWVSGITGSGGSTYYADSVKGRFTISRD |
| | | NSKNTLYLQMNSLRAEDTAVYYCAKDPGTTVIMSWFD |
| | | PWGQGTLVTVSSGGGGSGGGGSGGGGSGGGGSEIVLT |
| | | QSPGTLSLSPGERATLSCRASQSVRGRYLAWYQQKPGQ |
| | | APRLLIYGASSRATGIPDRFSGSGSGTDFTLTISRLEPEDF |
| | | AVFYCQQYGSSPRTFGCGTKVEIK |
| 180. | BAP0073 | EVQLVQSGAEVKKPGASVKVSCKASGYTFTDYEIHWV |
| | heavy chain | RQAPGKGLEWMGAIDPETGGTAYNQKFKGRVTMTTDT |
| | | STSTAYMELRSLRSDDTAVYYCYSYDYVTYWGQGTTV |
| | | TVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKKVEPKSCDKTHTCPPC |
| | | PAPEAAGGPSVFLFPPKPKDTLMISRTPEVTCVVVDVSH |
| | | EDPEVKFNWYVDGVEVHNAKTKPREEQYNSTYRVVSV |
| | | LTVLHQDWLNGKEYKCKVSNKALPAPIEKTISKAKGQP |
| | | REPQVYTLPPSRDELTKNQVSLTCLVKGFYPSDIAVEWE |
| | | SNGQPENNYKTTPPVLDSDGSFFLYSKLTVDKSRWQQG |
| | | NVFSCSVMHEALHNHYTQKSLSLSPGKGGGGSGGGGS |
| | | GGGGSEVQLLESGGGLVQPGGSLRLSCAASGFTFSSYA |
| | | MSWVRQAPGKCLEWVSGITGSGGSTYYADSVKGRFTIS |
| | | RDNSKNTLYLQMNSLRAEDTAVYYCAKDPGTTVIMSW |
| | | FDPWGQGTLVTVSSGGGGSGGGGSGGGGSGGGGSEIV |
| | | LTQSPGTLSLSPGERATLSCRASQSVRGRYLAWYQQKP |
| | | GQAPRLLIYGASSRATGIPDRFSGSGSGTDFTLTISRLEPE |
| | | DFAVFYCQQYGSSPRTFGCGTKVEIK |

Additional exemplary anti-DKK1 or anti-sclerostin sequences

| | | |
|---|---|---|
| 164. | 11H10 VH2 | EVQLVESGGGLVQPANSLKLSCAASGFTFSDYAMAWV |
| | | RQSPKKGLEWVATIIYDGSSTYYRDSVKGRFTISRDNAK |
| | | STLYLQMDSLRSEDTATYYCATGLGIATDYFDYWGQG |
| | | VLVTVSS |
| 165. | 11H10 VL2 | DIRMTQSPASLSASLGETVNIECLASEDIYSDLAWYQQK |
| | | PGKSPQLLIYNANSLQNGVPSRFSGSGSGTQYSLKINSL |
| | | QSEDVATYFCQQYNNYPPTFGGGTKLELK |
| 166. | 11H10 VH3 | EVQLVESGGGVVQPGRSLRLSCAASGFTFSDYAMAWV |
| | | RQAPGKCLEWVATIIYDGSSTYYRDSVKGRFTISRDNS |
| | | KNTLYLQMNSLRAEDTAVYYCATGLGIATDYFDYWG |
| | | QGTLVTVSS |
| 167. | 11H10 VL3 | DIRMTQSPFSLSASVGDRVTITCLASEDIYSDLAWYQQK |
| | | PAKAPKLFIYNANSLQNGVPSRFSGSGSGTDYTLTISSLQ |
| | | PEDFATYYCQQYNNYPPTFGCGTKVEIK |
| 168. | hAb VH2 | EVQLVQSGAEVKKPGASVKVSCKASGYTFTDYEIHWV |
| | | RQAPGKCLEWMGAIDPETGGTAYNQKFKGRVTMTTDT |
| | | STSTAYMELRSLRSDDTAVYYCYSYDYVTYWGQGTTV |
| | | TVSS |
| 169. | hAb1 VL2 | DVVMTQSPLSLSVSPGERASLSCKSSQSLLYSDGRTYL |
| | | NWYLQKPGQSPQRLIYLVSKLDSGVPDRFSGSGSGTDF |
| | | TLKISRVQSEDVGVYYCWQGTHLPHTFGCGTKVEIK |
| 170. | hAb2 VL2 | DIQMTQSPSSLSASVGDRVTITCRSSQSLLYSDGRTYLN |
| | | WYQQKPGKSPKRLIYLVSKLDSGVPDRFSGSGSGTDFT |
| | | LTISSLQPEDFATYYCWQGTHLPHTFGCGTKVEIK |

Peptide amino acid sequences on the second loop of Sclerostin

| | | |
|---|---|---|
| 185 | hScl 110-133/ | GPARLLPNAIGRGKWWRPSGPDFR |
| | pep1 | |
| 186 | hScl 119-126 | IGRGKWWR |
| 187 | pep5 | PNAIGRGKWWR |
| 188 | pep6 | IGRGKWWRPSGP |

Additional sequences

| | | |
|---|---|---|
| 183 | SOST_ | QGWQAFKNDATEIIPELGEYPEPPPELENNKTMNRAEN |
| | HUMAN_Q9BQB4 | GGRPPHHPFETKDVSEYSCRELHFTRYVTDGPCRSAKP |
| | | VTELVCSGQCGPARLLPNAIGRGKWWRPSGPDFRCIPD |
| | | RYRAQRVQLLCPGGEAPRARKVRLVASCKCKRLTRFH |
| | | NQSELKDFGTEAARPQKGRKPRPRARSAKANQAELEN |
| | | AY |
| 184 | SOST_ | QGWQAFKNDATEIIPELGEYPEPPPDLENNKTMNRAEN |
| | MACFA_ | GGRPPHHPFETKDVSEYSCRELHFTRYVTDGQCRSAKP |
| | A0A2K5VUE5 | VTELVCSGQCGPARLLPNAIGRGKWWRPSGPDFRCIPD |
| | | RYRAQRVQLLCPGGAAPRARKVRLVASCKCKRLTRFH |
| | | NQSELKDFGPEAARPQKGRKPRPRARGAKANQAELEN |
| | | AY |
| 189 | SOST_ | QGWQAFKNDATEIIPELGEYPEPPPELENNKTMNRAEN |
| | MACMU_F6WYL4 | GGRPPHHPFETKDVSEYSCRELHFTRYVTDGQCRSAKP |
| | | VTELVCSGQCGPARLLPNAIGRGKWWRPSGPDFRCIPD |
| | | RYRAQRVQLLCPGGAAPRARKVRLVASCKCKRLTRFH |
| | | NQSELKDFGPEAARPQKGRKPRPRARGAKANQAELEN |
| | | AY |
| 190 | SOST_ | QGWQAFRNDATEVIPGLGEYPEPPPENNQTMNRAENG |
| | MOUSE_Q99P68 | GRPPHHPYDAKDVSEYSCRELHYTRFLTDGQCRSAKPV |
| | | TELVCSGQCGPARLLPNAIGRVKWWRPNGPDFRCIPDR |
| | | YRAQRVQLLCPGGAAPRSRKVRLVASCKCKRLTRFHN |
| | | QSELKDFGPETARPQKGRKPRPGARGAKANQAELENA |
| | | Y |
| 191 | SOST_RAT_ | QGWQAFKNDATEVIPGLREYPEPPQELENNQTMNRAE |
| | Q99P67 | NGGRPPHHPYDTKDVSEYSCRELHYTRFVTDGQCRSAK |
| | | PVTELVCSGQCGPARLLPNAIGRVKWWRPNGPDFRCIP |
| | | DRYRAQRVQLLCPGGAAPRSRKVRLVASCKCKRLTRF |
| | | HNQSELKDFGPETARPQKGRKPRPRARGAKANQAELE |
| | | NAY |
| 192 | Pep2 mSOST | GPARLLPNAIGRVKWWRPNGPDFR |
| 193 | Pep3 | GPARLLPNAIGR |
| | h/mSOST | |
| 194 | Pep4 hSOST | RLLPNAIGRGK |
| 195 | Pep7 hSOST | KWWRPSGPDFR |
| 196 | Pep8 mSOST | PNAIGRVKWWR |
| 197 | Pep9 mSOST | IGRVKWWRPNGP |
| 198 | Pep10 mSOST | KWWRPNGPDFR |
| 199 | HC-CDR1 | DFEIH |
| 200 | HC-CDR2 | AIDPETGGSANNQKFKA |

1. An anti-Sclerostin construct, comprising:
a) a first polypeptide comprising a first light chain comprising, from N-terminus to C-terminus, i) a light chain variable region (V_{L}), and ii) a first light chain constant domain (“first CL domain”);
b) a second polypeptide comprising a first heavy chain comprising, from N-terminus to C-terminus, i) a heavy chain variable region (V_{H}), ii) a first heavy chain constant domain (“first CH1 domain”), and iii) a first Fc domain;
c) a third polypeptide comprising a second heavy chain comprising, from N-terminus to C-terminus, i) a second heavy chain variable region (V_{H-2}), ii) a second heavy chain constant domain (“second CH1 domain”), and iii) a second Fc domain; and
d) a fourth polypeptide comprising a second light chain comprising, from N-terminus to C-terminus, i) a second light chain variable region (V_{L-2}), and ii) a second light chain constant domain (“second CL domain”),
wherein the first and the second Fc domains form an Fc fragment, and
wherein the first polypeptide comprises the amino acid sequence of SEQ ID NO: 147, the second polypeptide comprises the amino acid sequence of SEQ ID NO: 146, the third polypeptide comprises the amino acid sequence of SEQ ID NO: 148, and the fourth polypeptide comprises the amino acid sequence of SEQ ID NO: 149.

2. A pharmaceutical composition comprising the anti-Sclerostin construct of claim 1 and a pharmaceutically acceptable carrier.


SEQUENCE LISTING (200 sequences)

SEQ ID NO: 1 - protein, 5 aa, synthetic construct
  REGION 1..5: note = Synthetic Construct
DYEIH

SEQ ID NO: 2 - protein, 5 aa, synthetic construct
  REGION 1..5: note = Synthetic Construct
DFEMH

SEQ ID NO: 3 - protein, 5 aa, synthetic construct
  REGION 1..5: note = Synthetic Construct
DYEMH

SEQ ID NO: 4 - protein, 5 aa, synthetic construct
  REGION 1..5: note = Synthetic Construct
SYWMH

SEQ ID NO: 5 - protein, 17 aa, synthetic construct
  REGION 1..17: note = Synthetic Construct
AIDPETGGTAYNQKFKG

SEQ ID NO: 6 - protein, 17 aa, synthetic construct
  REGION 1..17: note = Synthetic Construct
AIDPETGGTAYNQKFKA

SEQ ID NO: 7 - protein, 17 aa, synthetic construct
  REGION 1..17: note = Synthetic Construct
AIDPETGGTAYNQKFTA

SEQ ID NO: 8 - protein, 17 aa, synthetic construct
  REGION 1..17: note = Synthetic Construct
MIHPNSGSSNYNEKFKS

SEQ ID NO: 9 - protein, 6 aa, synthetic construct
  REGION 1..6: note = Synthetic Construct
YDYVTY

SEQ ID NO: 10 - protein, 6 aa, synthetic construct
  REGION 1..6: note = Synthetic Construct
YDYVSY

SEQ ID NO: 11 - protein, 9 aa, synthetic construct
  REGION 1..9: note = Synthetic Construct
DYDDEGFAY

SEQ ID NO: 12 - skipped

SEQ ID NO: 13 - protein, 17 aa, synthetic construct
  REGION 1..17: note = Synthetic Construct
  VARIANT 9: note = Xaa = T or S
  VARIANT 11: note = Xaa = Y or N
  VARIANT 16: note = Xaa = K or T
  VARIANT 17: note = Xaa = A, G, or S
AIDPETGGXAXNQKFXX

SEQ ID NO: 14 - protein, 6 aa, synthetic construct
  REGION 1..6: note = Synthetic Construct
  VARIANT 5: note = Xaa = T or S
YDYVXY

SEQ ID NO: 15 - protein, 16 aa, synthetic construct
  REGION 1..16: note = Synthetic Construct
KSSQSLLYSDGRTYLN

SEQ ID NO: 16 - protein, 16 aa, synthetic construct
  REGION 1..16: note = Synthetic Construct
KSSQSLLYSDGKTYLN

SEQ ID NO: 17 - protein, 11 aa, synthetic construct
  REGION 1..11: note = Synthetic Construct
KASQSVSNDVA

SEQ ID NO: 18 - protein, 7 aa, synthetic construct
  REGION 1..7: note = Synthetic Construct
LVSKLDS

SEQ ID NO: 19 - protein, 7 aa, synthetic construct
  REGION 1..7: note = Synthetic Construct
YASNRCT

SEQ ID NO: 20 - protein, 9 aa, synthetic construct
  REGION 1..9: note = Synthetic Construct
WQGTHLPHT

SEQ ID NO: 21 - protein, 9 aa, synthetic construct
  REGION 1..9: note = Synthetic Construct
QQDYSSPWT

SEQ ID NO: 22 - protein, 115 aa, synthetic construct
  REGION 1..115: note = Synthetic Construct
QAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSA

SEQ ID NO: 23 - protein, 112 aa, synthetic construct
  REGION 1..112: note = Synthetic Construct
DVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELK

SEQ ID NO: 24 - protein, 115 aa, synthetic construct
  REGION 1..115: note = Synthetic Construct
QVQLQQSGAELVRPGASVTLSCKASGYTFTDYEIHWVKQTPVHGLEWIGAIDPETGGTAYNQKFKAKAILTADRSSSTAYMELRSLTSEDSAVYYSFSYDYVSYWGQGTLVTVSA

SEQ ID NO: 25 - protein, 112 aa, synthetic construct
  REGION 1..112: note = Synthetic Construct
DVVMTQTPLTLSVTIGQPASISCKSSQSLLYSDGRTYLNWLLQRPGQSPKRLIYLVSKLDSGVPDRFAGSGSGTDFSLKISRVEAEDLGVYYCWQGTHLPHTFGAGTKLELK

SEQ ID NO: 26 - protein, 115 aa, synthetic construct
  REGION 1..115: note = Synthetic Construct
QAQLQQSGAELVRPGASVTLSCKASGYTFTDYEIHWVKQTPVHGLEWIGAIDPETGGTAYNQKFKGKAILTADRSSSTAYLELRSLTSEDSAVYYCFSYDYVTYWGQGTLVTVTA

SEQ ID NO: 27 - protein, 112 aa, synthetic construct
  REGION 1..112: note = Synthetic Construct
DVVMTQTPLTWSITIGQPASISCKSSQSLLYSDGRTYLNWLLQRPGQSPKRLIYLVSKLDSGVPDRFTGSGSGTDFTLKISRVEAEDLGFYYCWQGTHLPHTFGAGTKLELK

SEQ ID NO: 28 - protein, 115 aa, synthetic construct
  REGION 1..115: note = Synthetic Construct
QVQLQQSGAELVRPGASVTLSCKASGYTFTDFEMHWVKQTPVHGLEWIGAIDPETGGTAYNQKFTAKAILTADRSSSTAYMELRSLTSEDSAVYYCFSYDYVSYWGQGTLVTVSA

SEQ ID NO: 29 - protein, 112 aa, synthetic construct
  REGION 1..112: note = Synthetic Construct
DVMMTQTPLTLSVTIGQPASISCKSSQSLLYSDGRTYLNWLLQRPGQSPKRLIYLVSKLDSGVPDRFTGSGSGTDFTLKISRVEAEDLGVYYCWQGTHLPHTFGAGTKLELK

SEQ ID NO: 30 - protein, 115 aa, synthetic construct
  REGION 1..115: note = Synthetic Construct
QVQLQQSGAELVRPGASVTLSCKASGYTFTDYEMHWVKQTPVHGLEWIGAIDPETGGTAYNQKFKAKAILTADRSSSTAYMELRSLTSEDSAVYYCFSYDYVSYWGQGTLVTVSA

SEQ ID NO: 31 - protein, 112 aa, synthetic construct
  REGION 1..112: note = Synthetic Construct
DVVMTQTPLTLSVTIGQPASISCKSSQSLLYSDGRTYLNWLLQRPGQSPKRLIYLVSKLDSGVPDRFTGSGSGTDFTLKISRVEAEDLGVYYCWQGTHLPHTFGVGTKLELK

SEQ ID NO: 32 - protein, 115 aa, synthetic construct
  REGION 1..115: note = Synthetic Construct
QVQLQQSGAELVRPGASVTLSCKASGYTFTDFEIHWMKQTPVPGLEWIGAIDPETGGTAYNQKFKGKALLTADKSSSTAYMDLRSLTSEDSAVYFCFSYDYVSYWGQGTLVTVSA

SEQ ID NO: 33 - protein, 112 aa, synthetic construct
  REGION 1..112: note = Synthetic Construct
DVVMTQTPLTLSVTFGQPASISCKSSQSLLYSDGKTYLNWLLQRPGQSPKRLIYLVSKLDSGVPDRFTGSGSGTAFTLKISRVEAEDLGVYYCWQGTHLPHTFGAGTKLELR

SEQ ID NO: 34 - protein, 115 aa, synthetic construct
  REGION 1..115: note = Synthetic Construct
QVQLQQSGAELVRPGASVTLSCKASGYTFTDYEMHWVKQTPVHGLEWIGAIDPETGGSANNQKFKAKAILTADRSSSTAYMELRSLTSEDSAVYYCFSYDYVSYWGQGTLVTVSA

SEQ ID NO: 35 - protein, 112 aa, synthetic construct
  REGION 1..112: note = Synthetic Construct
DVVMTQTPLTLSVTIGQPASISCKSSQSLLYSDGRTYLNWLLQRPGQSPKRLIYLVSKLDSGVPDRFTGSGSGTDFTLKIGRVEAEDLGVYYCWQGTHLPHTFGAGTKLELK

SEQ ID NO: 36 - protein, 115 aa, synthetic construct; identical to SEQ ID NO: 30
  REGION 1..115: note = Synthetic Construct

SEQ ID NO: 37 - protein, 112 aa, synthetic construct
  REGION 1..112: note = Synthetic Construct
DVVMTQTPLTLSVTLGQPASISCKSSQSLLYSDGRTYLNWLLQRPGQSPKRLIYLVSKLDSGVPDRFTGSGSGTDFTLKISRVEAEDLGLYYCWQGTHLPHTFGAGTKLELK

SEQ ID NO: 38 - protein, 115 aa, synthetic construct
  REGION 1..115: note = Synthetic Construct
QVQLQQSGAELVRPGASVTLSCKASGYTFTDFEIHWLKQTPVPGLEWIGAIDPETGGTAYNQKFKGKALLTADKSSSTAYMELRSLTSEDSAVYYCFSYDYVSYWGQGTLVTVSA

SEQ ID NO: 39 - protein, 112 aa, synthetic construct
  REGION 1..112: note = Synthetic Construct
DVVMTQTPLTLSVTIGQPASISCKSSQSLLYSDGKTYLNWLLQRPGQSPKRLIYLVSKLDSGVPDRFTGSGSGTAFTLKISRVEAEDLGVYYCWQGTHLPHTFGAGTKLELR

SEQ ID NO: 40 - protein, 118 aa, synthetic construct
  REGION 1..118: note = Synthetic Construct
QVQLQQPGAELVKPGASVKLSCKASGYTFTSYWMHWVKQRPGQGLEWIGMIHPNSGSSNYNEKFKSKATLTVDKSSSTAYMQLSSLTSEDSAVYYCANDYDDEGFAYWGQGTLVTVSA

SEQ ID NO: 41 - protein, 107 aa, synthetic construct
  REGION 1..107: note = Synthetic Construct
SIVMTQTPKFLLVSAGDRVTITCKASQSVSNDVAWYQQKPGQSPKLLIYYASNRCTGVPDRFTGSGYGTDFTFTISTVQAEDLAVYFCQQDYSSPWTFGGGTKLEIK

SEQ ID NO: 42 - protein, 10 aa, synthetic construct
  REGION 1..10: note = Synthetic Construct
GFTFSDYAMA

SEQ ID NO: 43 - protein, 17 aa, synthetic construct
  REGION 1..17: note = Synthetic Construct
TIIYDGSSTYYRDSVKG

SEQ ID NO: 44 - protein, 11 aa, synthetic construct
  REGION 1..11: note = Synthetic Construct
GLGIATDYFDY

SEQ ID NO: 45 - protein, 11 aa, synthetic construct
  REGION 1..11: note = Synthetic Construct
LASEDIYSDLA

SEQ ID NO: 46 - protein, 7 aa, synthetic construct
  REGION 1..7: note = Synthetic Construct
NANSLQN

SEQ ID NO: 47 - protein, 9 aa, synthetic construct
  REGION 1..9: note = Synthetic Construct
QQYNNYPPT

SEQ ID NO: 48 - protein, 10 aa, synthetic construct
  REGION 1..10: note = Synthetic Construct
GYTFTDYYIH

SEQ ID NO: 49 - protein, 17 aa, synthetic construct
  REGION 1..17: note = Synthetic Construct
WIHSNSGATTYAQKFQA

SEQ ID NO: 50 - skipped

SEQ ID NO: 51 - protein, 13 aa, synthetic construct
  REGION 1..13: note = Synthetic Construct
TGSSNIGAGYDVH

SEQ ID NO: 52 - protein, 7 aa, synthetic construct
  REGION 1..7: note = Synthetic Construct
GYSNRPS

SEQ ID NO: 53 - protein, 11 aa, synthetic construct
  REGION 1..11: note = Synthetic Construct
QSYDNSLSSYV

SEQ ID NO: 54 - protein, 10 aa, synthetic construct
  REGION 1..10: note = Synthetic Construct
GFTFSSYAIS

SEQ ID NO: 55 - protein, 19 aa, synthetic construct
  REGION 1..19: note = Synthetic Construct
SVSGTGLGFQTYYPDSVKG

SEQ ID NO: 56 - protein, 9 aa, synthetic construct
  REGION 1..9: note = Synthetic Construct
SLENYAFDY

SEQ ID NO: 57 - protein, 15 aa, synthetic construct
  REGION 1..15: note = Synthetic Construct
RASESVDDFGISFIN

SEQ ID NO: 58 - protein, 7 aa, synthetic construct
  REGION 1..7: note = Synthetic Construct
AGSKQGS

SEQ ID NO: 59 - protein, 9 aa, synthetic construct
  REGION 1..9: note = Synthetic Construct
QQLKEVPPT

SEQ ID NO: 60 - protein, 120 aa, synthetic construct
  REGION 1..120: note = Synthetic Construct
EVQLVESGGGVVQPGRSLRLSCAASGFTFSDYAMAWVRQAPGKGLEWVATIIYDGSSTYYRDSVKGRFTISRDNSKNTLYLQMNSLRAEDTAVYYCATGLGIATDYFDYWGQGTLVTVSS

SEQ ID NO: 61 - protein, 107 aa, synthetic construct
  REGION 1..107: note = Synthetic Construct
DIRMTQSPFSLSASVGDRVTITCLASEDIYSDLAWYQQKPAKAPKLFIYNANSLQNGVPSRFSGSGSGTDYTLTISSLQPEDFATYYCQQYNNYPPTFGGGTKVEIK

SEQ ID NO: 62 - protein, 112 aa, synthetic construct
  REGION 1..112: note = Synthetic Construct
EVQLVQSGAEVKKPGASVKVSCKASGYTFTDYYIHWVRQAPGQGLEWMGWIHSNSGATTYAQKFQARVTMSRDTSSSTAYMELSRLESDDTAMYFCSREDYWGQGTLVTVSS

SEQ ID NO: 63 - protein, 110 aa, synthetic construct
  REGION 1..110: note = Synthetic Construct
QSVLTQPPSVSGAPGQRVTISCTGSSNIGAGYDVHWYQQLPGTAPKLLIYGYSNRPSGVPDRFSGSKSGASASLAITGLRPDDEADYYCQSYDNSLSSYVFGGGTQLTVL

SEQ ID NO: 64 - protein, 120 aa, synthetic construct
  REGION 1..120: note = Synthetic Construct
EVQLVESGGGLVQPGGSLRLSCAASGFTFSSYAISWVRQAPGKGLEWVASVSGTGLGFQTYYPDSVKGRFTISRDNAKNSLYLQMNSLRAEDTAVYYCATSLENYAFDYWGQGTTVTVSS

SEQ ID NO: 65 - protein, 111 aa, synthetic construct
  REGION 1..111: note = Synthetic Construct
EIVLTQSPATLSLSPGERATLSCRASESVDDFGISFINWYQQKPGQAPRLLIYAGSKQGSGIPARFSGSGSGTDFTLTISSLEPEDFAVYYCQQLKEVPPTFGGGTKVEIK

SEQ ID NO: 66 - protein, 10 aa, synthetic construct
  REGION 1..10: note = Synthetic Construct
GFTFSSYAMS

SEQ ID NO: 67 - protein, 17 aa, synthetic construct
  REGION 1..17: note = Synthetic Construct
GITGSGGSTYYADSVKG

SEQ ID NO: 68 - protein, 13 aa, synthetic construct
  REGION 1..13: note = Synthetic Construct
DPGTTVIMSWFDP

SEQ ID NO: 69 - protein, 12 aa, synthetic construct
  REGION 1..12: note = Synthetic Construct
RASQSVRGRYLA

SEQ ID NO: 70 - protein, 7 aa, synthetic construct
  REGION 1..7: note = Synthetic Construct
GASSRAT

SEQ ID NO: 71 - protein, 9 aa, synthetic construct
  REGION 1..9: note = Synthetic Construct
QQYGSSPRT

SEQ ID NO: 72 - protein, 122 aa, synthetic construct
  REGION 1..122: note = Synthetic Construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSS

SEQ ID NO: 73 - protein, 108 aa, synthetic construct
  REGION 1..108: note = Synthetic Construct
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIK

SEQ ID NO: 74 - protein, 5 aa, synthetic construct
  REGION 1..5: note = Synthetic Construct
GGGGS

SEQ ID NO: 75 - protein, 10 aa, synthetic construct
  REGION 1..10: note = Synthetic Construct
GGGGSGGGGS

SEQ ID NO: 76 - protein, 15 aa, synthetic construct
  REGION 1..15: note = Synthetic Construct
GGGGSGGGGSGGGGS

SEQ ID NO: 77 - protein, 20 aa, synthetic construct
  REGION 1..20: note = Synthetic Construct
GGGGSGGGGSGGGGSGGGGS

SEQ ID NO: 78 - protein, 4 aa, synthetic construct
  VARIANT 2..4: note = Can be absent
  VARIANT 1: note = Can be present in repeats of any integer
  REGION 1..4: note = Synthetic Construct
GGGG

SEQ ID NO: 79 - protein, 4 aa, synthetic construct
  VARIANT 3..4: note = Can be absent, or present in repeats of any integer up to 7
  REGION 1..4: note = Synthetic Construct
GSGS

SEQ ID NO: 80 - protein, 5 aa, synthetic construct
  REGION 1..5: note = Synthetic Construct
  VARIANT 1..5: note = Can be present in repeats of any integer up to 8
GSGGS

SEQ ID NO: 81 - protein, 5 aa, synthetic construct
  REGION 1..5: note = Synthetic Construct
  VARIANT 1..5: note = Can be present in repeats of any integer up to 8
GGGGS

SEQ ID NO: 82 - protein, 4 aa, synthetic construct
  REGION 1..4: note = Synthetic Construct
  VARIANT 1..4: note = Can be present in repeats of any integer up to 8
GGGS

SEQ ID NO: 83 - protein, 30 aa, synthetic construct
  REGION 1..30: note = Synthetic Construct
GGGGSGGGGSGGGGSGGGGSGGGGSGGGGS

SEQ ID NO: 84 - protein, 15 aa, synthetic construct
  REGION 1..15: note = Synthetic Construct
  VARIANT 1..15: note = Can be present in repeats of any integer up to 3
GSTSGSGKPGSGEGS

SEQ ID NO: 85 - protein, 16 aa, synthetic construct
  REGION 1..16: note = Synthetic Construct
RSSQSLLYSDGRTYLN

SEQ ID NO: 86 - protein, 16 aa, synthetic construct
  REGION 1..16: note = Synthetic Construct
RSSQSLLYSDSRTYLN

SEQ ID NO: 87 - protein, 115 aa, synthetic construct
  REGION 1..115: note = Synthetic Construct
EVQLVQSGAEVKKPGASVKVSCKASGYTFTDYEIHWVRQAPGKGLEWMGAIDPETGGTAYNQKFKGRVTMTTDTSTSTAYMELRSLRSDDTAVYYCYSYDYVTYWGQGTTVTVSS

SEQ ID NO: 88 - protein, 112 aa, synthetic construct
  REGION 1..112: note = Synthetic Construct
DVVMTQSPLSLSVSPGERASLSCKSSQSLLYSDGRTYLNWYLQKPGQSPQRLIYLVSKLDSGVPDRFSGSGSGTDFTLKISRVQSEDVGVYYCWQGTHLPHTFGGGTKVEIK

SEQ ID NO: 89 - protein, 112 aa, synthetic construct
  REGION 1..112: note = Synthetic Construct
DIQMTQSPSSLSASVGDRVTITCRSSQSLLYSDGRTYLNWYQQKPGKSPKRLIYLVSKLDSGVPDRFSGSGSGTDFTLTISSLQPEDFATYYCWQGTHLPHTFGGGTKVEIK

SEQ ID NO: 90 - protein, 112 aa, synthetic construct
  REGION 1..112: note = Synthetic Construct
DIQMTQSPSSLSASVGDRVTITCRSSQSLLYSDSRTYLNWLQQKPGKSPKRLIYLVSKLDSGVPDRFSGSGSGTDFTLTISSLQPEDFATYYCWQGTHLPHTFGGGTKVEIK

SEQ ID NO: 91 - protein, 708 aa, synthetic construct
  REGION 1..708: note = Synthetic Construct
EVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGKGGGGSGGGGSGGGGSQAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSAGGGGSGGGGSGGGGSGGGGSDVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELK

SEQ ID NO: 92 - protein, 708 aa, synthetic construct
  REGION 1..708: note = Synthetic Construct
EVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGKGGGGSGGGGSGGGGSDVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELKGGGGSGGGGSGGGGSGGGGSQAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSA

SEQ ID NO: 93 - protein, 214 aa, synthetic construct
  REGION 1..214: note = Synthetic Construct
DIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 94 - protein, 701 aa, synthetic construct
  REGION 1..701: note = Synthetic Construct
QAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSAAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGKGGGGSGGGGSGGGGSEVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSGGGGSGGGGSGGGGSGGGGSDIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELK

SEQ ID NO: 95 - protein, 701 aa, synthetic construct
  REGION 1..701: note = Synthetic Construct
QAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSAAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGKGGGGSGGGGSGGGGSDIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKGGGGSGGGGSGGGGSGGGGSEVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSS

SEQ ID NO: 96 - protein, 219 aa, synthetic construct
  REGION 1..219: note = Synthetic Construct
DVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 97 - protein, 708 aa, synthetic construct
  REGION 1..708: note = Synthetic Construct
QAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSAGGGGSGGGGSGGGGSGGGGSDVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELKGGGGSGGGGSGGGGSEVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 98 - protein, 708 aa, synthetic construct
  REGION 1..708: note = Synthetic Construct
DVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELKGGGGSGGGGSGGGGSGGGGSQAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSAGGGGSGGGGSGGGGSEVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 99 - protein, 214 aa, synthetic construct; identical to SEQ ID NO: 93
  REGION 1..214: note = Synthetic Construct

SEQ ID NO: 100 - protein, 701 aa, synthetic construct
  REGION 1..701: note = Synthetic Construct
EVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSGGGGSGGGGSGGGGSGGGGSDIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKGGGGSGGGGSGGGGSQAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSAAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 101 - protein, 701 aa, synthetic construct
  REGION 1..701: note = Synthetic Construct
DIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKGGGGSGGGGSGGGGSGGGGSEVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSGGGGSGGGGSGGGGSQAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSAAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 102 - protein, 219 aa, synthetic construct; identical to SEQ ID NO: 96
  REGION 1..219: note = Synthetic Construct

SEQ ID NO: 103 - protein, 446 aa, synthetic construct
  REGION 1..446: note = Synthetic Construct
EVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 104 - protein, 476 aa, synthetic construct
  REGION 1..476: note = Synthetic Construct
QAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSAGGGGSGGGGSGGGGSGGGGSDVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELKGGGGSGGGGSGGGGSDIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 105 - protein, 476 aa, synthetic construct
  REGION 1..476: note = Synthetic Construct
DVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELKGGGGSGGGGSGGGGSGGGGSQAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSAGGGGSGGGGSGGGGSDIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 106 - protein, 439 aa, synthetic construct
  REGION 1..439: note = Synthetic Construct
QAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSAAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 107 - protein, 481 aa, synthetic construct
  REGION 1..481: note = Synthetic Construct
EVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSGGGGSGGGGSGGGGSGGGGSDIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKGGGGSGGGGSGGGGSDVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 108 - protein, 481 aa, synthetic construct
  REGION 1..481: note = Synthetic Construct
DIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKGGGGSGGGGSGGGGSGGGGSEVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSGGGGSGGGGSGGGGSDVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 109 - protein, 446 aa, synthetic construct; identical to SEQ ID NO: 103
  REGION 1..446: note = Synthetic Construct

SEQ ID NO: 110 - protein, 476 aa, synthetic construct
  REGION 1..476: note = Synthetic Construct
DIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNECGGGGSGGGGSGGGGSQAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSAGGGGSGGGGSGGGGSGGGGSDVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELK

SEQ ID NO: 111 - protein, 476 aa, synthetic construct
  REGION 1..476: note = Synthetic Construct
DIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNECGGGGSGGGGSGGGGSDVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELKGGGGSGGGGSGGGGSGGGGSQAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSA

SEQ ID NO: 112 - protein, 439 aa, synthetic construct; identical to SEQ ID NO: 106
  REGION 1..439: note = Synthetic Construct

SEQ ID NO: 113 - protein, 481 aa, synthetic construct
  REGION 1..481: note = Synthetic Construct
DVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSEVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSGGGGSGGGGSGGGGSGGGGSDIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELK

SEQ ID NO: 114 - protein, 481 aa, synthetic construct
  REGION 1..481: note = Synthetic Construct
DVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSDIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKGGGGSGGGGSGGGGSGGGGSEVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSA

SEQ ID NO: 115 - protein, 709 aa, synthetic construct
  REGION 1..709: note = Synthetic Construct
EVQLVESGGGVVQPGRSLRLSCAASGFTFSDYAMAWVRQAPGKGLEWVATIIYDGSSTYYRDSVKGRFTISRDNSKNTLYLQMNSLRAEDTAVYYCATGLGIATDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSDIQMTQSPSSLSASVGDRVTITCRSSQSLLYSDGRTYLNWYQQKPGKSPKRLIYLVSKLDSGVPDRFSGSGSGTDFTLTISSLQPEDFATYYCWQGTHLPHTFGCGTKVEIKGGGGSGGGGSGGGGSGGGGSEVQLVQSGAEVKKPGASVKVSCKASGYTFTDYEIHWVRQAPGKCLEWMGAIDPETGGTAYNQKFKGRVTMTTDTSTSTAYMELRSLRSDDTAVYYCYSYDYVTYWGQGTTVTVSA

SEQ ID NO: 116 - protein, 709 aa, synthetic construct
  REGION 1..709: note = Synthetic Construct
EVQLVESGGGVVQPGRSLRLSCAASGFTFSDYAMAWVRQAPGKGLEWVATIIYDGSSTYYRDSVKGRFTISRDNSKNTLYLQMNSLRAEDTAVYYCATGLGIATDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEVQLVQSGAEVKKPGASVKVSCKASGYTFTDYEIHWVRQAPGKCLEWMGAIDPETGGTAYNQKFKGRVTMTTDTSTSTAYMELRSLRSDDTAVYYCYSYDYVTYWGQGTTVTVSSGGGGSGGGGSGGGGSGGGGSDIQMTQSPSSLSASVGDRVTITCRSSQSLLYSDGRTYLNWYQQKPGKSPKRLIYLVSKLDSGVPDRFSGSGSGTDFTLTISSLQPEDFATYYCWQGTHLPHTFGCGTKVEIK

SEQ ID NO: 117 - protein, 709 aa, synthetic construct
  REGION 1..709: note = Synthetic Construct
EVQLVESGGGVVQPGRSLRLSCAASGFTFSDYAMAWVRQAPGKGLEWVATIIYDGSSTYYRDSVKGRFTISRDNSKNTLYLQMNSLRAEDTAVYYCATGLGIATDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSDVVMTQSPLSLSVSPGERASLSCKSSQSLLYSDGRTYLNWYLQKPGQSPQRLIYLVSKLDSGVPDRFSGSGSGTDFTLKISRVQSEDVGVYYCWQGTHLPHTFGCGTKVEIKGGGGSGGGGSGGGGSGGGGSEVQLVQSGAEVKKPGASVKVSCKASGYTFTDYEIHWVRQAPGKCLEWMGAIDPETGGTAYNQKFKGRVTMTTDTSTSTAYMELRSLRSDDTAVYYCYSYDYVTYWGQGTTVTVSA

SEQ ID NO: 118 - protein, 709 aa, synthetic construct
  REGION 1..709: note = Synthetic Construct
EVQLVESGGGVVQPGRSLRLSCAASGFTFSDYAMAWVRQAPGKGLEWVATIIYDGSSTYYRDSVKGRFTISRDNSKNTLYLQMNSLRAEDTAVYYCATGLGIATDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEVQLVQSGAEVKKPGASVKVSCKASGYTFTDYEIHWVRQAPGKCLEWMGAIDPETGGTAYNQKFKGRVTMTTDTSTSTAYMELRSLRSDDTAVYYCYSYDYVTYWGQGTTVTVSSGGGGSGGGGSGGGGSGGGGSDVVMTQSPLSLSVSPGERASLSCKSSQSLLYSDGRTYLNWYLQKPGQSPQRLIYLVSKLDSGVPDRFSGSGSGTDFTLKISRVQSEDVGVYYCWQGTHLPHTFGCGTKVEIK

SEQ ID NO: 119 - protein, 214 aa, synthetic construct
  REGION 1..214: note = Synthetic Construct
DIRMTQSPFSLSASVGDRVTITCLASEDIYSDLAWYQQKPAKAPKLFIYNANSLQNGVPSRFSGSGSGTDYTLTISSLQPEDFATYYCQQYNNYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 704 aa, synthetic construct
  REGION 1..704: note = Synthetic Construct
EVQLVQSGAEVKKPGASVKVSCKASGYTFTDYEIHWVRQAPGKGLEWMGAIDPETGGTAYNQKFKGRVTMTTDTSTSTAYMELRSLRSDDTAVYYCYSYDYVTYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEVQLVESGGGVVQPGRSLRLSCAASGFTFSDYAMAWVRQAPGKCLEWVATIIYDGSSTYYRDSVKGRFTISRDNSKNTLYLQMNSLRAEDTAVYYCATGLGIATDYFDYWGQGTLVTVSSGGGGSGGGGSGGGGSGGGGSDIRMTQSPFSLSASVGDRVTITCLASEDIYSDLAWYQQKPAKAPKLFIYNANSLQNGVPSRFSGSGSGTDYTLTISSLQPEDFATYYCQQYNNYPPTFGCGTKVEIK

SEQ ID NO: 121 - protein, 704 aa, synthetic construct
  REGION 1..704: note = Synthetic Construct
EVQLVQSGAEVKKPGASVKVSCKASGYTFTDYEIHWVRQAPGKGLEWMGAIDPETGGTAYNQKFKGRVTMTTDTSTSTAYMELRSLRSDDTAVYYCYSYDYVTYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSDIRMTQSPFSLSASVGDRVTITCLASEDIYSDLAWYQQKPAKAPKLFIYNANSLQNGVPSRFSGSGSGTDYTLTISSLQPEDFATYYCQQYNNYPPTFGCGTKVEIKGGGGSGGGGSGGGGSGGGGSEVQLVESGGGVVQPGRSLRLSCAASGFTFSDYAMAWVRQAPGKCLEWVATIIYDGSSTYYRDSVKGRFTISRDNSKNTLYLQMNSLRAEDTAVYYCATGLGIATDYFDYWGQGTLVTVSA

SEQ ID NO: 122 - protein, 219 aa, synthetic construct
  REGION 1..219: note = Synthetic Construct
DIQMTQSPSSLSASVGDRVTITCRSSQSLLYSDGRTYLNWYQQKPGKSPKRLIYLVSKLDSGVPDRFSGSGSGTDFTLTISSLQPEDFATYYCWQGTHLPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 704 aa, synthetic construct; identical to SEQ ID NO: 120
  REGION 1..704: note = Synthetic Construct

SEQ ID NO: 124 - protein, 704 aa, synthetic construct; identical to SEQ ID NO: 121
  REGION 1..704: note = Synthetic Construct

SEQ ID NO: 125 - protein, 219 aa, synthetic construct
  REGION 1..219: note = Synthetic Construct
DVVMTQSPLSLSVSPGERASLSCKSSQSLLYSDGRTYLNWYLQKPGQSPQRLIYLVSKLDSGVPDRFSGSGSGTDFTLKISRVQSEDVGVYYCWQGTHLPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 710 aa, synthetic construct
  REGION 1..710: note = Synthetic Construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSDIQMTQSPSSLSASVGDRVTITCRSSQSLLYSDGRTYLNWYQQKPGKSPKRLIYLVSKLDSGVPDRFSGSGSGTDFTLTISSLQPEDFATYYCWQGTHLPHTFGCGTKVEIKGGGGSGGGGSGGGGSGGGGSEVQLVQSGAEVKKPGASVKVSCKASGYTFTDYEIHWVRQAPGKCLEWMGAIDPETGGTAYNQKFKGRVTMTTDTSTSTAYMELRSLRSDDTAVYYCYSYDYVTYWGQGTTVTVSA

SEQ ID NO: 127 - protein, 710 aa, synthetic construct
  REGION 1..710: note = Synthetic Construct
DIQMTQSPSSLSASVGDRVTITCRSSQSLLYSDGRTYLNWYQQKPGKSPKRLIYLVSKLDSGVPDRFSGSGSGTDFTLTISSLQPEDFATYYCWQGTHLPHTFGCGTKVEIKGGGGSGGGGSGGGGSGGGGSEVQLVQSGAEVKKPGASVKVSCKASGYTFTDYEIHWVRQAPGKCLEWMGAIDPETGGTAYNQKFKGRVTMTTDTSTSTAYMELRSLRSDDTAVYYCYSYDYVTYWGQGTTVTVSSGGGGSGGGGSGGGGSEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 128 - protein, 710 aa, synthetic construct
  REGION 1..710: note = Synthetic Construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSDVVMTQSPLSLSVSPGERASLSCKSSQSLLYSDGRTYLNWYLQKPGQSPQRLIYLVSKLDSGVPDRFSGSGSGTDFTLKISRVQSEDVGVYYCWQGTHLPHTFGCGTKVEIKGGGGSGGGGSGGGGSGGGGSEVQLVQSGAEVKKPGASVKVSCKASGYTFTDYEIHWVRQAPGKCLEWMGAIDPETGGTAYNQKFKGRVTMTTDTSTSTAYMELRSLRSDDTAVYYCYSYDYVTYWGQGTTVTVSA

SEQ ID NO: 129 - protein, 215 aa, synthetic construct
  REGION 1..215: note = Synthetic Construct
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 477 aa, synthetic construct
  REGION 1..477: note = Synthetic Construct
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDIQMTQSPSSLSASVGDRVTITCRSSQSLLYSDGRTYLNWYQQKPGKSPKRLIYLVSKLDSGVPDRFSGSGSGTDFTLTISSLQPEDFATYYCWQGTHLPHTFGCGTKVEIKGGGGSGGGGSGGGGSGGGGSEVQLVQSGAEVKKPGASVKVSCKASGYTFTDYEIHWVRQAPGKCLEWMGAIDPETGGTAYNQKFKGRVTMTTDTSTSTAYMELRSLRSDDTAVYYCYSYDYVTYWGQGTTVTVSA

SEQ ID NO: 131 - protein, 477 aa, synthetic construct
  REGION 1..477: note = Synthetic Construct
DIQMTQSPSSLSASVGDRVTITCRSSQSLLYSDGRTYLNWYQQKPGKSPKRLIYLVSKLDSGVPDRFSGSGSGTDFTLTISSLQPEDFATYYCWQGTHLPHTFGCGTKVEIKGGGGSGGGGSGGGGSGGGGSEVQLVQSGAEVKKPGASVKVSCKASGYTFTDYEIHWVRQAPGKCLEWMGAIDPETGGTAYNQKFKGRVTMTTDTSTSTAYMELRSLRSDDTAVYYCYSYDYVTYWGQGTTVTVSSGGGGSGGGGSGGGGSEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 477 aa, synthetic construct
  REGION 1..477: note = Synthetic Construct
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDVVMTQSPLSLSVSPGERASLSCKSSQSLLYSDGRTYLNWYLQKPGQSPQRLIYLVSKLDSGVPDRFSGSGSGTDFTLKISRVQSEDVGVYYCWQGTHLPHTFGCGTKVEIKGGGGSGGGGSGGGGSGGGGSEVQLVQSGAEVKKPGASVKVSCKASGYTFTDYEIHWVRQAPGKCLEWMGAIDPETGGTAYNQKFKGRVTMTTDTSTSTAYMELRSLRSDDTAVYYCYSYDYVTYWGQGTTVTVSA

SEQ ID NO: 133 - protein, 448 aa, synthetic construct
  REGION 1..448: note = Synthetic Construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 134 - protein, 442 aa, synthetic construct
  REGION 1..442: note = Synthetic Construct
QAQLQQSGAELVRPGASVTLSCKASGYTFTDYEIHWMKQTPVHGLEWIGAIDPETGGTAYNQKFKGKAILTADRSSSTAYLELRSLTSEDSAVYYCYSYDYVTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 135 - protein, 219 aa, synthetic construct
  REGION 1..219: note = Synthetic Construct
DVVMTQTPLTWSITIGQPASISCKSSQSLLYSDGRTYLNWLLQRPGQSPKRLIYLVSKLDSGVPDRFSGSGSGTDFTLKISRVEAEDLGFYYCWQGTHLPHTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 447 aa, synthetic construct
  REGION 1..447: note = Synthetic Construct
EVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSASTKGPSVCPLAPSSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 137 - protein, 214 aa, synthetic construct
  REGION 1..214: note = Synthetic Construct
DIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRTVAAPSVFIFPPCDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 138 - protein, 447 aa, synthetic construct
  REGION 1..447: note = Synthetic Construct
EVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSASTKGPSVFPCAPSSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 139 - protein, 214 aa, synthetic construct
  REGION 1..214: note = Synthetic Construct
DIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRTVAAPSVFICPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 140 - protein, 447 aa, synthetic construct
  REGION 1..447: note = Synthetic Construct
EVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSASTKGPSVFPLAPSSRSTSESTAALGCLVKDYFPEPVTVSWNSGALTSGVHTCPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 141 - protein, 214 aa, synthetic construct
  REGION 1..214: note = Synthetic Construct
DIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 142 - protein, 447 aa, synthetic construct
  REGION 1..447: note = Synthetic Construct
EVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSASTKGPSVFPLAPSSRSTSESTAALGCLVKDYFPEPVTVSWNSGALTSGVHTFPACLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 143 - protein, 214 aa, synthetic construct
  REGION 1..214: note = Synthetic Construct
DIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRTVAAPSVFIFPPSDEQLKSGTASVVCLLNNFYPREAKVQWKVDNALQSGNSCESVTEQDSKDSTYSLSSTLTLSKADYEKHKVYACEVTHQGLSSPVTKSFNRGES

SEQ ID NO: 144 - protein, 447 aa, synthetic construct; identical to SEQ ID NO: 142
  REGION 1..447: note = Synthetic Construct

SEQ ID NO: 145 - protein, 214 aa, synthetic construct; identical to SEQ ID NO: 143
  REGION 1..214: note = Synthetic Construct

SEQ ID NO: 146 - protein, 442 aa, synthetic construct
  REGION 1..442: note = Synthetic Construct
EVQLVQSGAEVKKPGASVKVSCKASGYTFTDYEIHWVRQAPGKGLEWMGAIDPETGGTAYNQKFKGRVTMTTDTSTSTAYMELRSLRSDDTAVYYCYSYDYVTYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 147 - protein, 219 aa, synthetic construct; identical to SEQ ID NO: 122
  REGION 1..219: note = Synthetic Construct

SEQ ID NO: 148 - protein, 447 aa, synthetic construct
  REGION 1..447: note = Synthetic Construct
EVQLVESGGGVVQPGRSLRLSCAASGFTFSDYAMAWVRQAPGKGLEWVATIIYDGSSTYYRDSVKGRFTISRDNSKNTLYLQMNSLRAEDTAVYYCATGLGIATDYFDYWGQGTLVTVSSASTKGPSVFPCAPSSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 149 - protein, 214 aa, synthetic construct
  REGION 1..214: note = Synthetic Construct
DIRMTQSPFSLSASVGDRVTITCLASEDIYSDLAWYQQKPAKAPKLFIYNANSLQNGVPSRFSGSGSGTDYTLTISSLQPEDFATYYCQQYNNYPPTFGGGTKVEIKRTVAAPSVFICPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 150 - protein, 447 aa, synthetic construct
  REGION 1..447: note = Synthetic Construct
EVQLVESGGGVVQPGRSLRLSCAASGFTFSDYAMAWVRQAPGKGLEWVATIIYDGSSTYYRDSVKGRFTISRDNSKNTLYLQMNSLRAEDTAVYYCATGLGIATDYFDYWGQGTLVTVSSASTKGPSVFPLAPSSRSTSESTAALGCLVKDYFPEPVTVSWNSGALTSGVHTCPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 151 - protein, 214 aa, synthetic construct
  REGION 1..214: note = Synthetic Construct
DIRMTQSPFSLSASVGDRVTITCLASEDIYSDLAWYQQKPAKAPKLFIYNANSLQNGVPSRFSGSGSGTDYTLTISSLQPEDFATYYCQQYNNYPPTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 152 - protein, 447 aa, synthetic construct
  REGION 1..447: note = Synthetic Construct
EVQLVESGGGVVQPGRSLRLSCAASGFTFSDYAMAWVRQAPGKGLEWVATIIYDGSSTYYRDSVKGRFTISRDNSKNTLYLQMNSLRAEDTAVYYCATGLGIATDYFDYWGQGTLVTVSSASTKGPSVFPLAPSSRSTSESTAALGCLVKDYFPEPVTVSWNSGALTSGVHTFPACLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 153 - protein, 214 aa, synthetic construct
  REGION 1..214: note = Synthetic Construct
DIRMTQSPFSLSASVGDRVTITCLASEDIYSDLAWYQQKPAKAPKLFIYNANSLQNGVPSRFSGSGSGTDYTLTISSLQPEDFATYYCQQYNNYPPTFGGGTKVEIKRTVAAPSVFIFPPSDEQLKSGTASVVCLLNNFYPREAKVQWKVDNALQSGNSCESVTEQDSKDSTYSLSSTLTLSKADYEKHKVYACEVTHQGLSSPVTKSFNRGES

SEQ ID NO: 154 - protein, 447 aa, synthetic construct
  REGION 1..447: note = Synthetic Construct
EVQLVESGGGVVQPGRSLRLSCAASGFTFSDYAMAWVRQAPGKGLEWVATIIYDGSSTYYRDSVKGRFTISRDNSKNTLYLQMNSLRAEDTAVYYCATGLGIATDYFDYWGQGTLVTVSSASTKGPSVCPLAPSSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 155 - protein, 214 aa, synthetic construct; identical to SEQ ID NO: 149
  REGION 1..214: note = Synthetic Construct

SEQ ID NO: 156 - protein, 449 aa, synthetic construct
  REGION 1..449: note = Synthetic Construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPCAPSSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 157 - protein, 215 aa, synthetic construct
  REGION 1..215: note = Synthetic Construct
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFICPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 158 - protein, 449 aa, synthetic construct
  REGION 1..449: note = Synthetic Construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPSSRSTSESTAALGCLVKDYFPEPVTVSWNSGALTSGVHTCPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 159 - protein, 215 aa, synthetic construct
  REGION 1..215: note = Synthetic Construct
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 160 - protein, 449 aa, synthetic construct
  REGION 1..449: note = Synthetic Construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVCPLAPSSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 161 - protein, 215 aa, synthetic construct; identical to SEQ ID NO: 157
  REGION 1..215: note = Synthetic Construct

SEQ ID NO: 162 - protein, 449 aa, synthetic construct
  REGION 1..449: note = Synthetic Construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPSSRSTSESTAALGCLVKDYFPEPVTVSWNSGALTSGVHTFPACLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 163 - protein, 215 aa, synthetic construct
  REGION 1..215: note = Synthetic Construct
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSCESVTEQDSKDSTYSLSSTLTLSKADYEKHKVYACEVTHQGLSSPVTKSFNRGES

SEQ ID NO: 164 - protein, 120 aa, synthetic construct
  REGION 1..120: note = Synthetic Construct
EVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSS

SEQ ID NO: 165 - protein, 107 aa, synthetic construct
  REGION 1..107: note = Synthetic Construct
DIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELK

SEQ ID NO: 166 - protein, 120 aa, synthetic construct
  REGION 1..120: note = Synthetic Construct
EVQLVESGGGVVQPGRSLRLSCAASGFTFSDYAMAWVRQAPGKCLEWVATIIYDGSSTYYRDSVKGRFTISRDNSKNTLYLQMNSLRAEDTAVYYCATGLGIATDYFDYWGQGTLVTVSS

SEQ ID NO: 167 - protein, 107 aa, synthetic construct
  REGION 1..107: note = Synthetic Construct
DIRMTQSPFSLSASVGDRVTITCLASEDIYSDLAWYQQKPAKAPKLFIYNANSLQNGVPSRFSGSGSGTDYTLTISSLQPEDFATYYCQQYNNYPPTFGCGTKVEIK

SEQ ID NO: 168 - protein, 115 aa, synthetic construct
  REGION 1..115: note = Synthetic Construct
EVQLVQSGAEVKKPGASVKVSCKASGYTFTDYEIHWVRQAPGKCLEWMGAIDPETGGTAYNQKFKGRVTMTTDTSTSTAYMELRSLRSDDTAVYYCYSYDYVTYWGQGTTVTVSS

SEQ ID NO: 169 - protein, 112 aa, synthetic construct
  REGION 1..112: note = Synthetic Construct
DVVMTQSPLSLSVSPGERASLSCKSSQSLLYSDGRTYLNWYLQKPGQSPQRLIYLVSKLDSGVPDRFSGSGSGTDFTLKISRVQSEDVGVYYCWQGTHLPHTFGCGTKVEIK

SEQ ID NO: 170 - protein, 112 aa, synthetic construct
  REGION 1..112: note = Synthetic Construct
DIQMTQSPSSLSASVGDRVTITCRSSQSLLYSDGRTYLNWYQQKPGKSPKRLIYLVSKLDSGVPDRFSGSGSGTDFTLTISSLQPEDFATYYCWQGTHLPHTFGCGTKVEIK

SEQ ID NO: 171 - protein, 441 aa, synthetic construct
  REGION 1..441: note = Synthetic Construct
EVQLVQSGAEVKKPGASVKVSCKASGYTFTDYEIHWVRQAPGKGLEWMGAIDPETGGTAYNQKFKGRVTMTTDTSTSTAYMELRSLRSDDTAVYYCYSYDYVTYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPCREEMTKNQVSLWCLVKGFYPSDIAVEWESNGQPENNYKTTPPMLDSDGSFFLYSKLTVDKSRWQQGNVFSCSVMHEALHNHYTQKSLSLSPGK

SEQ ID NO: 172 - protein, 448 aa, synthetic construct
  REGION 1..448: note = Synthetic Construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPSSRSTSESTAALGCLVKDYFPEPVTVSWNSGALTSGVHTFPACLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNRFTQKSLSLSPGK

SEQ ID NO: 173 - protein, 445 aa, synthetic construct
  REGION 1..445: note = Synthetic Construct
EVQLVQSGAEVKKPGASVKVSCKASGYTFTDYEIHWVRQAPGKGLEWMGAIDPETGGTAYNQKFKGRVTMTTDTSTSTAYMELRSLRSDDTAVYYCYSYDYVTYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 174 - protein, 452 aa, synthetic construct
  REGION 1..452: note = Synthetic Construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 445 aa, synthetic construct
  REGION 1..445: note = Synthetic Construct
EVQLVQSGAEVKKPGASVKVSCKASGYTFTDYEIHWVRQAPGKGLEWMGAIDPETGGTAYNQKFKGRVTMTTDTSTSTAYMELRSLRSDDTAVYYCYSYDYVTYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHARFTQKSLSLSPGK

SEQ ID NO: 176 - protein, 452 aa, synthetic construct
  REGION 1..452: note = Synthetic Construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHAHYTQKSLSLSPGK

SEQ ID NO: 177 - protein, 707 aa, synthetic construct
  REGION 1..707: note = Synthetic Construct
EVQLVQSGAEVKKPGASVKVSCKASGYTFTDYEIHWVRQAPGKGLEWMGAIDPETGGTAYNQKFKGRVTMTTDTSTSTAYMELRSLRSDDTAVYYCYSYDYVTYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSPGKGGGGSGGGGSGGGGSEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGCGTKVEIKGGGGSGGGGSGGGGSGGGGSEVQLLESGGGLVQPGGSLRLSCAASGFTFSSYAMSWVRQAPGKCLEWVSGITGSGGSTYYADSVKGRFTISRDNSKNTLYLQMNSLRAEDTAVYYCAKDPGTTVIMSWFDPWGQGTLVTVSA

SEQ ID NO: 178 - protein, 707 aa, synthetic construct
  REGION 1..707: note = Synthetic Construct
EVQLVQSGAEVKKPGASVKVSCKASGYTFTDYEIHWVRQAPGKGLEWMGAIDPETGGTAYNQKFKGRVTMTTDTSTSTAYMELRSLRSDDTAVYYCYSYDYVTYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSPGKGGGGSGGGGSGGGGSEVQLLESGGGLVQPGGSLRLSCAASGFTFSSYAMSWVRQAPGKCLEWVSGITGSGGSTYYADSVKGRFTISRDNSKNTLYLQMNSLRAEDTAVYYCAKDPGTTVIMSWFDPWGQGTLVTVSSGGGGSGGGGSGGGGSGGGGSEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGCGTKVEIK

SEQ ID NO: 179 - protein, 706 aa, synthetic construct
  REGION 1..706: note = Synthetic Construct
EVQLVQSGAEVKKPGASVKVSCKASGYTFTDYEIHWVRQAPGKGLEWMGAIDPETGGTAYNQKFKGRVTMTTDTSTSTAYMELRSLRSDDTAVYYCYSYDYVTYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSEVQLLESGGGLVQPGGSLRLSCAASGFTFSSYAMSWVRQAPGKCLEWVSGITGSGGSTYYADSVKGRFTISRDNSKNTLYLQMNSLRAEDTAVYYCAKDPGTTVIMSWFDPWGQGTLVTVSSGGGGSGGGGSGGGGSGGGGSEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGCGTKVEIK

SEQ ID NO: 180 - protein, 710 aa, synthetic construct
  REGION 1..710: note = Synthetic Construct
EVQLVQSGAEVKKPGASVKVSCKASGYTFTDYEIHWVRQAPGKGLEWMGAIDPETGGTAYNQKFKGRVTMTTDTSTSTAYMELRSLRSDDTAVYYCYSYDYVTYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLLESGGGLVQPGGSLRLSCAASGFTFSSYAMSWVRQAPGKCLEWVSGITGSGGSTYYADSVKGRFTISRDNSKNTLYLQMNSLRAEDTAVYYCAKDPGTTVIMSWFDPWGQGTLVTVSSGGGGSGGGGSGGGGSGGGGSEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGCGTKVEIK

SEQ ID NO: 181-182 - skipped (2 entries)

SEQ ID NO: 183 - protein, 190 aa, synthetic construct
  REGION 1..190: note = Synthetic Construct
QGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 184 - protein, 190 aa, synthetic construct
  REGION 1..190: note = Synthetic Construct
QGWQAFKNDATEIIPELGEYPEPPPDLENNKTMNRAENGGRPPHHPFETKDVSEYSCRELHFTRYVTDGQCRSAKPVTELVCSGQCGPARLLPNAIGRGKWWRPSGPDFRCIPDRYRAQRVQLLCPGGAAPRARKVRLVASCKCKRLTRFHNQSELKDFGPEAARPQKGRKPRPRARGAKANQAELENAY

SEQ ID NO: 185 - protein, 24 aa, synthetic construct
  REGION 1..24: note = Synthetic Construct
GPARLLPNAIGRGKWWRPSGPDFR

SEQ ID NO: 186 - protein, 8 aa, synthetic construct
  REGION 1..8: note = Synthetic Construct
IGRGKWWR

SEQ ID NO: 187 - protein, 11 aa, synthetic construct
  REGION 1..11: note = Synthetic Construct
PNAIGRGKWWR

SEQ ID NO: 188 - protein, 12 aa, synthetic construct
  REGION 1..12: note = Synthetic Construct
IGRGKWWRPSGP

SEQ ID NO: 189 - protein, 190 aa, synthetic construct
  REGION 1..190: note = Synthetic Construct
QGWQAFKNDATEIIPELGEYPEPPPELENNKTMNRAENGGRPPHHPFETKDVSEYSCRELHFTRYVTDGQCRSAKPVTELVCSGQCGPARLLPNAIGRGKWWRPSGPDFRCIPDRYRAQRVQLLCPGGAAPRARKVRLVASCKCKRLTRFHNQSELKDFGPEAARPQKGRKPRPRARGAKANQAELENAY

SEQ ID NO: 190 - protein, 188 aa, synthetic construct
  REGION 1..188: note = Synthetic Construct
QGWQAFRNDATEVIPGLGEYPEPPPENNQTMNRAENGGRPPHHPYDAKDVSEYSCRELHYTRFLTDGPCRSAKPVTELVCSGQCGPARLLPNAIGRVKWWRPNGPDFRCIPDRYRAQRVQLLCPGGAAPRSRKVRLVASCKCKRLTRFHNQSELKDFGPETARPQKGRKPRPGARGAKANQAELENAY

SEQ ID NO: 191 - protein, 190 aa, synthetic construct
  REGION 1..190: note = Synthetic Construct
QGWQAFKNDATEIIPGLREYPEPPQELENNQTMNRAENGGRPPHHPYDTKDVSEYSCRELHYTRFVTDGPCRSAKPVTELVCSGQCGPARLLPNAIGRVKWWRPNGPDFRCIPDRYRAQRVQLLCPGGAAPRSRKVRLVASCKCKRLTRFHNQSELKDFGPETARPQKGRKPRPRARGAKANQAELENAY

SEQ ID NO: 192 - protein, 24 aa, synthetic construct
  REGION 1..24: note = Synthetic Construct
GPARLLPNAIGRVKWWRPNGPDFR

SEQ ID NO: 193 - protein, 12 aa, synthetic construct
  REGION 1..12: note = Synthetic Construct
GPARLLPNAIGR

SEQ ID NO: 194 - protein, 11 aa, synthetic construct
  REGION 1..11: note = Synthetic Construct
RLLPNAIGRGK

SEQ ID NO: 195 - protein, 11 aa, synthetic construct
  REGION 1..11: note = Synthetic Construct
KWWRPSGPDFR

SEQ ID NO: 196 - protein, 11 aa, synthetic construct
  REGION 1..11: note = Synthetic Construct
PNAIGRVKWWR

SEQ ID NO: 197 - protein, 12 aa, synthetic construct
  REGION 1..12: note = Synthetic Construct
IGRVKWWRPNGP

SEQ ID NO: 198 - protein, 11 aa, synthetic construct
  REGION 1..11: note = Synthetic Construct
KWWRPNGPDFR

SEQ ID NO: 199 - protein, 5 aa, synthetic construct
  REGION 1..5: note = Synthetic Construct
DFEIH

SEQ ID NO: 200 - protein, 17 aa, synthetic construct
  REGION 1..17: note = Synthetic Construct
AIDPETGGSANNQKFKA